SEQUENCE LISTING (212 sequences)

SEQ ID NO: 1 - protein, 119 aa, Artificial Sequence
  HC VR
QSVEESGGRLVTPGTPLTLTCTVSGFSLSKYAMSWVRQAPGKGLEWIGIISSGGSTYYASWAKGRFTISKTSSTTVDLKITSPTTEDTATYFCARAGYPGYSPHYFNIWGPGTLVTVSL

SEQ ID NO: 2 - protein, 5 aa, Artificial Sequence
  CDR-H1
KYAMS

SEQ ID NO: 3 - protein, 16 aa, Artificial Sequence
  CDR-H2
IISSGGSTYYASWAKG

SEQ ID NO: 4 - protein, 13 aa, Artificial Sequence
  CDR-H3
AGYPGYSPHYFNI

SEQ ID NO: 5 - protein, 107 aa, Artificial Sequence
  LC VR
AIQMTQSPSSLAASVGDTVTITCKASEDIGYELNWYQQKLGIAPKLLIYGANTLESGVPSRFTGSGSETDYTLTISSVQAEDAGIYYCQQGYSTPPTFGAGTKVEIK

SEQ ID NO: 6 - protein, 11 aa, Artificial Sequence
  CDR-L1
KASEDIGYELN

SEQ ID NO: 7 - protein, 7 aa, Artificial Sequence
  CDR-L2
GANTLES

SEQ ID NO: 8 - protein, 9 aa, Artificial Sequence
  CDR-L3
QQGYSTPPT

SEQ ID NO: 9 - protein, 118 aa, Artificial Sequence
  HC VR
QSVEESGGRLVTPGTPLTLTCTVSGFSLSSYAMSWVRQAPGKGLEWIGIISSSGSTYYASWAKGRFTISKTSTTVDLKITSPTTEDTATYFCARAMYPGYSTGVYNIWGPGTLVTVSL

SEQ ID NO: 10 - protein, 5 aa, Artificial Sequence
  CDR-H1
SYAMS

SEQ ID NO: 11 - protein, 16 aa, Artificial Sequence
  CDR-H2
IISSSGSTYYASWAKG

SEQ ID NO: 12 - protein, 13 aa, Artificial Sequence
  CDR-H3
AMYPGYSTGVYNI

SEQ ID NO: 13 - protein, 107 aa, Artificial Sequence
  LC VR
AIQMTQSPSSLAASVGDTVTITCKASEDIGYELNWYQQKLGIAPKLLIYGANTLESGVPSRFSGSGSETDYTLTISSVQAEDAGIYYCQQGYNTPNTFGAGTKVEIK

SEQ ID NO: 14 - protein, 11 aa, Artificial Sequence
  CDR-L1
KASEDIGYELN

SEQ ID NO: 15 - protein, 7 aa, Artificial Sequence
  CDR-L2
GANTLES

SEQ ID NO: 16 - protein, 9 aa, Artificial Sequence
  CDR-L3
QQGYNTPNT

SEQ ID NO: 17 - protein, 120 aa, Artificial Sequence
  HC VR
QSLEESGGRLVTPGTPLTLTCTVSGFSLSSYAMSWVRQAPGKGLEWIGIISSSGSTYYASWAKGRFTISKTSTTVDLKITSPTTEDTATYFCARDASSRGYYYYYGMDPWGPGTLVTVSS

SEQ ID NO: 18 - protein, 5 aa, Artificial Sequence
  CDR-H1
SYAMS

SEQ ID NO: 19 - protein, 16 aa, Artificial Sequence
  CDR-H2
IISSSGSTYYASWAKG

SEQ ID NO: 20 - protein, 15 aa, Artificial Sequence
  CDR-H3
DASSRGYYYYYGMDP

SEQ ID NO: 21 - protein, 107 aa, Artificial Sequence
  LC VR
AIQMTQSPSSLAASVGDTVTITCKASEDIGYGLNWYQQKLGIAPKLLIYGANTLESGVPSRFSGSGSETDYTLTISSVQAEDAGIYYCQQGYSTPLTFGAGTKVEIK

SEQ ID NO: 22 - protein, 11 aa, Artificial Sequence
  CDR-L1
KASEDIGYGLN

SEQ ID NO: 23 - protein, 7 aa, Artificial Sequence
  CDR-L2
GANTLES

SEQ ID NO: 24 - protein, 9 aa, Artificial Sequence
  CDR-L3
QQGYSTPLT

SEQ ID NO: 25 - protein, 120 aa, Artificial Sequence
  HC VR
QSVEESGGRLVTPGTPLTLTCTVSGFSLSSYAMSWVRQAPGKGLEWIGIINSGRSTYYASWAKGRFTISKTSTTVDLKITSPTTEDTATYFCARDATSIGYYRYYGMDPWGPGTLVTVSS

SEQ ID NO: 26 - protein, 5 aa, Artificial Sequence
  CDR-H1
SYAMS

SEQ ID NO: 27 - protein, 16 aa, Artificial Sequence
  CDR-H2
IINSGRSTYYASWAKG

SEQ ID NO: 28 - protein, 15 aa, Artificial Sequence
  CDR-H3
DATSIGYYRYYGMDP

SEQ ID NO: 29 - protein, 107 aa, Artificial Sequence; identical to SEQ ID NO: 21
  LC VR

SEQ ID NO: 30 - protein, 11 aa, Artificial Sequence
  CDR-L1
KASEDIGYGLN

SEQ ID NO: 31 - protein, 7 aa, Artificial Sequence
  CDR-L2
GANTLES

SEQ ID NO: 32 - protein, 9 aa, Artificial Sequence
  CDR-L3
QQGYSTPLT

SEQ ID NO: 33 - protein, 123 aa, Artificial Sequence
  HC VR
QSVEESGGRLVTPGTPLTLTCTVSGFSLSSYAMNWVRQAPGKGLEWIGIISSSGSTYYASWAKGRFTISKTSTTVDLKITSPTTEDTATYFCARVGAGYAGYGYDNYYGMDPWGPGTLVTVSS

SEQ ID NO: 34 - protein, 5 aa, Artificial Sequence
  CDR-H1
SYAMN

SEQ ID NO: 35 - protein, 16 aa, Artificial Sequence
  CDR-H2
IISSSGSTYYASWAKG

SEQ ID NO: 36 - protein, 18 aa, Artificial Sequence
  CDR-H3
VGAGYAGYGYDNYYGMDP

SEQ ID NO: 37 - protein, 107 aa, Artificial Sequence; identical to SEQ ID NO: 21
  LC VR

SEQ ID NO: 38 - protein, 11 aa, Artificial Sequence
  CDR-L1
KASEDIGYGLN

SEQ ID NO: 39 - protein, 7 aa, Artificial Sequence
  CDR-L2
GANTLES

SEQ ID NO: 40 - protein, 9 aa, Artificial Sequence
  CDR-L3
QQGYSTPLT

SEQ ID NO: 41 - protein, 120 aa, Artificial Sequence
  HC VR
QSVEESGGRLVTPGTPLTLTCTVSGFSLSSYAMSWVRQAPGKGLEWIGIISSSGTTYYASWAKGPFTISKTSTTVDLKITSPTTEDTATYFCARGGAGYGYAHYYGMDPWGPGTLVTVSS

SEQ ID NO: 42 - protein, 5 aa, Artificial Sequence
  CDR-H1
SYAMS

SEQ ID NO: 43 - protein, 16 aa, Artificial Sequence
  CDR-H2
IISSSGTTYYASWAKG

SEQ ID NO: 44 - protein, 15 aa, Artificial Sequence
  CDR-H3
GGAGYGYAHYYGMDP

SEQ ID NO: 45 - protein, 107 aa, Artificial Sequence; identical to SEQ ID NO: 21
  LC VR

SEQ ID NO: 46 - protein, 11 aa, Artificial Sequence
  CDR-L1
KASEDIGYGLN

SEQ ID NO: 47 - protein, 7 aa, Artificial Sequence
  CDR-L2
GANTLES

SEQ ID NO: 48 - protein, 9 aa, Artificial Sequence
  CDR-L3
QQGYSTPLT

SEQ ID NO: 49 - protein, 119 aa, Artificial Sequence
  HC VR
QSVEESGGRLVTPGTPLTLTCTVSGFSLNNYAMNWVRQAPGKGLEWIGIISSSGNTYYANWAKGRFTISKTSTTVDLKITSPTTEDTATYFCARDSYPGYSPYYGMDPWGPGTLVTVSS

SEQ ID NO: 50 - protein, 5 aa, Artificial Sequence
  CDR-H1
NYAMN

SEQ ID NO: 51 - protein, 16 aa, Artificial Sequence
  CDR-H2
IISSSGNTYYANWAKG

SEQ ID NO: 52 - protein, 14 aa, Artificial Sequence
  CDR-H3
DSYPGYSPYYGMDP

SEQ ID NO: 53 - protein, 107 aa, Artificial Sequence
  LC VR
AIQMTQSPSSLAASVGDTVTITCKASEDIAYGLNWYQQKLGITPKLLIYGANTLESGVPSRFSGSGSETHYTLTISSVQAEDAGIYYCQQGYNTPPTFGAGTKVEIK

SEQ ID NO: 54 - protein, 11 aa, Artificial Sequence
  CDR-L1
KASEDIAYGLN

SEQ ID NO: 55 - protein, 7 aa, Artificial Sequence
  CDR-L2
GANTLES

SEQ ID NO: 56 - protein, 9 aa, Artificial Sequence
  CDR-L3
QQGYNTPPT

SEQ ID NO: 57 - protein, 132 aa, Artificial Sequence
  HC VR
QEELKETGGGLVQPGGSLTLSCKASGFDFSSYYMSWVRQAPGKGLEWIGVIYASKGSTDYASWVNGRFTISSDNAQNTVDLQMNSLTAADTATYYCARSKRFYTYGYAGYAYAHYYYGMDLWGPGTLVTVSS

SEQ ID NO: 58 - protein, 5 aa, Artificial Sequence
  CDR-H1
SYYMS

SEQ ID NO: 59 - protein, 17 aa, Artificial Sequence
  CDR-H2
VIYASKGSTDYASWVNG

SEQ ID NO: 60 - protein, 23 aa, Artificial Sequence
  CDR-H3
SKRFYTYGYAGYAYAHYYYGMDL

SEQ ID NO: 61 - protein, 107 aa, Artificial Sequence
  LC VR
AIQMTQSPSSLAASVGDTVTITCKASEDIGYGLNWYQQKLGIAPKLLIYGADTLESGVPSRFSGSGSETDYTLTISSVQAEDAGIYYCQQGYSTPLTFGAGTKVEIK

SEQ ID NO: 62 - protein, 11 aa, Artificial Sequence
  CDR-L1
KASEDIGYGLN

SEQ ID NO: 63 - protein, 7 aa, Artificial Sequence
  CDR-L2
GADTLES

SEQ ID NO: 64 - protein, 9 aa, Artificial Sequence
  CDR-L3
QQGYSTPLT

SEQ ID NO: 65 - protein, 123 aa, Artificial Sequence
  HC VR
QELVESGGGLVQAGESLKLSCKASGIDFSSYGISWVRQAPGKGLEWIAYIYPGFGITNYANSVKGRFTISSDNAQNTVFLQMTSLTASDTATYFCTRAYASSSGYYIHYFNLWGPGTLVTVSS

SEQ ID NO: 66 - protein, 5 aa, Artificial Sequence
  CDR-H1
SYGIS

SEQ ID NO: 67 - protein, 17 aa, Artificial Sequence
  CDR-H2
YIYPGFGITNYANSVKG

SEQ ID NO: 68 - protein, 15 aa, Artificial Sequence
  CDR-H3
AYASSSGYYIHYFNL

SEQ ID NO: 69 - protein, 107 aa, Artificial Sequence
  LC VR
AIQMTQSPSSLAASVGDTVTITCKASEDIGYGLNWYQQKLGIAPKLLIYGANTLESGVPSRFSGSGSETDYTLTISSVQAEDAGIYYCQQAYSTPLTFGAGTKVEIK

SEQ ID NO: 70 - protein, 11 aa, Artificial Sequence
  CDR-L1
KASEDIGYGLN

SEQ ID NO: 71 - protein, 7 aa, Artificial Sequence
  CDR-L2
GANTLES

SEQ ID NO: 72 - protein, 9 aa, Artificial Sequence
  CDR-L3
QQAYSTPLT

SEQ ID NO: 73 - protein, 117 aa, Artificial Sequence
  HC VR
QSLEESGGDLVKPGASLTLTCTASGFSFSSSYWICWVRQAPGKGLEWIGCIYTGSGSTWYASWAKGRFTISKTSSTTVTLQMTSLTAADTATYFCARGYGSYMYDFWGPGTLVTVSS

SEQ ID NO: 74 - protein, 6 aa, Artificial Sequence
  CDR-H1
SSYWIC

SEQ ID NO: 75 - protein, 17 aa, Artificial Sequence
  CDR-H2
CIYTGSGSTWYASWAKG

SEQ ID NO: 76 - protein, 9 aa, Artificial Sequence
  CDR-H3
GYGSYMYDF

SEQ ID NO: 77 - protein, 107 aa, Artificial Sequence
  LC VR
DVVMTQTPASVEAAVGGTVTIKCQASQSISTALAWYQQKPGQPPKLLIYSASTLESGVPSRFKGSGSGTEFTLAISDLESADAATYYCQGADGSSSSFGGGTEVVVK

SEQ ID NO: 78 - protein, 11 aa, Artificial Sequence
  CDR-L1
QASQSISTALA

SEQ ID NO: 79 - protein, 7 aa, Artificial Sequence
  CDR-L2
SASTLES

SEQ ID NO: 80 - protein, 9 aa, Artificial Sequence
  CDR-L3
QGADGSSSS

SEQ ID NO: 81 - protein, 121 aa, Artificial Sequence
  HC VR
QEQLEESGGDLVKPEGSLTLTCTASGFSFSSNYWICWVRQAPGKGLEWIGCIYIGSGDTYYASWAKGRFTISKTSSTTVTLQMTSLTAADTATYFCARSYTPGSYYNMNLWGPGTLVTVSS

SEQ ID NO: 82 - protein, 6 aa, Artificial Sequence
  CDR-H1
SNYWIC

SEQ ID NO: 83 - protein, 17 aa, Artificial Sequence
  CDR-H2
CIYIGSGDTYYASWAKG

SEQ ID NO: 84 - protein, 12 aa, Artificial Sequence
  CDR-H3
SYTPGSYYNMNL

SEQ ID NO: 85 - protein, 110 aa, Artificial Sequence
  LC VR
DVVMTQTPSSVSAAVGGTVTINCQASQQISNELSWYQQKSGQPPKLLIYDASDLASGVPSRFKGSGSGTEFTLTISDLESADAATYYCQSTGGASSDGNAFGGGTEVVVK

SEQ ID NO: 86 - protein, 11 aa, Artificial Sequence
  CDR-L1
QASQQISNELS

SEQ ID NO: 87 - protein, 7 aa, Artificial Sequence
  CDR-L2
DASDLAS

SEQ ID NO: 88 - protein, 12 aa, Artificial Sequence
  CDR-L3
QSTGGASSDGNA

SEQ ID NO: 89 - protein, 120 aa, Artificial Sequence
  HC VR
QSLEESGGGLVKPEGSLTLTCKASGFDLNTYYYMCWVRQAPGKGLEWIACIATSDSGHTAYASWARGRFTISKTSSTTVTLQMTGLTAADTATYFCARGGRSVGDGVDLWGPGTLVTVSS

SEQ ID NO: 90 - protein, 6 aa, Artificial Sequence
  CDR-H1
TYYYMC

SEQ ID NO: 91 - protein, 18 aa, Artificial Sequence
  CDR-H2
CIATSDSGHTAYASWARG

SEQ ID NO: 92 - protein, 11 aa, Artificial Sequence
  CDR-H3
GGRSVGDGVDL

SEQ ID NO: 93 - protein, 112 aa, Artificial Sequence
  LC VR
DIVMTQTPASVEAAVGGTVTIKCQASQSISSWLAWYQQKPGQPPKLLIYDASDLASGVPSRFKGSGSGTEFTLTISDLESADAATYYCQSYLYVESGSHGFGFGGGTEVVVR

SEQ ID NO: 94 - protein, 11 aa, Artificial Sequence
  CDR-L1
QASQSISSWLA

SEQ ID NO: 95 - protein, 7 aa, Artificial Sequence
  CDR-L2
DASDLAS

SEQ ID NO: 96 - protein, 14 aa, Artificial Sequence
  CDR-L3
QSYLYVESGSHGFG

SEQ ID NO: 97 - protein, 120 aa, Artificial Sequence
  HC VR
QSLEESGGGLVKPEGSLTLTCKASGFDLNTYYYMCWVRQAPGKGLEWIACIGTSDSGHTAYASWARGRFTISKTSSTTVTLQMTGLTAADTATYFCARGGRSVGDGVDLWGPGTLVTVSS

SEQ ID NO: 98 - protein, 6 aa, Artificial Sequence
  CDR-H1
TYYYMC

SEQ ID NO: 99 - protein, 18 aa, Artificial Sequence
  CDR-H2
CIGTSDSGHTAYASWARG

SEQ ID NO: 100 - protein, 11 aa, Artificial Sequence
  CDR-H3
GGRSVGDGVDL

SEQ ID NO: 101 - protein, 112 aa, Artificial Sequence
  LC VR
DIVMTQTPASVEAAVGGTVTIKCQASQSISSWLAWYQQKPGQPPKLLIYDAFDLASGVPSRFKGSGSGTEFTLTISDLESADAATYYCQSYLYGESGSHGFGFGGGTEVVVR

SEQ ID NO: 102 - protein, 11 aa, Artificial Sequence
  CDR-L1
QASQSISSWLA

SEQ ID NO: 103 - protein, 7 aa, Artificial Sequence
  CDR-L2
DAFDLAS

SEQ ID NO: 104 - protein, 14 aa, Artificial Sequence
  CDR-L3
QSYLYGESGSHGFG

SEQ ID NO: 105 - protein, 119 aa, Artificial Sequence
  HC VR
QSLEESGGDLVKPGASLTLTCTASGFSFSSDGMCWVRQAPGKGLEWIACIAAGSSGSTYYATWAQGRFTISKSSSTTVTLQMTSLTAADTATYFCAGEDYTGNNYYALWGPGTLVTVSS

SEQ ID NO: 106 - protein, 5 aa, Artificial Sequence
  CDR-H1
SDGMC

SEQ ID NO: 107 - protein, 18 aa, Artificial Sequence
  CDR-H2
CIAAGSSGSTYYATWAQG

SEQ ID NO: 108 - protein, 11 aa, Artificial Sequence
  CDR-H3
EDYTGNNYYAL

SEQ ID NO: 109 - protein, 110 aa, Artificial Sequence
  LC VR
AIDMTQTPSPVSAVVGDTVTINCQASENIYSFLAWYQQKPGQPPKLLIYFASKLASGVPSRFKGSGSGTQFTLTISDVHSDDAAIYYCQQTGTYSDADNTFGGGTEVVVK

SEQ ID NO: 110 - protein, 11 aa, Artificial Sequence
  CDR-L1
QASENIYSFLA

SEQ ID NO: 111 - protein, 7 aa, Artificial Sequence
  CDR-L2
FASKLAS

SEQ ID NO: 112 - protein, 12 aa, Artificial Sequence
  CDR-L3
QQTGTYSDADNT

SEQ ID NO: 113 - protein, 117 aa, Artificial Sequence
  HC VR
QSLEESGGRLVTPGTPLTLTCTASGFSLSSYWMSWVRQAPGKGLEWIGIIYVSGSTYYASWAKGRFTISKTSTTVDLKMTSLTTEDTATYFCARVDYSTGWPLFNIWGPGTLVTVSL

SEQ ID NO: 114 - protein, 5 aa, Artificial Sequence
  CDR-H1
SYWMS

SEQ ID NO: 115 - protein, 16 aa, Artificial Sequence
  CDR-H2
IIYVSGSTYYASWAKG

SEQ ID NO: 116 - protein, 12 aa, Artificial Sequence
  CDR-H3
VDYSTGWPLFNI

SEQ ID NO: 117 - protein, 110 aa, Artificial Sequence
  LC VR
AQVLTQTASPVSAAVGGTVTINCQSSQSVYDNNWLGWYQQKPGQPPKLLIYGASILASGVPSRFKGSGSGTQFTLTISDLESDDAATYYCQGGCSGNTFVFGGGTEVVVK

SEQ ID NO: 118 - protein, 13 aa, Artificial Sequence
  CDR-L1
QSSQSVYDNNWLG

SEQ ID NO: 119 - protein, 7 aa, Artificial Sequence
  CDR-L2
GASILAS

SEQ ID NO: 120 - protein, 10 aa, Artificial Sequence
  CDR-L3
QGGCSGNTFV

SEQ ID NO: 121 - protein, 117 aa, Artificial Sequence
  HC VR
QMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTV

SEQ ID NO: 122 - protein, 5 aa, Artificial Sequence
  CDR-H1
TYWIG

SEQ ID NO: 123 - protein, 17 aa, Artificial Sequence
  CDR-H2
IIYPGDSETRYSPSFQG

SEQ ID NO: 124 - protein, 10 aa, Artificial Sequence
  CDR-H3
GSGISTPMDV

SEQ ID NO: 125 - protein, 113 aa, Artificial Sequence
  LC VR
DIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIK

SEQ ID NO: 126 - protein, 17 aa, Artificial Sequence
  CDR-L1
KSSQSVLYSSINKNYLA

SEQ ID NO: 127 - protein, 7 aa, Artificial Sequence
  CDR-L2
WASTRES

SEQ ID NO: 128 - protein, 9 aa, Artificial Sequence
  CDR-L3
QQYYSTPYT

SEQ ID NO: 129 - protein, 598 aa, Artificial Sequence
  ACE2 ECD Mutant
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCLPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 130 - protein, 805 aa, Homo sapiens [sequence omitted]

SEQ ID NO: 131 - protein, 830 aa, Artificial Sequence
  Binding agent
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCLPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 830 aa, Artificial Sequence
  Binding agent
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCLPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 133 - protein, 447 aa, Artificial Sequence
  Binding agent (HC)
QMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 134 - protein, 838 aa, Artificial Sequence
  Binding agent (LC)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCLPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGGGSGGGGSGGGGSGGGGSDIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 447 aa, Artificial Sequence; identical to SEQ ID NO: 133
  Binding agent (HC)

SEQ ID NO: 136 - protein, 843 aa, Artificial Sequence
  Binding agent (LC)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCLPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGGGSGGGGSGGGGSGGGGSGGGGSDIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 447 aa, Artificial Sequence; identical to SEQ ID NO: 133
  Binding agent (HC)

SEQ ID NO: 138 - protein, 838 aa, Artificial Sequence
  Binding agent (LC)
DIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCLPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 139 - protein, 447 aa, Artificial Sequence; identical to SEQ ID NO: 133
  Binding agent (HC)

SEQ ID NO: 140 - protein, 842 aa, Artificial Sequence
  Binding agent (LC)
DIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGSQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGQFWTNLYSLTVPFGQKPNIDVTDAMVDQAWDAQRIFKEAEKFFVSVGLPNMTQGFWENSMLTDPGNVQKAVCLPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 141 - protein, 447 aa, Artificial Sequence; identical to SEQ ID NO: 133
  Binding agent (HC)

SEQ ID NO: 142 - protein, 220 aa, Artificial Sequence
  Binding agent (LC)
DIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 447 aa, Artificial Sequence
  Binding agent (HC)
QMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 838 aa, Artificial Sequence; identical to SEQ ID NO: 134
  Binding agent (LC)

SEQ ID NO: 145 - protein, 447 aa, Artificial Sequence; identical to SEQ ID NO: 143
  Binding agent (HC)

SEQ ID NO: 146 - protein, 843 aa, Artificial Sequence; identical to SEQ ID NO: 136
  Binding agent (LC)

SEQ ID NO: 147 - protein, 447 aa, Artificial Sequence; identical to SEQ ID NO: 143
  Binding agent (HC)

SEQ ID NO: 148 - protein, 838 aa, Artificial Sequence; identical to SEQ ID NO: 138
  Binding agent (LC)

SEQ ID NO: 149 - protein, 447 aa, Artificial Sequence; identical to SEQ ID NO: 143
  Binding agent (HC)

SEQ ID NO: 150 - protein, 842 aa, Artificial Sequence; identical to SEQ ID NO: 140
  Binding agent (LC)

SEQ ID NO: 151 - protein, 447 aa, Artificial Sequence; identical to SEQ ID NO: 143
  Binding agent (HC)

SEQ ID NO: 152 - protein, 220 aa, Artificial Sequence; identical to SEQ ID NO: 142
  Binding agent (LC)

SEQ ID NO: 153 - protein, 447 aa, Artificial Sequence
  Binding agent (HC)
QMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 154 - protein, 491 aa, Artificial Sequence
  Binding agent (LC)
DIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSGGGGSGGGGSGGGGSQSVEESGGRLVTPGTPLTLTCTVSGFSLSSYAMNWVRQAPGKCLEWIGIISSSGSTYYASWAKGRFTISKTSTTVDLKITSPTTEDTATYFCARVGAGYAGYGYDNYYGMDPWGPGTLVTVSSGGGGSGGGGSGGGGSGGGGSAIQMTQSPSSLAASVGDTVTITCKASEDIGYGLNWYQQKLGIAPKLLIYGANTLESGVPSRFSGSGSETDYTLTISSVQAEDAGIYYCQQGYSTPLTFGCGTKVEIK

SEQ ID NO: 155 - protein, 447 aa, Artificial Sequence; identical to SEQ ID NO: 153
  Binding agent (HC)

SEQ ID NO: 156 - protein, 487 aa, Artificial Sequence
  Binding agent (LC)
DIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSGGGGSGGGGSGGGGSQSVEESGGRLVTPGTPLTLTCTVSGFSLSKYAMSWVRQAPGKCLEWIGIISSGGSTYYASWAKGRFTISKTSSTTVDLKITSPTTEDTATYFCARAGYPGYSPHYFNIWGPGTLVTVSLGGGGSGGGGSGGGGSGGGGSAIQMTQSPSSLAASVGDTVTITCKASEDIGYELNWYQQKLGIAPKLLIYGANTLESGVPSRFTGSGSETDYTLTISSVQAEDAGIYYCQQGYSTPPTFGCGTKVEIK

SEQ ID NO: 157 - protein, 447 aa, Artificial Sequence; identical to SEQ ID NO: 153
  Binding agent (HC)

SEQ ID NO: 158 - protein, 486 aa, Artificial Sequence
  Binding agent (LC)
DIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSGGGGSGGGGSGGGGSQSVEESGGRLVTPGTPLTLTCTVSGFSLSSYAMSWVRQAPGKCLEWIGIISSSGSTYYASWAKGRFTISKTSTTVDLKITSPTTEDTATYFCARAMYPGYSTGVYNIWGPGTLVTVSLGGGGSGGGGSGGGGSGGGGSAIQMTQSPSSLAASVGDTVTITCKASEDIGYELNWYQQKLGIAPKLLIYGANTLESGVPSRFSGSGSETDYTLTISSVQAEDAGIYYCQQGYNTPNTFGCGTKVEIK

SEQ ID NO: 159 - protein, 447 aa, Artificial Sequence; identical to SEQ ID NO: 153
  Binding agent (HC)

SEQ ID NO: 160 - protein, 488 aa, Artificial Sequence
  Binding agent (LC)
DIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSGGGGSGGGGSGGGGSQSVEESGGRLVTPGTPLTLTCTVSGFSLSSYAMSWVRQAPGKCLEWIGIINSGRSTYYASWAKGRFTISKTSTTVDLKITSPTTEDTATYFCARDATSIGYYRYYGMDPWGPGTLVTVSSGGGGSGGGGSGGGGSGGGGSAIQMTQSPSSLAASVGDTVTITCKASEDIGYGLNWYQQKLGIAPKLLIYGANTLESGVPSRFSGSGSETDYTLTISSVQAEDAGIYYCQQGYSTPLTFGCGTKVEIK

SEQ ID NO: 161 - protein, 447 aa, Artificial Sequence; identical to SEQ ID NO: 153
  Binding agent (HC)

SEQ ID NO: 162 - protein, 488 aa, Artificial Sequence
  Binding agent (LC)
DIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSGGGGSGGGGSGGGGSQSVEESGGRLVTPGTPLTLTCTVSGFSLSSYAMSWVRQAPGKCLEWIGIISSSGTTYYASWAKGPFTISKTSTTVDLKITSPTTEDTATYFCARGGAGYGYAHYYGMDPWGPGTLVTVSSGGGGSGGGGSGGGGSGGGGSAIQMTQSPSSLAASVGDTVTITCKASEDIGYGLNWYQQKLGIAPKLLIYGANTLESGVPSRFSGSGSETDYTLTISSVQAEDAGIYYCQQGYSTPLTFGCGTKVEIK

SEQ ID NO: 163 - protein, 453 aa, Artificial Sequence
  Binding agent (HC)
QSVEESGGRLVTPGTPLTLTCTVSGFSLSSYAMNWVRQAPGKGLEWIGIISSSGSTYYASWAKGRFTISKTSTTVDLKITSPTTEDTATYFCARVGAGYAGYGYDNYYGMDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 164 - protein, 485 aa, Artificial Sequence
  Binding agent (LC)
AIQMTQSPSSLAASVGDTVTITCKASEDIGYGLNWYQQKLGIAPKLLIYGANTLESGVPSRFSGSGSETDYTLTISSVQAEDAGIYYCQQGYSTPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSGGGGSGGGGSGGGGSQMQLVQSGTEVKKPGESLKISCKGSGYGFITYWIGWVRQMPGKCLEWMGIIYPGDSETRYSPSFQGQVTISADKSINTAYLQWSSLKASDTAIYYCAGGSGISTPMDVWGQGTTVTVGGGGSGGGGSGGGGSGGGGSDIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGCGTKVEIK

SEQ ID NO: 165 - protein, 450 aa, Artificial Sequence
  Binding agent (HC)
QSVEESGGRLVTPGTPLTLTCTVSGFSLSSYAMSWVRQAPGKGLEWIGIINSGRSTYYASWAKGRFTISKTSTTVDLKITSPTTEDTATYFCARDATSIGYYRYYGMDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 166 - protein, 485 aa, Artificial Sequence; identical to SEQ ID NO: 164
  Binding agent (LC)

SEQ ID NO: 167 - protein, 450 aa, Artificial Sequence
  Binding agent (HC)
QSVEESGGRLVTPGTPLTLTCTVSGFSLSSYAMSWVRQAPGKGLEWIGIISSSGTTYYASWAKGPFTISKTSTTVDLKITSPTTEDTATYFCARGGAGYGYAHYYGMDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 168 - protein, 485 aa, Artificial Sequence; identical to SEQ ID NO: 164
  Binding agent (LC)

SEQ ID NO: 169 - protein, 449 aa, Artificial Sequence
  Binding agent (HC)
QSVEESGGRLVTPGTPLTLTCTVSGFSLSKYAMSWVRQAPGKGLEWIGIISSGGSTYYASWAKGRFTISKTSSTTVDLKITSPTTEDTATYFCARAGYPGYSPHYFNIWGPGTLVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 170 - protein, 485 aa, Artificial Sequence
  Binding agent (LC)
AIQMTQSPSSLAASVGDTVTITCKASEDIGYELNWYQQKLGIAPKLLIYGANTLESGVPSRFTGSGSETDYTLTISSVQAEDAGIYYCQQGYSTPPTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSGGGGSGGGGSGGGGSQMQLVQSGTEVKKPGESLKISCKGSGYGFITYWIGWVRQMPGKCLEWMGIIYPGDSETRYSPSFQGQVTISADKSINTAYLQWSSLKASDTAIYYCAGGSGISTPMDVWGQGTTVTVGGGGSGGGGSGGGGSGGGGSDIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGCGTKVEIK

SEQ ID NO: 171 - protein, 448 aa, Artificial Sequence
  Binding agent (HC)
QSVEESGGRLVTPGTPLTLTCTVSGFSLSSYAMSWVRQAPGKGLEWIGIISSSGSTYYASWAKGRFTISKTSTTVDLKITSPTTEDTATYFCARAMYPGYSTGVYNIWGPGTLVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 172 - protein, 485 aa, Artificial Sequence
  Binding agent (LC)
AIQMTQSPSSLAASVGDTVTITCKASEDIGYELNWYQQKLGIAPKLLIYGANTLESGVPSRFSGSGSETDYTLTISSVQAEDAGIYYCQQGYNTPNTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSGGGGSGGGGSGGGGSQMQLVQSGTEVKKPGESLKISCKGSGYGFITYWIGWVRQMPGKCLEWMGIIYPGDSETRYSPSFQGQVTISADKSINTAYLQWSSLKASDTAIYYCAGGSGISTPMDVWGQGTTVTVGGGGSGGGGSGGGGSGGGGSDIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGCGTKVEIK

SEQ ID NO: 173 - protein, 447 aa, Artificial Sequence; identical to SEQ ID NO: 153
  Binding agent (HC)

SEQ ID NO: 174 - protein, 488 aa, Artificial Sequence
  Binding agent (LC)
DIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSGGGGSGGGGSGGGGSQSLEESGGRLVTPGTPLTLTCTVSGFSLSSYAMSWVRQAPGKCLEWIGIISSSGSTYYASWAKGRFTISKTSTTVDLKITSPTTEDTATYFCARDASSRGYYYYYGMDPWGPGTLVTVSSGGGGSGGGGSGGGGSGGGGSAIQMTQSPSSLAASVGDTVTITCKASEDIGYGLNWYQQKLGIAPKLLIYGANTLESGVPSRFSGSGSETDYTLTISSVQAEDAGIYYCQQGYSTPLTFGCGTKVEIK

SEQ ID NO: 175 - protein, 447 aa, Artificial Sequence; identical to SEQ ID NO: 153
  Binding agent (HC)

SEQ ID NO: 176 - protein, 487 aa, Artificial Sequence
  Binding agent (LC)
DIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSGGGGSGGGGSGGGGSQSVEESGGRLVTPGTPLTLTCTVSGFSLNNYAMNWVRQAPGKCLEWIGIISSSGNTYYANWAKGRFTISKTSTTVDLKITSPTTEDTATYFCARDSYPGYSPYYGMDPWGPGTLVTVSSGGGGSGGGGSGGGGSGGGGSAIQMTQSPSSLAASVGDTVTITCKASEDIAYGLNWYQQKLGITPKLLIYGANTLESGVPSRFSGSGSETHYTLTISSVQAEDAGIYYCQQGYNTPPTFGCGTKVEIK

SEQ ID NO: 177 - protein, 447 aa, Artificial Sequence; identical to SEQ ID NO: 153
  Binding agent (HC)

SEQ ID NO: 178 - protein, 490 aa, Artificial Sequence
  Binding agent (LC)
DIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSGGGGSGGGGSGGGGSQSLEESGGDLVKPGASLTLTCTASGFSFSSDGMCWVRQAPGKCLEWIACIAAGSSGSTYYATWAQGRFTISKSSSTTVTLQMTSLTAADTATYFCAGEDYTGNNYYALWGPGTLVTVSSGGGGSGGGGSGGGGSGGGGSAIDMTQTPSPVSAVVGDTVTINCQASENIYSFLAWYQQKPGQPPKLLIYFASKLASGVPSRFKGSGSGTQFTLTISDVHSDDAAIYYCQQTGTYSDADNTFGCGTEVVVK

SEQ ID NO: 179 - protein, 450 aa, Artificial Sequence
  Binding agent (HC)
QSLEESGGRLVTPGTPLTLTCTVSGFSLSSYAMSWVRQAPGKGLEWIGIISSSGSTYYASWAKGRFTISKTSTTVDLKITSPTTEDTATYFCARDASSRGYYYYYGMDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 180 - protein, 485 aa, Artificial Sequence; identical to SEQ ID NO: 164
  Binding agent (LC)

SEQ ID NO: 181 - protein, 449 aa, Artificial Sequence
  Binding agent (HC)
QSVEESGGRLVTPGTPLTLTCTVSGFSLNNYAMNWVRQAPGKGLEWIGIISSSGNTYYANWAKGRFTISKTSTTVDLKITSPTTEDTATYFCARDSYPGYSPYYGMDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 182 - protein, 485 aa, Artificial Sequence
  Binding agent (LC)
AIQMTQSPSSLAASVGDTVTITCKASEDIAYGLNWYQQKLGITPKLLIYGANTLESGVPSRFSGSGSETHYTLTISSVQAEDAGIYYCQQGYNTPPTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSGGGGSGGGGSGGGGSQMQLVQSGTEVKKPGESLKISCKGSGYGFITYWIGWVRQMPGKCLEWMGIIYPGDSETRYSPSFQGQVTISADKSINTAYLQWSSLKASDTAIYYCAGGSGISTPMDVWGQGTTVTVGGGGSGGGGSGGGGSGGGGSDIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGCGTKVEIK

SEQ ID NO: 183 - protein, 449 aa, Artificial Sequence
  Binding agent (HC)
QSLEESGGDLVKPGASLTLTCTASGFSFSSDGMCWVRQAPGKGLEWIACIAAGSSGSTYYATWAQGRFTISKSSSTTVTLQMTSLTAADTATYFCAGEDYTGNNYYALWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 184 - protein, 488 aa, Artificial Sequence
  Binding agent (LC)
AIDMTQTPSPVSAVVGDTVTINCQASENIYSFLAWYQQKPGQPPKLLIYFASKLASGVPSRFKGSGSGTQFTLTISDVHSDDAAIYYCQQTGTYSDADNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSGGGGSGGGGSGGGGSQMQLVQSGTEVKKPGESLKISCKGSGYGFITYWIGWVRQMPGKCLEWMGIIYPGDSETRYSPSFQGQVTISADKSINTAYLQWSSLKASDTAIYYCAGGSGISTPMDVWGQGTTVTVGGGGSGGGGSGGGGSGGGGSDIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGCGTKVEIK

SEQ ID NO: 185 - protein, 449 aa, Artificial Sequence; identical to SEQ ID NO: 183
  Binding agent (HC)

SEQ ID NO: 186 - protein, 485 aa, Artificial Sequence
  Binding agent (LC)
AIDMTQTPSPVSAVVGDTVTINCQASENIYSFLAWYQQKPGQPPKLLIYFASKLASGVPSRFKGSGSGTQFTLTISDVHSDDAAIYYCQQTGTYSDADNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSGGGGSGGGGSGGGGSQSLEESGGRLVTPGTPLTLTCTVSGFSLSSYAMSWVRQAPGKCLEWIGIISSSGSTYYASWAKGRFTISKTSTTVDLKITSPTTEDTATYFCARDASSRGYYYYYGMDPWGPGTLVTVSSGGGGSGGGGSGGGGSGGGGSAIQMTQSPSSLAASVGDTVTITCKASEDIGYGLNWYQQKLGIAPKLLIYGANTLESGVPSRFSGSGSETDYTLTISSVQAEDAGIYYCQQGYSTPLTFGCGTKVEIK

SEQ ID NO: 187 - protein, 449 aa, Artificial Sequence; identical to SEQ ID NO: 183
  Binding agent (HC)

SEQ ID NO: 188 - protein, 484 aa, Artificial Sequence
  Binding agent (LC)
AIDMTQTPSPVSAVVGDTVTINCQASENIYSFLAWYQQKPGQPPKLLIYFASKLASGVPSRFKGSGSGTQFTLTISDVHSDDAAIYYCQQTGTYSDADNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSGGGGSGGGGSGGGGSQSVEESGGRLVTPGTPLTLTCTVSGFSLNNYAMNWVRQAPGKCLEWIGIISSSGNTYYANWAKGRFTISKTSTTVDLKITSPTTEDTATYFCARDSYPGYSPYYGMDPWGPGTLVTVSSGGGGSGGGGSGGGGSGGGGSAIQMTQSPSSLAASVGDTVTITCKASEDIAYGLNWYQQKLGITPKLLIYGANTLESGVPSRFSGSGSETHYTLTISSVQAEDAGIYYCQQGYNTPPTFGCGTKVEIK

SEQ ID NO: 189 - protein, 449 aa, Artificial Sequence; identical to SEQ ID NO: 183
  Binding agent (HC)

SEQ ID NO: 190 - protein, 485 aa, Artificial Sequence
  Binding agent (LC)
AIDMTQTPSPVSAVVGDTVTINCQASENIYSFLAWYQQKPGQPPKLLIYFASKLASGVPSRFKGSGSGTQFTLTISDVHSDDAAIYYCQQTGTYSDADNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSGGGGSGGGGSGGGGSQSVEESGGRLVTPGTPLTLTCTVSGFSLSSYAMSWVRQAPGKCLEWIGIISSSGTTYYASWAKGPFTISKTSTTVDLKITSPTTEDTATYFCARGGAGYGYAHYYGMDPWGPGTLVTVSSGGGGSGGGGSGGGGSGGGGSAIQMTQSPSSLAASVGDTVTITCKASEDIGYGLNWYQQKLGIAPKLLIYGANTLESGVPSRFSGSGSETDYTLTISSVQAEDAGIYYCQQGYSTPLTFGCGTKVEIK

SEQ ID NO: 191 - protein, 450 aa, Artificial Sequence; identical to SEQ ID NO: 179
  Binding agent (HC)

SEQ ID NO: 192 - protein, 484 aa, Artificial Sequence
  Binding agent (LC)
AIQMTQSPSSLAASVGDTVTITCKASEDIGYGLNWYQQKLGIAPKLLIYGANTLESGVPSRFSGSGSETDYTLTISSVQAEDAGIYYCQQGYSTPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSGGGGSGGGGSGGGGSQSLEESGGDLVKPGASLTLTCTASGFSFSSDGMCWVRQAPGKCLEWIACIAAGSSGSTYYATWAQGRFTISKSSSTTVTLQMTSLTAADTATYFCAGEDYTGNNYYALWGPGTLVTVSSGGGGSGGGGSGGGGSGGGGSAIDMTQTPSPVSAVVGDTVTINCQASENIYSFLAWYQQKPGQPPKLLIYFASKLASGVPSRFKGSGSGTQFTLTISDVHSDDAAIYYCQQTGTYSDADNTFGCGTEVVVK

SEQ ID NO: 193 - protein, 449 aa, Artificial Sequence; identical to SEQ ID NO: 181
  Binding agent (HC)

SEQ ID NO: 194 - protein, 484 aa, Artificial Sequence
  Binding agent (LC)
AIQMTQSPSSLAASVGDTVTITCKASEDIAYGLNWYQQKLGITPKLLIYGANTLESGVPSRFSGSGSETHYTLTISSVQAEDAGIYYCQQGYNTPPTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSGGGGSGGGGSGGGGSQSLEESGGDLVKPGASLTLTCTASGFSFSSDGMCWVRQAPGKCLEWIACIAAGSSGSTYYATWAQGRFTISKSSSTTVTLQMTSLTAADTATYFCAGEDYTGNNYYALWGPGTLVTVSSGGGGSGGGGSGGGGSGGGGSAIDMTQTPSPVSAVVGDTVTINCQASENIYSFLAWYQQKPGQPPKLLIYFASKLASGVPSRFKGSGSGTQFTLTISDVHSDDAAIYYCQQTGTYSDADNTFGCGTEVVVK

SEQ ID NO: 195 - protein, 450 aa, Artificial Sequence; identical to SEQ ID NO: 167
  Binding agent (HC)

SEQ ID NO: 196 - protein, 484 aa, Artificial Sequence; identical to SEQ ID NO: 192
  Binding agent (LC)

SEQ ID NO: 197 - protein, 1273 aa, Coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 198 - protein, 202 aa, Artificial Sequence
  S Protein RBD
VRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPK

SEQ ID NO: 199 - RNA, 47 nt, Artificial Sequence
  5′-UTR (hAg-Kozak)
aacuaguauucuucugguccccacagacucagagagaacccgccacc

SEQ ID NO: 200 - RNA, 311 nt, Artificial Sequence
  3′-UTR (2hBg)
cucgagagcucgcuuucuugcuguccaauuucuauuaaagguuccuuuguucccuaaguccaacuacuaaacugggggauauuaugaagggccuugagcaucuggauucugccuaauaaaaaacauuuauuuucauugcugcgucgagagcucgcuuucuugcuguccaauuucuauuaaagguuccuuuguucccuaaguccaacuacuaaacugggggauauuaugaagggccuugagcaucuggauucugccuaauaaaaaacauuuauuuucauugcugcgucgagaccugguccagagucgcuagc

SEQ ID NO: 201 - RNA, 278 nt, Artificial Sequence
  3′-UTR (FI element)
cugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacc

SEQ ID NO: 202 - RNA, 110 nt, Artificial Sequence
  A30L70
aaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 203 - protein, 20 aa, Artificial Sequence
  Linker
GGGGSGGGGSGGGGSGGGGS

SEQ ID NO: 204 - protein, 25 aa, Artificial Sequence
  Linker
GGGGSGGGGSGGGGSGGGGSGGGGS

SEQ ID NO: 205 - protein, 450 aa, Artificial Sequence; identical to SEQ ID NO: 179
  Binding agent (HC)

SEQ ID NO: 206 - protein, 486 aa, Artificial Sequence
  Binding agent (LC)
AIQMTQSPSSLAASVGDTVTITCKASEDIGYGLNWYQQKLGIAPKLLIYGANTLESGVPSRFSGSGSETDYTLTISSVQAEDAGIYYCQQGYSTPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSGGGGSGGGGSGGGGSQEQLEESGGDLVKPEGSLTLTCTASGFSFSSNYWICWVRQAPGKGLEWIGCIYIGSGDTYYASWAKGRFTISKTSSTTVTLQMTSLTAADTATYFCARSYTPGSYYNMNLWGPGTLVTVSSGGGGSGGGGSGGGGSGGGGSDVVMTQTPSSVSAAVGGTVTINCQASQQISNELSWYQQKSGQPPKLLIYDASDLASGVPSRFKGSGSGTEFTLTISDLESADAATYYCQSTGGASSDGNAFGGGTEVVVK

SEQ ID NO: 207 - protein, 451 aa, Artificial Sequence
  Binding agent (HC)
QEQLEESGGDLVKPEGSLTLTCTASGFSFSSNYWICWVRQAPGKGLEWIGCIYIGSGDTYYASWAKGRFTISKTSSTTVTLQMTSLTAADTATYFCARSYTPGSYYNMNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 208 - protein, 485 aa, Artificial Sequence
  Binding agent (LC)
DVVMTQTPSSVSAAVGGTVTINCQASQQISNELSWYQQKSGQPPKLLIYDASDLASGVPSRFKGSGSGTEFTLTISDLESADAATYYCQSTGGASSDGNAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSGGGGSGGGGSGGGGSQSLEESGGRLVTPGTPLTLTCTVSGFSLSSYAMSWVRQAPGKGLEWIGIISSSGSTYYASWAKGRFTISKTSTTVDLKITSPTTEDTATYFCARDASSRGYYYYYGMDPWGPGTLVTVSSGGGGSGGGGSGGGGSGGGGSAIQMTQSPSSLAASVGDTVTITCKASEDIGYGLNWYQQKLGIAPKLLIYGANTLESGVPSRFSGSGSETDYTLTISSVQAEDAGIYYCQQGYSTPLTFGAGTKVEIK

SEQ ID NO: 209 - protein, 451 aa, Artificial Sequence; identical to SEQ ID NO: 207
  Binding agent (HC)

SEQ ID NO: 210 - protein, 484 aa, Artificial Sequence
  Binding agent (LC)
DVVMTQTPSSVSAAVGGTVTINCQASQQISNELSWYQQKSGQPPKLLIYDASDLASGVPSRFKGSGSGTEFTLTISDLESADAATYYCQSTGGASSDGNAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSGGGGSGGGGSGGGGSQSVEESGGRLVTPGTPLTLTCTVSGFSLNNYAMNWVRQAPGKGLEWIGIISSSGNTYYANWAKGRFTISKTSTTVDLKITSPTTEDTATYFCARDSYPGYSPYYGMDPWGPGTLVTVSSGGGGSGGGGSGGGGSGGGGSAIQMTQSPSSLAASVGDTVTITCKASEDIAYGLNWYQQKLGITPKLLIYGANTLESGVPSRFSGSGSETHYTLTISSVQAEDAGIYYCQQGYNTPPTFGAGTKVEIK

SEQ ID NO: 211 - protein, 449 aa, Artificial Sequence; identical to SEQ ID NO: 181
  Binding agent (HC)

SEQ ID NO: 212 - protein, 486 aa, Artificial Sequence
  Binding agent (LC)
AIQMTQSPSSLAASVGDTVTITCKASEDIAYGLNWYQQKLGITPKLLIYGANTLESGVPSRFSGSGSETHYTLTISSVQAEDAGIYYCQQGYNTPPTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDVPGGSGGGGSGGGGSGGGGSQEQLEESGGDLVKPEGSLTLTCTASGFSFSSNYWICWVRQAPGKGLEWIGCIYIGSGDTYYASWAKGRFTISKTSSTTVTLQMTSLTAADTATYFCARSYTPGSYYNMNLWGPGTLVTVSSGGGGSGGGGSGGGGSGGGGSDVVMTQTPSSVSAAVGGTVTINCQASQQISNELSWYQQKSGQPPKLLIYDASDLASGVPSRFKGSGSGTEFTLTISDLESADAATYYCQSTGGASSDGNAFGGGTEVVVK

(19) United States
(12) Patent Application Publication
(10) Pub. No.: US 2023/0287088 A1
(43) Pub. Date: Sep. 14, 2023

(54) BINDING AGENTS FOR CORONAVIRUS S PROTEIN

(71) Applicant: BIONTECH SE, Mainz (DE)

(72) Inventors: Karsten Beckmann, Vaterstetten (DE); Anna Carle, Eichenau (DE); Sandra Prassl, Munich (DE); Claudia Paulmann, Geretsried (DE); Christiane Stadler, Bensheim (DE); Georg Falck, Mainz (DE); Leyla Fischer, Gau-Odernheim (DE); Alexander Muik, Seeheim-Jugenheim (DE); Ugur Sahin, Mainz (DE); Caroline Scharf, Mainz (DE)

(21) Appl. No.: 18/019,787

(22) PCT Filed: Jul. 29, 2021
(86) PCT No.: PCT/EP2021/071302

§ 371 Date: Feb. 3, 2023

(30) Foreign Application Priority Data
Aug. 6, 2020 (WO) PCT/EP2020/072199

(51) Int. Cl. C07K 16/10; A61P 31/14

(52) U.S. Cl. CPC C07K 16/10; A61P 31/14; C07K 2317/31; C07K 2317/92

(57) ABSTRACT

The present disclosure relates to a binding agent comprising a first and a second binding domain, wherein the first binding domain is capable of binding to a coronavirus spike protein (S protein) and the second binding domain is capable of binding to the coronavirus S protein, and wherein the first and second binding domains bind to different epitopes of the coronavirus S protein. Moreover, the disclosure relates to an antibody capable of binding to a coronavirus spike protein (S protein). The disclosure also relates to a nucleic acid such as RNA encoding the binding agent, in particular antibody, disclosed herein and a host cell transformed or transfected with said nucleic acid. Furthermore, the disclosure relates to a medical use of said binding agent, antibody, or nucleic acid. The agents and medical uses described herein are, in particular, useful for the prevention or treatment of coronavirus infection in a subject.

The present disclosure relates to a binding agent comprising a first and a second binding domain, wherein the first binding domain is capable of binding to a coronavirus spike protein (S protein) and the second binding domain is capable of binding to the coronavirus S protein, and wherein the first and second binding domains bind to different epitopes of the coronavirus S protein. Moreover, the disclosure relates to an antibody capable of binding to a coronavirus spike protein (S protein). In one embodiment, the binding agent, in particular the antibody described herein binds to the S1 subunit of the S protein, in particular to the receptor binding domain (RBD) of the S1 subunit of the S protein. The disclosure also relates to a nucleic acid such as RNA encoding the binding agent, in particular antibody, disclosed herein and a host cell transformed or transfected with said nucleic acid. Furthermore, the disclosure relates to a medical use of said binding agent, antibody, or nucleic acid. The agents and medical uses described herein are, in particular, useful for the prevention or treatment of coronavirus infection in a subject. Specifically, in one embodiment, the present disclosure relates to methods comprising administering to a subject RNA encoding the binding agent, in particular antibody, disclosed herein. Administering to the subject RNA encoding the binding agent or antibody disclosed herein may provide (following expression of the RNA by appropriate target cells) the binding agent disclosed herein for blocking or neutralizing coronavirus.

In December 2019, a pneumonia outbreak of unknown cause occurred in Wuhan, China and it became clear that a novel coronavirus (severe acute respiratory syndrome coronavirus 2; SARS-CoV-2) was the underlying cause. The genetic sequence of SARS-CoV-2 became available to the WHO and public (MN908947.3) and the virus was categorized into the betacoronavirus subfamily. By sequence analysis, the phylogenetic tree revealed a closer relationship to severe acute respiratory syndrome (SARS) virus isolates than to another coronavirus infecting humans, namely the Middle East respiratory syndrome (MERS) virus. On February 2nd, a total of 14,557 cases were globally confirmed in 24 countries including Germany and a subsequent self-sustaining, human-to-human virus spread resulted in that SARS-CoV-2 became a global pandemic.

Coronaviruses are positive-sense, single-stranded RNA ((+)ssRNA) enveloped viruses that encode for a total of four structural proteins, spike protein (S), envelope protein (E), membrane protein (M) and nucleocapsid protein (N). The spike protein (S protein) is responsible for receptor-recognition, attachment to the cell, infection via the endosomal pathway, and the genomic release driven by fusion of viral and endosomal membranes. Though sequences between the different family members vary, there are conserved regions and motifs within the S protein making it possible to divide the S protein into two subdomains: S1 and S2. While the S2, with its transmembrane domain, is responsible for membrane fusion, the S1 domain recognizes the virus-specific receptor and binds to the target host cell. Within several coronavirus isolates, the receptor binding domain (RBD) was identified.

Therapeutics against SARS-CoV-2 are currently not available, but urgently needed.

### SUMMARY

The present invention provides binding agents that are at least bispecific for the binding to coronavirus spike protein (S protein), i.e., they are capable of binding to at least two different epitopes of the coronavirus S protein. Additionally, the present invention provides antibodies such as monospecific, bivalent antibodies that bind to coronavirus S protein. The binding agents, including antibodies, described herein may block the interaction of coronavirus S protein with its target receptor, ACE2. The binding agents and nucleic acids encoding these binding agents may be used in the treatment or prevention of coronavirus infection in a subject. In particular, RNA encoding a binding agent disclosed herein may be administered to provide (following expression of the RNA by appropriate target cells) binding agent for targeting coronavirus S protein, in particular SARS-CoV-2 S protein.

Thus, the pharmaceutical composition described herein may comprise as the active principle single-stranded RNA that may be translated into the respective protein upon entering cells of a recipient. In addition to wildtype or codon-optimized sequences encoding the sequence of the binding agent, the RNA may contain one or more structural elements optimized for maximal efficacy of the RNA with respect to stability and translational efficiency (5′ cap, 5′ UTR, 3′ UTR, poly(A)-tail). In one embodiment, the RNA contains all of these elements.

The RNA described herein may be complexed with proteins and/or lipids, preferably lipids, to generate RNA-particles for administration. If a combination of different RNAs is used, the RNAs may be complexed together or complexed separately with proteins and/or lipids to generate RNA-particles for administration.

In one aspect, the present invention provides a binding agent comprising at least a first binding domain binding to a coronavirus spike protein (S protein) and a second binding domain binding to the coronavirus S protein, wherein the first and second binding domains bind to different epitopes of the coronavirus S protein.

In one embodiment, the binding agent is a multispecific such as a bispecific binding agent.

In one embodiment, the first binding domain comprises a heavy chain variable region (VH). In one embodiment, the VH comprises a HCDR3 comprising a sequence selected from the group consisting of SEQ ID NO: 4, 12, 20, 28, 36, 44, 52, 60, 68, 76, 84, 92, 100, 108, 116, and 124. In one embodiment, the VH comprises a HCDR2 comprising a sequence selected from the group consisting of SEQ ID NO: 3, 11, 19, 27, 35, 43, 51, 59, 67, 75, 83, 91, 99, 107, 115, and 123. In one embodiment, the VH comprises a HCDR1 comprising a sequence selected from the group consisting of SEQ ID NO: 2, 10, 18, 26, 34, 42, 50, 58, 66, 74, 82, 90, 98, 106, 114, and 122. In one embodiment, the VH is selected from the group consisting of:
(i) a VH comprising a HCDR1 comprising the sequence of SEQ ID NO: 2, a HCDR2 comprising the sequence of SEQ ID NO: 3, and a HCDR3 comprising the sequence of SEQ ID NO: 4;
(ii) a VH comprising a HCDR1 comprising the sequence of SEQ ID NO: 10, a HCDR2 comprising the sequence of SEQ ID NO: 11, and a HCDR3 comprising the sequence of SEQ ID NO: 12;
(iii) a VH comprising a HCDR1 comprising the sequence of SEQ ID NO: 18, a HCDR2 comprising the sequence of SEQ ID NO: 19, and a HCDR3 comprising the sequence of SEQ ID NO: 20;
(iv) a VH comprising a HCDR1 comprising the sequence of SEQ ID NO: 26, a HCDR2 comprising the sequence of SEQ ID NO: 27, and a HCDR3 comprising the sequence of SEQ ID NO: 28;
(v) a VH comprising a HCDR1 comprising the sequence of SEQ ID NO: 34, a HCDR2 comprising the sequence of SEQ ID NO: 35, and a HCDR3 comprising the sequence of SEQ ID NO: 36;
(vi) a VH comprising a HCDR1 comprising the sequence of SEQ ID NO: 42, a HCDR2 comprising the sequence of SEQ ID NO: 43, and a HCDR3 comprising the sequence of SEQ ID NO: 44;
(vii) a VH comprising a HCDR1 comprising the sequence of SEQ ID NO: 50, a HCDR2 comprising the sequence of SEQ ID NO: 51, and a HCDR3 comprising the sequence of SEQ ID NO: 52;
(viii) a VH comprising a HCDR1 comprising the sequence of SEQ ID NO: 58, a HCDR2 comprising the sequence of SEQ ID NO: 59, and a HCDR3 comprising the sequence of SEQ ID NO: 60;
(ix) a VH comprising a HCDR1 comprising the sequence of SEQ ID NO: 66, a HCDR2 comprising the sequence of SEQ ID NO: 67, and a HCDR3 comprising the sequence of SEQ ID NO: 68;
(x) a VH comprising a HCDR1 comprising the sequence of SEQ ID NO: 74, a HCDR2 comprising the sequence of SEQ ID NO: 75, and a HCDR3 comprising the sequence of SEQ ID NO: 76;
(xi) a VH comprising a HCDR1 comprising the sequence of SEQ ID NO: 82, a HCDR2 comprising the sequence of SEQ ID NO: 83, and a HCDR3 comprising the sequence of SEQ ID NO: 84;
(xii) a VH comprising a HCDR1 comprising the sequence of SEQ ID NO: 90, a HCDR2 comprising the sequence of SEQ ID NO: 91, and a HCDR3 comprising the sequence of SEQ ID NO: 92;
(xiii) a VH comprising a HCDR1 comprising the sequence of SEQ ID NO: 98, a HCDR2 comprising the sequence of SEQ ID NO: 99, and a HCDR3 comprising the sequence of SEQ ID NO: 100;
(xiv) a VH comprising a HCDR1 comprising the sequence of SEQ ID NO: 106, a HCDR2 comprising the sequence of SEQ ID NO: 107, and a HCDR3 comprising the sequence of SEQ ID NO: 108;
(xv) a VH comprising a HCDR1 comprising the sequence of SEQ ID NO: 114, a HCDR2 comprising the sequence of SEQ ID NO: 115, and a HCDR3 comprising the sequence of SEQ ID NO: 116; and
(xvi) a VH comprising a HCDR1 comprising the sequence of SEQ ID NO: 122, a HCDR2 comprising the sequence of SEQ ID NO: 123, and a HCDR3 comprising the sequence of SEQ ID NO: 124.

In one embodiment, the first binding domain comprises a light chain variable region (VL). In one embodiment, the VL comprises a LCDR3 comprising a sequence selected from the group consisting of SEQ ID NO: 8, 16, 24, 32, 40, 48, 56, 64, 72, 80, 88, 96, 104, 112, 120, and 128. In one embodiment, the VL comprises a LCDR2 comprising a sequence selected from the group consisting of SEQ ID NO: 7, 15, 23, 31, 39, 47, 55, 63, 71, 79, 87, 95, 103, 111, 119, and 127. In one embodiment, the VL comprises a LCDR1 comprising a sequence selected from the group consisting of SEQ ID NO: 6, 14, 22, 30, 38, 46, 54, 62, 70, 78, 86, 94, 102, 110, 118, and 126. In one embodiment, the VL is selected from the group consisting of:
(i) a VL comprising a LCDR1 comprising the sequence of SEQ ID NO: 6, a LCDR2 comprising the sequence of SEQ ID NO: 7, and a LCDR3 comprising the sequence of SEQ ID NO: 8;
(ii) a VL comprising a LCDR1 comprising the sequence of SEQ ID NO: 14, a LCDR2 comprising the sequence of SEQ ID NO: 15, and a LCDR3 comprising the sequence of SEQ ID NO: 16;
(iii) a VL comprising a LCDR1 comprising the sequence of SEQ ID NO: 22, a LCDR2 comprising the sequence of SEQ ID NO: 23, and a LCDR3 comprising the sequence of SEQ ID NO: 24;
(iv) a VL comprising a LCDR1 comprising the sequence of SEQ ID NO: 30, a LCDR2 comprising the sequence of SEQ ID NO: 31, and a LCDR3 comprising the sequence of SEQ ID NO: 32;
(v) a VL comprising a LCDR1 comprising the sequence of SEQ ID NO: 38, a LCDR2 comprising the sequence of SEQ ID NO: 39, and a LCDR3 comprising the sequence of SEQ ID NO: 40;
(vi) a VL comprising a LCDR1 comprising the sequence of SEQ ID NO: 46, a LCDR2 comprising the sequence of SEQ ID NO: 47, and a LCDR3 comprising the sequence of SEQ ID NO: 48;
(vii) a VL comprising a LCDR1 comprising the sequence of SEQ ID NO: 54, a LCDR2 comprising the sequence of SEQ ID NO: 55, and a LCDR3 comprising the sequence of SEQ ID NO: 56;
(viii) a VL comprising a LCDR1 comprising the sequence of SEQ ID NO: 62, a LCDR2 comprising the sequence of SEQ ID NO: 63, and a LCDR3 comprising the sequence of SEQ ID NO: 64;
(ix) a VL comprising a LCDR1 comprising the sequence of SEQ ID NO: 70, a LCDR2 comprising the sequence of SEQ ID NO: 71, and a LCDR3 comprising the sequence of SEQ ID NO: 72;
(x) a VL comprising a LCDR1 comprising the sequence of SEQ ID NO: 78, a LCDR2 comprising the sequence of SEQ ID NO: 79, and a LCDR3 comprising the sequence of SEQ ID NO: 80;
(xi) a VL comprising a LCDR1 comprising the sequence of SEQ ID NO: 86, a LCDR2 comprising the sequence of SEQ ID NO: 87, and a LCDR3 comprising the sequence of SEQ ID NO: 88;
(xii) a VL comprising a LCDR1 comprising the sequence of SEQ ID NO: 94, a LCDR2 comprising the sequence of SEQ ID NO: 95, and a LCDR3 comprising the sequence of SEQ ID NO: 96;
(xiii) a VL comprising a LCDR1 comprising the sequence of SEQ ID NO: 102, a LCDR2 comprising the sequence of SEQ ID NO: 103, and a LCDR3 comprising the sequence of SEQ ID NO: 104;
(xiv) a VL comprising a LCDR1 comprising the sequence of SEQ ID NO: 110, a LCDR2 comprising the sequence of SEQ ID NO: 111, and a LCDR3 comprising the sequence of SEQ ID NO: 112;
(xv) a VL comprising a LCDR1 comprising the sequence of SEQ ID NO: 118, a LCDR2 comprising the sequence of SEQ ID NO: 119, and a LCDR3 comprising the sequence of SEQ ID NO: 120; and
(xvi) a VL comprising a LCDR1 comprising the sequence of SEQ ID NO: 126, a LCDR2 comprising the sequence of SEQ ID NO: 127, and a LCDR3 comprising the sequence of SEQ ID NO: 128.

In one embodiment, the first binding domain comprises a VH and a VL selected from the group consisting of:
(i) a VH comprising a HCDR1 comprising the sequence of SEQ ID NO: 2, a HCDR2 comprising the sequence of SEQ ID NO: 3, and a HCDR3 comprising the sequence of SEQ ID NO: 4 and a VL comprising a LCDR1 comprising the sequence of SEQ ID NO: 6, a LCDR2 comprising the sequence of SEQ ID NO: 7, and a LCDR3 comprising the sequence of SEQ ID NO: 8;
(ii) a VH comprising a HCDR1 comprising the sequence of SEQ ID NO: 10, a HCDR2 comprising the sequence of SEQ ID NO: 11, and a HCDR3 comprising the sequence of SEQ ID NO: 12 and a VL comprising a LCDR1 comprising the sequence of SEQ ID NO: 14, a LCDR2 comprising the sequence of SEQ ID NO: 15, and a LCDR3 comprising the sequence of SEQ ID NO: 16;
(iii) a VH comprising a HCDR1 comprising the sequence of SEQ ID NO: 18, a HCDR2 comprising the sequence of SEQ ID NO: 19, and a HCDR3 comprising the sequence of SEQ ID NO: 20 and a VL comprising a LCDR1 comprising the sequence of SEQ ID NO: 22, a LCDR2 comprising the sequence of SEQ ID NO: 23, and a LCDR3 comprising the sequence of SEQ ID NO: 24;
(iv) a VH comprising a HCDR1 comprising the sequence of SEQ ID NO: 26, a HCDR2 comprising the sequence of SEQ ID NO: 27, and a HCDR3 comprising the sequence of SEQ ID NO: 28 and a VL comprising a LCDR1 comprising the sequence of SEQ ID NO: 30, a LCDR2 comprising the sequence of SEQ ID NO: 31, and a LCDR3 comprising the sequence of SEQ ID NO: 32;
(v) a VH comprising a HCDR1 comprising the sequence of SEQ ID NO: 34, a HCDR2 comprising the sequence of SEQ ID NO: 35, and a HCDR3 comprising the sequence of SEQ ID NO: 36 and a VL comprising a LCDR1 comprising the sequence of SEQ ID NO: 38, a LCDR2 comprising the sequence of SEQ ID NO: 39, and a LCDR3 comprising the sequence of SEQ ID NO: 40;
(vi) a VH comprising a HCDR1 comprising the sequence of SEQ ID NO: 42, a HCDR2 comprising the sequence of SEQ ID NO: 43, and a HCDR3 comprising the sequence of SEQ ID NO: 44 and a VL comprising a LCDR1 comprising the sequence of SEQ ID NO: 46, a LCDR2 comprising the sequence of SEQ ID NO: 47, and a LCDR3 comprising the sequence of SEQ ID NO: 48;
(vii) a VH comprising a HCDR1 comprising the sequence of SEQ ID NO: 50, a HCDR2 comprising the sequence of SEQ ID NO: 51, and a HCDR3 comprising the sequence of SEQ ID NO: 52 and a VL comprising a LCDR1 comprising the sequence of SEQ ID NO: 54, a LCDR2 comprising the sequence of SEQ ID NO: 55, and a LCDR3 comprising the sequence of SEQ ID NO: 56;
(viii) a VH comprising a HCDR1 comprising the sequence of SEQ ID NO: 58, a HCDR2 comprising the sequence of SEQ ID NO: 59, and a HCDR3 comprising the sequence of SEQ ID NO: 60 and a VL comprising a LCDR1 comprising the sequence of SEQ ID NO: 62, a LCDR2 comprising the sequence of SEQ ID NO: 63, and a LCDR3 comprising the sequence of SEQ ID NO: 64;
(ix) a VH comprising a HCDR1 comprising the sequence of SEQ ID NO: 66, a HCDR2 comprising the sequence of SEQ ID NO: 67, and a HCDR3 comprising the sequence of SEQ ID NO: 68 and a VL comprising a LCDR1 comprising the sequence of SEQ ID NO: 70, a LCDR2 comprising the sequence of SEQ ID NO: 71, and a LCDR3 comprising the sequence of SEQ ID NO: 72;
(x) a VH comprising a HCDR1 comprising the sequence of SEQ ID NO: 74, a HCDR2 comprising the sequence of SEQ ID NO: 75, and a HCDR3 comprising the sequence of SEQ ID NO: 76 and a VL comprising a LCDR1 comprising the sequence of SEQ ID NO: 78, a LCDR2 comprising the sequence of SEQ ID NO: 79, and a LCDR3 comprising the sequence of SEQ ID NO: 80;
(xi) a VH comprising a HCDR1 comprising the sequence of SEQ ID NO: 82, a HCDR2 comprising the sequence of SEQ ID NO: 83, and a HCDR3 comprising the sequence of SEQ ID NO: 84 and a VL comprising a LCDR1 comprising the sequence of SEQ ID NO: 86, a LCDR2 comprising the sequence of SEQ ID NO: 87, and a LCDR3 comprising the sequence of SEQ ID NO: 88;
(xii) a VH comprising a HCDR1 comprising the sequence of SEQ ID NO: 90, a HCDR2 comprising the sequence of SEQ ID NO: 91, and a HCDR3 comprising the sequence of SEQ ID NO: 92 and a VL comprising a LCDR1 comprising the sequence of SEQ ID NO: 94, a LCDR2 comprising the sequence of SEQ ID NO: 95, and a LCDR3 comprising the sequence of SEQ ID NO: 96;
(xiii) a VH comprising a HCDR1 comprising the sequence of SEQ ID NO: 98, a HCDR2 comprising the sequence of SEQ ID NO: 99, and a HCDR3 comprising the sequence of SEQ ID NO: 100 and a VL comprising a LCDR1 comprising the sequence of SEQ ID NO: 102, a LCDR2 comprising the sequence of SEQ ID NO: 103, and a LCDR3 comprising the sequence of SEQ ID NO: 104;
(xiv) a VH comprising a HCDR1 comprising the sequence of SEQ ID NO: 106, a HCDR2 comprising the sequence of SEQ ID NO: 107, and a HCDR3 comprising the sequence of SEQ ID NO: 108 and a VL comprising a LCDR1 comprising the sequence of SEQ ID NO: 110, a LCDR2 comprising the sequence of SEQ ID NO: 111, and a LCDR3 comprising the sequence of SEQ ID NO: 112;
(xv) a VH comprising a HCDR1 comprising the sequence of SEQ ID NO: 114, a HCDR2 comprising the sequence of SEQ ID NO: 115, and a HCDR3 comprising the sequence of SEQ ID NO: 116 and a VL comprising a LCDR1 comprising the sequence of SEQ ID NO: 118, a LCDR2 comprising the sequence of SEQ ID NO: 119, and a LCDR3 comprising the sequence of SEQ ID NO: 120; and
(xvi) a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128.

In one embodiment, the first binding domain comprises a VH comprising a sequence having at least 70%, at least 75%, at least 80%, at least 85%, at least 90%, at least 95%, at least 97%, at least 99%, or 100% identity to a sequence selected from the group consisting of SEQ ID NO: 1, 9, 17, 25, 33, 41, 49, 57, 65, 73, 81, 89, 97, 105, 113, and 121.

In one embodiment, the first binding domain comprises a VL comprising a sequence having at least 70%, at least 75%, at least 80%, at least 85%, at least 90%, at least 95%, at least 97%, at least 99%, or 100% identity to a sequence selected from the group consisting of SEQ ID NO: 5, 13, 21, 29, 37, 45, 53, 61, 69, 77, 85, 93, 101, 109, 117, and 125.

In one embodiment, the first binding domain comprises a VH and a VL selected from the group consisting of:
(i) a VH comprising a sequence having at least 70%, at least 75%, at least 80%, at least 85%, at least 90%, at least 95%, at least 97%, at least 99%, or 100% identity to the sequence of SEQ ID NO: 1 and a VL comprising a sequence having at least 70%, at least 75%, at least 80%, at least 85%, at least 90%, at least 95%, at least 97%, at least 99%, or 100% identity to the sequence of SEQ ID NO: 5;
(ii) a VH comprising a sequence having at least 70%, at least 75%, at least 80%, at least 85%, at least 90%, at least 95%, at least 97%, at least 99%, or 100% identity to the sequence of SEQ ID NO: 9 and a VL comprising a sequence having at least 70%, at least 75%, at least 80%, at least 85%, at least 90%, at least 95%, at least 97%, at least 99%, or 100% identity to the sequence of SEQ ID NO: 13;
(iii) a VH comprising a sequence having at least 70%, at least 75%, at least 80%, at least 85%, at least 90%, at least 95%, at least 97%, at least 99%, or 100% identity to the sequence of SEQ ID NO: 17 and a VL comprising a sequence having at least 70%, at least 75%, at least 80%, at least 85%, at least 90%, at least 95%, at least 97%, at least 99%, or 100% identity to the sequence of SEQ ID NO: 21;
(iv) a VH comprising a sequence having at least 70%, at least 75%, at least 80%, at least 85%, at least 90%, at least 95%, at least 97%, at least 99%, or 100% identity to the sequence of SEQ ID NO: 25 and a VL comprising a sequence having at least 70%, at least 75%, at least 80%, at least 85%, at least 90%, at least 95%, at least 97%, at least 99%, or 100% identity to the sequence of SEQ ID NO: 29;
(v) a VH comprising a sequence having at least 70%, at least 75%, at least 80%, at least 85%, at least 90%, at least 95%, at least 97%, at least 99%, or 100% identity to the sequence of SEQ ID NO: 33 and a VL comprising a sequence having at least 70%, at least 75%, at least 80%, at least 85%, at least 90%, at least 95%, at least 97%, at least 99%, or 100% identity to the sequence of SEQ ID NO: 37;
(vi) a VH comprising a sequence having at least 70%, at least 75%, at least 80%, at least 85%, at least 90%, at least 95%, at least 97%, at least 99%, or 100% identity to the sequence of SEQ ID NO: 41 and a VL comprising a sequence having at least 70%, at least 75%, at least 80%, at least 85%, at least 90%, at least 95%, at least 97%, at least 99%, or 100% identity to the sequence of SEQ ID NO: 45;
(vii) a VH comprising a sequence having at least 70%, at least 75%, at least 80%, at least 85%, at least 90%, at least 95%, at least 97%, at least 99%, or 100% identity to the sequence of SEQ ID NO: 49 and a VL comprising a sequence having at least 70%, at least 75%, at least 80%, at least 85%, at least 90%, at least 95%, at least 97%, at least 99%, or 100% identity to the sequence of SEQ ID NO: 53;
(viii) a VH comprising a sequence having at least 70%, at least 75%, at least 80%, at least 85%, at least 90%, at least 95%, at least 97%, at least 99%, or 100% identity to the sequence of SEQ ID NO: 57 and a VL comprising a sequence having at least 70%, at least 75%, at least 80%, at least 85%, at least 90%, at least 95%, at least 97%, at least 99%, or 100% identity to the sequence of SEQ ID NO: 61;
(ix) a VH comprising a sequence having at least 70%, at least 75%, at least 80%, at least 85%, at least 90%, at least 95%, at least 97%, at least 99%, or 100% identity to the sequence of SEQ ID NO: 65 and a VL comprising a sequence having at least 70%, at least 75%, at least 80%, at least 85%, at least 90%, at least 95%, at least 97%, at least 99%, or 100% identity to the sequence of SEQ ID NO: 69;
(x) a VH comprising a sequence having at least 70%, at least 75%, at least 80%, at least 85%, at least 90%, at least 95%, at least 97%, at least 99%, or 100% identity to the sequence of SEQ ID NO: 73 and a VL comprising a sequence having at least 70%, at least 75%, at least 80%, at least 85%, at least 90%, at least 95%, at least 97%, at least 99%, or 100% identity to the sequence of SEQ ID NO: 77;
(xi) a VH comprising a sequence having at least 70%, at least 75%, at least 80%, at least 85%, at least 90%, at least 95%, at least 97%, at least 99%, or 100% identity to the sequence of SEQ ID NO: 81 and a VL comprising a sequence having at least 70%, at least 75%, at least 80%, at least 85%, at least 90%, at least 95%, at least 97%, at least 99%, or 100% identity to the sequence of SEQ ID NO: 85;
(xii) a VH comprising a sequence having at least 70%, at least 75%, at least 80%, at least 85%, at least 90%, at least 95%, at least 97%, at least 99%, or 100% identity to the sequence of SEQ ID NO: 89 and a VL comprising a sequence having at least 70%, at least 75%, at least 80%, at least 85%, at least 90%, at least 95%, at least 97%, at least 99%, or 100% identity to the sequence of SEQ ID NO: 93;
(xiii) a VH comprising a sequence having at least 70%, at least 75%, at least 80%, at least 85%, at least 90%, at least 95%, at least 97%, at least 99%, or 100% identity to the sequence of SEQ ID NO: 97 and a VL comprising a sequence having at least 70%, at least 75%, at least 80%, at least 85%, at least 90%, at least 95%, at least 97%, at least 99%, or 100% identity to the sequence of SEQ ID NO: 101;
(xiv) a VH comprising a sequence having at least 70%, at least 75%, at least 80%, at least 85%, at least 90%, at least 95%, at least 97%, at least 99%, or 100% identity to the sequence of SEQ ID NO: 105 and a VL comprising a sequence having at least 70%, at least 75%, at least 80%, at least 85%, at least 90%, at least 95%, at least 97%, at least 99%, or 100% identity to the sequence of SEQ ID NO: 109;
(xv) a VH comprising a sequence having at least 70%, at least 75%, at least 80%, at least 85%, at least 90%, at least 95%, at least 97%, at least 99%, or 100% identity to the sequence of SEQ ID NO: 113 and a VL comprising a sequence having at least 70%, at least 75%, at least 80%, at least 85%, at least 90%, at least 95%, at least 97%, at least 99%, or 100% identity to the sequence of SEQ ID NO: 117; and
(xvi) a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125.

In one embodiment, the first binding domain comprises a VH and a VL of an antibody which competes for coronavirus S protein binding with and/or has the specificity for coronavirus S protein of an antibody comprising a VH or a VL, or a combination thereof as set forth above.

In one embodiment, the second binding domain comprises an extracellular domain (ECD) of ACE2 protein or a variant thereof, or a fragment of the ECD of ACE2 protein or the variant thereof. In one embodiment, the variant of the ECD of ACE2 protein or the fragment of the ECD of ACE2 protein or the variant thereof binds to the coronavirus S protein. In one embodiment, the second binding domain comprises a sequence having at least 70%, at least 75%, at least 80%, at least 85%, at least 90%, at least 95%, at least 97%, at least 99%, or 100% identity to the sequence of SEQ ID NO: 129.

In one embodiment, the second binding domain comprises a heavy chain variable region (VH). In one embodiment, the VH of the second binding domain comprises a HCDR3 comprising a sequence selected from the group consisting of SEQ ID NO: 4, 12, 20, 28, 36, 44, 52, 60, 68, 76, 84, 92, 100, 108, 116, and 124. In one embodiment, the VH of the second binding domain comprises a HCDR2 comprising a sequence selected from the group consisting of SEQ ID NO: 3, 11, 19, 27, 35, 43, 51, 59, 67, 75, 83, 91, 99, 107, 115, and 123. In one embodiment, the VH of the second binding domain comprises a HCDR1 comprising a sequence selected from the group consisting of SEQ ID NO: 2, 10, 18, 26, 34, 42, 50, 58, 66, 74, 82, 90, 98, 106, 114, and 122. In one embodiment, the VH of the second binding domain is selected from the group consisting of:
(i) a VH comprising a HCDR1 comprising the sequence of SEQ ID NO: 2, a HCDR2 comprising the sequence of SEQ ID NO: 3, and a HCDR3 comprising the sequence of SEQ ID NO: 4;
(ii) a VH comprising a HCDR1 comprising the sequence of SEQ ID NO: 10, a HCDR2 comprising the sequence of SEQ ID NO: 11, and a HCDR3 comprising the sequence of SEQ ID NO: 12;
(iii) a VH comprising a HCDR1 comprising the sequence of SEQ ID NO: 18, a HCDR2 comprising the sequence of SEQ ID NO: 19, and a HCDR3 comprising the sequence of SEQ ID NO: 20;
(iv) a VH comprising a HCDR1 comprising the sequence of SEQ ID NO: 26, a HCDR2 comprising the sequence of SEQ ID NO: 27, and a HCDR3 comprising the sequence of SEQ ID NO: 28;
(v) a VH comprising a HCDR1 comprising the sequence of SEQ ID NO: 34, a HCDR2 comprising the sequence of SEQ ID NO: 35, and a HCDR3 comprising the sequence of SEQ ID NO: 36;
(vi) a VH comprising a HCDR1 comprising the sequence of SEQ ID NO: 42, a HCDR2 comprising the sequence of SEQ ID NO: 43, and a HCDR3 comprising the sequence of SEQ ID NO: 44;
(vii) a VH comprising a HCDR1 comprising the sequence of SEQ ID NO: 50, a HCDR2 comprising the sequence of SEQ ID NO: 51, and a HCDR3 comprising the sequence of SEQ ID NO: 52;
(viii) a VH comprising a HCDR1 comprising the sequence of SEQ ID NO: 58, a HCDR2 comprising the sequence of SEQ ID NO: 59, and a HCDR3 comprising the sequence of SEQ ID NO: 60;
(ix) a VH comprising a HCDR1 comprising the sequence of SEQ ID NO: 66, a HCDR2 comprising the sequence of SEQ ID NO: 67, and a HCDR3 comprising the sequence of SEQ ID NO: 68;
(x) a VH comprising a HCDR1 comprising the sequence of SEQ ID NO: 74, a HCDR2 comprising the sequence of SEQ ID NO: 75, and a HCDR3 comprising the sequence of SEQ ID NO: 76;
(xi) a VH comprising a HCDR1 comprising the sequence of SEQ ID NO: 82, a HCDR2 comprising the sequence of SEQ ID NO: 83, and a HCDR3 comprising the sequence of SEQ ID NO: 84;
(xii) a VH comprising a HCDR1 comprising the sequence of SEQ ID NO: 90, a HCDR2 comprising the sequence of SEQ ID NO: 91, and a HCDR3 comprising the sequence of SEQ ID NO: 92;
(xiii) a VH comprising a HCDR1 comprising the sequence of SEQ ID NO: 98, a HCDR2 comprising the sequence of SEQ ID NO: 99, and a HCDR3 comprising the sequence of SEQ ID NO: 100;
(xiv) a VH comprising a HCDR1 comprising the sequence of SEQ ID NO: 106, a HCDR2 comprising the sequence of SEQ ID NO: 107, and a HCDR3 comprising the sequence of SEQ ID NO: 108;
(xv) a VH comprising a HCDR1 comprising the sequence of SEQ ID NO: 114, a HCDR2 comprising the sequence of SEQ ID NO: 115, and a HCDR3 comprising the sequence of SEQ ID NO: 116; and
(xvi) a VH comprising a HCDR1 comprising the sequence of SEQ ID NO: 122, a HCDR2 comprising the sequence of SEQ ID NO: 123, and a HCDR3 comprising the sequence of SEQ ID NO: 124.

In one embodiment, the second binding domain comprises a light chain variable region (VL). In one embodiment, the VL of the second binding domain comprises a LCDR3 comprising a sequence selected from the group consisting of SEQ ID NO: 8, 16, 24, 32, 40, 48, 56, 64, 72, 80, 88, 96, 104, 112, 120, and 128. In one embodiment, the VL of the second binding domain comprises a LCDR2 comprising a sequence selected from the group consisting of SEQ ID NO: 7, 15, 23, 31, 39, 47, 55, 63, 71, 79, 87, 95, 103, 111, 119, and 127. In one embodiment, the VL of the second binding domain comprises a LCDR1 comprising a sequence selected from the group consisting of SEQ ID NO: 6, 14, 22, 30, 38, 46, 54, 62, 70, 78, 86, 94, 102, 110, 118, and 126. In one embodiment, the VL of the second binding domain is selected from the group consisting of:
(i) a VL comprising a LCDR1 comprising the sequence of SEQ ID NO: 6, a LCDR2 comprising the sequence of SEQ ID NO: 7, and a LCDR3 comprising the sequence of SEQ ID NO: 8;
(ii) a VL comprising a LCDR1 comprising the sequence of SEQ ID NO: 14, a LCDR2 comprising the sequence of SEQ ID NO: 15, and a LCDR3 comprising the sequence of SEQ ID NO: 16;
(iii) a VL comprising a LCDR1 comprising the sequence of SEQ ID NO: 22, a LCDR2 comprising the sequence of SEQ ID NO: 23, and a LCDR3 comprising the sequence of SEQ ID NO: 24;
(iv) a VL comprising a LCDR1 comprising the sequence of SEQ ID NO: 30, a LCDR2 comprising the sequence of SEQ ID NO: 31, and a LCDR3 comprising the sequence of SEQ ID NO: 32;
(v) a VL comprising a LCDR1 comprising the sequence of SEQ ID NO: 38, a LCDR2 comprising the sequence of SEQ ID NO: 39, and a LCDR3 comprising the sequence of SEQ ID NO: 40;
(vi) a VL comprising a LCDR1 comprising the sequence of SEQ ID NO: 46, a LCDR2 comprising the sequence of SEQ ID NO: 47, and a LCDR3 comprising the sequence of SEQ ID NO: 48;
(vii) a VL comprising a LCDR1 comprising the sequence of SEQ ID NO: 54, a LCDR2 comprising the sequence of SEQ ID NO: 55, and a LCDR3 comprising the sequence of SEQ ID NO: 56;
(viii) a VL comprising a LCDR1 comprising the sequence of SEQ ID NO: 62, a LCDR2 comprising the sequence of SEQ ID NO: 63, and a LCDR3 comprising the sequence of SEQ ID NO: 64;
(ix) a VL comprising a LCDR1 comprising the sequence of SEQ ID NO: 70, a LCDR2 comprising the sequence of SEQ ID NO: 71, and a LCDR3 comprising the sequence of SEQ ID NO: 72;
(x) a VL comprising a LCDR1 comprising the sequence of SEQ ID NO: 78, a LCDR2 comprising the sequence of SEQ ID NO: 79, and a LCDR3 comprising the sequence of SEQ ID NO: 80;
(xi) a VL comprising a LCDR1 comprising the sequence of SEQ ID NO: 86, a LCDR2 comprising the sequence of SEQ ID NO: 87, and a LCDR3 comprising the sequence of SEQ ID NO: 88;
(xii) a VL comprising a LCDR1 comprising the sequence of SEQ ID NO: 94, a LCDR2 comprising the sequence of SEQ ID NO: 95, and a LCDR3 comprising the sequence of SEQ ID NO: 96;
(xiii) a VL comprising a LCDR1 comprising the sequence of SEQ ID NO: 102, a LCDR2 comprising the sequence of SEQ ID NO: 103, and a LCDR3 comprising the sequence of SEQ ID NO: 104;
(xiv) a VL comprising a LCDR1 comprising the sequence of SEQ ID NO: 110, a LCDR2 comprising the sequence of SEQ ID NO: 111, and a LCDR3 comprising the sequence of SEQ ID NO: 112;
(xv) a VL comprising a LCDR1 comprising the sequence of SEQ ID NO: 118, a LCDR2 comprising the sequence of SEQ ID NO: 119, and a LCDR3 comprising the sequence of SEQ ID NO: 120; and
(xvi) a VL comprising a LCDR1 comprising the sequence of SEQ ID NO: 126, a LCDR2 comprising the sequence of SEQ ID NO: 127, and a LCDR3 comprising the sequence of SEQ ID NO: 128.

In one embodiment, the second binding domain comprises a VH and a VL selected from the group consisting of:
(i) a VH comprising a HCDR1 comprising the sequence of SEQ ID NO: 2, a HCDR2 comprising the sequence of SEQ ID NO: 3, and a HCDR3 comprising the sequence of SEQ ID NO: 4 and a VL comprising a LCDR1 comprising the sequence of SEQ ID NO: 6, a LCDR2 comprising the sequence of SEQ ID NO: 7, and a LCDR3 comprising the sequence of SEQ ID NO: 8;
(ii) a VH comprising a HCDR1 comprising the sequence of SEQ ID NO: 10, a HCDR2 comprising the sequence of SEQ ID NO: 11, and a HCDR3 comprising the sequence of SEQ ID NO: 12 and a VL comprising a LCDR1 comprising the sequence of SEQ ID NO: 14, a LCDR2 comprising the sequence of SEQ ID NO: 15, and a LCDR3 comprising the sequence of SEQ ID NO: 16;
(iii) a VH comprising a HCDR1 comprising the sequence of SEQ ID NO: 18, a HCDR2 comprising the sequence of SEQ ID NO: 19, and a HCDR3 comprising the sequence of SEQ ID NO: 20 and a VL comprising a LCDR1 comprising the sequence of SEQ ID NO: 22, a LCDR2 comprising the sequence of SEQ ID NO: 23, and a LCDR3 comprising the sequence of SEQ ID NO: 24;
(iv) a VH comprising a HCDR1 comprising the sequence of SEQ ID NO: 26, a HCDR2 comprising the sequence of SEQ ID NO: 27, and a HCDR3 comprising the sequence of SEQ ID NO: 28 and a VL comprising a LCDR1 comprising the sequence of SEQ ID NO: 30, a LCDR2 comprising the sequence of SEQ ID NO: 31, and a LCDR3 comprising the sequence of SEQ ID NO: 32;
(v) a VH comprising a HCDR1 comprising the sequence of SEQ ID NO: 34, a HCDR2 comprising the sequence of SEQ ID NO: 35, and a HCDR3 comprising the sequence of SEQ ID NO: 36 and a VL comprising a LCDR1 comprising the sequence of SEQ ID NO: 38, a LCDR2 comprising the sequence of SEQ ID NO: 39, and a LCDR3 comprising the sequence of SEQ ID NO: 40;
(vi) a VH comprising a HCDR1 comprising the sequence of SEQ ID NO: 42, a HCDR2 comprising the sequence of SEQ ID NO: 43, and a HCDR3 comprising the sequence of SEQ ID NO: 44 and a VL comprising a LCDR1 comprising the sequence of SEQ ID NO: 46, a LCDR2 comprising the sequence of SEQ ID NO: 47, and a LCDR3 comprising the sequence of SEQ ID NO: 48;
(vii) a VH comprising a HCDR1 comprising the sequence of SEQ ID NO: 50, a HCDR2 comprising the sequence of SEQ ID NO: 51, and a HCDR3 comprising the sequence of SEQ ID NO: 52 and a VL comprising a LCDR1 comprising the sequence of SEQ ID NO: 54, a LCDR2 comprising the sequence of SEQ ID NO: 55, and a LCDR3 comprising the sequence of SEQ ID NO: 56;
(viii) a VH comprising a HCDR1 comprising the sequence of SEQ ID NO: 58, a HCDR2 comprising the sequence of SEQ ID NO: 59, and a HCDR3 comprising the sequence of SEQ ID NO: 60 and a VL comprising a LCDR1 comprising the sequence of SEQ ID NO: 62, a LCDR2 comprising the sequence of SEQ ID NO: 63, and a LCDR3 comprising the sequence of SEQ ID NO: 64;
(ix) a VH comprising a HCDR1 comprising the sequence of SEQ ID NO: 66, a HCDR2 comprising the sequence of SEQ ID NO: 67, and a HCDR3 comprising the sequence of SEQ ID NO: 68 and a VL comprising a LCDR1 comprising the sequence of SEQ ID NO: 70, a LCDR2 comprising the sequence of SEQ ID NO: 71, and a LCDR3 comprising the sequence of SEQ ID NO: 72;
(x) a VH comprising a HCDR1 comprising the sequence of SEQ ID NO: 74, a HCDR2 comprising the sequence of SEQ ID NO: 75, and a HCDR3 comprising the sequence of SEQ ID NO: 76 and a VL comprising a LCDR1 comprising the sequence of SEQ ID NO: 78, a LCDR2 comprising the sequence of SEQ ID NO: 79, and a LCDR3 comprising the sequence of SEQ ID NO: 80;
(xi) a VH comprising a HCDR1 comprising the sequence of SEQ ID NO: 82, a HCDR2 comprising the sequence of SEQ ID NO: 83, and a HCDR3 comprising the sequence of SEQ ID NO: 84 and a VL comprising a LCDR1 comprising the sequence of SEQ ID NO: 86, a LCDR2 comprising the sequence of SEQ ID NO: 87, and a LCDR3 comprising the sequence of SEQ ID NO: 88;
(xii) a VH comprising a HCDR1 comprising the sequence of SEQ ID NO: 90, a HCDR2 comprising the sequence of SEQ ID NO: 91, and a HCDR3 comprising the sequence of SEQ ID NO: 92 and a VL comprising a LCDR1 comprising the sequence of SEQ ID NO: 94, a LCDR2 comprising the sequence of SEQ ID NO: 95, and a LCDR3 comprising the sequence of SEQ ID NO: 96;
(xiii) a VH comprising a HCDR1 comprising the sequence of SEQ ID NO: 98, a HCDR2 comprising the sequence of SEQ ID NO: 99, and a HCDR3 comprising the sequence of SEQ ID NO: 100 and a VL comprising a LCDR1 comprising the sequence of SEQ ID NO: 102, a LCDR2 comprising the sequence of SEQ ID NO: 103, and a LCDR3 comprising the sequence of SEQ ID NO: 104;
(xiv) a VH comprising a HCDR1 comprising the sequence of SEQ ID NO: 106, a HCDR2 comprising the sequence of SEQ ID NO: 107, and a HCDR3 comprising the sequence of SEQ ID NO: 108 and a VL comprising a LCDR1 comprising the sequence of SEQ ID NO: 110, a LCDR2 comprising the sequence of SEQ ID NO: 111, and a LCDR3 comprising the sequence of SEQ ID NO: 112;
(xv) a VH comprising a HCDR1 comprising the sequence of SEQ ID NO: 114, a HCDR2 comprising the sequence of SEQ ID NO: 115, and a HCDR3 comprising the sequence of SEQ ID NO: 116 and a VL comprising a LCDR1 comprising the sequence of SEQ ID NO: 118, a LCDR2 comprising the sequence of SEQ ID NO: 119, and a LCDR3 comprising the sequence of SEQ ID NO: 120; and
(xvi) a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128.

In one embodiment, the second binding domain comprises a VH comprising a sequence having at least 70%, at least 75%, at least 80%, at least 85%, at least 90%, at least 95%, at least 97%, at least 99%, or 100% identity to a sequence selected from the group consisting of SEQ ID NO: 1, 9, 17, 25, 33, 41, 49, 57, 65, 73, 81, 89, 97, 105, 113, and 121.

In one embodiment, the second binding domain comprises a VL comprising a sequence having at least 70%, at least 75%, at least 80%, at least 85%, at least 90%, at least 95%, at least 97%, at least 99%, or 100% identity to a sequence selected from the group consisting of SEQ ID NO: 5, 13, 21, 29, 37, 45, 53, 61, 69, 77, 85, 93, 101, 109, 117, and 125.

In one embodiment, the second binding domain comprises a VH and a VL selected from the group consisting of:
(i) a VH comprising a sequence having at least 70%, at least 75%, at least 80%, at least 85%, at least 90%, at least 95%, at least 97%, at least 99%, or 100% identity to the sequence of SEQ ID NO: 1 and a VL comprising a sequence having at least 70%, at least 75%, at least 80%, at least 85%, at least 90%, at least 95%, at least 97%, at least 99%, or 100% identity to the sequence of SEQ ID NO: 5;
(ii) a VH comprising a sequence having at least 70%, at least 75%, at least 80%, at least 85%, at least 90%, at least 95%, at least 97%, at least 99%, or 100% identity to the sequence of SEQ ID NO: 9 and a VL comprising a sequence having at least 70%, at least 75%, at least 80%, at least 85%, at least 90%, at least 95%, at least 97%, at least 99%, or 100% identity to the sequence of SEQ ID NO: 13;
(iii) a VH comprising a sequence having at least 70%, at least 75%, at least 80%, at least 85%, at least 90%, at least 95%, at least 97%, at least 99%, or 100% identity to the sequence of SEQ ID NO: 17 and a VL comprising a sequence having at least 70%, at least 75%, at least 80%, at least 85%, at least 90%, at least 95%, at least 97%, at least 99%, or 100% identity to the sequence of SEQ ID NO: 21;
(iv) a VH comprising a sequence having at least 70%, at least 75%, at least 80%, at least 85%, at least 90%, at least 95%, at least 97%, at least 99%, or 100% identity to the sequence of SEQ ID NO: 25 and a VL comprising a sequence having at least 70%, at least 75%, at least 80%, at least 85%, at least 90%, at least 95%, at least 97%, at least 99%, or 100% identity to the sequence of SEQ ID NO: 29;
(v) a VH comprising a sequence having at least 70%, at least 75%, at least 80%, at least 85%, at least 90%, at least 95%, at least 97%, at least 99%, or 100% identity to the sequence of SEQ ID NO: 33 and a VL comprising a sequence having at least 70%, at least 75%, at least 80%, at least 85%, at least 90%, at least 95%, at least 97%, at least 99%, or 100% identity to the sequence of SEQ ID NO: 37;
(vi) a VH comprising a sequence having at least 70%, at least 75%, at least 80%, at least 85%, at least 90%, at least 95%, at least 97%, at least 99%, or 100% identity to the sequence of SEQ ID NO: 41 and a VL comprising a sequence having at least 70%, at least 75%, at least 80%, at least 85%, at least 90%, at least 95%, at least 97%, at least 99%, or 100% identity to the sequence of SEQ ID NO: 45;
(vii) a VH comprising a sequence having at least 70%, at least 75%, at least 80%, at least 85%, at least 90%, at least 95%, at least 97%, at least 99%, or 100% identity to the sequence of SEQ ID NO: 49 and a VL comprising a sequence having at least 70%, at least 75%, at least 80%, at least 85%, at least 90%, at least 95%, at least 97%, at least 99%, or 100% identity to the sequence of SEQ ID NO: 53;
(viii) a VH comprising a sequence having at least 70%, at least 75%, at least 80%, at least 85%, at least 90%, at least 95%, at least 97%, at least 99%, or 100% identity to the sequence of SEQ ID NO: 57 and a VL comprising a sequence having at least 70%, at least 75%, at least 80%, at least 85%, at least 90%, at least 95%, at least 97%, at least 99%, or 100% identity to the sequence of SEQ ID NO: 61;
(ix) a VH comprising a sequence having at least 70%, at least 75%, at least 80%, at least 85%, at least 90%, at least 95%, at least 97%, at least 99%, or 100% identity to the sequence of SEQ ID NO: 65 and a VL comprising a sequence having at least 70%, at least 75%, at least 80%, at least 85%, at least 90%, at least 95%, at least 97%, at least 99%, or 100% identity to the sequence of SEQ ID NO: 69;
(x) a VH comprising a sequence having at least 70%, at least 75%, at least 80%, at least 85%, at least 90%, at least 95%, at least 97%, at least 99%, or 100% identity to the sequence of SEQ ID NO: 73 and a VL comprising a sequence having at least 70%, at least 75%, at least 80%, at least 85%, at least 90%, at least 95%, at least 97%, at least 99%, or 100% identity to the sequence of SEQ ID NO: 77;
(xi) a VH comprising a sequence having at least 70%, at least 75%, at least 80%, at least 85%, at least 90%, at least 95%, at least 97%, at least 99%, or 100% identity to the sequence of SEQ ID NO: 81 and a VL comprising a sequence having at least 70%, at least 75%, at least 80%, at least 85%, at least 90%, at least 95%, at least 97%, at least 99%, or 100% identity to the sequence of SEQ ID NO: 85;
(xii) a VH comprising a sequence having at least 70%, at least 75%, at least 80%, at least 85%, at least 90%, at least 95%, at least 97%, at least 99%, or 100% identity to the sequence of SEQ ID NO: 89 and a VL comprising a sequence having at least 70%, at least 75%, at least 80%, at least 85%, at least 90%, at least 95%, at least 97%, at least 99%, or 100% identity to the sequence of SEQ ID NO: 93;
(xiii) a VH comprising a sequence having at least 70%, at least 75%, at least 80%, at least 85%, at least 90%, at least 95%, at least 97%, at least 99%, or 100% identity to the sequence of SEQ ID NO: 97 and a VL comprising a sequence having at least 70%, at least 75%, at least 80%, at least 85%, at least 90%, at least 95%, at least 97%, at least 99%, or 100% identity to the sequence of SEQ ID NO: 101;
(xiv) a VH comprising a sequence having at least 70%, at least 75%, at least 80%, at least 85%, at least 90%, at least 95%, at least 97%, at least 99%, or 100% identity to the sequence of SEQ ID NO: 105 and a VL comprising a sequence having at least 70%, at least 75%, at least 80%, at least 85%, at least 90%, at least 95%, at least 97%, at least 99%, or 100% identity to the sequence of SEQ ID NO: 109;
(xv) a VH comprising a sequence having at least 70%, at least 75%, at least 80%, at least 85%, at least 90%, at least 95%, at least 97%, at least 99%, or 100% identity to the sequence of SEQ ID NO: 113 and a VL comprising a sequence having at least 70%, at least 75%, at least 80%, at least 85%, at least 90%, at least 95%, at least 97%, at least 99%, or 100% identity to the sequence of SEQ ID NO: 117; and
(xvi) a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125.

In one embodiment, the second binding domain comprises a VH and a VL of an antibody which competes for coronavirus S protein binding with and/or has the specificity for coronavirus S protein of an antibody comprising a VH or a VL, or a combination thereof as set forth above.

In one embodiment of the binding agent described herein:
(i) the first binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128,
and the second binding domain comprises a VH comprising a HCDR1 comprising the sequence of SEQ ID NO: 34, a HCDR2 comprising the sequence of SEQ ID NO: 35, and a HCDR3 comprising the sequence of SEQ ID NO: 36 and a VL comprising a LCDR1 comprising the sequence of SEQ ID NO: 38, a LCDR2 comprising the sequence of SEQ ID NO: 39, and a LCDR3 comprising the sequence of SEQ ID NO: 40;
(ii) the first binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128,
and the second binding domain comprises a VH comprising a HCDR1 comprising the sequence of SEQ ID NO: 2, a HCDR2 comprising the sequence of SEQ ID NO: 3, and a HCDR3 comprising the sequence of SEQ ID NO: 4 and a VL comprising a LCDR1 comprising the sequence of SEQ ID NO: 6, a LCDR2 comprising the sequence of SEQ ID NO: 7, and a LCDR3 comprising the sequence of SEQ ID NO: 8;
(iii) the first binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128,
and the second binding domain comprises a VH comprising a HCDR1 comprising the sequence of SEQ ID NO: 10, a HCDR2 comprising the sequence of SEQ ID NO: 11, and a HCDR3 comprising the sequence of SEQ ID NO: 12 and a VL comprising a LCDR1 comprising the sequence of SEQ ID NO: 14, a LCDR2 comprising the sequence of SEQ ID NO: 15, and a LCDR3 comprising the sequence of SEQ ID NO: 16;
(iv) the first binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128,
and the second binding domain comprises a VH comprising a HCDR1 comprising the sequence of SEQ ID NO: 26, a HCDR2 comprising the sequence of SEQ ID NO: 27, and a HCDR3 comprising the sequence of SEQ ID NO: 28 and a VL comprising a LCDR1 comprising the sequence of SEQ ID NO: 30, a LCDR2 comprising the sequence of SEQ ID NO: 31, and a LCDR3 comprising the sequence of SEQ ID NO: 32;
(v) the first binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128,
and the second binding domain comprises a VH comprising a HCDR1 comprising the sequence of SEQ ID NO: 42, a HCDR2 comprising the sequence of SEQ ID NO: 43, and a HCDR3 comprising the sequence of SEQ ID NO: 44 and a VL comprising a LCDR1 comprising the sequence of SEQ ID NO: 46, a LCDR2 comprising the sequence of SEQ ID NO: 47, and a LCDR3 comprising the sequence of SEQ ID NO: 48;
(vi) the first binding domain comprises a VH comprising a HCDR1 comprising the sequence of SEQ ID NO: 34, a HCDR2 comprising the sequence of SEQ ID NO: 35, and a HCDR3 comprising the sequence of SEQ ID NO: 36 and a VL comprising a LCDR1 comprising the sequence of SEQ ID NO: 38, a LCDR2 comprising the sequence of SEQ ID NO: 39, and a LCDR3 comprising the sequence of SEQ ID NO: 40,
and the second binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128;
(vii) the first binding domain comprises a VH comprising a HCDR1 comprising the sequence of SEQ ID NO: 26, a HCDR2 comprising the sequence of SEQ ID NO: 27, and a HCDR3 comprising the sequence of SEQ ID NO: 28 and a VL comprising a LCDR1 comprising the sequence of SEQ ID NO: 30, a LCDR2 comprising the sequence of SEQ ID NO: 31, and a LCDR3 comprising the sequence of SEQ ID NO: 32,
and the second binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128;
(viii) the first binding domain comprises a VH comprising a HCDR1 comprising the sequence of SEQ ID NO: 42, a HCDR2 comprising the sequence of SEQ ID NO: 43, and a HCDR3 comprising the sequence of SEQ ID NO: 44 and a VL comprising a LCDR1 comprising the sequence of SEQ ID NO: 46, a LCDR2 comprising the sequence of SEQ ID NO: 47, and a LCDR3 comprising the sequence of SEQ ID NO: 48,
and the second binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128;
(ix) the first binding domain comprises a VH comprising a HCDR1 comprising the sequence of SEQ ID NO: 2, a HCDR2 comprising the sequence of SEQ ID NO: 3, and a HCDR3 comprising the sequence of SEQ ID NO: 4 and a VL comprising a LCDR1 comprising the sequence of SEQ ID NO: 6, a LCDR2 comprising the sequence of SEQ ID NO: 7, and a LCDR3 comprising the sequence of SEQ ID NO: 8,
and the second binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128;
(x) the first binding domain comprises a VH comprising a HCDR1 comprising the sequence of SEQ ID NO: 10, a HCDR2 comprising the sequence of SEQ ID NO: 11, and a HCDR3 comprising the sequence of SEQ ID NO: 12 and a VL comprising a LCDR1 comprising the sequence of SEQ ID NO: 14, a LCDR2 comprising the sequence of SEQ ID NO: 15, and a LCDR3 comprising the sequence of SEQ ID NO: 16,
and the second binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128;
(xi) the first binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128,
and the second binding domain comprises a VH comprising a HCDR1 comprising the sequence of SEQ ID NO: 18, a HCDR2 comprising the sequence of SEQ ID NO: 19, and a HCDR3 comprising the sequence of SEQ ID NO: 20 and a VL comprising a LCDR1 comprising the sequence of SEQ ID NO: 22, a LCDR2 comprising the sequence of SEQ ID NO: 23, and a LCDR3 comprising the sequence of SEQ ID NO: 24;
(xii) the first binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128,
and the second binding domain comprises a VH comprising a HCDR1 comprising the sequence of SEQ ID NO: 50, a HCDR2 comprising the sequence of SEQ ID NO: 51, and a HCDR3 comprising the sequence of SEQ ID NO: 52 and a VL comprising a LCDR1 comprising the sequence of SEQ ID NO: 54, a LCDR2 comprising the sequence of SEQ ID NO: 55, and a LCDR3 comprising the sequence of SEQ ID NO: 56;
(xiii) the first binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128,
and the second binding domain comprises a VH comprising a HCDR1 comprising the sequence of SEQ ID NO: 106, a HCDR2 comprising the sequence of SEQ ID NO: 107, and a HCDR3 comprising the sequence of SEQ ID NO: 108 and a VL comprising a LCDR1 comprising the sequence of SEQ ID NO: 110, a LCDR2 comprising the sequence of SEQ ID NO: 111, and a LCDR3 comprising the sequence of SEQ ID NO: 112;
(xiv) the first binding domain comprises a VH comprising a HCDR1 comprising the sequence of SEQ ID NO: 18, a HCDR2 comprising the sequence of SEQ ID NO: 19, and a HCDR3 comprising the sequence of SEQ ID NO: 20 and a VL comprising a LCDR1 comprising the sequence of SEQ ID NO: 22, a LCDR2 comprising the sequence of SEQ ID NO: 23, and a LCDR3 comprising the sequence of SEQ ID NO: 24,
and the second binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128;
(xv) the first binding domain comprises a VH comprising a HCDR1 comprising the sequence of SEQ ID NO: 50, a HCDR2 comprising the sequence of SEQ ID NO: 51, and a HCDR3 comprising the sequence of SEQ ID NO: 52 and a VL comprising a LCDR1 comprising the sequence of SEQ ID NO: 54, a LCDR2 comprising the sequence of SEQ ID NO: 55, and a LCDR3 comprising the sequence of SEQ ID NO: 56,
and the second binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128;
(xvi) the first binding domain comprises a VH comprising a HCDR1 comprising the sequence of SEQ ID NO: 106, a HCDR2 comprising the sequence of SEQ ID NO: 107, and a HCDR3 comprising the sequence of SEQ ID NO: 108 and a VL comprising a LCDR1 comprising the sequence of SEQ ID NO: 110, a LCDR2 comprising the sequence of SEQ ID NO: 111, and a LCDR3 comprising the sequence of SEQ ID NO: 112,
and the second binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128;
(xvii) the first binding domain comprises a VH comprising a HCDR1 comprising the sequence of SEQ ID NO: 106, a HCDR2 comprising the sequence of SEQ ID NO: 107, and a HCDR3 comprising the sequence of SEQ ID NO: 108 and a VL comprising a LCDR1 comprising the sequence of SEQ ID NO: 110, a LCDR2 comprising the sequence of SEQ ID NO: 111, and a LCDR3 comprising the sequence of SEQ ID NO: 112,
and the second binding domain comprises a VH comprising a HCDR1 comprising the sequence of SEQ ID NO: 18, a HCDR2 comprising the sequence of SEQ ID NO: 19, and a HCDR3 comprising the sequence of SEQ ID NO: 20 and a VL comprising a LCDR1 comprising the sequence of SEQ ID NO: 22, a LCDR2 comprising the sequence of SEQ ID NO: 23, and a LCDR3 comprising the sequence of SEQ ID NO: 24;
(xviii) the first binding domain comprises a VH comprising a HCDR1 comprising the sequence of SEQ ID NO: 106, a HCDR2 comprising the sequence of SEQ ID NO: 107, and a HCDR3 comprising the sequence of SEQ ID NO: 108 and a VL comprising a LCDR1 comprising the sequence of SEQ ID NO: 110, a LCDR2 comprising the sequence of SEQ ID NO: 111, and a LCDR3 comprising the sequence of SEQ ID NO: 112,
and the second binding domain comprises a VH comprising a HCDR1 comprising the sequence of SEQ ID NO: 50, a HCDR2 comprising the sequence of SEQ ID NO: 51, and a HCDR3 comprising the sequence of SEQ ID NO: 52 and a VL comprising a LCDR1 comprising the sequence of SEQ ID NO: 54, a LCDR2 comprising the sequence of SEQ ID NO: 55, and a LCDR3 comprising the sequence of SEQ ID NO: 56;
(xix) the first binding domain comprises a VH comprising a HCDR1 comprising the sequence of SEQ ID NO: 106, a HCDR2 comprising the sequence of SEQ ID NO: 107, and a HCDR3 comprising the sequence of SEQ ID NO: 108 and a VL comprising a LCDR1 comprising the sequence of SEQ ID NO: 110, a LCDR2 comprising the sequence of SEQ ID NO: 111, and a LCDR3 comprising the sequence of SEQ ID NO: 112,
and the second binding domain comprises a VH comprising a HCDR1 comprising the sequence of SEQ ID NO: 42, a HCDR2 comprising the sequence of SEQ ID NO: 43, and a HCDR3 comprising the sequence of SEQ ID NO: 44 and a VL comprising a LCDR1 comprising the sequence of SEQ ID NO: 46, a LCDR2 comprising the sequence of SEQ ID NO: 47, and a LCDR3 comprising the sequence of SEQ ID NO: 48;
(xx) the first binding domain comprises a VH comprising a HCDR1 comprising the sequence of SEQ ID NO: 18, a HCDR2 comprising the sequence of SEQ ID NO: 19, and a HCDR3 comprising the sequence of SEQ ID NO: 20 and a VL comprising a LCDR1 comprising the sequence of SEQ ID NO: 22, a LCDR2 comprising the sequence of SEQ ID NO: 23, and a LCDR3 comprising the sequence of SEQ ID NO: 24,
and the second binding domain comprises a VH comprising a HCDR1 comprising the sequence of SEQ ID NO: 106, a HCDR2 comprising the sequence of SEQ ID NO: 107, and a HCDR3 comprising the sequence of SEQ ID NO: 108 and a VL comprising a LCDR1 comprising the sequence of SEQ ID NO: 110, a LCDR2 comprising the sequence of SEQ ID NO: 111, and a LCDR3 comprising the sequence of SEQ ID NO: 112;
(xxi) the first binding domain comprises a VH comprising a HCDR1 comprising the sequence of SEQ ID NO: 50, a HCDR2 comprising the sequence of SEQ ID NO: 51, and a HCDR3 comprising the sequence of SEQ ID NO: 52 and a VL comprising a LCDR1 comprising the sequence of SEQ ID NO: 54, a LCDR2 comprising the sequence of SEQ ID NO: 55, and a LCDR3 comprising the sequence of SEQ ID NO: 56,
and the second binding domain comprises a VH comprising a HCDR1 comprising the sequence of SEQ ID NO: 106, a HCDR2 comprising the sequence of SEQ ID NO: 107, and a HCDR3 comprising the sequence of SEQ ID NO: 108 and a VL comprising a LCDR1 comprising the sequence of SEQ ID NO: 110, a LCDR2 comprising the sequence of SEQ ID NO: 111, and a LCDR3 comprising the sequence of SEQ ID NO: 112;
(xxii) the first binding domain comprises a VH comprising a HCDR1 comprising the sequence of SEQ ID NO: 42, a HCDR2 comprising the sequence of SEQ ID NO: 43, and a HCDR3 comprising the sequence of SEQ ID NO: 44 and a VL comprising a LCDR1 comprising the sequence of SEQ ID NO: 46, a LCDR2 comprising the sequence of SEQ ID NO: 47, and a LCDR3 comprising the sequence of SEQ ID NO: 48,
and the second binding domain comprises a VH comprising a HCDR1 comprising the sequence of SEQ ID NO: 106, a HCDR2 comprising the sequence of SEQ ID NO: 107, and a HCDR3 comprising the sequence of SEQ ID NO: 108 and a VL comprising a LCDR1 comprising the sequence of SEQ ID NO: 110, a LCDR2 comprising the sequence of SEQ ID NO: 111, and a LCDR3 comprising the sequence of SEQ ID NO: 112;
(xxiii) the first binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128,
and the second binding domain comprises a sequence having at least 70%, at least 75%, at least 80%, at least 85%, at least 90%, at least 95%, at least 97%, at least 99%, or 100% identity to the sequence of SEQ ID NO: 129;
(xxiv) the first binding domain comprises a VH comprising a HCDR1 comprising the sequence of SEQ ID NO: 18, a HCDR2 comprising the sequence of SEQ ID NO: 19, and a HCDR3 comprising the sequence of SEQ ID NO: 20 and a VL comprising a LCDR1 comprising the sequence of SEQ ID NO: 22, a LCDR2 comprising the sequence of SEQ ID NO: 23, and a LCDR3 comprising the sequence of SEQ ID NO: 24,
and the second binding domain comprises a VH comprising a HCDR1 comprising the sequence of SEQ ID NO: 82, a HCDR2 comprising the sequence of SEQ ID NO: 83, and a HCDR3 comprising the sequence of SEQ ID NO: 84 and a VL comprising a LCDR1 comprising the sequence of SEQ ID NO: 86, a LCDR2 comprising the sequence of SEQ ID NO: 87, and a LCDR3 comprising the sequence of SEQ ID NO: 88;
(xxv) the first binding domain comprises a VH comprising a HCDR1 comprising the sequence of SEQ ID NO: 82, a HCDR2 comprising the sequence of SEQ ID NO: 83, and a HCDR3 comprising the sequence of SEQ ID NO: 84 and a VL comprising a LCDR1 comprising the sequence of SEQ ID NO: 86, a LCDR2 comprising the sequence of SEQ ID NO: 87, and a LCDR3 comprising the sequence of SEQ ID NO: 88,
and the second binding domain comprises a VH comprising a HCDR1 comprising the sequence of SEQ ID NO: 18, a HCDR2 comprising the sequence of SEQ ID NO: 19, and a HCDR3 comprising the sequence of SEQ ID NO: 20 and a VL comprising a LCDR1 comprising the sequence of SEQ ID NO: 22, a LCDR2 comprising the sequence of SEQ ID NO: 23, and a LCDR3 comprising the sequence of SEQ ID NO: 24;
(xxvi) the first binding domain comprises a VH comprising a HCDR1 comprising the sequence of SEQ ID NO: 82, a HCDR2 comprising the sequence of SEQ ID NO: 83, and a HCDR3 comprising the sequence of SEQ ID NO: 84 and a VL comprising a LCDR1 comprising the sequence of SEQ ID NO: 86, a LCDR2 comprising the sequence of SEQ ID NO: 87, and a LCDR3 comprising the sequence of SEQ ID NO: 88,
and the second binding domain comprises a VH comprising a HCDR1 comprising the sequence of SEQ ID NO: 50, a HCDR2 comprising the sequence of SEQ ID NO: 51, and a HCDR3 comprising the sequence of SEQ ID NO: 52 and a VL comprising a LCDR1 comprising the sequence of SEQ ID NO: 54, a LCDR2 comprising the sequence of SEQ ID NO: 55, and a LCDR3 comprising the sequence of SEQ ID NO: 56; or
(xxvii) the first binding domain comprises a VH comprising a HCDR1 comprising the sequence of SEQ ID NO: 50, a HCDR2 comprising the sequence of SEQ ID NO: 51, and a HCDR3 comprising the sequence of SEQ ID NO: 52 and a VL comprising a LCDR1 comprising the sequence of SEQ ID NO: 54, a LCDR2 comprising the sequence of SEQ ID NO: 55, and a LCDR3 comprising the sequence of SEQ ID NO: 56,
and the second binding domain comprises a VH comprising a HCDR1 comprising the sequence of SEQ ID NO: 82, a HCDR2 comprising the sequence of SEQ ID NO: 83, and a HCDR3 comprising the sequence of SEQ ID NO: 84 and a VL comprising a LCDR1 comprising the sequence of SEQ ID NO: 86, a LCDR2 comprising the sequence of SEQ ID NO: 87, and a LCDR3 comprising the sequence of SEQ ID NO: 88.

In one embodiment of the binding agent described herein:
(i) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 33 and a VL comprising a sequence having at least 70%, at least 75%, at least 80%, at least 85%, at least 90%, at least 95%, at least 97%, at least 99%, or 100% identity to the sequence of SEQ ID NO: 37;
(ii) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 1 and a VL comprising a sequence having at least 70%, at least 75%, at least 80%, at least 85%, at least 90%, at least 95%, at least 97%, at least 99%, or 100% identity to the sequence of SEQ ID NO: 5;
(iii) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 9 and a VL comprising a sequence having at least 70%, at least 75%, at least 80%, at least 85%, at least 90%, at least 95%, at least 97%, at least 99%, or 100% identity to the sequence of SEQ ID NO: 13;
(iv) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 25 and a VL comprising a sequence having at least 70%, at least 75%, at least 80%, at least 85%, at least 90%, at least 95%, at least 97%, at least 99%, or 100% identity to the sequence of SEQ ID NO: 29;
(v) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 41 and a VL comprising a sequence having at least 70%, at least 75%, at least 80%, at least 85%, at least 90%, at least 95%, at least 97%, at least 99%, or 100% identity to the sequence of SEQ ID NO: 45;
(vi) the first binding domain comprises, a VH comprising a sequence having at least 70%, at least 75%, at least 80%, at least 85%, at least 90%, at least 95%, at least 97%, at least 99%, or 100% identity to the sequence of SEQ ID NO: 33 and a VL comprising a sequence having at least 70%, at least 75%, at least 80%, at least 85%, at least 90%, at least 95%, at least 97%, at least 99%, or 100% identity to the sequence of SEQ ID NO: 37,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125;
(vii) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 25 and a VL comprising a sequence having at least 70%, at least 75%, at least 80%, at least 85%, at least 90%, at least 95%, at least 97%, at least 99%, or 100% identity to the sequence of SEQ ID NO: 29,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125;
(viii) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 41 and a VL comprising a sequence having at least 70%, at least 75%, at least 80%, at least 85%, at least 90%, at least 95%, at least 97%, at least 99%, or 100% identity to the sequence of SEQ ID NO: 45,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125;
(ix) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 1 and a VL comprising a sequence having at least 70%, at least 75%, at least 80%, at least 85%, at least 90%, at least 95%, at least 97%, at least 99%, or 100% identity to the sequence of SEQ ID NO: 5,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125;
(x) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 9 and a VL comprising a sequence having at least 70%, at least 75%, at least 80%, at least 85%, at least 90%, at least 95%, at least 97%, at least 99%, or 100% identity to the sequence of SEQ ID NO: 13,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125;
(xi) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 17 and a VL comprising a sequence having at least 70%, at least 75%, at least 80%, at least 85%, at least 90%, at least 95%, at least 97%, at least 99%, or 100% identity to the sequence of SEQ ID NO: 21;
(xii) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 49 and a VL comprising a sequence having at least 70%, at least 75%, at least 80%, at least 85%, at least 90%, at least 95%, at least 97%, at least 99%, or 100% identity to the sequence of SEQ ID NO: 53;
(xiii) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 105 and a VL comprising a sequence having at least 70%, at least 75%, at least 80%, at least 85%, at least 90%, at least 95%, at least 97%, at least 99%, or 100% identity to the sequence of SEQ ID NO: 109;
(xiv) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 17 and a VL comprising a sequence having at least 70%, at least 75%, at least 80%, at least 85%, at least 90%, at least 95%, at least 97%, at least 99%, or 100% identity to the sequence of SEQ ID NO: 21,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125;
(xv) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 49 and a VL comprising a sequence having at least 70%, at least 75%, at least 80%, at least 85%, at least 90%, at least 95%, at least 97%, at least 99%, or 100% identity to the sequence of SEQ ID NO: 53,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125;
(xvi) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 105 and a VL comprising a sequence having at least 70%, at least 75%, at least 80%, at least 85%, at least 90%, at least 95%, at least 97%, at least 99%, or 100% identity to the sequence of SEQ ID NO: 109,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125;
(xvii) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 105 and a VL comprising a sequence having at least 70%, at least 75%, at least 80%, at least 85%, at least 90%, at least 95%, at least 97%, at least 99%, or 100% identity to the sequence of SEQ ID NO: 109,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 17 and a VL comprising a sequence having at least 70%, at least 75%, at least 80%, at least 85%, at least 90%, at least 95%, at least 97%, at least 99%, or 100% identity to the sequence of SEQ ID NO: 21;
(xviii) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 105 and a VL comprising a sequence having at least 70%, at least 75%, at least 80%, at least 85%, at least 90%, at least 95%, at least 97%, at least 99%, or 100% identity to the sequence of SEQ ID NO: 109,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 49 and a VL comprising a sequence having at least 70%, at least 75%, at least 80%, at least 85%, at least 90%, at least 95%, at least 97%, at least 99%, or 100% identity to the sequence of SEQ ID NO: 53;
(xix) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 105 and a VL comprising a sequence having at least 70%, at least 75%, at least 80%, at least 85%, at least 90%, at least 95%, at least 97%, at least 99%, or 100% identity to the sequence of SEQ ID NO: 109,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 41 and a VL comprising a sequence having at least 70%, at least 75%, at least 80%, at least 85%, at least 90%, at least 95%, at least 97%, at least 99%, or 100% identity to the sequence of SEQ ID NO: 45;
(xx) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 17 and a VL comprising a sequence having at least 70%, at least 75%, at least 80%, at least 85%, at least 90%, at least 95%, at least 97%, at least 99%, or 100% identity to the sequence of SEQ ID NO: 21,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 105 and a VL comprising a sequence having at least 70%, at least 75%, at least 80%, at least 85%, at least 90%, at least 95%, at least 97%, at least 99%, or 100% identity to the sequence of SEQ ID NO: 109;
(xxi) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 49 and a VL comprising a sequence having at least 70%, at least 75%, at least 80%, at least 85%, at least 90%, at least 95%, at least 97%, at least 99%, or 100% identity to the sequence of SEQ ID NO: 53,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 105 and a VL comprising a sequence having at least 70%, at least 75%, at least 80%, at least 85%, at least 90%, at least 95%, at least 97%, at least 99%, or 100% identity to the sequence of SEQ ID NO: 109;
(xxii) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 41 and a VL comprising a sequence having at least 70%, at least 75%, at least 80%, at least 85%, at least 90%, at least 95%, at least 97%, at least 99%, or 100% identity to the sequence of SEQ ID NO: 45,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 105 and a VL comprising a sequence having at least 70%, at least 75%, at least 80%, at least 85%, at least 90%, at least 95%, at least 97%, at least 99%, or 100% identity to the sequence of SEQ ID NO: 109;
(xxiii) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125,
and the second binding domain comprises a sequence having at least 70%, at least 75%, at least 80%, at least 85%, at least 90%, at least 95%, at least 97%, at least 99%, or 100% identity to the sequence of SEQ ID NO: 129;
(xxiv) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 17 and a VL comprising a sequence having at least 70%, at least 75%, at least 80%, at least 85%, at least 90%, at least 95%, at least 97%, at least 99%, or 100% identity to the sequence of SEQ ID NO: 21,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 81 and a VL comprising a sequence having at least 70%, at least 75%, at least 80%, at least 85%, at least 90%, at least 95%, at least 97%, at least 99%, or 100% identity to the sequence of SEQ ID NO: 85;
(xxv) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 81 and a VL comprising a sequence having at least 70%, at least 75%, at least 80%, at least 85%, at least 90%, at least 95%, at least 97%, at least 99%, or 100% identity to the sequence of SEQ ID NO: 85,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 17 and a VL comprising a sequence having at least 70%, at least 75%, at least 80%, at least 85%, at least 90%, at least 95%, at least 97%, at least 99%, or 100% identity to the sequence of SEQ ID NO: 21;
(xxvi) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 81 and a VL comprising a sequence having at least 70%, at least 75%, at least 80%, at least 85%, at least 90%, at least 95%, at least 97%, at least 99%, or 100% identity to the sequence of SEQ ID NO: 85,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 49 and a VL comprising a sequence having at least 70%, at least 75%, at least 80%, at least 85%, at least 90%, at least 95%, at least 97%, at least 99%, or 100% identity to the sequence of SEQ ID NO: 53; or
(xxvii) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 49 and a VL comprising a sequence having at least 70%, at least 75%, at least 80%, at least 85%, at least 90%, at least 95%, at least 97%, at least 99%, or 100% identity to the sequence of SEQ ID NO: 53,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 81 and a VL comprising a sequence having at least 70%, at least 75%, at least 80%, at least 85%, at least 90%, at least 95%, at least 97%, at least 99%, or 100% identity to the sequence of SEQ ID NO: 85.

In one embodiment, the binding agent described herein comprises a heavy chain and a light chain forming the first binding domain. In one embodiment, the binding agent described herein comprises two heavy chains and two light chains, wherein each of the heavy chains together with one of the light chains forms a first binding domain. In one embodiment, the heavy chain comprises a VH. In one embodiment, the light chain comprises a VL. In one embodiment, the heavy chain comprises a fragment crystallizable (Fc) region. In one embodiment, a heavy chain is associated with a light chain. In one embodiment, the heavy chains are covalently and/or non-covalently associated. In one embodiment, the two heavy chains are identical and the two light chains are identical.

In one embodiment, the binding agent comprises a full-length antibody or a full-length antibody-like molecule comprising first binding domains.

In one embodiment, the binding agent comprises two first binding domains. In one embodiment, the two first binding domains bind to the same epitope.

In one embodiment, the second binding domain comprises a single-chain variable fragment (scFv).

In one embodiment, the first and second binding domains are covalently linked, either directly or through a linker. In one embodiment, the linker comprises a glycine-serine (GS) linker. In one embodiment, the glycine-serine linker comprises a (G₄S)₁ linker. In one embodiment, the glycine-serine linker comprises a (G₄S)₂ linker. In one embodiment, the glycine-serine linker comprises a (G₄S)₃ linker. In one embodiment, the glycine-serine linker comprises a (G₄S)₄ linker. In one embodiment, the glycine-serine linker comprises a (G₄S)₅ linker.

In one embodiment, the binding agent comprises two heavy chains and two light chains forming a full-length antibody or a full-length antibody-like molecule comprising two first binding domains, wherein each of the light chains is linked to a second binding domain. In one embodiment, the C-terminus of each of the light chains is linked to the N-terminus of a second binding domain. In one embodiment, the N-terminus of each of the light chains is linked to the C-terminus of a second binding domain.

In one embodiment, the binding agent comprises:
(i) a first polypeptide comprising a heavy chain (HC) and
(ii) a second polypeptide comprising a light chain (LC) and further comprising an extracellular domain (ECD) of ACE2 protein or a variant thereof, or a fragment of the ECD of ACE2 protein or the variant thereof.

In one embodiment, the binding agent comprises:
(i) a first polypeptide comprising a heavy chain (HC) and
(ii) a second polypeptide comprising a light chain (LC) and further comprising a scFv.

In one embodiment, the binding agent comprises an antibody comprising a first binding arm and a second binding arm, wherein
a. the first binding arm comprises:
(i) a first polypeptide comprising a heavy chain (HC) and
(ii) a second polypeptide comprising a light chain (LC) and further comprising an extracellular domain (ECD) of ACE2 protein or a variant thereof, or a fragment of the ECD of ACE2 protein or the variant thereof, and;
b. the second binding arm comprises:
(i) a first polypeptide comprising a heavy chain (HC) and
(ii) a second polypeptide comprising a light chain (LC) and further comprising an extracellular domain (ECD) of ACE2 protein or a variant thereof, or a fragment of the ECD of ACE2 protein or the variant thereof.

In one embodiment, the binding agent comprises an antibody comprising a first binding arm and a second binding arm, wherein
a. the first binding arm comprises:
(i) a first polypeptide comprising a heavy chain (HC) and
(ii) a second polypeptide comprising a light chain (LC) and further comprising a scFv, and;
b. the second binding arm comprises:
(i) a first polypeptide comprising a heavy chain (HC) and
(ii) a second polypeptide comprising a light chain (LC) and further comprising a scFv.

In one embodiment, the first polypeptide of the first binding arm and the first polypeptide of the second binding arm are identical. In one embodiment, the second polypeptide of the first binding arm and the second polypeptide of the second binding arm are identical.

In certain preferred embodiments of the binding agent:
(i) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 133 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 134 or a fragment thereof;
(ii) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 135 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 136 or a fragment thereof;
(iii) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 137 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 138 or a fragment thereof;
(iv) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 139 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 140 or a fragment thereof;
(v) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 143 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 144 or a fragment thereof;
(vi) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 145 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 146 or a fragment thereof;
(vii) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 147 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 148 or a fragment thereof;
(viii) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 149 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 150 or a fragment thereof;
(ix) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 153 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 154 or a fragment thereof;
(x) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 155 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 156 or a fragment thereof;
(xi) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 157 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 158 or a fragment thereof;
(xii) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 159 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 160 or a fragment thereof;
(xiii) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 161 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 162 or a fragment thereof;
(xiv) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 163 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 164 or a fragment thereof;
(xv) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 165 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 166 or a fragment thereof;
(xvi) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 167 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 168 or a fragment thereof;
(xvii) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 169 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 170 or a fragment thereof;
(xviii) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 171 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 172 or a fragment thereof;
(xix) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 173 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 174 or a fragment thereof;
(xx) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 175 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 176 or a fragment thereof;
(xxi) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 177 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 178 or a fragment thereof;
(xxii) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 179 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 180 or a fragment thereof;
(xxiii) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 181 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 182 or a fragment thereof;
(xxiv) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 183 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 184 or a fragment thereof;
(xxv) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 185 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 186 or a fragment thereof;
(xxvi) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 187 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 188 or a fragment thereof;
(xxvii) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 189 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 190 or a fragment thereof;
(xxviii) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 191 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 192 or a fragment thereof;
(xxix) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 193 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 194 or a fragment thereof;
(xxx) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 195 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 196 or a fragment thereof;
(xxxi) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 205 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 206 or a fragment thereof;
(xxxii) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 207 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 208 or a fragment thereof;
(xxxiii) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 209 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 210 or a fragment thereof; or
(xxxiv) the first polypeptide comprises an amino acid sequence having at least 70%, at least 75%, at least 80%, at least 85%, at least 90%, at least 95%, at least 97%, at least 99%, or 100% identity to the sequence of SEQ ID NO: 211 or a fragment thereof,
and the second polypeptide comprises an amino acid sequence having at least 70%, at least 75%, at least 80%, at least 85%, at least 90%, at least 95%, at least 97%, at least 99%, or 100% identity to the sequence of SEQ ID NO: 212 or a fragment thereof.

In one embodiment, the heavy chain (HC) comprises a heavy chain variable region (VH) and a heavy chain constant region (CH). In one embodiment, the heavy chain constant region (CH) comprises a constant region domain 1 region (CH1), a hinge region, a constant region domain 2 region (CH2), and a constant region domain 3 region (CH3). In one embodiment, the light chain (LC) comprises a light chain variable region (VL) and a light chain constant region (CL).

In one embodiment, a heavy chain variable region (VH) and a light chain variable region (VL) together provide a first binding domain. In one embodiment, a heavy chain variable region (VH) and a light chain variable region (VL) on the same binding arm together provide a first binding domain. In one embodiment, an extracellular domain (ECD) of ACE2 protein or a variant thereof, or a fragment of the ECD of ACE2 protein or the variant thereof provides a second binding domain. In one embodiment, a scFv provides a second binding domain.

In a further aspect, the present invention provides an antibody, comprising a heavy chain variable region (VH), wherein the VH comprises one or more selected from the group consisting of:
(i) a HCDR3 comprising a sequence selected from the group consisting of SEQ ID NO: 4, 12, 20, 28, 36, 44, 52, 60, 68, 76, 84, 92, 100, 108, and 116;
(ii) a HCDR2 comprising a sequence selected from the group consisting of SEQ ID NO: 3, 11, 19, 27, 35, 43, 51, 59, 67, 75, 83, 91, 99, 107, and 115; and
(iii) a HCDR1 comprising a sequence selected from the group consisting of SEQ ID NO: 2, 10, 18, 26, 34, 42, 50, 58, 66, 74, 82, 90, 98, 106, and 114.

In one embodiment, the VH is selected from the group consisting of:
(i) a VH comprising a HCDR1 comprising the sequence of SEQ ID NO: 2, a HCDR2 comprising the sequence of SEQ ID NO: 3, and a HCDR3 comprising the sequence of SEQ ID NO: 4;
(ii) a VH comprising a HCDR1 comprising the sequence of SEQ ID NO: 10, a HCDR2 comprising the sequence of SEQ ID NO: 11, and a HCDR3 comprising the sequence of SEQ ID NO: 12;
(iii) a VH comprising a HCDR1 comprising the sequence of SEQ ID NO: 18, a HCDR2 comprising the sequence of SEQ ID NO: 19, and a HCDR3 comprising the sequence of SEQ ID NO: 20;
(iv) a VH comprising a HCDR1 comprising the sequence of SEQ ID NO: 26, a HCDR2 comprising the sequence of SEQ ID NO: 27, and a HCDR3 comprising the sequence of SEQ ID NO: 28;
(v) a VH comprising a HCDR1 comprising the sequence of SEQ ID NO: 34, a HCDR2 comprising the sequence of SEQ ID NO: 35, and a HCDR3 comprising the sequence of SEQ ID NO: 36;
(vi) a VH comprising a HCDR1 comprising the sequence of SEQ ID NO: 42, a HCDR2 comprising the sequence of SEQ ID NO: 43, and a HCDR3 comprising the sequence of SEQ ID NO: 44;
(vii) a VH comprising a HCDR1 comprising the sequence of SEQ ID NO: 50, a HCDR2 comprising the sequence of SEQ ID NO: 51, and a HCDR3 comprising the sequence of SEQ ID NO: 52;
(viii) a VH comprising a HCDR1 comprising the sequence of SEQ ID NO: 58, a HCDR2 comprising the sequence of SEQ ID NO: 59, and a HCDR3 comprising the sequence of SEQ ID NO: 60;
(ix) a VH comprising a HCDR1 comprising the sequence of SEQ ID NO: 66, a HCDR2 comprising the sequence of SEQ ID NO: 67, and a HCDR3 comprising the sequence of SEQ ID NO: 68;
(x) a VH comprising a HCDR1 comprising the sequence of SEQ ID NO: 74, a HCDR2 comprising the sequence of SEQ ID NO: 75, and a HCDR3 comprising the sequence of SEQ ID NO: 76;
(xi) a VH comprising a HCDR1 comprising the sequence of SEQ ID NO: 82, a HCDR2 comprising the sequence of SEQ ID NO: 83, and a HCDR3 comprising the sequence of SEQ ID NO: 84;
(xii) a VH comprising a HCDR1 comprising the sequence of SEQ ID NO: 90, a HCDR2 comprising the sequence of SEQ ID NO: 91, and a HCDR3 comprising the sequence of SEQ ID NO: 92;
(xiii) a VH comprising a HCDR1 comprising the sequence of SEQ ID NO: 98, a HCDR2 comprising the sequence of SEQ ID NO: 99, and a HCDR3 comprising the sequence of SEQ ID NO: 100;
(xiv) a VH comprising a HCDR1 comprising the sequence of SEQ ID NO: 106, a HCDR2 comprising the sequence of SEQ ID NO: 107, and a HCDR3 comprising the sequence of SEQ ID NO: 108; and
(xv) a VH comprising a HCDR1 comprising the sequence of SEQ ID NO: 114, a HCDR2 comprising the sequence of SEQ ID NO: 115, and a HCDR3 comprising the sequence of SEQ ID NO: 116.

In a further aspect, the present invention provides an antibody, comprising a light chain variable region (VL), wherein the VL comprises one or more selected from the group consisting of:
(i) a LCDR3 comprising a sequence selected from the group consisting of SEQ ID NO: 8, 16, 24, 32, 40, 48, 56, 64, 72, 80, 88, 96, 104, 112, and 120;
(ii) a LCDR2 comprising a sequence selected from the group consisting of SEQ ID NO: 7, 15, 23, 31, 39, 47, 55, 63, 71, 79, 87, 95, 103, 111, and 119; and
(iii) a LCDR1 comprising a sequence selected from the group consisting of SEQ ID NO: 6, 14, 22, 30, 38, 46, 54, 62, 70, 78, 86, 94, 102, 110, and 118.

In one embodiment, the VL is selected from the group consisting of:
(i) a VL comprising a LCDR1 comprising the sequence of SEQ ID NO: 6, a LCDR2 comprising the sequence of SEQ ID NO: 7, and a LCDR3 comprising the sequence of SEQ ID NO: 8;
(ii) a VL comprising a LCDR1 comprising the sequence of SEQ ID NO: 14, a LCDR2 comprising the sequence of SEQ ID NO: 15, and a LCDR3 comprising the sequence of SEQ ID NO: 16;
(iii) a VL comprising a LCDR1 comprising the sequence of SEQ ID NO: 22, a LCDR2 comprising the sequence of SEQ ID NO: 23, and a LCDR3 comprising the sequence of SEQ ID NO: 24;
(iv) a VL comprising a LCDR1 comprising the sequence of SEQ ID NO: 30, a LCDR2 comprising the sequence of SEQ ID NO: 31, and a LCDR3 comprising the sequence of SEQ ID NO: 32;
(v) a VL comprising a LCDR1 comprising the sequence of SEQ ID NO: 38, a LCDR2 comprising the sequence of SEQ ID NO: 39, and a LCDR3 comprising the sequence of SEQ ID NO: 40;
(vi) a VL comprising a LCDR1 comprising the sequence of SEQ ID NO: 46, a LCDR2 comprising the sequence of SEQ ID NO: 47, and a LCDR3 comprising the sequence of SEQ ID NO: 48;
(vii) a VL comprising a LCDR1 comprising the sequence of SEQ ID NO: 54, a LCDR2 comprising the sequence of SEQ ID NO: 55, and a LCDR3 comprising the sequence of SEQ ID NO: 56;
(viii) a VL comprising a LCDR1 comprising the sequence of SEQ ID NO: 62, a LCDR2 comprising the sequence of SEQ ID NO: 63, and a LCDR3 comprising the sequence of SEQ ID NO: 64;
(ix) a VL comprising a LCDR1 comprising the sequence of SEQ ID NO: 70, a LCDR2 comprising the sequence of SEQ ID NO: 71, and a LCDR3 comprising the sequence of SEQ ID NO: 72;
(x) a VL comprising a LCDR1 comprising the sequence of SEQ ID NO: 78, a LCDR2 comprising the sequence of SEQ ID NO: 79, and a LCDR3 comprising the sequence of SEQ ID NO: 80;
(xi) a VL comprising a LCDR1 comprising the sequence of SEQ ID NO: 86, a LCDR2 comprising the sequence of SEQ ID NO: 87, and a LCDR3 comprising the sequence of SEQ ID NO: 88;
(xii) a VL comprising a LCDR1 comprising the sequence of SEQ ID NO: 94, a LCDR2 comprising the sequence of SEQ ID NO: 95, and a LCDR3 comprising the sequence of SEQ ID NO: 96;
(xiii) a VL comprising a LCDR1 comprising the sequence of SEQ ID NO: 102, a LCDR2 comprising the sequence of SEQ ID NO: 103, and a LCDR3 comprising the sequence of SEQ ID NO: 104;
(xiv) a VL comprising a LCDR1 comprising the sequence of SEQ ID NO: 110, a LCDR2 comprising the sequence of SEQ ID NO: 111, and a LCDR3 comprising the sequence of SEQ ID NO: 112; and
(xv) a VL comprising a LCDR1 comprising the sequence of SEQ ID NO: 118, a LCDR2 comprising the sequence of SEQ ID NO: 119, and a LCDR3 comprising the sequence of SEQ ID NO: 120.

In one embodiment of the antibody described herein, the antibody comprises a VH and a VL selected from the group consisting of:
(i) a VH comprising a HCDR1 comprising the sequence of SEQ ID NO: 2, a HCDR2 comprising the sequence of SEQ ID NO: 3, and a HCDR3 comprising the sequence of SEQ ID NO: 4 and a VL comprising a LCDR1 comprising the sequence of SEQ ID NO: 6, a LCDR2 comprising the sequence of SEQ ID NO: 7, and a LCDR3 comprising the sequence of SEQ ID NO: 8;
(ii) a VH comprising a HCDR1 comprising the sequence of SEQ ID NO: 10, a HCDR2 comprising the sequence of SEQ ID NO: 11, and a HCDR3 comprising the sequence of SEQ ID NO: 12 and a VL comprising a LCDR1 comprising the sequence of SEQ ID NO: 14, a LCDR2 comprising the sequence of SEQ ID NO: 15, and a LCDR3 comprising the sequence of SEQ ID NO: 16;
(iii) a VH comprising a HCDR1 comprising the sequence of SEQ ID NO: 18, a HCDR2 comprising the sequence of SEQ ID NO: 19, and a HCDR3 comprising the sequence of SEQ ID NO: 20 and a VL comprising a LCDR1 comprising the sequence of SEQ ID NO: 22, a LCDR2 comprising the sequence of SEQ ID NO: 23, and a LCDR3 comprising the sequence of SEQ ID NO: 24;
(iv) a VH comprising a HCDR1 comprising the sequence of SEQ ID NO: 26, a HCDR2 comprising the sequence of SEQ ID NO: 27, and a HCDR3 comprising the sequence of SEQ ID NO: 28 and a VL comprising a LCDR1 comprising the sequence of SEQ ID NO: 30, a LCDR2 comprising the sequence of SEQ ID NO: 31, and a LCDR3 comprising the sequence of SEQ ID NO: 32;
(v) a VH comprising a HCDR1 comprising the sequence of SEQ ID NO: 34, a HCDR2 comprising the sequence of SEQ ID NO: 35, and a HCDR3 comprising the sequence of SEQ ID NO: 36 and a VL comprising a LCDR1 comprising the sequence of SEQ ID NO: 38, a LCDR2 comprising the sequence of SEQ ID NO: 39, and a LCDR3 comprising the sequence of SEQ ID NO: 40;
(vi) a VH comprising a HCDR1 comprising the sequence of SEQ ID NO: 42, a HCDR2 comprising the sequence of SEQ ID NO: 43, and a HCDR3 comprising the sequence of SEQ ID NO: 44 and a VL comprising a LCDR1 comprising the sequence of SEQ ID NO: 46, a LCDR2 comprising the sequence of SEQ ID NO: 47, and a LCDR3 comprising the sequence of SEQ ID NO: 48;
(vii) a VH comprising a HCDR1 comprising the sequence of SEQ ID NO: 50, a HCDR2 comprising the sequence of SEQ ID NO: 51, and a HCDR3 comprising the sequence of SEQ ID NO: 52 and a VL comprising a LCDR1 comprising the sequence of SEQ ID NO: 54, a LCDR2 comprising the sequence of SEQ ID NO: 55, and a LCDR3 comprising the sequence of SEQ ID NO: 56;
(viii) a VH comprising a HCDR1 comprising the sequence of SEQ ID NO: 58, a HCDR2 comprising the sequence of SEQ ID NO: 59, and a HCDR3 comprising the sequence of SEQ ID NO: 60 and a VL comprising a LCDR1 comprising the sequence of SEQ ID NO: 62, a LCDR2 comprising the sequence of SEQ ID NO: 63, and a LCDR3 comprising the sequence of SEQ ID NO: 64;
(ix) a VH comprising a HCDR1 comprising the sequence of SEQ ID NO: 66, a HCDR2 comprising the sequence of SEQ ID NO: 67, and a HCDR3 comprising the sequence of SEQ ID NO: 68 and a VL comprising a LCDR1 comprising the sequence of SEQ ID NO: 70, a LCDR2 comprising the sequence of SEQ ID NO: 71, and a LCDR3 comprising the sequence of SEQ ID NO: 72;
(x) a VH comprising a HCDR1 comprising the sequence of SEQ ID NO: 74, a HCDR2 comprising the sequence of SEQ ID NO: 75, and a HCDR3 comprising the sequence of SEQ ID NO: 76 and a VL comprising a LCDR1 comprising the sequence of SEQ ID NO: 78, a LCDR2 comprising the sequence of SEQ ID NO: 79, and a LCDR3 comprising the sequence of SEQ ID NO: 80;
(xi) a VH comprising a HCDR1 comprising the sequence of SEQ ID NO: 82, a HCDR2 comprising the sequence of SEQ ID NO: 83, and a HCDR3 comprising the sequence of SEQ ID NO: 84 and a VL comprising a LCDR1 comprising the sequence of SEQ ID NO: 86, a LCDR2 comprising the sequence of SEQ ID NO: 87, and a LCDR3 comprising the sequence of SEQ ID NO: 88;
(xii) a VH comprising a HCDR1 comprising the sequence of SEQ ID NO: 90, a HCDR2 comprising the sequence of SEQ ID NO: 91, and a HCDR3 comprising the sequence of SEQ ID NO: 92 and a VL comprising a LCDR1 comprising the sequence of SEQ ID NO: 94, a LCDR2 comprising the sequence of SEQ ID NO: 95, and a LCDR3 comprising the sequence of SEQ ID NO: 96;
(xiii) a VH comprising a HCDR1 comprising the sequence of SEQ ID NO: 98, a HCDR2 comprising the sequence of SEQ ID NO: 99, and a HCDR3 comprising the sequence of SEQ ID NO: 100 and a VL comprising a LCDR1 comprising the sequence of SEQ ID NO: 102, a LCDR2 comprising the sequence of SEQ ID NO: 103, and a LCDR3 comprising the sequence of SEQ ID NO: 104;
(xiv) a VH comprising a HCDR1 comprising the sequence of SEQ ID NO: 106, a HCDR2 comprising the sequence of SEQ ID NO: 107, and a HCDR3 comprising the sequence of SEQ ID NO: 108 and a VL comprising a LCDR1 comprising the sequence of SEQ ID NO: 110, a LCDR2 comprising the sequence of SEQ ID NO: 111, and a LCDR3 comprising the sequence of SEQ ID NO: 112; and
(xv) a VH comprising a HCDR1 comprising the sequence of SEQ ID NO: 114, a HCDR2 comprising the sequence of SEQ ID NO: 115, and a HCDR3 comprising the sequence of SEQ ID NO: 116 and a VL comprising a LCDR1 comprising the sequence of SEQ ID NO: 118, a LCDR2 comprising the sequence of SEQ ID NO: 119, and a LCDR3 comprising the sequence of SEQ ID NO: 120.

In one embodiment of the antibody described herein, the antibody comprises a VH comprising a sequence having at least 70%, at least 75%, at least 80%, at least 85%, at least 90%, at least 95%, at least 97%, at least 99%, or 100% identity to a sequence selected from the group consisting of SEQ ID NO: 1, 9, 17, 25, 33, 41, 49, 57, 65, 73, 81, 89, 97, 105, and 113.

In one embodiment of the antibody described herein, the antibody comprises a VL comprising a sequence having at least 70%, at least 75%, at least 80%, at least 85%, at least 90%, at least 95%, at least 97%, at least 99%, or 100% identity to a sequence selected from the group consisting of SEQ ID NO: 5, 13, 21, 29, 37, 45, 53, 61, 69, 77, 85, 93, 101, 109, and 117.

In one embodiment of the antibody described herein, the antibody comprises a VH and a VL selected from the group consisting of:
(i) a VH comprising a sequence having at least 70%, at least 75%, at least 80%, at least 85%, at least 90%, at least 95%, at least 97%, at least 99%, or 100% identity to the sequence of SEQ ID NO: 1 and a VL comprising a sequence having at least 70%, at least 75%, at least 80%, at least 85%, at least 90%, at least 95%, at least 97%, at least 99%, or 100% identity to the sequence of SEQ ID NO: 5;
(ii) a VH comprising a sequence having at least 70%, at least 75%, at least 80%, at least 85%, at least 90%, at least 95%, at least 97%, at least 99%, or 100% identity to the sequence of SEQ ID NO: 9 and a VL comprising a sequence having at least 70%, at least 75%, at least 80%, at least 85%, at least 90%, at least 95%, at least 97%, at least 99%, or 100% identity to the sequence of SEQ ID NO: 13;
(iii) a VH comprising a sequence having at least 70%, at least 75%, at least 80%, at least 85%, at least 90%, at least 95%, at least 97%, at least 99%, or 100% identity to the sequence of SEQ ID NO: 17 and a VL comprising a sequence having at least 70%, at least 75%, at least 80%, at least 85%, at least 90%, at least 95%, at least 97%, at least 99%, or 100% identity to the sequence of SEQ ID NO: 21;
(iv) a VH comprising a sequence having at least 70%, at least 75%, at least 80%, at least 85%, at least 90%, at least 95%, at least 97%, at least 99%, or 100% identity to the sequence of SEQ ID NO: 25 and a VL comprising a sequence having at least 70%, at least 75%, at least 80%, at least 85%, at least 90%, at least 95%, at least 97%, at least 99%, or 100% identity to the sequence of SEQ ID NO: 29;
(v) a VH comprising a sequence having at least 70%, at least 75%, at least 80%, at least 85%, at least 90%, at least 95%, at least 97%, at least 99%, or 100% identity to the sequence of SEQ ID NO: 33 and a VL comprising a sequence having at least 70%, at least 75%, at least 80%, at least 85%, at least 90%, at least 95%, at least 97%, at least 99%, or 100% identity to the sequence of SEQ ID NO: 37;
(vi) a VH comprising a sequence having at least 70%, at least 75%, at least 80%, at least 85%, at least 90%, at least 95%, at least 97%, at least 99%, or 100% identity to the sequence of SEQ ID NO: 41 and a VL comprising a sequence having at least 70%, at least 75%, at least 80%, at least 85%, at least 90%, at least 95%, at least 97%, at least 99%, or 100% identity to the sequence of SEQ ID NO: 45;
(vii) a VH comprising a sequence having at least 70%, at least 75%, at least 80%, at least 85%, at least 90%, at least 95%, at least 97%, at least 99%, or 100% identity to the sequence of SEQ ID NO: 49 and a VL comprising a sequence having at least 70%, at least 75%, at least 80%, at least 85%, at least 90%, at least 95%, at least 97%, at least 99%, or 100% identity to the sequence of SEQ ID NO: 53;
(viii) a VH comprising a sequence having at least 70%, at least 75%, at least 80%, at least 85%, at least 90%, at least 95%, at least 97%, at least 99%, or 100% identity to the sequence of SEQ ID NO: 57 and a VL comprising a sequence having at least 70%, at least 75%, at least 80%, at least 85%, at least 90%, at least 95%, at least 97%, at least 99%, or 100% identity to the sequence of SEQ ID NO: 61;
(ix) a VH comprising a sequence having at least 70%, at least 75%, at least 80%, at least 85%, at least 90%, at least 95%, at least 97%, at least 99%, or 100% identity to the sequence of SEQ ID NO: 65 and a VL comprising a sequence having at least 70%, at least 75%, at least 80%, at least 85%, at least 90%, at least 95%, at least 97%, at least 99%, or 100% identity to the sequence of SEQ ID NO: 69;
(x) a VH comprising a sequence having at least 70%, at least 75%, at least 80%, at least 85%, at least 90%, at least 95%, at least 97%, at least 99%, or 100% identity to the sequence of SEQ ID NO: 73 and a VL comprising a sequence having at least 70%, at least 75%, at least 80%, at least 85%, at least 90%, at least 95%, at least 97%, at least 99%, or 100% identity to the sequence of SEQ ID NO: 77;
(xi) a VH comprising a sequence having at least 70%, at least 75%, at least 80%, at least 85%, at least 90%, at least 95%, at least 97%, at least 99%, or 100% identity to the sequence of SEQ ID NO: 81 and a VL comprising a sequence having at least 70%, at least 75%, at least 80%, at least 85%, at least 90%, at least 95%, at least 97%, at least 99%, or 100% identity to the sequence of SEQ ID NO: 85;
(xii) a VH comprising a sequence having at least 70%, at least 75%, at least 80%, at least 85%, at least 90%, at least 95%, at least 97%, at least 99%, or 100% identity to the sequence of SEQ ID NO: 89 and a VL comprising a sequence having at least 70%, at least 75%, at least 80%, at least 85%, at least 90%, at least 95%, at least 97%, at least 99%, or 100% identity to the sequence of SEQ ID NO: 93;
(xiii) a VH comprising a sequence having at least 70%, at least 75%, at least 80%, at least 85%, at least 90%, at least 95%, at least 97%, at least 99%, or 100% identity to the sequence of SEQ ID NO: 97 and a VL comprising a sequence having at least 70%, at least 75%, at least 80%, at least 85%, at least 90%, at least 95%, at least 97%, at least 99%, or 100% identity to the sequence of SEQ ID NO: 101;
(xiv) a VH comprising a sequence having at least 70%, at least 75%, at least 80%, at least 85%, at least 90%, at least 95%, at least 97%, at least 99%, or 100% identity to the sequence of SEQ ID NO: 105 and a VL comprising a sequence having at least 70%, at least 75%, at least 80%, at least 85%, at least 90%, at least 95%, at least 97%, at least 99%, or 100% identity to the sequence of SEQ ID NO: 109; and
(xv) a VH comprising a sequence having at least 70%, at least 75%, at least 80%, at least 85%, at least 90%, at least 95%, at least 97%, at least 99%, or 100% identity to the sequence of SEQ ID NO: 113 and a VL comprising a sequence having at least 70%, at least 75%, at least 80%, at least 85%, at least 90%, at least 95%, at least 97%, at least 99%, or 100% identity to the sequence of SEQ ID NO: 117.

In one embodiment, the antibody comprises:
(i) a first polypeptide comprising a heavy chain (HC) and
(ii) a second polypeptide comprising a light chain (LC).

In one embodiment, the antibody comprises a first binding arm and a second binding arm, wherein
a. the first binding arm comprises:
(i) a first polypeptide comprising a heavy chain (HC) and
(ii) a second polypeptide comprising a light chain (LC) and;
b. the second binding arm comprises:
(i) a first polypeptide comprising a heavy chain (HC) and
(ii) a second polypeptide comprising a light chain (LC).

In one embodiment, the first polypeptide of the first binding arm and the first polypeptide of the second binding arm are identical. In one embodiment, the second polypeptide of the first binding arm and the second polypeptide of the second binding arm are identical.

In one embodiment, the heavy chain (HC) comprises a heavy chain variable region (VH) and a heavy chain constant region (CH). In one embodiment, the heavy chain constant region (CH) comprises a constant region domain 1 region (CH1), a hinge region, a constant region domain 2 region (CH2), and a constant region domain 3 region (CH3). In one embodiment, the light chain (LC) comprises a light chain variable region (VL) and a light chain constant region (CL).

In one embodiment, a heavy chain variable region (VH) and a light chain variable region (VL) together provide a first binding domain. In one embodiment, a heavy chain variable region (VH) and a light chain variable region (VL) on the same binding arm together provide a first binding domain.

In one embodiment, an antibody described herein which may be part of a binding molecule described herein may be modified to induce Fc-mediated effector function to a lesser extent compared to a parental antibody. In one embodiment of the invention, the heavy chain constant regions are modified so that the antibody induces Fc-mediated effector function to a lesser extent compared to an antibody which is identical except for comprising non-modified heavy chains. In one embodiment, the Fc-mediated effector function is measured by binding to IgG Fc (Fcγ) receptors, binding to C1q, or induction of Fc-mediated cross-linking of FcRs. In one embodiment, said Fc-mediated effector function is measured by binding to C1q. In one embodiment, the heavy chain constant regions have been modified so that binding of C1q to said antibody is reduced compared to a parental antibody, preferably reduced by at least 70%, at least 80%, at least 90%, at least 95%, at least 97%, or 100%, wherein C1q binding is preferably determined by ELISA. In one embodiment, in at least one of said first and second heavy chain constant regions one or more amino acids in the positions corresponding to positions L234 and L235 in a human IgG1 heavy chain according to EU numbering, are not L and L, respectively. In one embodiment, the positions corresponding to positions L234 and L235 in a human IgG1 heavy chain according to EU numbering are A and A, respectively, in said first and second heavy chain constant regions.

In a further aspect, the present invention provides a recombinant nucleic acid which encodes a binding agent described herein or an antibody described herein. In one embodiment, the recombinant nucleic acid is RNA.

In a further aspect, the present invention provides a cell transfected with a recombinant nucleic acid described herein. In one embodiment, the cell expresses the binding agent or the antibody.

In a further aspect, the present invention provides a pharmaceutical composition comprising a binding agent described herein, an antibody described herein, or a recombinant nucleic acid described herein.

In a further aspect, the present invention provides the binding agent described herein, the antibody described herein, or the recombinant nucleic acid described herein for therapeutic use. In one embodiment, the therapeutic use comprises a therapeutic or prophylactic treatment of a coronavirus infection in a subject. In one embodiment, the therapeutic use comprises neutralizing coronavirus in a subject. In one embodiment, the subject is human.

In one embodiment of the binding agent described herein, the antibody described herein, the recombinant nucleic acid described herein, the cell described herein, or the pharmaceutical composition described herein, the coronavirus is a betacoronavirus.

In one embodiment of the binding agent described herein, the antibody described herein, the recombinant nucleic acid described herein, the cell described herein, or the pharmaceutical composition described herein, the coronavirus is a sarbecovirus.

In one embodiment of the binding agent described herein, the antibody described herein, the recombinant nucleic acid described herein, the cell described herein, or the pharmaceutical composition described herein, the coronavirus is SARS-CoV-1 and/or SARS-CoV-2.

In a further aspect, the present invention provides a method of treating or preventing a coronavirus infection comprising administering to a subject the binding agent described herein, the antibody described herein, the recombinant nucleic acid described herein, or the pharmaceutical composition described herein. Embodiments of the coronavirus are as described herein.

In one aspect, the invention relates to an agent or composition described herein for use in a method described herein.

It is also demonstrated herein that IgG-scFv bispecific binding agents can be expressed in vivo by administering RNA and that IgG-scFv bispecific binding agents were correctly assembled and folded. Thus, the invention also relates to the following exemplary embodiments:

1. A composition or medical preparation comprising:
(i) a first RNA encoding a first polypeptide chain comprising an immunoglobulin heavy chain; and
(ii) a second RNA encoding a second polypeptide chain comprising an immunoglobulin light chain and a single chain Fv (scFv).

2. The composition or medical preparation of embodiment 1, wherein the scFv is linked to the N-terminus or C-terminus of the light chain.

3. The composition or medical preparation of embodiment 1 or 2, wherein the scFv is linked to the C-terminus of the light chain.

4. The composition or medical preparation of any one of embodiments 1 to 3, wherein the immunoglobulin heavy chain comprises a variable region of a heavy chain (VH) and the immunoglobulin light chain comprises a variable region of a light chain (VL).

5. The composition or medical preparation of any one of embodiments 1 to 4, wherein the immunoglobulin heavy chain interacts with the immunoglobulin light chain to form a first binding domain.

6. The composition or medical preparation of any one of embodiments 1 to 5, wherein the variable region of a heavy chain (VH) of the immunoglobulin heavy chain interacts with the variable region of a light chain (VL) of the immunoglobulin light chain to form a first binding domain.

7. The composition or medical preparation of any one of embodiments 1 to 6, wherein two of the immunoglobulin heavy chains and two of the immunoglobulin light chains form a full-length antibody.

8. The composition or medical preparation of any one of embodiments 1 to 7, wherein the scFv comprises a variable region of a heavy chain (VH) of an immunoglobulin and a variable region of a light chain (VL) of an immunoglobulin.

9. The composition or medical preparation of any one of embodiments 1 to 8, wherein the VH of the scFv interacts with the VL of the scFv to form a second binding domain.

10. The composition or medical preparation of embodiment 9, wherein the first binding domain and the second binding domain bind to different epitopes, wherein the different epitopes are present on the same or on different antigens.

11. The composition or medical preparation of any one of embodiments 1 to 10, wherein two of the first polypeptide chains and two of the second polypeptide chains form a full-length antibody, wherein an scFv is linked to each of the light chains.

12. The composition or medical preparation of any one of embodiments 1 to 11, wherein the first polypeptide chain comprises a constant region 1 of a heavy chain (CH1) or a functional variant thereof and the second polypeptide chain comprises a constant region of a light chain (CL) or a functional variant thereof.

13. The composition or medical preparation of embodiment 12, wherein the first polypeptide chain further comprises a constant region 2 of a heavy chain (CH2) or a functional variant thereof and optionally further comprises a constant region 3 of a heavy chain (CH3) or a functional variant thereof.

14. The composition or medical preparation of any one of embodiments 1 to 13, wherein the immunoglobulin is an antibody.

15. The composition or medical preparation of any one of embodiments 1 to 14, wherein the immunoglobulin is IgG.

16. The composition or medical preparation of embodiment 15, wherein the IgG is human IgG.

17. The composition or medical preparation of any one of embodiments 1 to 16, wherein the first polypeptide chain comprises, from N-terminus to C-terminus, in the order VH-CH1, wherein the CH may optionally be modified.

18. The composition or medical preparation of any one of embodiments 1 to 17, wherein the first polypeptide chain comprises, from N-terminus to C-terminus, in the order VH-CH1-CH2, wherein the CH may optionally be modified.

19. The composition or medical preparation of any one of embodiments 1 to 18, wherein the first polypeptide chain comprises, from N-terminus to C-terminus, in the order VH-CH1-CH2-CH3, wherein the CH may optionally be modified.

20. The composition or medical preparation of any one of embodiments 1 to 19, wherein the second polypeptide chain comprises, from N-terminus to C-terminus, in the order
VL-CL-VH(scFv)-VL(scFv); or
VL-CL-VL(scFv)-VH(scFv).

21. The composition or medical preparation of embodiment 20, wherein VH interacts with VL to form a binding domain and VH(scFv) interacts with VL(scFv) to form a binding domain.

22. The composition or medical preparation of any one of embodiments 1 to 21, wherein the scFv is linked to the light chain by a peptide linker.

23. The composition or medical preparation of any one of embodiments 20 to 22, wherein the CL is connected to the VH(scFv) or VL(scFv) by a peptide linker.

24. The composition or medical preparation of embodiment 22 or 23, wherein the peptide linker comprises a GS linker.

25. The composition or medical preparation of any one of embodiments 1 to 24, wherein the VH and the VL of the scFv are connected to one another by a peptide linker.

26. The composition or medical preparation of embodiment 25, wherein the peptide linker comprises a GS linker.

27. The composition or medical preparation of embodiment 26, wherein the peptide linker comprises the amino acid sequence (G₄S)₄ or a functional variant thereof.

28. The composition or medical preparation of any one of embodiments 20 to 27, wherein the CH1 on the first polypeptide chain interacts with the CL on the second polypeptide chain.

29. The composition or medical preparation of any one of embodiments 1 to 28, wherein the first RNA and/or the second RNA comprises a modified nucleoside in place of uridine.

30. The composition or medical preparation of embodiment 29, wherein the modified nucleoside is selected from pseudouridine (ψ), N1-methyl-pseudouridine (m1ψ), and 5-methyl-uridine (m5U).

31. The composition or medical preparation of any one of embodiments 1 to 30, wherein the first RNA and/or the second RNA comprises a cap.

32. The composition or medical preparation of embodiment 31, wherein the cap comprises m₂^{7,3′-O}Gppp(m₁^{2′-O})ApG.

33. The composition or medical preparation of any one of embodiments 1 to 32, wherein the first RNA and/or the second RNA comprises a 5′ UTR.

34. The composition or medical preparation of embodiment 33, wherein the 5′ UTR comprises the nucleotide sequence of SEQ ID NO: 199, or a nucleotide sequence having at least 99%, 98%, 97%, 96%, 95%, 90%, 85%, or 80% identity to the nucleotide sequence of SEQ ID NO: 199.

35. The composition or medical preparation of any one of embodiments 1 to 34, wherein the first RNA and/or the second RNA comprises a 3′ UTR.

36. The composition or medical preparation of embodiment 35, wherein the 3′ UTR comprises the nucleotide sequence of SEQ ID NO: 201, or a nucleotide sequence having at least 99%, 98%, 97%, 96%, 95%, 90%, 85%, or 80% identity to the nucleotide sequence of SEQ ID NO: 201.

37. The composition or medical preparation of any one of embodiments 1 to 36, wherein the first RNA and/or the second RNA comprises a poly-A sequence.

38. The composition or medical preparation of embodiment 37, wherein the poly-A sequence comprises at least 100 nucleotides.

39. The composition or medical preparation of embodiment 37 or 38, wherein the poly-A sequence comprises the nucleotide sequence of SEQ ID NO: 202.

40. The composition or medical preparation of any one of embodiments 1 to 39, wherein the RNA is formulated as a liquid, formulated as a solid, or a combination thereof.

41. The composition or medical preparation of any one of embodiments 1 to 40, wherein the RNA is formulated or is to be formulated for injection.

42. The composition or medical preparation of any one of embodiments 1 to 41, wherein the RNA is formulated or is to be formulated for intravenous administration.

43. The composition or medical preparation of any one of embodiments 1 to 42, wherein the RNA is formulated or is to be formulated as particles.

44. The composition or medical preparation of embodiment 50, wherein the particles are lipid nanoparticles (LNP).

45. The composition or medical preparation of embodiment 44, wherein the LNP particles comprise a cationic lipid, a neutral lipid (e.g., a phospholipid), a polymer-conjugated lipid (e.g., a pegylated lipid), and a steroid (e.g., cholesterol).

46. The composition or medical preparation of any one of embodiments 1 to 45, which is a pharmaceutical composition.

47. The composition or medical preparation of embodiment 46, wherein the pharmaceutical composition further comprises one or more pharmaceutically acceptable carriers, diluents and/or excipients.

48. The composition or medical preparation of any one of embodiments 1 to 47, wherein the medical preparation is a kit.

49. The composition or medical preparation of embodiment 48, wherein the RNA and optionally the particle forming components are in separate vials.

50. The composition or medical preparation of any one of embodiments 1 to 49 for pharmaceutical use.

51. The composition or medical preparation of embodiment 50, wherein the pharmaceutical use comprises a therapeutic or prophylactic treatment of a disease or disorder.

52. The composition or medical preparation of any one of embodiments 1 to 51, for use in expressing a binding agent in a subject.

53. The composition or medical preparation of embodiment 52, wherein the binding agent is a binding agent that is at least bispecific.

54. The composition or medical preparation of embodiment 52 or 53, wherein the binding agent is a binding agent as described herein.

55. A method for expressing a binding agent in a subject comprising administering a first RNA and a second RNA as set forth in any one of embodiments 1 to 54 to the subject.

56. The method of embodiment 54, wherein the first RNA and the second RNA are administered simultaneously or sequentially.

57. A method for expressing a binding agent in a subject comprising administering the composition or medical preparation of any one of embodiments 1 to 54 to the subject.

58. The method of any one of embodiments 55 to 57, wherein the binding agent is a binding agent as described herein.

59. A first RNA and a second RNA as set forth in any one of embodiments 1 to 54 or a composition or medical preparation of any one of embodiments 1 to 54 for use in a method of any one of embodiments 55 to 58.

### BRIEF DESCRIPTION OF THE DRAWINGS

FIG. 1: Overview of anti-S1-antibody fusion constructs with human ACE-2 extracellular domain

The ECD of ACE-2 (aa 18-615) containing mutations R273Q, H345L, H374N, H378N is either fused N- or C-terminally to the light chain of the anti-S1 antibody and the anti-S1 heavy chain either contains the LS (M428L/N434S) mutation or not (A). In other constructs, the modified ACE-2 ACD is fused to an Fc (CH2-CH3) domain either containing the LS (M428L/N4345) mutation or not (B).

FIG. 2: SARS-CoV2-S1-RBD ELISA with anti-S1-antibody-ACE2 fusion proteins

Binding of anti-S1-antibody-ACE2 fusion proteins to immobilized SARS-CoV2-S1-RBD protein was tested in an ELISA. (A) The anti-S1-antibody (408/413), the anti-S1-antibody-ACE2 fusion proteins and ACE-2-hFc (402/403) as a control were tested in a serial dilution covering a concentration range from 20,000 to 0.013 ng/ml. (B) EC50 values are shown as determined after curve fitting using XLfit.

FIG. 3: SARS-CoV2-S1-RBD—ACE-2 neutralization assay with anti-S1-antibody-ACE2 fusion proteins

The inhibition of the ACE-2—SARS-CoV2-S1-RBD interaction by anti-S1-antibody-ACE2 fusion proteins was tested in a neutralization ELISA. (A) The anti-S1-antibody (408/413), the anti-S1-antibody-ACE2 fusion proteins and ACE-2-hFc (402/403) as a control were tested in a serial dilution covering a concentration range from 30,000 to 3.7 or 0.019 ng/ml, respectively. (B) IC50 values are shown as determined after curve fitting using XLfit.

FIG. 4: Pseudovirus neutralization activity by anti-S1-antibody-ACE2 fusion constructs

The anti-S1-antibody (413), ACE-2-hFc (402) and the anti-S1-antibody-ACE2 fusion proteins were tested in a pseudovirus neutralization test (pVNT). The graph shows the number of infected cells as measured by the expression of GFP (Y-axis). Concentrations of test samples ranged from 100 to 0.046 μg/ml. IC50 values are displayed as determined after curve fitting using GraphPad Prism.

FIG. 5: Affinities of anti-S1-antibody-ACE2 fusion proteins to active trimer SARS-CoV-2 S and SARS-CoV-2 S1-RBD protein

Shown are the on- and off-rates and the K_{D} of the anti-S1-antibody (413), ACE-2-hFc (402) and the anti-S1-antibody-ACE2 fusion proteins as measured by SPR using two different densities of SARS-CoV-2 S active trimer protein (A) or SARS-CoV-2 S1-RBD protein (B).

FIG. 6: SARS-CoV2-S1 ELISA with antibodies in B-cell supernatants

Binding of anti-SARS-CoV2-S1 antibodies in B-cell supernatants to immobilized SARS-CoV2-S1 protein was tested in an ELISA. (A) Shown are the rabbit antibody concentrations for each B-cell supernatant measured by a quantification ELISA. (B) B-cell supernatants and ACE-2-mFc as a control were tested in a serial dilution of 1:3 and concentrations are plotted according to the determined rIgG content (see example 6A). (C) Table 1: EC50 data of anti-S1 rabbit antibodies from B-cell supernatants in SARS-CoV-2 S1 Binding ELISA with a coating concentration of 3 μg/ml. Table 2: EC50 data of anti-S1 rabbit antibodies from B-cell supernatants in SARS-CoV-2 S1 Binding ELISA with a coating concentration of 0.85 μg/ml. EC50 values are shown as determined after curve fitting using XLfit.

FIG. 7: SARS-CoV2-S1—ACE-2 neutralization assay with antibodies in B-cell supernatants

The inhibition of the ACE-2—SARS-CoV2-S1 interaction by anti-SARS-CoV2-S1 antibodies in B-cell supernatants was tested in a neutralization ELISA. (A) B-cell supernatants and ACE-2-mFc as a control were tested in a serial dilution of 1:3 and concentrations are plotted according to the determined rIgG content (see example 6A). (B) IC50 values are shown as determined after curve fitting using XLfit.

FIG. 8: SARS-CoV2-S, SARS-CoV2-S1-RBD and SARS-CoV-S1-RBD ELISA with purified antibodies of the invention

The binding of the purified chimeric antibodies of the invention to immobilized SARS-CoV2-S active trimer, SARS-CoV2-S1-RBD or SARS-CoV-S1-RBD was tested in an ELISA. (A) Purified chimeric antibodies as well as ACE-2-hFc (402/403), the anti-S1-antibody-ACE2 fusion protein (406) and anti-S1-antibody (408) as controls were tested in serial dilutions covering concentrations from 1,000 to 0.0006 ng/ml. (B) EC50 values are shown as determined after curve fitting using XLfit.

FIG. 9: SARS-CoV2-S1-RBD—ACE-2 neutralization assay with purified antibodies of the invention

The inhibition of the ACE-2—SARS-CoV2-S1-RBD interaction by purified, chimeric antibodies of the invention was tested in a neutralization ELISA. (A) Purified chimeric antibodies as well as ACE-2-hFc (402/403), the anti-S1-antibody-ACE2 fusion protein (406) and anti-S1-antibody (408) as controls were tested in serial dilution covering concentrations from 30,000 to 0.019 ng/ml. (B) IC50 values are shown as determined after curve fitting using XLfit.

FIG. 10: Pseudovirus neutralization activity by purified antibodies of the invention

The purified, chimeric antibodies of the invention were tested in a pseudovirus neutralization test (pVNT). (A) The chimeric antibodies as well as anti-S1-antibody (413), ACE2-hFc (403) and the anti-S1-antibody-ACE2 fusion protein (411) were tested in serial dilution in concentrations from 100 to 0.049 μg/ml or 30 to 0.01 μg/ml. The graph shows the number of infected cells per well as measured by the expression of GFP (Y-axis). The IC50 and IC90 values for selected antibodies of the invention are summarized in (B).

FIG. 11: SARS-CoV2-S1-RBD epitope competition among antibodies of the invention

The competition of purified, chimeric antibodies for epitope binding on SARS-CoV2-S1-RBD was tested in a competition ELISA. Different combinations of one chimeric antibody with another are summarized in the table. Competing antibodies are marked as +, non-competing as − and not determined pairs as nd.

FIG. 12: Overview of bispecific anti-S1 antibodies

scFvs derived from either the anti-S1 antibody or antibodies of the invention (New-scFv) are coupled to the light chain of either the anti-S1 antibody or the antibodies of the invention, respectively. Alternatively, New-scFvs are coupled to the light chain of another antibody of the invention.

FIG. 13: Modular schemes illustrating the RNA-constructs and the encoded anti-S1-antibody-ACE2 fusion RiboMabs.

(A) Design of the heavy chain (HC, top) and light chain ACE2 extra cellular domain (ECD) fusion (LC-ACE2, bottom) on IVT-mRNA level. (B) Illustration of the translated anti-S1-antibody-ACE2-RiboMabs, left, RiboMab_411, right, RiboMab_406. Curved lines symbolize the glycine-serine [(Gly4Ser)4] linker.

ACE2, angiotensin-converting enzyme 2; a-S1, anti-spike protein 1; CL, constant light chain region; CH, constant heavy chain region; ECD, extracellular domain; Fc, fragment crystallizable region; GS, (Gly4Ser)4 linker; HC, heavy chain; LC, light chain; LS, methionine 428 leucine and asparagine 434 serine substitution; Poly(A), poly(A) tail; Sec, secretion signal; UTR, untranslated region; VH, variable heavy chain domain; VL, variable light chain domain.

FIG. 14: Expression of anti-S1-antibody-ACE2 fusion RiboMabs in vitro.

Human embryonic kidney cell line HEK 293T/17 was transiently transfected via electroporation with the indicated IVT-mRNA mass-related ratios of the heavy chain (HC) and the light chain ACE2 ECD fusion (LC-ACE2) for RiboMab_411 or RiboMab_406 or with RNA buffer only (Mock). HEK 293T/17 cell culture supernatants (SN) containing secreted RiboMab were harvested 48 hours post electroporation and subjected to (A) Gyros immunoassay quantitation and (B, C) Western Blot analyses. (A) RiboMab concentration in SN after transfection of HC:LC-ACE2 ratios as indicated (x-axis) was analyzed with a fluorescently-labeled anti-human IgG detection antibody via Gyros sandwich immunoassay. (B, C) Western Blot analysis was performed for the detection of translated RiboMabs. 22.5 ng of purified reference protein ID 411 (Ref. protein), 7.5 μL Mock or 7.5 μL RiboMab-containing SN was loaded and separated by polyacrylamide gradient gel electrophoresis (4-15% polyacrylamide) under (B) non-reducing and (C) reducing conditions. Two different molecular weight standards (MW std. 1 and 2) were applied. Proteins were detected with a mixture of two polyclonal horseradish peroxidase-conjugated goat anti-human IgG, Fcγ-fragment specific (1:2,000) and kappa LC-specific (1:200) antibodies.

Fc, fragment crystallizable region of IgG; HC, heavy chain; IB, immunoblot; IgG; immunoglobulin G; LC, light chain; MW std., molecular weight standard; SN, supernatant.

FIG. 15: Estimation of in vivo pharmacokinetics of anti-S1-antibody-ACE2 fusion RiboMabs.

12 female Balb/cJRj mice per group received a single intravenous injection of 30 μg RNA-LNP encoding RiboMab_406, RiboMab_411 or luciferase (negative control). Blood samples from four mice per time point were drawn. RiboMab concentrations were measured via Gyros immunoassay in serum samples 6, 24, 48, 96 and 240 hours (0.25 to 10 days) post administration as shown. The concentration is plotted in log 10 scale on the y-axis. Error bars are standard errors of the mean (n=4).

FIG. 16: Western Blot analysis of in vivo expressed anti-S1-antibody-ACE2 fusion RiboMabs.

Serum was sampled from female Balb/cJRj mice injected with 30 μg RNA-LNP encoding RiboMab_406 or RiboMab_411 six hours post administration and subjected to Western Blot analysis. Purified reference protein ID 411 diluted in buffer (Ref. protein) or serum of untreated mice (Ref. protein in serum) served as positive control. Serum of mice injected with luciferase-encoding RNA-LNP was used as negative control. 10 ng reference protein or 5 μL of serum sample was separated by polyacrylamide gradient gel electrophoresis (4-15%) under (A) non-reducing and (B) reducing conditions. Two different molecular weight standards (MW std. 1 and 2) were applied. Proteins were detected with a mixture of two polyclonal horseradish peroxidase-conjugated goat anti-human IgG, Fcγ-fragment specific (1:2,000) and kappa LC-specific (1:200) antibodies.

Fc, fragment crystallizable region of IgG; h, human; HC, heavy chain; IB, immunoblot; IgG; immunoglobulin G; LC, light chain; MW std., molecular weight standard; SN, supernatant.

FIG. 17: Pseudovirus neutralization activity by RiboMab_411 and 406.

RiboMab_406 and RiboMab_411 in HEK 2931/17 cell culture SN were tested in a pseudovirus neutralization test. SN of cells transfected with the HC only were used as Mock control (Mock 1, HC of RiboMab_406, Mock 2, HC of RiboMab_411). The samples were tested in a serial dilution with final concentrations ranging from 30 to 0.23 μg/mL. The graph shows the number of infected cells per well as measured by the expression of GFP (Y-axis). The table below shows the IC50 values (μg/mL).

FIG. 18: Binding of bispecific anti-S1-antibody-scFv fusion proteins to recombinant SARS-CoV2 S1-RBD protein

Binding of anti-S1-antibody-scFv fusion proteins to immobilized SARS-CoV2-S1-RBD protein was tested in an ELISA. (A) The anti-S1-antibody (408), the antibodies of the invention and the anti-S1-antibody-scFv fusion proteins were tested in a serial dilution covering a concentration range from 1,000 to 0.001 ng/ml. (B) EC50 values are shown as determined after curve fitting using XLfit.

FIG. 19: SARS-CoV2-S1-RBD—ACE-2 neutralization assay with anti-S1-antibody-scFv fusion proteins

The inhibition of the ACE-2—SARS-CoV2-S1-RBD interaction by anti-S1-antibody-scFv fusion proteins was tested in a neutralization ELISA. (A) The anti-S1-antibody (408), the antibodies of the invention and the anti-S1-antibody-scFv fusion proteins were tested in a serial dilution covering a concentration range from 30,000 to 0.019 ng/ml, respectively. (B) IC50 and IC90 values are shown as determined after curve fitting using XLfit.

FIG. 20: Affinities of S1 targeting antibodies of the invention to SARS-CoV-2 S1-RBD protein

Shown are the on- and off-rates and the K_{D} of the anti-S1-antibody (413) and the antibodies of the invention as measured by SPR using immobilized SARS-CoV-2 S1-RBD protein.

FIG. 21: Pseudovirus neutralization of bispecific antibodies The purified bispecific antibody constructs 465 and 467 were tested in a pseudovirus neutralization test (pVNT). (A) The graph shows the number of infected cells per well as measured by the expression of GFP (Y-axis). The IC50 and IC90 values for selected antibodies of the invention are summarized in (B).

FIG. 22: Pseudovirus neutralization of bispecific antibodies

Purified bispecific antibody constructs were tested in a pseudovirus neutralization test (pVNT). (A) The graph shows the number of infected cells per well as measured by the expression of GFP (Y-axis). The IC50 and IC90 values for antibodies of the invention are summarized in (B).

FIG. 23: Pseudovirus neutralization of bispecific antibodies

Purified bispecific antibody constructs 498, 500, 501 and 502 were tested in a pseudovirus neutralization test (pVNT). (A) The graph shows the number of infected cells per well as measured by the expression of GFP (Y-axis). The IC50 and IC90 values for antibodies of the invention are summarized in (B).

FIG. 24: Affinity of S1 targeting antibody 470 of the invention to SARS-CoV-2 S1-RBD protein

Shown are the on- and off-rates and the K_{D} of the anti-S1-antibody 470 of the invention as measured in a multicycle kinetic SPR analysis using immobilized SARS-CoV-2 S1-RBD protein. Multicyle kinetics were recorded on two different flow cells and in three replicates each.

FIG. 25: Modular schemes illustrating the RNA-constructs encoding IgG RiboMabs.

(A) Design of the heavy chain (HC, top) and light chain (LC, bottom) anti-SARS-CoV-2 encoding IgG RiboMabs on IVT-mRNA level. (B) Illustration of the translated IgG RiboMab protein. CL, constant light chain region; CH, constant heavy chain region; Fc, fragment crystallizable region; HC, heavy chain; LC, light chain; LALA, leucine-to-alanine (codon 234) and leucine-to-alanine (codon 235) substitutions; LS, methionine-to-leucine (codon 428) and asparagine-to-serine (codon 434) substitutions; Poly(A), poly(A) tail; Sec, secretion signal; UTR, untranslated region; VH, variable heavy chain domain; VL, variable light chain domain.

FIG. 26: Expression of anti-SARS-CoV-2 IgG RiboMabs in vitro.

Human embryonic kidney cell line HEK 293T/17 was transiently transfected via electroporation with IVT-mRNA mass-related ratios of the heavy chain (HC) and the light chain of 1.5:1 for the indicated RiboMabs. HEK 2931/17 cell culture supernatants (SN) containing secreted RiboMab were harvested 48 hours post electroporation and subjected to Gyros immunoassay quantitation. RiboMab concentration in SN after transfection of HC:LC ratios as indicated (x-axis) was analyzed with a fluorescently-labeled anti-human IgG detection antibody via Gyros sandwich immunoassay.

Ref., reference (anti-S1 IgG antibody including LALA and LS mutations).

FIG. 27: Pseudovirus neutralization activity by in vitro expressed anti-SARS-CoV-2 IgG RiboMab candidates.

HEK 2931/17 cell culture SN containing anti-SARS-CoV-2 RiboMabs as indicated on the x-axis of the respective graphs were tested in pseudovirus neutralization tests. The samples were tested in a serial dilution with final concentrations ranging from 30,000 to 7.3 ng/mL. (A) Graphs show the number of infected cells per well as measured by the expression of luciferase (Y-axis) in relative luminescence units (RLU). Error bars are standard errors of the mean (technical triplicates). Horizontal dotted lines indicate the benchmark (in RLU) for virus control. (B) Table showing the IC50 and IC90 values (ng/mL) of RiboMabs and the RiboMab IgG reference protein.

IC50, half maximal inhibitory concentration; IC90, concentration required to inhibit 90% of pseudovirus replication; ID, identification number; IgG, immunoglobulin gamma; NC, not calculable; ref., reference; RLU, relative luminescence units.

FIG. 28: Estimation of in vivo pharmacokinetics of selected anti-SARS-CoV-2 IgG RiboMabs.

Four female Balb/cJRj mice per group received a single intravenous injection of 30 μg RNA-LNP encoding RiboMab_445, RiboMab_447, RiboMab_470, RiboMab_472, anti-SARS-CoV-2 RiboMab IgG reference or luciferase (negative control). An anti-S1 antibody (protein ID 408) reference was administered as protein reference at a dose of 100 μg. Blood samples from four mice per time point were drawn. RiboMab concentrations were measured via Gyros immunoassay in serum samples prepared 24, 96, 168, 216, 336 and 504 hours (Days 1 to 21 as indicated on the x-axis) after administration. No protein was detected in the negative control group (luciferase RNA-LNP, data not shown). The concentration is plotted in log 10 scale on the y-axis. Error bars represent standard error of the mean (biological quadruplices). ID, identification number; IgG, immunoglobulin gamma.

FIG. 29: Pseudovirus neutralization activity by in vivo expressed anti-SARS-CoV-2 IgG RiboMab candidates.

Balb/cJRj mouse serum samples containing anti-SARS-CoV-2 RiboMabs as indicated on the x-axis of the respective graphs were tested in pseudovirus neutralization tests using the wild-type SARS-CoV-2 spike protein. The samples were tested in 12-point, 2-fold (RiboMab_447) or 3-fold (RiboMab_445/470/472) serial dilutions. The starting concentration varied from sample to sample and was in the range of approximately 30 to 60 μg/mL. Graphs show the number of infected cells per well as measured by the expression of luciferase (Y-axis). Error bars represent the standard error of the mean (technical triplicates). (B) Table showing the IC50 and IC90 values (ng/mL) of RiboMabs and the protein reference (protein ID 408) in serum. IC50, half maximal inhibitory concentration; IC90, concentration required to inhibit 90% of pseudovirus replication; ID, identification number; ref., reference; RLU, relative luminescence units.

FIG. 30: Modular schemes illustrating the RNA-constructs and the encoded bispecific IgG-scFv RiboMab.

(A) Design of the heavy chain (HC, top) and light chain (LC-scFv, bottom) anti-SARS-CoV-2 encoding bispecific IgG-scFv RiboMabs on IVT-mRNA level. VH #1 and VL #1 use the coding sequences from the first anti-SARS-CoV-2 antibody, while VH #2 and VL #2 coding sequences derive from the second anti-SARS-CoV-2 specific antibody. (B) Illustration of the translated IgG-scFv RiboMab protein. Curved lines symbolize the glycine-serine (GS) linkers. The bold lines between VH #2 and VL #2 indicate the disulfide bridge stabilizing the scFv. CL, constant light chain region; CH, constant heavy chain region; ds, disulfide bridge; Fc, fragment crystallizable region; HC, heavy chain; GS, glycine-serine linker; LC, light chain; LALA, leucine-to-alanine (codon 234) and leucine-to-alanine (codon 235) substitutions; LS, methionine-to-leucine (codon 428) and asparagine-to-serine (codon 434) substitutions; Poly(A), poly(A) tail; Sec, secretion signal; scFv, single-chain variable fragment; UTR, untranslated region; VH, variable heavy chain domain; VL, variable light chain domain.

FIG. 31: Estimation of in vivo pharmacokinetics of selected anti-SARS-CoV-2 bispecific IgG-scFv RiboMabs.

Four female Balb/cJRj mice per group received a single intravenous injection of 30 μg RNA-LNP encoding RiboMab_498, RiboMab_500, RiboMab_502 or luciferase (negative control). An anti-S1 antibody (protein ID 408) reference was administered as protein reference at a dose of 250 μg. Blood samples from four mice per time point were drawn. RiboMab concentrations were measured via Gyros immunoassay in serum samples prepared 6, 24, 48, 96, 168, 336 and 504 hours (Days 0.25 to 21 as indicated on the x-axis) after administration. No protein was detected in the negative control group (luciferase RNA-LNP, data not shown). The concentration is plotted in log 10 scale on the y-axis. Error bars represent standard error of the mean (biological quadruplices).

ID, identification number; IgG, immunoglobulin gamma.

FIG. 32: Pseudovirus neutralization activity by in vivo expressed anti-SARS-CoV-2 bispecific IgG-scFv RiboMab candidates.

Two female Balb/cJRj mice per group received a single intravenous injection of 30 μg RNA-LNP encoding RiboMab_498, RiboMab_500, RiboMab_502 or luciferase (negative control). An anti-S1 antibody (protein ID 408) reference was administered as protein reference at a dose of 250 μg. Blood samples from two mice were drawn 24 hours after administration. Mouse serum samples containing anti-SARS-CoV-2 RiboMabs were tested in pseudovirus neutralization tests using the SARS-CoV-2 spike protein variant of the wild-type, 8.1.1.7, B.1.351 and B.1.617. The samples of the wild-type, B.1.1.7 and B.1.351 variants were tested in 12-point, 4-fold serial dilutions with final concentrations ranging from 5,000 to 0.0012 ng/mL. The samples of the B.1.617 variant were tested in a 14-point, 4-fold serial dilution with concentrations ranging from 20,000 to 0.0003 ng/mL. (A) Graph showing the IC50 values from pVNT assay of the indicated RiboMabs and against the indicated pseudovirus variants. Error bars are standard errors of the mean (technical triplicates). (B) Table showing the IC50 and IC90 values (ng/mL) of RiboMabs in serum against the indicated pseudovirus variants. IC50, half maximal inhibitory concentration; IC90, concentration required to inhibit 90% of pseudovirus replication; RLU, relative luminescence units; WT, wild-type.

FIG. 33: Bispecific IgG-scFv RiboMab protein integrity in mouse serum Serum was sampled from female Balb/cJRj mice 24 hours after administration with 30 μg RNA-LNP encoding RiboMab_498, RiboMab_500 or RiboMab_502. (A) Western Blot analysis of the serum samples. Serum of mice injected with luciferase-encoding RNA-LNP was used as negative control (neg. control). 10 ng reference protein or 11.25 μL of each serum sample was separated by polyacrylamide gradient gel (4-15%) electrophoresis under non-reducing conditions. Proteins were detected with a polyclonal horseradish peroxidase-conjugated goat anti-human IgG antibody (H+L). (B) Table showing the quality analysis (monomeric vs. high molecule weight vs low molecule weight protein species) of the in vivo-expressed RiboMab IgG-scFv protein based on quantification with ImageLab software. Monomeric IgG-scFv is the desired protein product.

H, heavy chain; ID, identification number; IgG, immunoglobulin gamma; kD, kilo dalton; L, light chain; MW, molecular weight; scFv, single chain variable fragment; std., standard.

### DESCRIPTION OF THE SEQUENCES

The following tables provide a listing of certain sequences referenced herein.

TABLE 1
List of antibodies described herein based on SEQ ID NOs of heavy chain and light chain
variable regions (HC VR, LC VR), and complementarity-determining regions (CDRs).

| Antibody | Protein | Heavy chain SEQ ID NO | | | | Light chain SEQ ID NO | | | |
|---|---|---|---|---|---|---|---|---|---|
| Sample ID | Sample ID | HC VR | HCDR1 | HCDR2 | HCDR3 | LC VR | LCDR1 | LCDR2 | LCDR3 |
| P043.A.00023.A08 | 446 | 1 | 2 | 3 | 4 | 5 | 6 | 7 | 8 |
| P043.A.00017.E07 | 449 | 9 | 10 | 11 | 12 | 13 | 14 | 15 | 16 |
| P043.A.00023.E05 | 443 | 17 | 18 | 19 | 20 | 21 | 22 | 23 | 24 |
| P043.A.00018.C02 | 450 | 25 | 26 | 27 | 28 | 29 | 30 | 31 | 32 |
| P043.A.00020.D06 | 444 | 33 | 34 | 35 | 36 | 37 | 38 | 39 | 40 |
| P043.A.00024.C11 | 451 | 41 | 42 | 43 | 44 | 45 | 46 | 47 | 48 |
| P043.A.00017.E04 | 445 | 49 | 50 | 51 | 52 | 53 | 54 | 55 | 56 |
| P043.A.00092.C12 | 472 | 57 | 58 | 59 | 60 | 61 | 62 | 63 | 64 |
| P043.A.00097.B02 | 471 | 65 | 66 | 67 | 68 | 69 | 70 | 71 | 72 |
| P043.A.00036.C07 | 448 | 73 | 74 | 75 | 76 | 77 | 78 | 79 | 80 |
| P043.A.00095.D06 | 470 | 81 | 82 | 83 | 84 | 85 | 86 | 87 | 88 |
| P043.A.00109.B12 | 468 | 89 | 90 | 91 | 92 | 93 | 94 | 95 | 96 |
| P043.A.00117.C08 | 469 | 97 | 98 | 99 | 100 | 101 | 102 | 103 | 104 |
| P043.A.00047.H08 | 447 | 105 | 106 | 107 | 108 | 109 | 110 | 111 | 112 |
| P043.A.00032.C04 | 452 | 113 | 114 | 115 | 116 | 117 | 118 | 119 | 120 |
| anti-S1 antibody | 408/413 | 121 | 122 | 123 | 124 | 125 | 126 | 127 | 128 |

TABLE 2
List of binding agents described here based on heavy chain and
light chain SEQ ID NOs and a short decription of final protein.

| Protein | Heavy Chain | Light Chain | |
|---|---|---|---|
| Sample ID | SEQ ID NO | SEQ ID NO | Final Protein Description |
| 402 | 131 | N/A | mutant, short ACE2 ECD - wt Fc fusion |
| 403 | 132 | N/A | mutant, short ACE2 ECD - Fc (LS) fusion |
| 404 | 133 | 134 | mut. ACE2 ECD-(G4S)4 -anti-S1 antibody LC & anti-S1 antibody HC (LS) |
| 405 | 135 | 136 | mut. ACE2 ECD-(G4S)5 -anti-S1 antibody LC & anti-S1 antibody HC (LS) |
| 406 | 137 | 138 | anti-S1 antibody LC-(G4S)4 -mut. ACE2 ECD & anti-S1 antibody HC (LS) |
| 407 | 139 | 140 | anti-S1 antibody LC-(G4S)5 -mut. ACE2 ECD & anti-S1 antibody HC (LS) |
| 408 | 141 | 142 | Antibody anti-S1 antibody (LS) |
| 409 | 143 | 144 | mut. ACE2 ECD-(G4S)4 -anti-S1 antibody LC & anti-S1 antibody wt HC |
| 410 | 145 | 146 | mut. ACE2 ECD-(G4S)5 -anti-S1 antibody LC & anti-S1 antibody wt HC |
| 411 | 147 | 148 | anti-S1 antibody LC-(G4S)4 -mut. ACE2 ECD & anti-S1 antibody wt HC |
| 412 | 149 | 150 | anti-S1 antibody LC-(G4S)5 -mut. ACE2 ECD & anti-S1 antibody wt HC |
| 413 | 151 | 152 | anti-S1 antibody (wt) |
| 463 | 153 | 154 | LC of anti-S1 antibody with a C-terminal fusion of a scFv of P043.A.00020.D06; HC of |
| | | | anti-S1 antibody as IgG1-LALA-LS |
| 464 | 155 | 156 | LC of anti-S1 antibody with a C-terminal fusion of a scFv of P043.A.00023.A08; HC of |
| | | | anti-S1 antibody as IgG1-LALA-LS |
| 465 | 157 | 158 | LC of anti-S1 antibody with a C-terminal fusion of a scFv of P043.A.00017.E07; HC of |
| | | | anti-S1 antibody as IgG1-LALA-LS |
| 466 | 159 | 160 | LC of anti-S1 antibody with a C-terminal fusion of a scFv of P043.A.00018.C02; HC of |
| | | | anti-S1 antibody as IgG1-LALA-LS |
| 467 | 161 | 162 | LC of anti-S1 antibody with a C-terminal fusion of a scFv of P043.A.00024.C11; HC of |
| | | | anti-S1 antibody as IgG1-LALA-LS |
| 460 | 163 | 164 | LC of P043.A.00020.D06 with a C-terminal fusion of a scFv of anti-S1 antibody; HC of |
| | | | P043.A.00020.D06 as IgG1-LALA-LS |
| 461 | 165 | 166 | LC of P043.A.00018.CO2 with a C-terminal fusion of a scFv of anti-S1 antibody; HC of |
| | | | P043.A.00018.C02 as IgG1-LALA-LS |
| 462 | 167 | 168 | LC of P043.A.00024.C11 with a C-terminal fusion of a scFv of anti-S1 antibody; HC of |
| | | | P043.A.00024.C11 as IgG1-LALA-LS |
| 454 | 169 | 170 | LC of P043.A.00023.A08 with a C-terminal fusion of a scFv of anti-S1 antibody; HC of |
| | | | P043.A.00023.A08 as IgG1-LALA-LS |
| 455 | 171 | 172 | LC of P043.A.00017.E07 with a C-terminal fusion of a scFv of anti-S1 antibody; HC of |
| | | | P043.A.00017.E07 as IgG1-LALA-LS |
| 473 | 173 | 174 | LC of anti-S1 antibody with a C-terminal fusion of a scFv of P043.A.00023.E05; HC of |
| | | | anti-S1 antibody as IgG1-LALA-LS |
| 474 | 175 | 176 | LC of anti-S1 antibody with a C-terminal fusion of a scFv of P043.A.00017.E04; HC of |
| | | | anti-S1 antibody as IgG1-LALA-LS |
| 475 | 177 | 178 | LC of anti-S1 antibody with a C-terminal fusion of a scFv of P043.A.00047.H08; HC of |
| | | | anti-S1 antibody as IgG1-LALA-LS |
| 453 | 179 | 180 | LC of P043.A.00023.E05 with a C-terminal fusion of a scFv of anti-S1 antibody; HC of |
| | | | P043.A.00023.E05 as IgG1-LALA-LS |
| 476 | 181 | 182 | LC of P043.A.00017.E04 with a C-terminal fusion of a scFv of anti-S1 antibody; HC of |
| | | | P043.A.00017.E04 as IgG1-LALA-LS |
| 477 | 183 | 184 | LC of P043.A.00047.H08 with a C-terminal fusion of a scFv of anti-S1 antibody; HC of |
| | | | P043.A.00047.H08 as IgG1-LALA-LS |
| 478 | 185 | 186 | LC of P043.A.00047.H08 with a C-terminal fusion of a scFv of P043.A.00023.E05; HC of |
| | | | P043.A.00047.H08 as IgG1-LALA-LS |
| 479 | 187 | 188 | LC of P043.A.00047.H08 with a C-terminal fusion of a scFv of P043.A.00017.E04; HC of |
| | | | P043.A.00047.H08 as IgG1-LALA-LS |
| 480 | 189 | 190 | LC of P043.A.00047.H08 with a C-terminal fusion of a scFv of P043.A.00024.C11; HC of |
| | | | P043.A.00047.H08 as IgG1-LALA-LS |
| 481 | 191 | 192 | LC of P043.A.00023.E05 with a C-terminal fusion of a scFv of P043.A.00047.H08; HC of |
| | | | P043.A.00023.E05 as IgG1-LALA-LS |
| 482 | 193 | 194 | LC of P043.A.00017.E04 with a C-terminal fusion of a scFv of P043.A.00047.H08; HC of |
| | | | P043.A.00017.E04 as IgG1-LALA-LS |
| 483 | 195 | 196 | LC of P043.A.00024.C11 with a C-terminal fusion of a scFv of P043.A.00047.H08; HC of |
| | | | P043.A.00024.C11 as IgG1-LALA-LS |
| 498 | 205 | 206 | LC of P043.A.00023.E05 with a C-terminal fusion of a scFv of P043.A.00095.D06; HC of |
| | | | P043.A.00023.E05 as IgG1-LALA-LS |
| 500 | 207 | 208 | LC of P043.A.00095.D06 with a C-terminal fusion of a scFv of P043.A.00023.E05; HC of |
| | | | P043.A.00095.D06 as IgG1-LALA-LS |
| 501 | 209 | 210 | LC of P043.A.00095.D06 with a C-terminal fusion of a scFv of P043.A.00017.E04; HC of |
| | | | P043.A.00095.D06 as IgG1-LALA-LS |
| 502 | 211 | 212 | LC of P043.A.00017.E04 with a C-terminal fusion of a scFv of P043.A.00095.D06; HC of |
| | | | P043.A.00017.E04 as IgG1-LALA-LS |

TABLE 3
DESCRIPTION OF OTHER SEQUENCES

| | | |
|---|---|---|
| SEQ | | |
| ID | | |
| NO: | Description | SEQUENCE |

ACE2 sequences

| | | |
|---|---|---|
| 129 | ACE2 ECD | QSTIEEQAKTFLDKFNHEAEDLFYQSSLASWNYNTNITEENVQNMNNAGDKWSAFLKEQST |
| | (Modified) | LAQMYPLQEIQNLTVKLQLQALQQNGSSVLSEDKSKRLNTILNTMSTIYSTGKVCNPDNPQ |
| | | ECLLLEPGLNEIMANSLDYNERLWAWESWRSEVGKQLRPLYEEYVVLKNEMARANHYEDYG |
| | | DYWRGDYEVNGVDGYDYSRGQLIEDVEHTFEEIKPLYEHLHAYVRAKLMNAYPSYISPIGC |
| | | LPAHLLGDMWGQFWTNLYSLTVPFGQKPNIDVTDAMVDQAWDAQRIFKEAEKFFVSVGLPN |
| | | MTQGFWENSMLTDPGNVQKAVCLPTAWDLGKGDFRILMCTKVTMDDFLTAHNEMGNIQYDM |
| | | AYAAQPFLLRNGANEGFHEAVGEIMSLSAATPKHLKSIGLLSPDFQEDNETEINFLLKQAL |
| | | TIVGTLPFTYMLEKWRWMVFKGEIPKDQWMKKWWEMKREIVGVVEPVPHDETYCDPASLFH |
| | | VSNDYSFIRYYTRTLYQFQFQEALCQAAKHEGPLHKCDISNSTEAGQKLFNMLRLGKSEPW |
| | | TLALENVVGAKNMNVRPLLNYFEPLFTWLKDQNKNSFVGWSTDWSPYAD |
| 130 | ACE2 | MSSSSWLLLSLVAVTAAQSTIEEQAKTFLDKFNHEAEDLFYQSSLASWNYNTNITEENVQN |
| | | MNNAGDKWSAFLKEQSTLAQMYPLQEIQNLTVKLQLQALQQNGSSVLSEDKSKRLNTILNT |
| | | MSTIYSTGKVCNPDNPQECLLLEPGLNEIMANSLDYNERLWAWESWRSEVGKQLRPLYEEY |
| | | VVLKNEMARANHYEDYGDYWRGDYEVNGVDGYDYSRGQLIEDVEHTFEEIKPLYEHLHAYV |
| | | RAKLMNAYPSYISPIGCLPAHLLGDMWGRFWTNLYSLTVPFGQKPNIDVTDAMVDQAWDAQ |
| | | RIFKEAEKFFVSVGLPNMTQGFWENSMLTDPGNVQKAVCHPTAWDLGKGDFRILMCTKVTM |
| | | DDFLTAHHEMGHIQYDMAYAAQPFLLRNGANEGFHEAVGEIMSLSAATPKHLKSIGLLSPD |
| | | FQEDNETEINFLLKQALTIVGTLPFTYMLEKWRWMVFKGEIPKDQWMKKWWEMKREIVGVV |
| | | EPVPHDETYCDPASLFHVSNDYSFIRYYTRTLYQFQFQEALCQAAKHEGPLHKCDISNSTE |
| | | AGQKLFNMLRLGKSEPWTLALENVVGAKNMNVRPLLNYFEPLFTWLKDQNKNSFVGWSTDW |
| | | SPYADQSIKVRISLKSALGDKAYEWNDNEMYLFRSSVAYAMRQYFLKVKNQMILFGEEDVR |
| | | VANLKPRISFNFFVTAPKNVSDIIPRTEVEKAIRMSRSRINDAFRLNDNSLEFLGIQPTLG |
| | | PPNQPPVSIWLIVFGVVMGVIVVGIVILIFTGIRDRKKKNKARSGENPYASIDISKGENNP |
| | | GFQNTDDVQTSF |

S protein sequences

| | | |
|---|---|---|
| 197 | S protein | MFVFLVLLPLVSSQCVNLTTRTQLPPAYTNSFTRGVYYPDKVFRSSVLHSTQDLFLPFFSN |
| | (amino | VTWFHAIHVSGTNGTKRFDNPVLPFNDGVYFASTEKSNIIRGWIFGTTLDSKTQSLLIVNN |
| | acid) | ATNVVIKVCEFQFCNDPFLGVYYHKNNKSWMESEFRVYSSANNCTFEYVSQPFLMDLEGKQ |
| | | GNFKNLREFVFKNIDGYFKIYSKHTPINLVRDLPQGFSALEPLVDLPIGINITRFQTLLAL |
| | | HRSYLTPGDSSSGWTAGAAAYYVGYLQPRTFLLKYNENGTITDAVDCALDPLSETKCTLKS |
| | | FTVEKGIYQTSNFRVQPTESIVRFPNITNLCPFGEVFNATRFASVYAWNRKRISNCVADYS |
| | | VLYNSASFSTFKCYGVSPTKLNDLCFTNVYADSFVIRGDEVRQIAPGQTGKIADYNYKLPD |
| | | DFTGCVIAWNSNNLDSKVGGNYNYLYRLFRKSNLKPFERDISTEIYQAGSTPCNGVEGFNC |
| | | YFPLQSYGFQPTNGVGYQPYRVVVLSFELLHAPATVCGPKKSTNLVKNKCVNFNFNGLTGT |
| | | GVLTESNKKFLPFQQFGRDIADTTDAVRDPQTLEILDITPCSFGGVSVITPGTNTSNQVAV |
| | | LYQDVNCTEVPVAIHADQLTPTWRVYSTGSNVFQTRAGCLIGAEHVNNSYECDIPIGAGIC |
| | | ASYQTQTNSPRRARSVASQSIIAYTMSLGAENSVAYSNNSIAIPTNFTISVTTEILPVSMT |
| | | KTSVDCTMYICGDSTECSNLLLQYGSFCTQLNRALTGIAVEQDKNTQEVFAQVKQIYKTPP |
| | | IKDFGGFNFSQILPDPSKPSKRSFIEDLLFNKVTLADAGFIKQYGDCLGDIAARDLICAQK |
| | | FNGLTVLPPLLTDEMIAQYTSALLAGTITSGWTFGAGAALQIPFAMQMAYRFNGIGVTQNV |
| | | LYENQKLIANQFNSAIGKIQDSLSSTASALGKLQDVVNQNAQALNTLVKQLSSNFGAISSV |
| | | LNDILSRLDKVEAEVQIDRLITGRLQSLQTYVTQQLIRAAEIRASANLAATKMSECVLGQS |
| | | KRVDFCGKGYHLMSFPQSAPHGVVFLHVTYVPAQEKNFTTAPAICHDGKAHFPREGVFVSN |
| | | GTHWFVTQRNFYEPQIITTDNTFVSGNCDVVIGIVNNTVYDPLQPELDSFKEELDKYFKNH |
| | | TSPDVDLGDISGINASVVNIQKEIDRLNEVAKNLNESLIDLQELGKYEQYIKWPWYIWLGF |
| | | IAGLIAIVMVTIMLCCMTSCCSCLKGCCSCGSCCKFDEDDSEPVLKGVKLHYT |
| 198 | S protein | VRFPNITNLCPFGEVFNATRFASVYAWNRKRISNCVADYSVLYNSASFSTFKCYGVSPTKL |
| | RBD | NDLCFTNVYADSFVIRGDEVRQIAPGQTGKIADYNYKLPDDFTGCVIAWNSNNLDSKVGGN |
| | (amino | YNYLYRLFRKSNLKPFERDAISTEIYQAGSTPCNGVEGFNCYFPLQSYGFQPTNGVGYQPY |
| | acid) | RVVVLSFELLHPATVCGPK |

5′-UTR (hAg-Kozak)

| | | |
|---|---|---|
| 199 | 5′-UTR | AACUAGUAUUCUUCUGGUCCCCACAGACUCAGAGAGAACCCGCCACC |

3′-UTR (2hBg)

| | | |
|---|---|---|
| 200 | 3′-UTR | CUCGAGAGCUCGCUUUCUUGCUGUCCAAUUUCUAUUAAAGGUUCCUUUGUUCCCUAAGUCC |
| | | AACUACUAAACUGGGGGAUAUUAUGAAGGGCCUUGAGCAUCUGGAUUCUGCCUAAUAAAAA |
| | | ACAUUUAUUUUCAUUGCUGCGUCGAGAGCUCGCUUUCUUGCUGUCCAAUUUCUAUUAAAGG |
| | | UUCCUUUGUUCCCUAAGUCCAACUACUAAACUGGGGGAUAUUAUGAAGGGCCUUGAGCAUC |
| | | UGGAUUCUGCCUAAUAAAAAACAUUUAUUUUCAUUGCUGCGUCGAGACCUGGUCCAGAGUC |
| | | GCUAGC |

3′-UTR (FI element)

| | | |
|---|---|---|
| 201 | 3'-UTR | CUGGUACUGCAUGCACGCAAUGCUAGCUGCCCCUUUCCCGUCCUGGGUACCCCGAGUCUCC |
| | | CCCGACCUCGGGUCCCAGGUAUGCUCCCACCUCCACCUGCCCCACUCACCACCUCUGCUAG |
| | | UUCCAGACACCUCCCAAGCACGCAGCAAUGCAGCUCAAAACGCUUAGCCUAGCCACACCCC |
| | | CACGGGAAACAGCAGUGAUUAACCUUUAGCAAUAAACGAAAGUUUAACUAAGCUAUACUAA |
| | | CCCCAGGGUUGGUCAAUUUCGUGCCAGCCACACC |

A30L70

| | | |
|---|---|---|
| 202 | A30L70 | AAAAAAAAAAAAAAAAAAAAAAAAAAAAAAGCAUAUGACUAAAAAAAAAAAAAAAAAAAAA |
| | | AAAAAAAAAAAAAAAAAAAAAAAAAAAAAAAAAAAAAAAAAAAAAAAAA |

Linker

| | | |
|---|---|---|
| 203 | (Gly4Ser)4 | GGGGSGGGGSGGGGSGGGGS |
| | linker | |
| 204 | (Gly4Ser)5 | GGGGSGGGGSGGGGSGGGGSGGGGS |
| | linker | |

### DETAILED DESCRIPTION

Although the present disclosure is described in detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present disclosure will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, the elements of the present disclosure will be described. These elements are listed with specific embodiments, however, it should be understood that they may be combined in any manner and in any number to create additional embodiments. The variously described examples and embodiments should not be construed to limit the present disclosure to only the explicitly described embodiments. This description should be understood to disclose and encompass embodiments which combine the explicitly described embodiments with any number of the disclosed elements. Furthermore, any permutations and combinations of all described elements should be considered disclosed by this description unless the context indicates otherwise.

The term “about” means approximately or nearly, and in the context of a numerical value or range set forth herein in one embodiment means±20%, ±10%, ±5%, or ±3% of the numerical value or range recited or claimed.

The terms “a” and “an” and “the” and similar reference used in the context of describing the disclosure (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the disclosure and does not pose a limitation on the scope of the claims. No language in the specification should be construed as indicating any non-claimed element essential to the practice of the disclosure.

Unless expressly specified otherwise, the term “comprising” is used in the context of the present document to indicate that further members may optionally be present in addition to the members of the list introduced by “comprising”. It is, however, contemplated as a specific embodiment of the present disclosure that the term “comprising” encompasses the possibility of no further members being present, i.e., for the purpose of this embodiment “comprising” is to be understood as having the meaning of “consisting of” or “consisting essentially of”. Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present disclosure was not entitled to antedate such disclosure.

### Definitions

In the following, definitions will be provided which apply to all aspects of the present disclosure. The following terms have the following meanings unless otherwise indicated. Any undefined terms have their art recognized meanings.

Terms such as “reduce”, “decrease”, “inhibit” or “impair” as used herein relate to an overall reduction or the ability to cause an overall reduction, preferably of at least 5%, at least 10%, at least 20%, at least 50%, at least 75% or even more, in the level. These terms include a complete or essentially complete inhibition, i.e., a reduction to zero or essentially to zero.

Terms such as “increase”, “enhance” or “exceed” preferably relate to an increase or enhancement by at least 10%, at least 20%, at least 30%, at least 40%, at least 50%, at least 80%, at least 100%, at least 200%, at least 500%, or even more.

According to the disclosure, the term “peptide” comprises oligo- and polypeptides and refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “protein” or “polypeptide” refers to large peptides, in particular peptides having at least about 150 amino acids, but the terms “peptide”, “protein” and “polypeptide” are used herein usually as synonyms.

“Fragment”, with reference to an amino acid sequence (peptide or protein), relates to a part of an amino acid sequence, i.e. a sequence which represents the amino acid sequence shortened at the N-terminus and/or C-terminus. A fragment shortened at the C-terminus (N-terminal fragment) is obtainable e.g. by translation of a truncated open reading frame that lacks the 3′-end of the open reading frame. A fragment shortened at the N-terminus (C-terminal fragment) is obtainable e.g. by translation of a truncated open reading frame that lacks the 5′-end of the open reading frame, as long as the truncated open reading frame comprises a start codon that serves to initiate translation. A fragment of an amino acid sequence comprises e.g. at least 50%, at least 60%, at least 70%, at least 80%, at least 90% of the amino acid residues from an amino acid sequence. A fragment of an amino acid sequence preferably comprises at least 6, in particular at least 8, at least 12, at least 15, at least 20, at least 30, at least 50, or at least 100 consecutive amino acids from an amino acid sequence.

By “variant” herein is meant an amino acid sequence that differs from a parent amino acid sequence by virtue of at least one amino acid modification. The parent amino acid sequence may be a naturally occurring or wild type (WT) amino acid sequence, or may be a modified version of a wild type amino acid sequence. Preferably, the variant amino acid sequence has at least one amino acid modification compared to the parent amino acid sequence, e.g., from 1 to about 20 amino acid modifications, and preferably from 1 to about 10 or from 1 to about 5 amino acid modifications compared to the parent.

By “wild type” or “WT” or “native” herein is meant an amino acid sequence that is found in nature, including allelic variations. A wild type amino acid sequence, peptide or protein has an amino acid sequence that has not been intentionally modified.

For the purposes of the present disclosure, “variants” of an amino acid sequence (peptide, protein or polypeptide) comprise amino acid insertion variants, amino acid addition variants, amino acid deletion variants and/or amino acid substitution variants. The term “variant” includes all mutants, splice variants, posttranslationally modified variants, conformations, isoforms, allelic variants, species variants, and species homologs, in particular those which are naturally occurring. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible. Amino acid addition variants comprise amino- and/or carboxy-terminal fusions of one or more amino acids, such as 1, 2, 3, 5, 10, 20, 30, 50, or more amino acids. Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants. Amino acid substitution variants are characterized by at least one residue in the sequence being removed and another residue being inserted in its place. Preference is given to the modifications being in positions in the amino acid sequence which are not conserved between homologous proteins or peptides and/or to replacing amino acids with other ones having similar properties. Preferably, amino acid changes in peptide and protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In one embodiment, conservative amino acid substitutions include substitutions within the following groups:
glycine, alanine;
valine, isoleucine, leucine;
aspartic acid, glutamic acid;
asparagine, glutamine;
serine, threonine;
lysine, arginine; and
phenylalanine, tyrosine.

Preferably the degree of similarity, preferably identity between a given amino acid sequence and an amino acid sequence which is a variant of said given amino acid sequence will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, in some embodiments continuous amino acids. In some embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS::needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g., at blast. ncbi.nlm.nih.gov/Blast.cgi?PAGE_TYPE=BlastSearch&BLAST_SPEC=blast2seq&LINK_LOC=align2seq). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, -2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment.

Percentage identity is obtained by determining the number of identical positions at which the sequences to be compared correspond, dividing this number by the number of positions compared (e.g., the number of positions in the reference sequence) and multiplying this result by 100.

In some embodiments, the degree of similarity or identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments continuous nucleotides. In some embodiments, the degree of similarity or identity is given for the entire length of the reference sequence. Homologous amino acid sequences exhibit according to the disclosure at least 40%, in particular at least 50%, at least 60%, at least 70%, at least 80%, at least 90% and preferably at least 95%, at least 98 or at least 99% identity of the amino acid residues.

The amino acid sequence variants described herein may readily be prepared by the skilled person, for example, by recombinant DNA manipulation. The manipulation of DNA sequences for preparing peptides or proteins having substitutions, additions, insertions or deletions, is described in detail in Sambrook et al. (1989), for example. Furthermore, the peptides and amino acid variants described herein may be readily prepared with the aid of known peptide synthesis techniques such as, for example, by solid phase synthesis and similar methods.

In one embodiment, a fragment or variant of an amino acid sequence (peptide or protein) is preferably a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to sequences of binding agents such as antibodies, one particular function is one or more binding activities displayed by the amino acid sequence from which the fragment or variant is derived. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., binding to a target molecule. In one embodiment, the modifications in the amino acid sequence of the parent molecule or sequence do not significantly affect or alter the characteristics of the molecule or sequence. In different embodiments, the function of the functional fragment or functional variant may be reduced but still significantly present, e.g., binding of the functional variant may be at least 50%, at least 60%, at least 70%, at least 80%, or at least 90% of the parent molecule or sequence. However, in other embodiments, binding of the functional fragment or functional variant may be enhanced compared to the parent molecule or sequence.

An amino acid sequence (peptide, protein or polypeptide) “derived from” a designated amino acid sequence (peptide, protein or polypeptide) refers to the origin of the first amino acid sequence. Preferably, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the sequences suitable for use herein may be altered such that they vary in sequence from the naturally occurring or native sequences from which they were derived, while retaining the desirable activity of the native sequences.

As used herein, an “instructional material” or “instructions” includes a publication, a recording, a diagram, or any other medium of expression which can be used to communicate the usefulness of the compositions and methods of the invention. The instructional material of the kit of the invention may, for example, be affixed to a container which contains the compositions of the invention or be shipped together with a container which contains the compositions. Alternatively, the instructional material may be shipped separately from the container with the intention that the instructional material and the compositions be used cooperatively by the recipient.

“Isolated” means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell.

The term “recombinant” in the context of the present invention means “made through genetic engineering”. Preferably, a “recombinant object” such as a recombinant nucleic acid in the context of the present invention is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring.

“Physiological pH” as used herein refers to a pH of about 7.5.

The term “genetic modification” or simply “modification” includes the transfection of cells with nucleic acid. The term “transfection” relates to the introduction of nucleic acids, in particular RNA, into a cell. For purposes of the present invention, the term “transfection” also includes the introduction of a nucleic acid into a cell or the uptake of a nucleic acid by such cell, wherein the cell may be present in a subject, e.g., a patient. Thus, according to the present invention, a cell for transfection of a nucleic acid described herein can be present in vitro or in vivo, e.g. the cell can form part of an organ, a tissue and/or an organism of a patient. According to the invention, transfection can be transient or stable. For some applications of transfection, it is sufficient if the transfected genetic material is only transiently expressed. RNA can be transfected into cells to transiently express its coded protein. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can be achieved by using virus-based systems or transposon-based systems for transfection. Generally, nucleic acid encoding a binding agent such as an antibody is transiently transfected into cells. RNA can be transfected into cells to transiently express its coded protein.

Coronavirus Coronaviruses are enveloped, positive-sense, single-stranded RNA ((+) ssRNA) viruses. They have the largest genomes (26-32 kb) among known RNA viruses and are phylogenetically divided into four genera (α, β, γ, and δ), with betacoronaviruses further subdivided into four lineages (A, B, C, and D). Coronaviruses infect a wide range of avian and mammalian species, including humans. Some human coronaviruses generally cause mild respiratory diseases, although severity can be greater in infants, the elderly, and the immunocompromised. Middle East respiratory syndrome coronavirus (MERS-CoV) and severe acute respiratory syndrome coronavirus (SARS-CoV), belonging to betacoronavirus lineages C and B, respectively, are highly pathogenic. Both viruses emerged into the human population from animal reservoirs within the last 15 years and caused outbreaks with high case-fatality rates. The outbreak of severe acute respiratory syndrome coronavirus-2 (SARS-CoV-2) that causes atypical pneumonia (coronavirus disease 2019; COVID-19) has raged in China since mid-December 2019, and has developed to be a public health emergency of international concern. SARS-CoV-2 (MN908947.3) belongs to betacoronavirus lineage B. It has at least 70% sequence similarity to SARS-CoV.

In general, coronaviruses have four structural proteins, namely, envelope (E), membrane (M), nucleocapsid (N), and spike (S). The E and M proteins have important functions in the viral assembly, and the N protein is necessary for viral RNA synthesis. The critical glycoprotein S is responsible for virus binding and entry into target cells. The S protein is synthesized as a single-chain inactive precursor that is cleaved by furin-like host proteases in the producing cell into two noncovalently associated subunits, S1 and S2. The S1 subunit contains the receptor-binding domain (RBD), which recognizes the host-cell receptor. The S2 subunit contains the fusion peptide, two heptad repeats, and a transmembrane domain, all of which are required to mediate fusion of the viral and host-cell membranes by undergoing a large conformational rearrangement. The S1 and S2 subunits trimerize to form a large prefusion spike.

The S precursor protein of SARS-CoV-2 can be proteolytically cleaved into S1 (685 aa) and S2 (588 aa) subunits. The S1 subunit comprises the receptor-binding domain (RBD), which mediates virus entry into sensitive cells through the host angiotensin-converting enzyme 2 (ACE2) receptor. The “RBD domain” generally comprises the amino acid sequence of amino acids 327 to 528 of SEQ ID NO: 197.

Binding Agents

The present disclosure describes antibodies such as monospecific, bivalent antibodies capable of binding to an epitope of coronavirus spike protein (S protein). Moreover, the disclosure describes bispecific or multispecific binding agents comprising a first and a second binding domain, wherein the first binding domain is capable of binding to a coronavirus spike protein (S protein) and the second binding domain is capable of binding to the coronavirus S protein, and wherein the first and second binding domains bind to different epitopes of the coronavirus S protein. The binding agents, including antibodies described herein bind, in particular, to the RBD domain of coronavirus S protein.

In one embodiment, the binding agent or antibody described herein is isolated. In one embodiment, the binding agent or antibody described herein is a recombinant molecule.

The term “epitope” refers to a part or fragment of a molecule or antigen such as coronavirus S protein that is recognized by a binding agent. For example, the epitope may be recognized by an antibody or any other binding protein. An epitope may include a continuous or discontinuous portion of the antigen and may be between about 5 and about 100, such as between about 5 and about 50, more preferably between about 8 and about 30, most preferably between about 8 and about 25 amino acids in length, for example, the epitope may be preferably 9, 10, 11, 12, 13, 14, 15, 16, 17, 18, 19, 20, 21, 22, 23, 24, or 25 amino acids in length. In one embodiment, an epitope is between about 10 and about 25 amino acids in length. The term “epitope” includes structural epitopes.

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as VH or VH) and a heavy chain constant region (abbreviated herein as CH or CH). The heavy chain constant region typically is comprised of three domains, CH1, CH2, and CH3. The hinge region is the region between the CH1 and CH2 domains of the heavy chain and is highly flexible. Disulphide bonds in the hinge region are part of the interactions between two heavy chains in an IgG molecule. Each light chain typically is comprised of a light chain variable region (abbreviated herein as VL or VL) and a light chain constant region (abbreviated herein as CL or CL). The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)). Unless otherwise stated or contradicted by context, reference to amino acid positions in the constant regions in the present invention is according to the EU-numbering (Edelman et al., Proc Natl Acad Sci USA. 1969 May; 63(1):78-85; Kabat et al., Sequences of Proteins of Immunological Interest, Fifth Edition. 1991 NIH Publication No. 91-3242). In general, CDRs described herein are Kabat defined.

The term “amino acid corresponding to position . . . ” as used herein refers to an amino acid position number in a human IgG1 heavy chain. Corresponding amino acid positions in other immunoglobulins may be found by alignment with human IgG1. Thus, an amino acid or segment in one sequence that “corresponds to” an amino acid or segment in another sequence is one that aligns with the other amino acid or segment using a standard sequence alignment program such as ALIGN, ClustalW or similar, typically at default settings and has at least 50%, at least 80%, at least 90%, or at least 95% identity to a human IgG1 heavy chain. It is considered well-known in the art how to align a sequence or segment in a sequence and thereby determine the corresponding position in a sequence to an amino acid position according to the present invention.

The term “antibody” (Ab) in the context of the present invention refers to an immunoglobulin molecule, a fragment of an immunoglobulin molecule, or a derivative of either thereof, which has the ability to bind, preferably specifically bind to an antigen. In one embodiment, binding takes place under typical physiological conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen). The variable regions of the heavy and light chains of the immunoglobulin molecule contain a binding domain that interacts with an antigen. The term “antigen-binding region”, “binding region” or “binding domain”, as used herein, refers to the region or domain which interacts with the antigen and typically comprises both a VH region and a VL region. The term antibody when used herein comprises not only monospecific antibodies, but also multispecific antibodies which comprise multiple, such as two or more, e.g. three or more, different antigen-binding regions. The constant regions of the antibodies (Abs) may mediate the binding of the immunoglobulin to host tissues or factors, including various cells of the immune system (such as effector cells) and components of the complement system such as C1q, the first component in the classical pathway of complement activation. As indicated above, the term antibody as used herein, unless otherwise stated or clearly contradicted by context, includes fragments of an antibody that are antigen-binding fragments, i.e., retain the ability to specifically bind to the antigen, and antibody derivatives, i.e., constructs that are derived from an antibody. It has been shown that the antigen-binding function of an antibody may be performed by fragments of a full-length antibody. Examples of antigen-binding fragments encompassed within the term “antibody” include (i) a Fab′ or Fab fragment, a monovalent fragment consisting of the VL, VH, CL and CH1 domains, or a monovalent antibody as described in WO2007059782 (Genmab); (ii) F(ab′)₂ fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, (v) a dAb fragment (Ward et al., Nature 341, 544-546 (1989)), which consists essentially of a VH domain and also called domain antibodies (Holt et al; Trends Biotechnol-. 2003 November; 21(11):484-90); (vi) camelid or Nanobody molecules (Revets et al; Expert Opin Biol Ther. 2005 January; 5(1):111-24) and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single chain antibodies are encompassed within the term antibody unless otherwise noted or clearly indicated by context. Although such fragments are generally included within the meaning of antibody, they collectively and each independently are unique features of the present invention, exhibiting different biological properties and utility. These and other useful antibody fragments in the context of the present invention, as well as bispecific formats of such fragments, are discussed further herein. It also should be understood that the term antibody, unless specified otherwise, also includes polyclonal antibodies, monoclonal antibodies (mAbs), antibody-like polypeptides, such as chimeric antibodies and humanized antibodies, and antibody fragments retaining the ability to specifically bind to the antigen (antigen-binding fragments) provided by any known technique, such as enzymatic cleavage, peptide synthesis, and recombinant techniques.

The phrase “single chain Fv” or “scFv” refers to an antibody in which the variable domains of the heavy chain and of the light chain (VH and VI) of a traditional two chain antibody have been joined to form one chain. Optionally, a linker (usually a peptide) is inserted between the two chains to allow for proper folding and creation of an active binding site.

An antibody can possess any isotype. As used herein, the term “isotype” refers to the immunoglobulin class (for instance IgG1, IgG2, IgG3, IgG4, IgD, IgA, IgE, or IgM) that is encoded by heavy chain constant region genes. When a particular isotype, e.g. IgG1, is mentioned herein, the term is not limited to a specific isotype sequence, e.g. a particular IgG1 sequence, but is used to indicate that the antibody is closer in sequence to that isotype, e.g. IgG1, than to other isotypes. Thus, e.g. an IgG1 antibody of the invention may be a sequence variant of a naturally-occurring IgG1 antibody, including variations in the constant regions.

In various embodiments, an antibody is an IgG1 antibody, more particularly an IgG1, kappa or IgG1, lambda isotype (i.e. IgG1, κ, λ), an IgG2a antibody (e.g. IgG2a, κ, λ), an IgG2b antibody (e.g. IgG2b, κ, λ), an IgG3 antibody (e.g. IgG3, κ, λ) or an IgG4 antibody (e.g. IgG4, κ, λ). The term “monoclonal antibody” as used herein refers to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal non-human animal, such as a transgenic mouse, having a genome comprising a human heavy chain transgene and a light chain transgene, fused to an immortalized cell.

The term “chimeric antibody” as used herein, refers to an antibody wherein the variable region is derived from a non-human species (e.g. derived from rodents) and the constant region is derived from a different species, such as human. Chimeric monoclonal antibodies for therapeutic applications are developed to reduce antibody immunogenicity. The terms “variable region” or “variable domain” as used in the context of chimeric antibodies, refer to a region which comprises the CDRs and framework regions of both the heavy and light chains of the immunoglobulin. Chimeric antibodies may be generated by using standard DNA techniques as described in Sambrook et al., 1989, Molecular Cloning: A laboratory Manual, New York: Cold Spring Harbor Laboratory Press, Ch. 15. The chimeric antibody may be a genetically or an enzymatically engineered recombinant antibody. It is within the knowledge of the skilled person to generate a chimeric antibody, and thus, generation of the chimeric antibody according to the present invention may be performed by other methods than described herein.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “human antibody” as used herein, refers to antibodies having variable and constant regions derived from human germline immunoglobulin sequences. Human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse or rat, have been grafted onto human framework sequences. Human monoclonal antibodies can be produced by a variety of techniques, including conventional monoclonal antibody methodology, e.g., the standard somatic cell hybridization technique of Kohler and Milstein, Nature 256: 495 (1975). Although somatic cell hybridization procedures are preferred, in principle, other techniques for producing monoclonal antibody can be employed, e.g., viral or oncogenic transformation of B-lymphocytes or phage display techniques using libraries of human antibody genes. A suitable animal system for preparing hybridomas that secrete human monoclonal antibodies is the murine system. Hybridoma production in the mouse is a very well established procedure. Immunization protocols and techniques for isolation of immunized splenocytes for fusion are known in the art. Fusion partners (e.g., murine myeloma cells) and fusion procedures are also known. Human monoclonal antibodies can thus e.g. be generated using transgenic or transchromosomal mice or rats carrying parts of the human immune system rather than the mouse or rat system. Accordingly, in one embodiment, a human antibody is obtained from a transgenic animal, such as a mouse or a rat, carrying human germline immunoglobulin sequences instead of animal immunoglobulin sequences. In such embodiments, the antibody originates from human germline immunoglobulin sequences introduced in the animal, but the final antibody sequence is the result of said human germline immunoglobulin sequences being further modified by somatic hypermutations and affinity maturation by the endogeneous animal antibody machinery, see e.g. Mendez et al. 1997 Nat Genet. 15(2):146-56.

When used herein, unless contradicted by context, the term “Fab-arm”, “binding arm” or “arm” includes one heavy chain-light chain pair and is used interchangeably with “half-molecule” herein.

The term “full-length” when used in the context of an antibody indicates that the antibody is not a fragment, but contains all of the domains of the particular isotype normally found for that isotype in nature, e.g. the VH, CH1, CH2, CH3, hinge, VL and CL domains for an IgG1 antibody.

When used herein, unless contradicted by context, the term “Fc region” refers to an antibody region consisting of the two Fc sequences of the heavy chains of an immunoglobulin, wherein said Fc sequences comprise at least a hinge region, a CH2 domain, and a CH3 domain.

As used herein, the terms “binding” or “capable of binding” in the context of the binding of an antibody to a predetermined antigen or epitope typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less, when determined using Bio-Layer Interferometry (BLI), or, for instance, when determined using surface plasmon resonance (SPR) technology in a BIAcore 3000 instrument using the antigen as the ligand and the antibody as the analyte. The antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its affinity for binding to a non-specific antigen (e.g., BSA, casein) other than the predetermined antigen or a closely-related antigen. The amount with which the affinity is lower is dependent on the K_{D} of the antibody, so that when the K_{D} of the antibody is very low (that is, the antibody is highly specific), then the degree to which the affinity for the antigen is lower than the affinity for a non-specific antigen may be at least 10,000-fold.

The term “k_{d}” (sec⁻¹), as used herein, refers to the dissociation rate constant of a particular antibody-antigen interaction. Said value is also referred to as the k_{off} value.

The term “K_{D}” (M), as used herein, refers to the dissociation equilibrium constant of a particular antibody-antigen interaction.

The present invention also envisions antibodies comprising functional variants of the VL regions, VH regions, or one or more CDRs of the antibodies described herein. A functional variant of a VL, VH, or CDR used in the context of an antibody still allows the antibody to retain at least a substantial proportion (at least about 50%, 60%, 70%, 80%, 90%, 95% or more) of the affinity and/or the specificity/selectivity of the “reference” or “parent” antibody and in some cases, such an antibody may be associated with greater affinity, selectivity and/or specificity than the parent antibody.

Such functional variants typically retain significant sequence identity to the parent antibody. Exemplary variants include those which differ from VH and/or VL and/or CDR regions of the parent antibody sequences mainly by conservative substitutions; for instance, up to 10, such as 9, 8, 7, 6, 5, 4, 3, 2 or 1 of the substitutions in the variant are conservative amino acid residue replacements.

Functional variants of antibody sequences described herein such as VL regions, or VH regions, or antibody sequences having a certain degree of homology or identity to antibody sequences described herein such as VL regions, or VH regions preferably comprise modifications or variations in the non-CDR sequences, while the CDR sequences preferably remain unchanged.

The term “specificity” as used herein is intended to have the following meaning unless contradicted by context. Two antibodies have the “same specificity” if they bind to the same antigen and the same epitope.

The term “competes” and “competition” may refer to the competition between a first antibody and a second antibody to the same antigen. Alternatively “competes” and “competition” may also refer to the competition between an antibody and an endogenous ligand for binding to the corresponding receptor of the endogenous ligand. If an antibody prevents the binding of the endogenous ligand to its receptor, such an antibody is said to block the endogenous interaction of the ligand with its receptor and therefore is competing with the endogenous ligand. It is well known to a person skilled in the art how to test for competition of antibodies for binding to a target antigen. An example of such a method is a so-called cross-competition assay, which may e.g. be performed as an ELISA or by flow-cytometry.

Alternatively, competition may be determined using biolayer interferometry.

Antibodies which compete for binding to a target antigen may bind different epitopes on the antigen, wherein the epitopes are so close to each other that a first antibody binding to one epitope prevents binding of a second antibody to the other epitope. In other situations, however, two different antibodies may bind the same epitope on the antigen and would compete for binding in a competition binding assay. Such antibodies binding to the same epitope are considered to have the same specificity herein. Thus, in one embodiment, antibodies binding to the same epitope are considered to bind to the same amino acids on the target molecule. That antibodies bind to the same epitope on a target antigen may be determined by standard alanine scanning experiments or antibody-antigen crystallization experiments known to a person skilled in the art. Preferably, antibodies or binding domains binding to different epitopes of coronavirus S protein are not competing with each other for binding to their respective epitopes.

As described above, various formats of antibodies have been described in the art. The binding agent of the invention can in principle comprise an antibody of any isotype. The choice of isotype typically will be guided by the desired Fc-mediated effector functions, such as ADCC induction, or the requirement for an antibody devoid of Fc-mediated effector function (“inert” antibody). Exemplary isotypes are IgG1, IgG2, IgG3, and IgG4. Either of the human light chain constant regions, kappa or lambda, may be used. The effector function of the antibodies of the present invention may be changed by isotype switching to, e.g., an IgG1, IgG2, IgG3, IgG4, IgD, IgA, IgE, or IgM antibody for various therapeutic uses. In one embodiment, both heavy chains of an antibody of the present invention are of the IgG1 isotype, for instance an IgG1,κ.

Optionally, the heavy chain may be modified in the hinge and/or CH3 region as described elsewhere herein.

Preferably, each of the antigen-binding regions or domains comprises a heavy chain variable region (VH) and a light chain variable region (VL), and wherein said variable regions each comprise three CDR sequences, CDR1, CDR2 and CDR3, respectively, and four framework sequences, FR1, FR2, FR3 and FR4, respectively. Furthermore, preferably, the antibody comprises two heavy chain constant regions (CH), and two light chain constant regions (CL).

In one embodiment, the binding agent comprises a full-length antibody, such as a full-length IgG1 antibody.

In other embodiment, the binding agent comprises an antibody fragment, such as a Fab′ or Fab fragment, a monovalent fragment consisting of the VL, VH, CL and CH1 domains, a monovalent antibody as described in WO2007059782 (Genmab), a F(ab′)₂ fragment, a Fd fragment, a Fv fragment, a dAb fragment, camelid or nanobodies, or an isolated complementarity determining region (CDR).

The term “binding agent” in the context of the present invention refers to any agent capable of binding to desired antigens. In certain embodiments of the invention, the binding agent is or comprises an antibody, antibody fragment, or any other binding protein, or any combination thereof. One preferred combination is a combination of an antibody, e.g., a full-length antibody, binding to a first epitope of the coronavirus S protein coupled, in particularly covalently, to one or more, such as two binding proteins binding to a different epitope of the coronavirus S protein. In one embodiment, the binding protein comprises an extracellular domain (ECD) of ACE2 protein or a variant thereof, or a fragment of the ECD of ACE2 protein or the variant thereof. In one embodiment, the binding protein comprises an antibody fragment such as scFv. The binding agent may also comprise synthetic, modified or non-naturally occurring moieties, in particular non-peptide moieties. Such moieties may, for example, link desired antigen-binding functionalities or regions such as antibodies or antibody fragments. In one embodiment, the binding agent is a synthetic construct comprising antigen-binding CDRs or variable regions.

Naturally occurring antibodies are generally monospecific, i.e. they bind to a single antigen. The present invention provides binding agents binding to different epitopes on coronavirus S protein. Such binding agents are at least bispecific or multispecific such as trispecific, tetraspecific and so on. Thus, the binding agent may comprise two or more antibodies as described herein or fragments thereof. In particular, a binding agent described herein may be an artificial protein that is composed of two different antibodies, an antibody and a fragment of a different antibody, and fragments of two different antibodies (said fragments of two different antibodies forming two binding domains).

According to the invention, a bispecific binding agent, in particular a bispecific protein, such as a bispecific antibody is a molecule that has two different binding specificities and thus may bind to two epitopes. Particularly, the term “bispecific antibody” as used herein refers to an antibody comprising two antigen-binding sites, a first binding site having affinity for a first epitope and a second binding site having binding affinity for a second epitope distinct from the first.

The term “bispecific” in the context of the present invention refers to an agent having two different antigen-binding regions binding to different epitopes, in particular different epitopes on the same antigen, e.g. coronavirus S protein.

“Multispecific binding agents” are molecules which have more than two different binding specificities.

Many different formats and uses of bispecific antibodies are known in the art, and were reviewed by Kontermann; Drug Discov Today, 2015 July; 20(7):838-47 and; MAbs, 2012 March-April; 4(2):182-97.

A bispecific binding agent according to the present invention is not limited to any particular bispecific format or method of producing it.

Examples of bispecific antibody molecules which may be used in the present invention comprise (i) a single antibody that has two arms comprising different antigen-binding regions; (ii) a single chain antibody that has specificity to two different epitopes, e.g., via two scFvs linked in tandem by an extra peptide linker; (iii) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (iv) a chemically-linked bispecific (Fab′)2 fragment; (v) a Tandab, which is a fusion of two single chain diabodies resulting in a tetravalent bispecific antibody that has two binding sites for each of the target antigens; (vi) a flexibody, which is a combination of scFvs with a diabody resulting in a multivalent molecule; (vii) a so-called “dock and lock” molecule, based on the “dimerization and docking domain” in Protein Kinase A, which, when applied to Fabs, can yield a trivalent bispecific binding protein consisting of two identical Fab fragments linked to a different Fab fragment; (viii) a so-called Scorpion molecule, comprising, e.g., two scFvs fused to both termini of a human Fab-arm; and (ix) a diabody.

In one embodiment of the invention, the binding agent of the present invention is a diabody or a cross-body. In one embodiment, the binding agent of the invention is a bispecific antibody obtained via a controlled Fab-arm exchange (such as described in WO2011131746 (Genmab)). Examples of different classes of binding agents according to the present invention include but are not limited to (i) IgG-like molecules with complementary CH3 domains to force heterodimerization; (ii) recombinant IgG-like dual targeting molecules, wherein the two sides of the molecule each contain the Fab fragment or part of the Fab fragment of at least two different antibodies; (iii) IgG fusion molecules, wherein full length IgG antibodies are fused to extra Fab fragment or parts of Fab fragment; (iv) Fc fusion molecules, wherein single chain Fv molecules or stabilized diabodies are fused to heavy-chain constant-domains, Fc regions or parts thereof; (v) Fab fusion molecules, wherein different Fab-fragments are fused together, fused to heavy-chain constant-domains, Fc regions or parts thereof; and (vi) ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule fused to heavy-chain constant-domains, Fc regions or parts thereof.

Examples of IgG-like molecules with complementary CH3 domain molecules include but are not limited to the Triomab/Quadroma molecules (Trion Pharma/Fresenius Biotech; Roche, WO2011069104), the so-called Knobs-into-Holes molecules (Genentech, WO9850431), CrossMAbs (Roche, WO2011117329) and the electrostatically-matched molecules (Amgen, EP1870459 and WO2009089004; Chugai, US201000155133; Oncomed, WO2010129304), the LUZ-Y molecules (Genentech, Wranik et al. J. Biol. Chem. 2012, 287(52): 43331-9, doi: 10.1074/jbc.M112.397869. Epub 2012 Nov. 1), DIG-body and PIG-body molecules (Pharmabcine, WO2010134666, WO2014081202), the Strand Exchange Engineered Domain body (SEEDbody) molecules (EMD Serono, WO2007110205), the Biclonics molecules (Merus, WO2013157953), FcΔAdp molecules (Regeneron, WO201015792), bispecific IgG1 and IgG2 molecules (Pfizer/Rinat, WO11143545), Azymetric scaffold molecules (Zymeworks/Merck, WO2012058768), mAb-Fv molecules (Xencor, WO2011028952), bivalent bispecific antibodies (WO2009080254) and the DuoBody® molecules (Genmab, WO2011131746).

Examples of recombinant IgG-like dual targeting molecules include but are not limited to Dual Targeting (DT)-Ig molecules (WO2009058383), Two-in-one Antibody (Genentech; Bostrom, et al 2009. Science 323, 1610-1614.), Cross-linked Mabs (Karmanos Cancer Center), mAb2 (F-Star, WO2008003116), Zybody molecules (Zyngenia; LaFleur et al. MAbs. 2013 March-April; 5(2):208-18), approaches with common light chain (Crucell/Merus, U.S. Pat. No. 7,262,028), KABodies (NovImmune, WO2012023053) and CovX-body (CovX/Pfizer; Doppalapudi, V. R., et al 2007. Bioorg. Med. Chem. Lett. 17, 501-506.).

Examples of IgG fusion molecules include but are not limited to Dual Variable Domain (DVD)-Ig molecules (Abbott, U.S. Pat. No. 7,612,181), Dual domain double head antibodies (Unilever; Sanofi Aventis, WO20100226923), IgG-like Bispecific molecules (ImClone/Eli Lilly, Lewis et al. Nat Biotechnol. 2014 February; 32(2):191-8), Ts2Ab (MedImmune/AZ; Dimasi et al. J Mol Biol. 2009 Oct. 30; 393(3):672-92) and BsAb molecules (Zymogenetics, WO2010111625), HERCULES molecules (Biogen Idec, US007951918), scFv fusion molecules (Novartis), scFv fusion molecules (Changzhou Adam Biotech Inc, CN 102250246) and TvAb molecules (Roche, WO2012025525, WO2012025530).

Examples of Fc fusion molecules include but are not limited to ScFv/Fc Fusions (Pearce et al., Biochem Mol Biol Int. 1997 September; 42(6):1179-88), SCORPION molecules (Emergent BioSolutions/Trubion, Blankenship J W, et al. AACR 100th Annual meeting 2009 (Abstract #5465); Zymogenetics/BMS, WO2010111625), Dual Affinity Retargeting Technology (Fc-DART) molecules (MacroGenics, WO2008157379, WO2010080538) and Dual(ScFv)2-Fab molecules (National Research Center for Antibody Medicine—China).

Examples of Fab fusion bispecific antibodies include but are not limited to F(ab)2 molecules (Medarex/AMGEN; Deo et al J Immunol. 1998 Feb. 15; 160(4):1677-86.), Dual-Action or Bis-Fab molecules (Genentech, Bostrom, et al 2009. Science 323, 1610-1614.), Dock-and-Lock (DNL) molecules (ImmunoMedics, WO2003074569, WO2005004809), Bivalent Bispecific molecules (Biotecnol, Schoonjans, J Immunol. 2000 Dec. 15; 165(12):7050-7.) and Fab-Fv molecules (UCB-Celltech, WO 2009040562 A1).

Examples of ScFv-, diabody-based and domain antibodies include but are not limited to Bispecific T Cell Engager (BiTE) molecules (Micromet, WO2005061547), Tandem Diabody molecules (TandAb) (Affimed) Le Gall et al., Protein Eng Des Sel. 2004 April; 17(4):357-66.), Dual Affinity Retargeting Technology (DART) molecules (MacroGenics, WO2008157379, WO2010080538), Single-chain Diabody molecules (Lawrence, FEBS Lett. 1998 Apr. 3; 425(3):479-84), TCR-like Antibodies (AIT, ReceptorLogics), Human Serum Albumin ScFv Fusion (Merrimack, WO2010059315) and COMBODY molecules (Epigen Biotech, Zhu et al. Immunol Cell Biol. 2010 August; 88(6):667-75.), dual targeting nanobodies (Ablynx, Hmila et al., FASEB J. 2010) and dual targeting heavy chain only domain antibodies.

In one aspect, the bispecific antibody of the invention comprises a first Fc sequence comprising a first CH3 region, and a second Fc sequence comprising a second CH3 region, wherein the sequences of the first and second CH3 regions are different and are such that the heterodimeric interaction between said first and second CH3 regions is stronger than each of the homodimeric interactions of said first and second CH3 regions. More details on these interactions and how they can be achieved are provided in WO2011131746 and WO2013060867 (Genmab), which are hereby incorporated by reference.

Traditional methods such as the hybrid hybridoma and chemical conjugation methods (Marvin and Zhu (2005) Acta Pharmacol Sin 26:649) can be used in the preparation of the bispecific antibodies of the invention. Co-expression in a host cell of two antibodies, consisting of different heavy and light chains, leads to a mixture of possible antibody products in addition to the desired bispecific antibody, which can then be isolated by, e.g., affinity chromatography or similar methods.

Strategies favoring the formation of a functional bispecific, product, upon co-expression of different antibody constructs can also be used, e.g., the method described by Lindhofer et al. (1995 J Immunol 155:219). Fusion of rat and mouse hybridomas producing different antibodies leads to a limited number of heterodimeric proteins because of preferential species-restricted heavy/light chain pairing. Another strategy to promote formation of heterodimers over homodimers is a “knob-into-hole” strategy in which a protuberance is introduced on a first heavy-chain polypeptide and a corresponding cavity in a second heavy-chain polypeptide, such that the protuberance can be positioned in the cavity at the interface of these two heavy chains so as to promote heterodimer formation and hinder homodimer formation. “Protuberances” are constructed by replacing small amino-acid side-chains from the interface of the first polypeptide with larger side chains. Compensatory “cavities” of identical or similar size to the protuberances are created in the interface of the second polypeptide by replacing large amino-acid side-chains with smaller ones (U.S. Pat. No. 5,731,168). EP1870459 (Chugai) and WO2009089004 (Amgen) describe other strategies for favoring heterodimer formation upon co-expression of different antibody domains in a host cell. In these methods, one or more residues that make up the CH3-CH3 interface in both CH3 domains are replaced with a charged amino acid such that homodimer formation is electrostatically unfavorable and heterodimerization is electrostatically favorable. WO2007110205 (Merck) describe yet another strategy, wherein differences between IgA and IgG CH3 domains are exploited to promote heterodimerization.

Another in vitro method for producing bispecific antibodies has been described in WO2008119353 (Genmab), wherein a bispecific antibody is formed by “Fab-arm” or “half-molecule” exchange (swapping of a heavy chain and attached light chain) between two monospecific IgG4- or IgG4-like antibodies upon incubation under reducing conditions. The resulting product is a bispecific antibody having two Fab arms which may comprise different sequences.

The term “bispecific antibody” includes diabodies. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90: 6444-6448; Poljak, R. J., et al. (1994) Structure 2: 1121-1123). Bispecific antibodies also include bispecific single chain antibodies. The term “bispecific single chain antibody” denotes a single polypeptide chain comprising two binding domains. In particular, the term “bispecific single chain antibody” or “single chain bispecific antibody” or related terms in accordance with the present invention preferably mean antibody constructs resulting from joining at least two antibody variable regions in a single polypeptide chain devoid of the constant and/or Fc portion(s) present in full immunoglobulins. For example, a bispecific single chain antibody may be a construct with a total of two antibody variable regions, for example two VH regions, each capable of specifically binding to a separate epitope, and connected with one another through a short polypeptide spacer such that the two antibody variable regions with their interposed spacer exist as a single contiguous polypeptide chain. Another example of a bispecific single chain antibody may be a single polypeptide chain with three antibody variable regions. Here, two antibody variable regions, for example one VH and one VL, may make up an scFv, wherein the two antibody variable regions are connected to one another via a synthetic polypeptide linker, the latter often being genetically engineered so as to be minimally immunogenic while remaining maximally resistant to proteolysis. This scFv is capable of specifically binding to a particular epitope, and is connected to a further antibody variable region, for example a VH region, capable of binding to a different epitope than that bound by the scFv. Yet another example of a bispecific single chain antibody may be a single polypeptide chain with four antibody variable regions. Here, the first two antibody variable regions, for example a VH region and a VL region, may form one scFv capable of binding to one epitope, whereas the second VH region and VL region may form a second scFv capable of binding to another epitope. Within a single contiguous polypeptide chain, individual antibody variable regions of one specificity may advantageously be separated by a synthetic polypeptide linker, whereas the respective scFvs may advantageously be separated by a short polypeptide spacer as described above. According to one embodiment, the first binding domain of the bispecific antibody comprises one antibody variable domain, preferably a VHH domain. According to one embodiment of the invention, the first binding domain of the bispecific antibody comprises two antibody variable domains, preferably a scFv, i.e. VH-VL or VL-VH. According to one embodiment of the invention, the second binding domain of the bispecific antibody comprises one antibody variable domain, preferably a VHH domain. According to one embodiment of the invention, the second binding domain of the bispecific antibody comprises two antibody variable domains, preferably a scFv, i.e. VH-VL or VL-VH. In its minimal form, the total number of antibody variable regions in the bispecific antibody according to the invention is thus only two. For example, such an antibody could comprise two VH or two VHH domains. According to one embodiment, the first binding domain and the second binding domain of the bispecific antibody each comprise one antibody variable domain, preferably a VHH domain.

According to one embodiment, the first binding domain and the second binding domain of the bispecific antibody each comprise two antibody variable domains, preferably a scFv, i.e. VH-VL or VL-VH. In this embodiment, the binding agent preferably comprises (i) a heavy chain variable domain (VH) of a first antibody, (ii) a light chain variable domain (VL) of a first antibody, (iii) a heavy chain variable domain (VH) of a second antibody and (iv) a light chain variable domain (VL) of a second antibody.

In one embodiment, the bispecific molecules according to the invention comprises two Fab regions, each being directed against different epitopes of coronavirus S protein. In one embodiment, the molecule of the invention is an antigen binding fragment (Fab)2 complex. The Fab2 complex is composed of two Fab fragments, one Fab fragment comprising a Fv domain, i.e. VH and VL domains, specific for one epitope of coronavirus S protein, and the other Fab fragment comprising a Fv domain specific for another epitope of coronavirus S protein. Each of the Fab fragments may be composed of two single chains, a VL-CL module and a VH-CH module. Alternatively, each of the individual Fab fragments may be arranged in a single chain, preferably, VL-CL-CH-VH, and the individual variable and constant domains may be connected with a peptide linker. In general, the individual single chains and Fab fragments may be connected via disulfide bonds, adhesive domains, chemically linked and/or peptide linker. The bispecific molecule may also comprise more than two Fab fragments, in particular, the molecule may be a Fab3, Fab4, or a multimeric Fab complex with specificity for 2, 3, 4, or more different epitopes. The invention also includes chemically linked Fabs.

In one embodiment, the binding agent according to the invention includes various types of bivalent and trivalent single-chain variable fragments (scFvs), fusion proteins mimicking the variable domains of two antibodies. Divalent (or bivalent) single-chain variable fragments (di-scFvs, bi-scFvs) can be engineered by linking two scFvs. This can be done by producing a single peptide chain with two VH and two VL regions, yielding tandem scFvs. The invention also includes multispecific molecules comprising more than two scFvs binding domains. This makes it possible that the molecule comprises either multiple antigen specificities and is a trispecific, tetraspecific, or multispecific molecule, or the molecule is a bispecific molecule comprising more than one scFv binding domain with specificity for the same antigen. In particular, the molecule of the invention may be a multispecific single chain Fv.

Another possibility is the creation of scFvs with linker peptides that are too short for the two variable regions to fold together (about five amino acids), forcing scFvs to dimerize. This type is known as diabodies. Still shorter linkers (one or two amino acids) lead to the formation of trimers, so-called triabodies or tribodies. Tetrabodies have also been produced. They exhibit an even higher affinity to their targets than diabodies.

A particularly preferred example of a bispecific antibody fragment is a diabody (Kipriyanov, Int. J. Cancer 77 (1998), 763-772), which is a small bivalent and bispecific antibody fragment. Diabodies comprise a heavy chain variable domain (VH) and a light chain variable domain (VL) on the same polypeptide chain (VH-VL) connected by a peptide linker that is too short to allow pairing between the two domains on the same chain. This forces pairing with the complementary domains of another chain and promotes the assembly of a dimeric molecule with two functional antigen binding sites.

In one embodiment, the bispecific or multispecific molecule according to the invention comprises variable (VH, VL) and constant domains (C) of immunoglobulins. In one embodiment the bispecific molecule is a minibody, preferably, a minibody comprising two single VH-VL-C chains that are connected with each other via the constant domains (C) of each chain. According to this aspect, the corresponding variable heavy chain regions (VH), corresponding variable light chain regions (VL) and constant domains (C) are arranged, from N-terminus to C-terminus, in the order VH(Epitope 1)-VL(Epitope 1)-(C) and VH(Epitope 2)-VL(Epitope 2)-C, wherein C is preferably a CH3 domain, Epitope 1 refers to a first epitope of coronavirus S protein and Epitope 2 refers to a second epitope of coronavirus S protein. Pairing of the constant domains results in formation of the minibody.

According to another aspect, the bispecific binding agent of the invention is in the format of a bispecific single chain antibody construct, whereby said construct comprises or consists of at least two binding domains. In one embodiment, each binding domain comprises one variable region from an antibody heavy chain (“VH region”), wherein the VH region of the first binding domain specifically binds to Epitope 1 of a coronavirus S protein, and the VH region of the second binding domain specifically binds to Epitope 2 of a coronavirus S protein. The two binding domains are optionally linked to one another by a short polypeptide spacer. Each binding domain may additionally comprise one variable region from an antibody light chain (“VL region”), the VH region and VL region within each of the first and second binding domains being linked to one another via a polypeptide linker long enough to allow the VH region and VL region of the first binding domain and the VH region and VL region of the second binding domain to pair with one another.

In one embodiment, the binding agent described herein comprises an antibody, e.g., a full-length antibody, comprising the first binding domain. In one embodiment, the binding agent described herein comprises an antibody fragment such as scFv comprising the second binding domain which is covalently linked to the antibody comprising the first binding domain. In one embodiment, the binding agent comprises the antibody fragment such as scFv covalently linked to the N-terminus or C-terminus of the light chain of the antibody.

In one embodiment, the binding agent described herein comprises an antibody, e.g., a full-length antibody, comprising the first binding domain. In one embodiment, the binding agent described herein comprises an extracellular domain (ECD) of ACE2 protein or a variant thereof, or a fragment of the ECD of ACE2 protein or the variant thereof comprising the second binding domain which is covalently linked to the antibody comprising the first binding domain. In one embodiment, the binding agent comprises the extracellular domain (ECD) of ACE2 protein or a variant thereof, or a fragment of the ECD of ACE2 protein or the variant thereof covalently linked to the N-terminus or C-terminus of the light chain of the antibody.

The antibody and antibody fragment or extracellular domain (ECD) of ACE2 protein or a variant thereof, or a fragment of the ECD of ACE2 protein or the variant thereof may be linked by a GS-linker such as (Gly4Ser)1, (Gly4Ser)2, (Gly4Ser)3, (Gly4Ser)4 or (Gly4Ser)5.

Angiotensin-converting enzyme 2 (ACE2) belongs to the angiotensin-converting enzyme family of dipeptidyl carboxypeptidases and is a transmembrane protein that is present in most organs, with the highest levels of ACE2 being detected in the cardiovascular system, gut, kidneys, and lungs. Most predominantly, ACE2 is attached to the cell membrane of lung type II alveolar cells, enterocytes of the small intestine, arterial and venous endothelial cells, and arterial smooth muscle cells. ACE2 is a key regulator of the renin-angiotensin system (RAS) and serves as a counterbalance to Angiotensin-converting enzyme 1 (ACE) activity. Cleavage of angiotensin I by ACE activity results in the production of angiotensin II, which triggers potent vasoconstriction, inflammation, cell proliferation, hypertrophy, and fibrosis. ACE2 catalyzes the cleavage of angiotensin II into angiotensin 1-7, with angiotensin 1-7 activity resulting in a counterbalance to the detrimental effects of angiotensin II by promoting vasodilation and cardioprotection. Therefore, ACE2 protects against RAS-induced injuries, and partial loss of ACE2 has been linked to increased susceptibility to heart disease, while clinical trials with intravenous infusion of recombinant human ACE2 in patients with pulmonary arterial hypertension results in a decrease in plasma angiotensin II/angiotensin 1-7 ratios and a therapeutic effect. In addition, ACE2 has been shown to play a protective role in lung injury. Murine acute respiratory distress syndrome (ARDS) models have shown that loss of ACE2 expression results in enhanced vascular permeability, increased lung edema, and worsened lung function, while treatment with catalytically active recombinant ACE2 protein improves symptoms of acute lung injury in wild-type and in ACE2 knockout mice. Furthermore, ACE2 and other components of the renin-angiotensin system may play a central role in controlling the severity of acute lung failure once a respiratory disease process has started.

ACE2 is a type I transmembrane protein of 805 amino acids and contains a short cytoplasmic domain, a transmembrane domain, and a large ectodomain. The catalytic domain of ACE2 is found in the extracellular domain (ECD) of the ectodomain, resulting in the ACE2 active site being poised to metabolize circulating peptides such as angiotensin II. The extracellular region of human ACE2 is comprised of two domains, a zinc metallopeptidase domain (residues 19 to 611) and a second domain located at the C-terminus (residues 612 to 740). The metallopeptidase domain can be further divided into two catalytic subdomains, an N-terminal subdomain I and a C-terminal subdomain II, with these two subdomains being connected at the floor of the active site cleft. Multiple amino acid residues have been identified as playing an important role in ACE2 substrate binding and activity. For example, Arg273 makes a salt-bridge with the C-terminus of ACE2 inhibitor, MLN-4760, and is therefore proposed to be involved in binding the C-terminus of ACE2 substrates. Mutating Arg273 to a glutamine (R273Q) results in a change from a positive to neutral charge in the side chain at this position, resulting in loss of ACE2 activity and showing that a positive side chain of Arg273 is critical to substrate binding. Another amino acid residue, His345 plays an important role in ACE2 activity by acting as a key hydrogen bond donor/acceptor and has been shown to form hydrogen bonds with both the C-terminus and the secondary amine group of MLN-4760. Mutating His345 to a leucine (H345L), results in approximately 300-fold less activity when compared to wild-type ACE2. Finally, two other amino acid residues, His374 and His378, are two of three amino acids that comprise the zinc coordination sphere, and thus play a crucial role in coordinating the zinc binding site of ACE2.

In 2003, ACE2 was identified as a receptor for the severe acute respiratory syndrome coronavirus (SARS-CoV-1) and an important factor in severe acute respiratory syndrome (SARS) pathogenesis. A region of the ACE2 ECD, which includes the first α-helix and Lys353 and proximal residues of the N-terminus of β-sheet 5, interacts with high affinity with the receptor-binding domain (RBD) of a SARS-CoV-1 spike protein. This interaction between a SARS-CoV-1 spike protein and a cell-associated ACE2 protein is a key component of SARS-CoV-1 activity, and correlates with infection of human airway epithelia by SARS-CoV-1. Furthermore, binding of SARS-CoV-1 to ACE2 leads to a reduction of cell surface ACE2 by ACE2 endocytosis with SARS-CoV-1 and ACE2 shedding, thus resulting in a loss of ACE2-mediated tissue protection. The novel, severe acute respiratory syndrome coronavirus 2 (SARS-CoV-2) shows a 73% similarity in its RBD when compared to the SARS-CoV-1 RBD, and has also been shown to bind to ACE2 to promote viral entry into cells. Similar to SARS-CoV-1, SARS-CoV-2 binds to the ACE2 protein through the RBD of its spike protein. However, there is a structural difference between the receptor binding motifs (RBMs) of SARS-CoV-1 and SARS-CoV-2 in the conformation of the loops in the ACE2-binding ridge. These structural differences result in an additional main-chain hydrogen bond forming between Asn487 and Ala475 in the SARS-CoV-2 RBM, causing the ridge to take a more compact conformation and the loop containing Ala475 to move closer to ACE2. This difference creates more contact between the SARS-CoV-2 RBM with the N-terminal helix of ACE2 through even more additional hydrogen bonds when compared to the SARS-CoV-1 RBM. As a result, in comparison to the SARS-CoV-1 RBM interaction with ACE2, the SARS-CoV-2 RBM forms a larger binding interface and more contacts with ACE2 and more favourable binding, with some studies suggesting that it has a 10-20 fold higher binding affinity for ACE2 when compared to SARS-CoV-1 RBM.

In one embodiment, the term “ACE2” or “ACE2 protein” relates to human ACE2 or a variant thereof, or a fragment of the ACE2 or the variant thereof. In one embodiment, “ACE2” or “ACE2 protein” comprises the amino acid sequence of SEQ ID NO: 130, an amino acid sequence having at least 99%, 98%, 97%, 96%, 95%, 90%, 85%, or 80% identity to the amino acid sequence of SEQ ID NO: 130, or a fragment of the amino acid sequence of SEQ ID NO: 130, or the amino acid sequence having at least 99%, 98%, 97%, 96%, 95%, 90%, 85%, or 80% identity to the amino acid sequence of SEQ ID NO: 130. In one embodiment, “ACE2” or “ACE2 protein” comprises the amino acid sequence of SEQ ID NO: 130.

In one embodiment, the term “extracellular domain of ACE2”, “extracellular domain of ACE2 protein”, “ACE2 extracellular domain” or similar terms relate to an extracellular domain of ACE2 or a variant thereof, or a fragment of the extracellular domain of ACE2 or the variant thereof.

In one embodiment, the term “extracellular domain of ACE2” relates to an extracellular domain of human ACE2 or a variant thereof, or a fragment of the extracellular domain of human ACE2 or the variant thereof. In one embodiment, an extracellular domain of ACE2 comprises the amino acid sequence of amino acids 18 to 615 of SEQ ID NO: 130, an amino acid sequence having at least 99%, 98%, 97%, 96%, 95%, 90%, 85%, or 80% identity to the amino acid sequence of amino acids 18 to 615 of SEQ ID NO: 130, or a fragment of the amino acid sequence of amino acids 18 to 615 of SEQ ID NO: 130, or the amino acid sequence having at least 99%, 98%, 97%, 96%, 95%, 90%, 85%, or 80% identity to the amino acid sequence of amino acids 18 to 615 of SEQ ID NO: 130. In one embodiment, an extracellular domain of ACE2 comprises the amino acid sequence of amino acids 18 to 615 of SEQ ID NO: 130.

An extracellular domain of ACE2 or a variant thereof, or a fragment of the extracellular domain of ACE2 or the variant thereof may comprise modifications that, for example, avoid enzymatic activity and/or substrate binding.

Thus, in one embodiment, an extracellular domain of ACE2 or a variant thereof, or a fragment of the extracellular domain of ACE2 or the variant thereof is modified so that the extracellular domain of ACE2 or a variant thereof, or a fragment of the extracellular domain of ACE2 or the variant thereof exerts enzymatic activity and/or binds substrate to a lesser extent relative to an extracellular domain of ACE2 or a variant thereof, or a fragment of the extracellular domain of ACE2 or the variant thereof which is identical, except for not comprising the modifications. Examples of amino acid positions that may be modified include positions R273, H345, H374, and H378.

Hence, in one embodiment, the amino acid in at least one position corresponding to R273, H345, H374, and H378 may be Q, L, N, and N, respectively. In one embodiment, the amino acids in the positions corresponding to R273, H345, H374, and H378 are 0, L, N, and N. In one embodiment, an extracellular domain of ACE2 comprises the amino acid sequence of SEQ ID NO: 129, an amino acid sequence having at least 99%, 98%, 97%, 96%, 95%, 90%, 85%, or 80% identity to the amino acid sequence of SEQ ID NO: 129, or a fragment of the amino acid sequence of SEQ ID NO: 129, or the amino acid sequence having at least 99%, 98%, 97%, 96%, 95%, 90%, 85%, or 80% identity to the amino acid sequence of SEQ ID NO: 129. In one embodiment, an extracellular domain of ACE2 comprises the amino acid sequence of SEQ ID NO: 129.

In one embodiment, an extracellular domain of ACE2 or a variant thereof, or a fragment of the extracellular domain of ACE2 or the variant thereof binds to a coronavirus S protein.

In some embodiments of the invention, the binding agent according to the present invention comprises, in addition to the antigen-binding regions, an Fc region consisting of the Fc sequences of the two heavy chains.

The first and second Fc sequences may each be of any isotype, including, but not limited to, IgG1, IgG2, IgG3 and IgG4, and may comprise one or more mutations or modifications. In one embodiment, each of the first and second Fc sequences is of the IgG4 isotype or derived therefrom, optionally with one or more mutations or modifications. In another embodiment, each of the first and second Fc sequences is of the IgG1 isotype or derived therefrom, optionally with one or more mutations or modifications. In another embodiment, one of the Fc sequences is of the IgG1 isotype and the other of the IgG4 isotype, or is derived from such respective isotypes, optionally with one or more mutations or modifications.

In one embodiment of the invention, one or both Fc sequences are effector-function-deficient. For example, the Fc sequence(s) may be of an IgG4 isotype, or a non-IgG4 type, e.g. IgG1, IgG2 or IgG3, which has been mutated such that the ability to mediate effector functions, such as ADCC, has been reduced or even eliminated. Such mutations have e.g. been described in Dall'Acqua W F et al., J Immunol. 177(2):1129-1138 (2006) and Hezareh M, 1 Virol.; 75(24):12161-12168 (2001). In another embodiment, one or both Fc sequences comprise an IgG1 wildtype sequence.

The term “effector functions” in the context of the present invention includes any functions mediated by components of the immune system that result, for example, in the killing of diseased cells such as tumor cells, or in the inhibition of tumor growth and/or inhibition of tumor development, including inhibition of tumor dissemination and metastasis. Preferably, the effector functions in the context of the present invention are T cell mediated effector functions. Such functions comprise ADCC, ADCP or CDC.

Antibody-dependent cell-mediated cytotoxicity (ADCC) is the killing of an antibody-coated target cell by a cytotoxic effector cell through a nonphagocytic process, characterised by the release of the content of cytotoxic granules or by the expression of cell death-inducing molecules. ADCC is independent of the immune complement system that also lyses targets but does not require any other cell. ADCC is triggered through interaction of target-bound antibodies (belonging to IgG or IgA or IgE classes) with certain Fc receptors (FcRs), glycoproteins present on the effector cell surface that bind the Fc region of immunoglobulins (Ig). Effector cells that mediate ADCC include natural killer (NK) cells, monocytes, macrophages, neutrophils, eosinophils and dendritic cells. ADCC is a rapid effector mechanism whose efficacy is dependent on a number of parameters (density and stability of the antigen on the surface of the target cell; antibody affinity and FcR-binding affinity). ADCC involving human IgG1, the most used IgG subclass for therapeutic antibodies, is highly dependent on the glycosylation profile of its Fc portion and on the polymorphism of Fcγ receptors.

Antibody-dependent cellular phagocytosis (ADCP) is one crucial mechanism of action of many antibody therapies. It is defined as a highly regulated process by which antibodies eliminate bound targets via connecting its Fc domain to specific receptors on phagocytic cells, and eliciting phagocytosis. Unlike ADCC, ADCP can be mediated by monocytes, macrophages, neutrophils, and dendritic cells, through FcγRIIa, FcγRI, and FcγRIIIa, of which FcγRIIa (CD32a) on macrophages represent the predominant pathway.

Complement-dependent cytotoxicity (CDC) is another cell-killing method that can be directed by antibodies. IgM is the most effective isotype for complement activation. IgG1 and IgG3 are also both very effective at directing CDC via the classical complement-activation pathway. Preferably, in this cascade, the formation of antigen-antibody complexes results in the uncloaking of multiple C1q binding sites in close proximity on the C_{H}2 domains of participating antibody molecules such as IgG molecules (C1q is one of three subcomponents of complement C1). Preferably these uncloaked C1q binding sites convert the previously low-affinity C1q-IgG interaction to one of high avidity, which triggers a cascade of events involving a series of other complement proteins and leads to the proteolytic release of the effector-cell chemotactic/activating agents C3a and C5a. Preferably, the complement cascade ends in the formation of a membrane attack complex, which creates pores in the cell membrane that facilitate free passage of water and solutes into and out of the cell.

Antibodies (optionally as part of a bispecific or multispecific binding agent) according to the present invention may comprise modifications in the Fc region. When an antibody comprises such modifications, it may become an inert, or non-activating, antibody. The term “inertness”, “inert” or “non-activating” as used herein, refers to an Fc region which is at least not able to bind any Fcγ receptors, induce Fc-mediated cross-linking of FcRs, or induce FcR-mediated cross-linking of target antigens via two Fc regions of individual antibodies, or is not able to bind C1q. The inertness of an Fc region of a humanized or chimeric CD137 or PD-L1 antibody is advantageously tested using the antibody in a monospecific format.

Several variants can be constructed to make the Fc region of an antibody inactive for interactions with Fcγ (gamma) receptors and C1q for therapeutic antibody development. Examples of such variants are described herein.

Thus, in one embodiment, an antibody comprises a first and a second heavy chain, wherein one or both heavy chains are modified so that the antibody induces Fc-mediated effector function to a lesser extent relative to an antibody which is identical, except for comprising non-modified first and second heavy chains. Said Fc-mediated effector function may be measured by determining binding to Fcγ receptors, binding to C1q, or induction of Fc-mediated cross-linking of FcRs.

In one embodiment, the heavy and light chain constant sequences have been modified so that binding of C1q to said antibody is reduced compared to an unmodified antibody by at least 70%, at least 80%, at least 90%, at least 95%, at least 97%, or 100%, wherein C1q binding is determined by ELISA.

Thus, amino acids in the Fc region that play a dominant role in the interactions with C1q and the Fcγ receptors may be modified.

Examples of amino acid positions that may be modified, e.g. in an IgG1 isotype antibody, include positions L234, and L235.

Hence, in one embodiment, the amino acid in at least one position corresponding to L234, and L235 may be A, and A, respectively. Also, L234F and L235E amino acid substitutions can result in Fc regions with abrogated interactions with Fcγ receptors and C1q (Canfield et al., 1991, J. Exp. Med. (173):1483-91; Duncan et al., 1988, Nature (332):738-40). Hence, in one embodiment, the amino acids in the positions corresponding to L234 and L235, may be F and E, respectively. A D265A amino acid substitution can decrease binding to all Fcγ receptors and prevent ADCC (Shields et al., 2001, J. Biol. Chem. (276):6591-604). Hence, in one embodiment, the amino acid in the position corresponding to D265 may be A. Binding to C1q can be abrogated by mutating positions D270, K322, P329, and P331. Mutating these positions to either D270A or K322A or P329A or P331A can make the antibody deficient in CDC activity (Idusogie E E, et al., 2000, J Immunol. 164: 4178-84). Hence, in one embodiment, the amino acids in at least one position corresponding to D270, K322, P329 and P331, may be A, A, A, and A, respectively.

An alternative approach to minimize the interaction of the Fc region with Fcγ receptors and C1q is by removal of the glycosylation site of an antibody. Mutating position N297 to e.g. Q, A, or E removes a glycosylation site which is critical for IgG-Fc gamma Receptor interactions. Hence, in one embodiment, the amino acid in a position corresponding to N297, may be G, Q, A or E (Leabman et al., 2013, MAbs; 5(6):896-903). Another alternative approach to minimize interaction of the Fc region with Fcγ receptors may be obtained by the following mutations; P238A, A327Q, P329A or E233P/L234V/L235A/G236del (Shields et al., 2001, J. Biol. Chem. (276):6591-604).

Alternatively, human IgG2 and IgG4 subclasses are considered naturally compromised in their interactions with C1q and Fc gamma receptors although interactions with Fcγ receptors were reported (Parren et al., 1992, J. Clin Invest. 90: 1537-1546; Bruhns et al., 2009, Blood 113: 3716-3725). Mutations abrogating these residual interactions can be made in both isotypes, resulting in reduction of unwanted side-effects associated with FcR binding. For IgG2, these include L234A and G237A, and for IgG4, L235E. Hence, in one embodiment, the amino acid in a position corresponding to L234 and G237 in a human IgG2 heavy chain, may be A and A, respectively. In one embodiment, the amino acid in a position corresponding to L235 in a human IgG4 heavy chain, may be E.

Other approaches to further minimize the interaction with Fcγ receptors and C1q in IgG2 antibodies include those described in WO2011066501 and Lightle, S., et al., 2010, Protein Science (19):753-62.

The hinge region of the antibody can also be of importance with respect to interactions with Fcγ receptors and complement (Brekke et al., 2006, J Immunol 177:1129-1138; Dall'Acqua W F, et al., 2006, J Immunol 177:1129-1138). Accordingly, mutations in or deletion of the hinge region can influence effector functions of an antibody.

In one embodiment, the antibody comprises a first and a second immunoglobulin heavy chain, wherein in at least one of said first and second immunoglobulin heavy chains one or more amino acids in the positions corresponding to positions L234, L235, D265, N297, and P331 in a human IgG1 heavy chain, are not L, L, D, N, and P, respectively.

In one embodiment, in both the first and second heavy chains one or more amino acids in the position corresponding to positions L234, L235, D265, N297, and P331 in a human IgG1 heavy chain, are not L, L, D, N, and P, respectively.

In one embodiment of the invention, in both said first and second heavy chains the amino acid in the position corresponding to position D265 in a human IgG1 heavy chain, is not D.

Thus, in one embodiment of the invention, in both said first and second heavy chains the amino acid in the position corresponding to position D265 in a human IgG1 heavy chain are selected from the group consisting of: A and E.

In a further embodiment of the invention, in at least one of said first and second heavy chains the amino acids in the positions corresponding to positions L234 and L235 in a human IgG1 heavy chain, are not L and L, respectively.

In a particular embodiment of the invention, in at least one of said first and second heavy chains the amino acids in the positions corresponding to positions L234 and L235 in a human IgG1 heavy chain, are F and E, respectively.

In one embodiment of the invention, in both said first and second heavy chains the amino acids in the positions corresponding to positions L234 and L235 in a human IgG1 heavy chain, are F and E, respectively.

In a particular embodiment of the invention, in at least one of said first and second heavy chains the amino acids in the positions corresponding to positions L234, L235, and D265 in a human IgG1 heavy chain, are F, E, and A, respectively.

In a particularly preferred embodiment of the invention, in both said first and second heavy chains the amino acids in the positions corresponding to positions L234, L235, and D265 in a human IgG1 heavy chain, are F, E, and A, respectively.

Antibodies according to the present invention may comprise modifications, in particular in the Fc region, increasing stability of the antibody. Thus, in one embodiment, an antibody comprises a first and a second heavy chain, wherein one or both heavy chains are modified so that stability of the antibody is increased relative to an antibody which is identical, except for comprising non-modified first and second heavy chains. Examples of amino acid positions that may be modified, e.g. in an IgG1 isotype antibody, include positions M428, and N434. Hence, in one embodiment, the amino acid in at least one position corresponding to M428, and N434 may be L, and S, respectively. In one embodiment, the amino acid in positions corresponding to M428, and N434 are L, and S.

According to certain embodiments, the polypeptide chain(s) of a binding agent or antibody described herein may comprise a signal peptide.

Such signal peptides are sequences, which typically exhibit a length of about 15 to 30 amino acids and are preferably located at the N-terminus of a polypeptide chain, without being limited thereto. Signal peptides as defined herein preferably allow the transport of the polypeptide chain(s), e.g., as encoded by RNA, into a defined cellular compartment, preferably the cell surface, the endoplasmic reticulum (ER) or the endosomal-lysosomal compartment.

The signal peptide sequence as defined herein includes, without being limited thereto, the signal peptide sequence of an immunoglobulin, e.g., the signal peptide sequence of an immunoglobulin heavy chain variable region or the signal peptide sequence of an immunoglobulin light chain variable region, wherein the immunoglobulin may be human immunoglobulin.

In a further embodiment, the binding agents or antibodies described herein are linked or conjugated to one or more therapeutic moieties, such as a cytokine, an immune-suppressant, an immune-stimulatory molecule and/or a radioisotope. Such conjugates are referred to herein as “immunoconjugates” or “drug conjugates”. Immunoconjugates which include one or more cytotoxins are referred to as “immunotoxins”.

In one embodiment, the first and/or second Fc sequence is conjugated to a drug or a prodrug or contains an acceptor group for the same. Such acceptor group may e.g. be an unnatural amino acid.

Nucleic Acids

The term “polynucleotide” or “nucleic acid”, as used herein, is intended to include DNA and RNA such as genomic DNA, cDNA, mRNA, recombinantly produced and chemically synthesized molecules. A nucleic acid may be single-stranded or double-stranded. RNA includes in vitro transcribed RNA (IVT RNA) or synthetic RNA. According to the invention, a polynucleotide is preferably isolated.

Nucleic acids may be comprised in a vector. The term “vector” as used herein includes any vectors known to the skilled person including plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

In one embodiment of all aspects of the invention, the RNA encoding the binding agent, e.g., antibody or bispecific or multispecific binding agent, described herein is expressed in cells of the subject treated to provide the binding agent. If a binding agent comprises more than one polypeptide chain the different polypeptide chains may be encoded by the same or different RNA molecules.

The nucleic acids described herein may be recombinant and/or isolated molecules.

In the present disclosure, the term “RNA” relates to a nucleic acid molecule which includes ribonucleotide residues. In preferred embodiments, the RNA contains all or a majority of ribonucleotide residues. As used herein, “ribonucleotide” refers to a nucleotide with a hydroxyl group at the 2′-position of a β-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered RNAs are considered analogs of naturally-occurring RNA.

In certain embodiments of the present disclosure, the RNA is messenger RNA (mRNA) that relates to a RNA transcript which encodes a peptide or protein. As established in the art, mRNA generally contains a 5′ untranslated region (5′-UTR), a peptide coding region and a 3′ untranslated region (3′-UTR). In some embodiments, the RNA is produced by in vitro transcription or chemical synthesis. In one embodiment, the mRNA is produced by in vitro transcription using a DNA template where DNA refers to a nucleic acid that contains deoxyribonucleotides.

In one embodiment, RNA is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

In certain embodiments of the present disclosure, the RNA is “replicon RNA” or simply a “replicon”, in particular “self-replicating RNA” or “self-amplifying RNA”. In one particularly preferred embodiment, the replicon or self-replicating RNA is derived from or comprises elements derived from a ssRNA virus, in particular a positive-stranded ssRNA virus such as an alphavirus. Alphaviruses are typical representatives of positive-stranded RNA viruses. Alphaviruses replicate in the cytoplasm of infected cells (for review of the alphaviral life cycle see José et al., Future Microbiol., 2009, vol. 4, pp. 837-856). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFS) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124). Following infection, i.e. at early stages of the viral life cycle, the (+) stranded genomic RNA directly acts like a messenger RNA for the translation of the open reading frame encoding the non-structural poly-protein (nsP1234). Alphavirus-derived vectors have been proposed for delivery of foreign genetic information into target cells or target organisms. In simple approaches, the open reading frame encoding alphaviral structural proteins is replaced by an open reading frame encoding a protein of interest. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

In one embodiment, the RNA described herein may have modified nucleosides. In some embodiments, the RNA comprises a modified nucleoside in place of at least one (e.g. every) uridine.

The term “uracil,” as used herein, describes one of the nucleobases that can occur in the nucleic acid of RNA. The structure of uracil is:

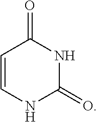

The term “uridine,” as used herein, describes one of the nucleosides that can occur in RNA.

The structure of uridine is:

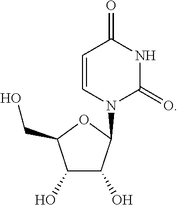 [SMILES: O=c1ccn([C@@H]2O[C@H](CO)[C@@H](O)[C@H]2O)c(=O)[nH]1]

UTP (uridine 5′-triphosphate) has the following structure:

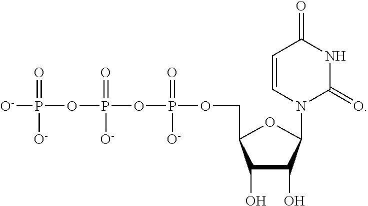

Pseudo-UTP (pseudouridine 5′-triphosphate) has the following structure:

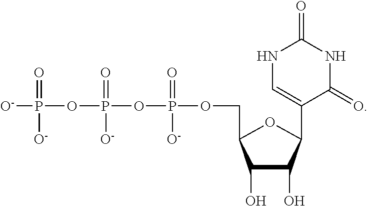

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N1-methyl-pseudouridine (m1ψ), which has the structure:

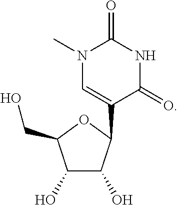 [SMILES: Cn1cc([C@@H]2O[C@H](CO)[C@@H](O)[C@H]2O)c(=O)[nH]c1=O]

N1-methyl-pseudo-UTP has the following structure:

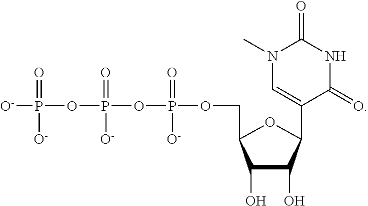

Another exemplary modified nucleoside is 5-methyl-uridine (m5U), which has the structure:

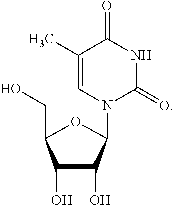

In some embodiments, one or more uridine in the RNA described herein is replaced by a modified nucleoside. In some embodiments, the modified nucleoside is a modified uridine.

In some embodiments, RNA comprises a modified nucleoside in place of at least one uridine.

In some embodiments, RNA comprises a modified nucleoside in place of each uridine.

In some embodiments, the modified nucleoside is independently selected from pseudouridine (ψ), N1-methyl-pseudouridine (m1ψ), and 5-methyl-uridine (m5U). In some embodiments, the modified nucleoside comprises pseudouridine (ψ). In some embodiments, the modified nucleoside comprises N1-methyl-pseudouridine (m1ψ). In some embodiments, the modified nucleoside comprises 5-methyl-uridine (m5U). In some embodiments, RNA may comprise more than one type of modified nucleoside, and the modified nucleosides are independently selected from pseudouridine (ψ), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (ψ) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ), N1-methyl-pseudouridine (m1ψ), and 5-methyl-uridine (m5U).

In some embodiments, the modified nucleoside replacing one or more, e.g., all, uridine in the RNA may be any one or more of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵ s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵ s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine (τm5s2U), 1-taurinomethyl-4-thio-pseudouridine), 5-methyl-2-thio-uridine (m⁵ s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ ψ), 5-(isopentenylaminomethyl)uridine (inm⁵U), 5-(isopentenylaminomethyl)-2-thio-uridine (inm⁵ s²U), a-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

In one embodiment, the RNA comprises other modified nucleosides or comprises further modified nucleosides, e.g., modified cytidine. For example, in one embodiment, in the RNA 5-methylcytidine is substituted partially or completely, preferably completely, for cytidine. In one embodiment, the RNA comprises 5-methylcytidine and one or more selected from pseudouridine (ψ), N1-methyl-pseudouridine (m1ψ), and 5-methyl-uridine (m5U). In one embodiment, the RNA comprises 5-methylcytidine and N1-methyl-pseudouridine (m1ψ). In some embodiments, the RNA comprises 5-methylcytidine in place of each cytidine and N1-methyl-pseudouridine (m1ψ) in place of each uridine.

In some embodiments, the RNA according to the present disclosure comprises a 5′-cap. In one embodiment, the RNA of the present disclosure does not have uncapped 5′-triphosphates. In one embodiment, the RNA may be modified by a 5′-cap analog. The term “5′-cap” refers to a structure found on the 5′-end of an mRNA molecule and generally consists of a guanosine nucleotide connected to the mRNA via a 5′- to 5′-triphosphate linkage. In one embodiment, this guanosine is methylated at the 7-position. Providing an RNA with a 5′-cap or 5′-cap analog may be achieved by in vitro transcription, in which the 5′-cap is co-transcriptionally expressed into the RNA strand, or may be attached to RNA post-transcriptionally using capping enzymes.

In some embodiments, the building block cap for RNA is m₂^{7,3′-O}Gppp(m₁^{2′-O})ApG (also sometimes referred to as m₂^{7,3′-O}G(5′)ppp(5′)m^{2′-O}ApG), which has the following structure:

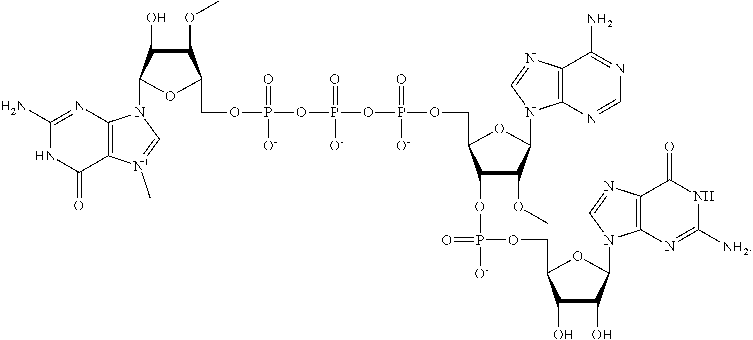

Below is an exemplary Cap1 RNA, which comprises RNA and m₂^{7,3′-O}G(5′)ppp(5′)m^{2′-O}ApG:

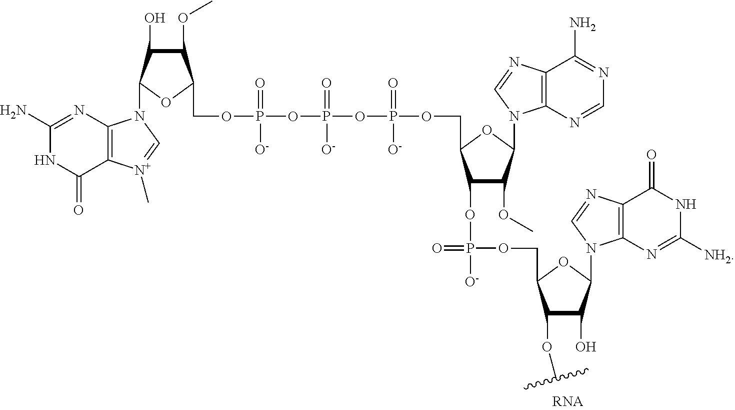

Below is another exemplary Cap1 RNA (no cap analog):

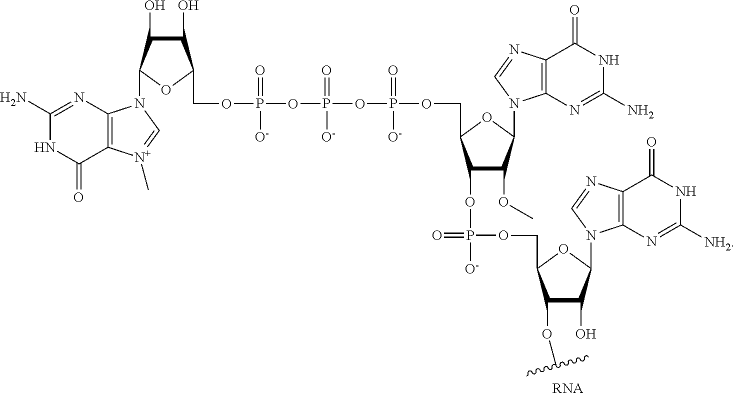

In some embodiments, the RNA is modified with “Cap0” structures using, in one embodiment, the cap analog anti-reverse cap (ARCA Cap (m₂^{7,3′-O}G(5′)ppp(5′)G)) with the structure:

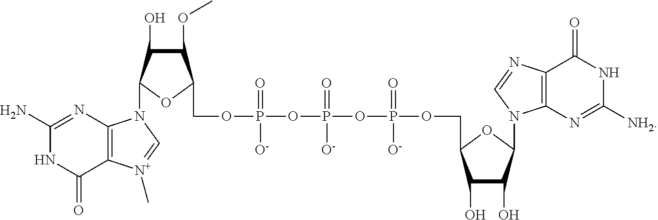

Below is an exemplary Cap0 RNA comprising RNA and m₂^{7,3′-O}G(5′)ppp(5′)G:

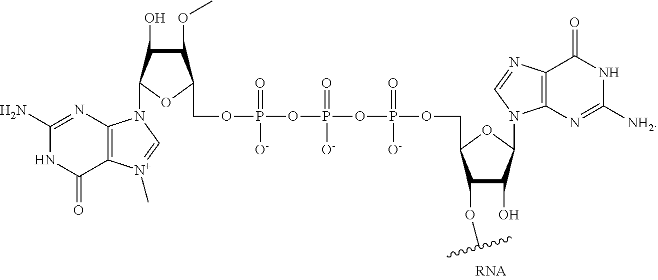

In some embodiments, the “Cap0” structures are generated using the cap analog Beta-S-ARCA (m₂^{7,2′O}G(5′)ppSp(5′)G) with the structure:

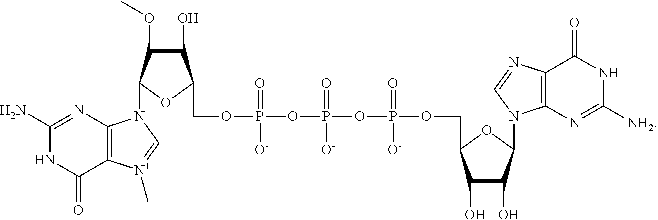

Below is an exemplary Cap0 RNA comprising Beta-S-ARCA (m₂^{7,2′O}G(5′)ppSp(5′)G) and RNA:

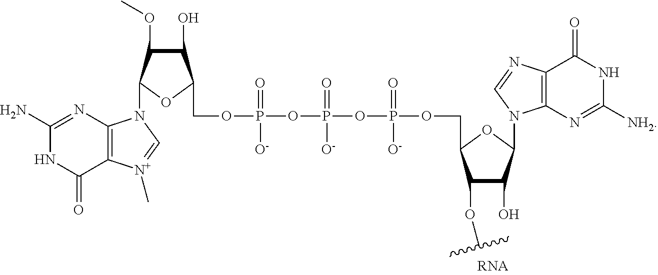

The “D1” diastereomer of beta-S-ARCA or “beta-S-ARCA(D1)” is the diastereomer of beta-S-ARCA which elutes first on an HPLC column compared to the D2 diastereomer of beta-S-ARCA (beta-S-ARCA(D2)) and thus exhibits a shorter retention time (cf., WO 2011/015347, herein incorporated by reference).

A particularly preferred cap is beta-S-ARCA(D1) (m₂^{7,2′-O}GppSpG) or m₂^{7,3′-O}Gppp(m₁^{2′-O})ApG.

In some embodiments, RNA according to the present disclosure comprises a 5′-UTR and/or a 3′-UTR. The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′ end, upstream of the start codon of a protein-encoding region. A 5′-UTR is downstream of the 5′-cap (if present), e.g. directly adjacent to the 5′-cap. A 3′-UTR, if present, is located at the 3′ end, downstream of the termination codon of a protein-encoding region, but the term “3′-UTR” does preferably not include the poly(A) sequence. Thus, the 3′-UTR is upstream of the poly(A) sequence (if present), e.g. directly adjacent to the poly(A) sequence.

In some embodiments, RNA comprises a 5′-UTR comprising the nucleotide sequence of SEQ ID NO: 199, or a nucleotide sequence having at least 99%, 98%, 97%, 96%, 95%, 90%, 85%, or 80% identity to the nucleotide sequence of SEQ ID NO: 199.

In some embodiments, RNA comprises a 3′-UTR comprising the nucleotide sequence of SEQ ID NO: 200 or 201, or a nucleotide sequence having at least 99%, 98%, 97%, 96%, 95%, 90%, 85%, or 80% identity to the nucleotide sequence of SEQ ID NO: 200 or 201.

A particularly preferred 5′-UTR comprises the nucleotide sequence of SEQ ID NO: 199. A particularly preferred 3′-UTR comprises the nucleotide sequence of SEQ ID NO: 200 or 201. In some embodiments, the RNA according to the present disclosure comprises a 3′-poly(A) sequence.

As used herein, the term “poly(A) sequence” or “poly-A tail” refers to an uninterrupted or interrupted sequence of adenylate residues which is typically located at the 3′-end of an RNA molecule. Poly(A) sequences are known to those of skill in the art and may follow the 3′-UTR in the RNAs described herein. An uninterrupted poly(A) sequence is characterized by consecutive adenylate residues. In nature, an uninterrupted poly(A) sequence is typical. RNAs disclosed herein can have a poly(A) sequence attached to the free 3′-end of the RNA by a template-independent RNA polymerase after transcription or a poly(A) sequence encoded by DNA and transcribed by a template-dependent RNA polymerase.

It has been demonstrated that a poly(A) sequence of about 120 A nucleotides has a beneficial influence on the levels of RNA in transfected eukaryotic cells, as well as on the levels of protein that is translated from an open reading frame that is present upstream (5′) of the poly(A) sequence (Holtkamp et al., 2006, Blood, vol. 108, pp. 4009-4017).

The poly(A) sequence may be of any length. In some embodiments, a poly(A) sequence comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly(A) sequence, typically at least 75%, at least 80%, at least 85%, at least 90%, at least 95%, at least 96%, at least 97%, at least 98%, or at least 99% by number of nucleotides in the poly(A) sequence are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly(A) sequence, i.e., 100% by number of nucleotides in the poly(A) sequence, are A nucleotides. The term “A nucleotide” or “A” refers to adenylate.

In some embodiments, a poly(A) sequence is attached during RNA transcription, e.g., during preparation of in vitro transcribed RNA, based on a DNA template comprising repeated dT nucleotides (deoxythymidylate) in the strand complementary to the coding strand. The DNA sequence encoding a poly(A) sequence (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present invention. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. Consequently, in some embodiments, the poly(A) sequence contained in an RNA molecule described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly(A) sequence at its 3′-end, i.e., the poly(A) sequence is not masked or followed at its 3′-end by a nucleotide other than A.

In some embodiments, the poly(A) sequence may comprise at least 20, at least 30, at least 40, at least 80, or at least 100 and up to 500, up to 400, up to 300, up to 200, or up to 150 nucleotides. In some embodiments, the poly(A) sequence may essentially consist of at least 20, at least 30, at least 40, at least 80, or at least 100 and up to 500, up to 400, up to 300, up to 200, or up to 150 nucleotides. In some embodiments, the poly(A) sequence may consist of at least 20, at least 30, at least 40, at least 80, or at least 100 and up to 500, up to 400, up to 300, up to 200, or up to 150 nucleotides. In some embodiments, the poly(A) sequence comprises at least 100 nucleotides. In some embodiments, the poly(A) sequence comprises about 150 nucleotides. In some embodiments, the poly(A) sequence comprises about 120 nucleotides.

In some embodiments, RNA comprises a poly(A) sequence comprising the nucleotide sequence of SEQ ID NO: 202, or a nucleotide sequence having at least 99%, 98%, 97%, 96%, 95%, 90%, 85%, or 80% identity to the nucleotide sequence of SEQ ID NO: 202.

A particularly preferred poly(A) sequence comprises the nucleotide sequence of SEQ ID NO: 202.

According to the disclosure, a binding agent is preferably administered as single-stranded, 5′-capped mRNA that is translated into the respective protein upon entering cells of a subject being administered the RNA. Preferably, the RNA contains structural elements optimized for maximal efficacy of the RNA with respect to stability and translational efficiency (5′-cap, 5′-UTR, 3′-UTR, poly(A) sequence).

In one embodiment, beta-S-ARCA(D1) is utilized as specific capping structure at the 5′-end of the RNA. In one embodiment, m₂^{7,3′-O}Gppp(m₁^{2′-O})ApG is utilized as specific capping structure at the 5′-end of the RNA. In one embodiment, the 5′-UTR sequence is derived from the human alpha-globin mRNA and optionally has an optimized ‘Kozak sequence’ to increase translational efficiency. In one embodiment, a combination of two sequence elements (FI element) derived from the “amino terminal enhancer of split” (AES) mRNA (called F) and the mitochondrial encoded 12S ribosomal RNA (called I) are placed between the coding sequence and the poly(A) sequence to assure higher maximum protein levels and prolonged persistence of the mRNA. These were identified by an ex vivo selection process for sequences that confer RNA stability and augment total protein expression (see WO 2017/060314, herein incorporated by reference). In one embodiment, two re-iterated 3′-UTRs derived from the human beta-globin mRNA are placed between the coding sequence and the poly(A) sequence to assure higher maximum protein levels and prolonged persistence of the mRNA. In one embodiment, a poly(A) sequence measuring 110 nucleotides in length, consisting of a stretch of 30 adenosine residues, followed by a 10 nucleotide linker sequence and another 70 adenosine residues is used. This poly(A) sequence was designed to enhance RNA stability and translational efficiency.

In one embodiment of all aspects of the invention, RNA encoding a binding agent is expressed in cells of the subject treated to provide the binding agent. In one embodiment of all aspects of the invention, the RNA is transiently expressed in cells of the subject. In one embodiment of all aspects of the invention, the RNA is in vitro transcribed RNA. In one embodiment of all aspects of the invention, expression of the binding agent is into the extracellular space, i.e., the binding agent is secreted.

In the context of the present disclosure, the term “transcription” relates to a process, wherein the genetic code in a DNA sequence is transcribed into RNA. Subsequently, the RNA may be translated into peptide or protein.

According to the present invention, the term “transcription” comprises “in vitro transcription”, wherein the term “in vitro transcription” relates to a process wherein RNA, in particular mRNA, is in vitro synthesized in a cell-free system, preferably using appropriate cell extracts. Preferably, cloning vectors are applied for the generation of transcripts. These cloning vectors are generally designated as transcription vectors and are according to the present invention encompassed by the term “vector”. According to the present invention, the RNA used in the present invention preferably is in vitro transcribed RNA (IVT-RNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription according to the invention is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

With respect to RNA, the term “expression” or “translation” relates to the process in the ribosomes of a cell by which a strand of mRNA directs the assembly of a sequence of amino acids to make a peptide or protein.

In one embodiment, after administration of the RNA described herein, e.g., formulated as RNA lipid particles, at least a portion of the RNA is delivered to a target cell. In one embodiment, at least a portion of the RNA is delivered to the cytosol of the target cell. In one embodiment, the RNA is translated by the target cell to produce the peptide or protein it enodes. Accordingly, the present disclosure also relates to a method for delivering RNA to a target cell in a subject comprising the administration of the RNA particles described herein to the subject.

In one embodiment, the RNA is delivered to the cytosol of the target cell. In one embodiment, the RNA is translated by the target cell to produce the peptide or protein encoded by the RNA.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

In one embodiment, the RNA encoding binding agent to be administered according to the invention is non-immunogenic.

The term “non-immunogenic RNA” as used herein refers to RNA that does not induce a response by the immune system upon administration, e.g., to a mammal, or induces a weaker response than would have been induced by the same RNA that differs only in that it has not been subjected to the modifications and treatments that render the non-immunogenic RNA non-immunogenic, i.e., than would have been induced by standard RNA (stdRNA). In one preferred embodiment, non-immunogenic RNA, which is also termed modified RNA (modRNA) herein, is rendered non-immunogenic by incorporating modified nucleosides suppressing RNA-mediated activation of innate immune receptors into the RNA and removing double-stranded RNA (dsRNA).

For rendering the non-immunogenic RNA non-immunogenic by the incorporation of modified nucleosides, any modified nucleoside may be used as long as it lowers or suppresses immunogenicity of the RNA. Particularly preferred are modified nucleosides that suppress RNA-mediated activation of innate immune receptors. In one embodiment, the modified nucleosides comprises a replacement of one or more uridines with a nucleoside comprising a modified nucleobase. In one embodiment, the modified nucleobase is a modified uracil. In one embodiment, the nucleoside comprising a modified nucleobase is selected from the group consisting of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵ s²U), 5-aminomethyl-2-thio-uridine (nm⁵ s²U), 5-methylaminomethyl-uridine (mnm⁵U), 1-ethyl-pseudouridine, 5-methylaminomethyl-2-thio-uridine (mnm⁵ s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine (τm⁵s²U), 1-taurinomethyl-4-thio-pseudouridine), 5-methyl-2-thio-uridine (m⁵ s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵ s²U), a-thio-uridine, 2′-O-methyl-uridine (Urn), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)uridine. In one particularly preferred embodiment, the nucleoside comprising a modified nucleobase is pseudouridine 4), N1-methyl-pseudouridine (m1ψ) or 5-methyl-uridine (m5U), in particular N1-methyl-pseudouridine.

In one embodiment, the replacement of one or more uridines with a nucleoside comprising a modified nucleobase comprises a replacement of at least 1%, at least 2%, at least 3%, at least 4%, at least 5%, at least 10%, at least 25%, at least 50%, at least 75%, at least 90%, at least 95%, at least 96%, at least 97%, at least 98%, at least 99% or 100% of the uridines. During synthesis of mRNA by in vitro transcription (IVT) using T7 RNA polymerase significant amounts of aberrant products, including double-stranded RNA (dsRNA) are produced due to unconventional activity of the enzyme. dsRNA induces inflammatory cytokines and activates effector enzymes leading to protein synthesis inhibition. dsRNA can be removed from RNA such as IVT RNA, for example, by ion-pair reversed phase HPLC using a non-porous or porous C-18 polystyrene-divinylbenzene (PS-DVB) matrix. Alternatively, an enzymatic based method using E. coli RNaseIII that specifically hydrolyzes dsRNA but not ssRNA, thereby eliminating dsRNA contaminants from IVT RNA preparations can be used. Furthermore, dsRNA can be separated from ssRNA by using a cellulose material. In one embodiment, an RNA preparation is contacted with a cellulose material and the ssRNA is separated from the cellulose material under conditions which allow binding of dsRNA to the cellulose material and do not allow binding of ssRNA to the cellulose material.

As the term is used herein, “remove” or “removal” refers to the characteristic of a population of first substances, such as non-immunogenic RNA, being separated from the proximity of a population of second substances, such as dsRNA, wherein the population of first substances is not necessarily devoid of the second substance, and the population of second substances is not necessarily devoid of the first substance. However, a population of first substances characterized by the removal of a population of second substances has a measurably lower content of second substances as compared to the non-separated mixture of first and second substances.

In one embodiment, the removal of dsRNA from non-immunogenic RNA comprises a removal of dsRNA such that less than 10%, less than 5%, less than 4%, less than 3%, less than 2%, less than 1%, less than 0.5%, less than 0.3%, or less than 0.1% of the RNA in the non-immunogenic RNA composition is dsRNA. In one embodiment, the non-immunogenic RNA is free or essentially free of dsRNA. In some embodiments, the non-immunogenic RNA composition comprises a purified preparation of single-stranded nucleoside modified RNA. For example, in some embodiments, the purified preparation of single-stranded nucleoside modified RNA is substantially free of double stranded RNA (dsRNA). In some embodiments, the purified preparation is at least 90%, at least 91%, at least 92%, at least 93%, at least 94%, at least 95%, at least 96%, at least 97%, at least 98%, at least 99%, at least 99.5%, or at least 99.9% single stranded nucleoside modified RNA, relative to all other nucleic acid molecules (DNA, dsRNA, etc.).

In one embodiment, the non-immunogenic RNA is translated in a cell more efficiently than standard RNA with the same sequence. In one embodiment, translation is enhanced by a factor of 2-fold relative to its unmodified counterpart. In one embodiment, translation is enhanced by a 3-fold factor. In one embodiment, translation is enhanced by a 4-fold factor. In one embodiment, translation is enhanced by a 5-fold factor. In one embodiment, translation is enhanced by a 6-fold factor. In one embodiment, translation is enhanced by a 7-fold factor. In one embodiment, translation is enhanced by an 8-fold factor. In one embodiment, translation is enhanced by a 9-fold factor. In one embodiment, translation is enhanced by a 10-fold factor. In one embodiment, translation is enhanced by a 15-fold factor. In one embodiment, translation is enhanced by a 20-fold factor. In one embodiment, translation is enhanced by a 50-fold factor. In one embodiment, translation is enhanced by a 100-fold factor. In one embodiment, translation is enhanced by a 200-fold factor. In one embodiment, translation is enhanced by a 500-fold factor. In one embodiment, translation is enhanced by a 1000-fold factor. In one embodiment, translation is enhanced by a 2000-fold factor. In one embodiment, the factor is 104000-fold. In one embodiment, the factor is 10-100-fold. In one embodiment, the factor is 10-200-fold. In one embodiment, the factor is 10-300-fold. In one embodiment, the factor is 10-500-fold. In one embodiment, the factor is 20-1000-fold. In one embodiment, the factor is 30-1000-fold. In one embodiment, the factor is 50-1000-fold. In one embodiment, the factor is 100-1000-fold. In one embodiment, the factor is 200-1000-fold. In one embodiment, translation is enhanced by any other significant amount or range of amounts.

In one embodiment, the non-immunogenic RNA exhibits significantly less innate immunogenicity than standard RNA with the same sequence. In one embodiment, the non-immunogenic RNA exhibits an innate immune response that is 2-fold less than its unmodified counterpart. In one embodiment, innate immunogenicity is reduced by a 3-fold factor. In one embodiment, innate immunogenicity is reduced by a 4-fold factor. In one embodiment, innate immunogenicity is reduced by a 5-fold factor. In one embodiment, innate immunogenicity is reduced by a 6-fold factor. In one embodiment, innate immunogenicity is reduced by a 7-fold factor. In one embodiment, innate immunogenicity is reduced by a 8-fold factor. In one embodiment, innate immunogenicity is reduced by a 9-fold factor. In one embodiment, innate immunogenicity is reduced by a 10-fold factor. In one embodiment, innate immunogenicity is reduced by a 15-fold factor. In one embodiment, innate immunogenicity is reduced by a 20-fold factor. In one embodiment, innate immunogenicity is reduced by a 50-fold factor. In one embodiment, innate immunogenicity is reduced by a 100-fold factor. In one embodiment, innate immunogenicity is reduced by a 200-fold factor. In one embodiment, innate immunogenicity is reduced by a 500-fold factor. In one embodiment, innate immunogenicity is reduced by a 1000-fold factor. In one embodiment, innate immunogenicity is reduced by a 2000-fold factor.

The term “exhibits significantly less innate immunogenicity” refers to a detectable decrease in innate immunogenicity. In one embodiment, the term refers to a decrease such that an effective amount of the non-immunogenic RNA can be administered without triggering a detectable innate immune response. In one embodiment, the term refers to a decrease such that the non-immunogenic RNA can be repeatedly administered without eliciting an innate immune response sufficient to detectably reduce production of the protein encoded by the non-immunogenic RNA. In one embodiment, the decrease is such that the non-immunogenic RNA can be repeatedly administered without eliciting an innate immune response sufficient to eliminate detectable production of the protein encoded by the non-immunogenic RNA.

“Immunogenicity” is the ability of a foreign substance, such as RNA, to provoke an immune response in the body of a human or other animal. The innate immune system is the component of the immune system that is relatively unspecific and immediate. It is one of two main components of the vertebrate immune system, along with the adaptive immune system.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence.

As used herein, the terms “linked,” “fused”, or “fusion” are used interchangeably. These terms refer to the joining together of two or more elements or components or domains.

Codon-Optimization/Increase in G/C Content

In some embodiment, the amino acid sequence of a binding agent described herein is encoded by a coding sequence which is codon-optimized and/or the G/C content of which is increased compared to wild type coding sequence. This also includes embodiments, wherein one or more sequence regions of the coding sequence are codon-optimized and/or increased in the G/C content compared to the corresponding sequence regions of the wild type coding sequence. In one embodiment, the codon-optimization and/or the increase in the G/C content preferably does not change the sequence of the encoded amino acid sequence.

The term “codon-optimized” refers to the alteration of codons in the coding region of a nucleic acid molecule to reflect the typical codon usage of a host organism without preferably altering the amino acid sequence encoded by the nucleic acid molecule. Within the context of the present invention, coding regions are preferably codon-optimized for optimal expression in a subject to be treated using the RNA molecules described herein. Codon-optimization is based on the finding that the translation efficiency is also determined by a different frequency in the occurrence of tRNAs in cells. Thus, the sequence of RNA may be modified such that codons for which frequently occurring tRNAs are available are inserted in place of “rare codons”.

In some embodiments of the invention, the guanosine/cytosine (G/C) content of the coding region of the RNA described herein is increased compared to the G/C content of the corresponding coding sequence of the wild type RNA, wherein the amino acid sequence encoded by the RNA is preferably not modified compared to the amino acid sequence encoded by the wild type RNA. This modification of the RNA sequence is based on the fact that the sequence of any RNA region to be translated is important for efficient translation of that mRNA. Sequences having an increased G (guanosine)/C (cytosine) content are more stable than sequences having an increased A (adenosine)/U (uracil) content. In respect to the fact that several codons code for one and the same amino acid (so-called degeneration of the genetic code), the most favourable codons for the stability can be determined (so-called alternative codon usage). Depending on the amino acid to be encoded by the RNA, there are various possibilities for modification of the RNA sequence, compared to its wild type sequence. In particular, codons which contain A and/or U nucleotides can be modified by substituting these codons by other codons, which code for the same amino acids but contain no A and/or U or contain a lower content of A and/or U nucleotides.

In various embodiments, the G/C content of the coding region of the RNA described herein is increased by at least 10%, at least 20%, at least 30%, at least 40%, at least 50%, at least 55%, or even more compared to the G/C content of the coding region of the wild type RNA.

Nucleic Acid Containing Particles

Nucleic acids described herein such as RNA encoding a binding agent may be administered formulated as particles.

In the context of the present disclosure, the term “particle” relates to a structured entity formed by molecules or molecule complexes. In one embodiment, the term “particle” relates to a micro- or nano-sized structure, such as a micro- or nano-sized compact structure dispersed in a medium. In one embodiment, a particle is a nucleic acid containing particle such as a particle comprising DNA, RNA or a mixture thereof.

Electrostatic interactions between positively charged molecules such as polymers and lipids and negatively charged nucleic acid are involved in particle formation. This results in complexation and spontaneous formation of nucleic acid particles. In one embodiment, a nucleic acid particle is a nanoparticle.

As used in the present disclosure, “nanoparticle” refers to a particle having an average diameter suitable for parenteral administration.

A “nucleic acid particle” can be used to deliver nucleic acid to a target site of interest (e.g., cell, tissue, organ, and the like). A nucleic acid particle may be formed from at least one cationic or cationically ionizable lipid or lipid-like material, at least one cationic polymer such as protamine, or a mixture thereof and nucleic acid. Nucleic acid particles include lipid nanoparticle (LNP)-based and lipoplex (LPX)-based formulations.

Without intending to be bound by any theory, it is believed that the cationic or cationically ionizable lipid or lipid-like material and/or the cationic polymer combine together with the nucleic acid to form aggregates, and this aggregation results in colloidally stable particles.

In one embodiment, particles described herein further comprise at least one lipid or lipid-like material other than a cationic or cationically ionizable lipid or lipid-like material, at least one polymer other than a cationic polymer, or a mixture thereof

In some embodiments, nucleic acid particles comprise more than one type of nucleic acid molecules, where the molecular parameters of the nucleic acid molecules may be similar or different from each other, like with respect to molar mass or fundamental structural elements such as molecular architecture, capping, coding regions or other features,

Nucleic acid particles described herein may have an average diameter that in one embodiment ranges from about 30 nm to about 1000 nm, from about 50 nm to about 800 nm, from about 70 nm to about 600 nm, from about 90 nm to about 400 nm, or from about 100 nm to about 300 nm.

Nucleic acid particles described herein may exhibit a polydispersity index less than about 0.5, less than about 0.4, less than about 0.3, or about 0.2 or less. By way of example, the nucleic acid particles can exhibit a polydispersity index in a range of about 0.1 to about 0.3 or about 0.2 to about 0.3.

With respect to RNA lipid particles, the N/P ratio gives the ratio of the nitrogen groups in the lipid to the number of phosphate groups in the RNA. It is correlated to the charge ratio, as the nitrogen atoms (depending on the pH) are usually positively charged and the phosphate groups are negatively charged. The N/P ratio, where a charge equilibrium exists, depends on the pH. Lipid formulations are frequently formed at N/P ratios larger than four up to twelve, because positively charged nanoparticles are considered favorable for transfection. In that case, RNA is considered to be completely bound to nanoparticles.

Nucleic acid particles described herein can be prepared using a wide range of methods that may involve obtaining a colloid from at least one cationic or cationically ionizable lipid or lipid-like material and/or at least one cationic polymer and mixing the colloid with nucleic acid to obtain nucleic acid particles.

The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

For the preparation of colloids comprising at least one cationic or cationically ionizable lipid or lipid-like material and/or at least one cationic polymer methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media).

In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the RNA lipoplex particles described herein are obtainable by adding RNA to a colloidal liposome dispersion. Using the ethanol injection technique, such colloidal liposome dispersion is, in one embodiment, formed as follows: an ethanol solution comprising lipids, such as cationic lipids and additional lipids, is injected into an aqueous solution under stirring. In one embodiment, the RNA lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

Other methods having organic solvent free characteristics may also be used according to the present disclosure for preparing a colloid.

LNPs typically comprise four components: ionizable cationic lipids, neutral lipids such as phospholipids, a steroid such as cholesterol, and a polymer conjugated lipid such as polyethylene glycol (PEG)-lipids. Each component is responsible for payload protection, and enables effective intracellular delivery. LNPs may be prepared by mixing lipids dissolved in ethanol rapidly with nucleic acid in an aqueous buffer.

The term “average diameter” refers to the mean hydrodynamic diameter of particles as measured by dynamic laser light scattering (DLS) with data analysis using the so-called cumulant algorithm, which provides as results the so-called Z_{average} with the dimension of a length, and the polydispersity index (PI), which is dimensionless (Koppel, D., J. Chem. Phys. 57, 1972, pp 4814-4820, ISO 13321). Here “average diameter”, “diameter” or “size” for particles is used synonymously with this value of the Z_{average}.

The “polydispersity index” is preferably calculated based on dynamic light scattering measurements by the so-called cumulant analysis as mentioned in the definition of the “average diameter”. Under certain prerequisites, it can be taken as a measure of the size distribution of an ensemble of nanoparticles.

Different types of nucleic acid containing particles have been described previously to be suitable for delivery of nucleic acid in particulate form (e.g. Kaczmarek, J. C. et al., 2017, Genome Medicine 9, 60). For non-viral nucleic acid delivery vehicles, nanoparticle encapsulation of nucleic acid physically protects nucleic acid from degradation and, depending on the specific chemistry, can aid in cellular uptake and endosomal escape.

The present disclosure describes particles comprising nucleic acid, at least one cationic or cationically ionizable lipid or lipid-like material, and/or at least one cationic polymer which associate with nucleic acid to form nucleic acid particles and compositions comprising such particles. The nucleic acid particles may comprise nucleic acid which is complexed in different forms by non-covalent interactions to the particle. The particles described herein are not viral particles, in particular infectious viral particles, i.e., they are not able to virally infect cells. Suitable cationic or cationically ionizable lipids or lipid-like materials and cationic polymers are those that form nucleic acid particles and are included by the term “particle forming components” or “particle forming agents”. The term “particle forming components” or “particle forming agents” relates to any components which associate with nucleic acid to form nucleic acid particles. Such components include any component which can be part of nucleic acid particles.

Cationic Polymer

Given their high degree of chemical flexibility, polymers are commonly used materials for nanoparticle-based delivery. Typically, cationic polymers are used to electrostatically condense the negatively charged nucleic acid into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic polymers herein. In addition, some investigators have synthesized polymers specifically for nucleic acid delivery. Poly(β-amino esters), in particular, have gained widespread use in nucleic acid delivery owing to their ease of synthesis and biodegradability. Such synthetic polymers are also suitable as cationic polymers herein.

A “polymer,” as used herein, is given its ordinary meaning, i.e., a molecular structure comprising one or more repeat units (monomers), connected by covalent bonds. The repeat units can all be identical, or in some cases, there can be more than one type of repeat unit present within the polymer. In some cases, the polymer is biologically derived, i.e., a biopolymer such as a protein. In some cases, additional moieties can also be present in the polymer, for example targeting moieties such as those described herein.

If more than one type of repeat unit is present within the polymer, then the polymer is said to be a “copolymer.” It is to be understood that the polymer being employed herein can be a copolymer. The repeat units forming the copolymer can be arranged in any fashion. For example, the repeat units can be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In certain embodiments, the polymer is biocompatible. Biocompatible polymers are polymers that typically do not result in significant cell death at moderate concentrations. In certain embodiments, the biocompatible polymer is biodegradable, i.e., the polymer is able to degrade, chemically and/or biologically, within a physiological environment, such as within the body.

In certain embodiments, polymer may be protamine or polyalkyleneimine, in particular protamine.

The term “protamine” refers to any of various strongly basic proteins of relatively low molecular weight that are rich in arginine and are found associated especially with DNA in place of somatic histones in the sperm cells of various animals (as fish). In particular, the term “protamine” refers to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

According to the disclosure, the term “protamine” as used herein is meant to comprise any protamine amino acid sequence obtained or derived from natural or biological sources including fragments thereof and multimeric forms of said amino acid sequence or fragment thereof as well as (synthesized) polypeptides which are artificial and specifically designed for specific purposes and cannot be isolated from native or biological sources.

In one embodiment, the polyalkyleneimine comprises polyethylenimine and/or polypropylenimine, preferably polyethyleneimine. A preferred polyalkyleneimine is polyethyleneimine (PEI). The average molecular weight of PEI is preferably 0.75·10² to 10⁷ Da, preferably 1000 to 10⁵ Da, more preferably 10000 to 40000 Da, more preferably 15000 to 30000 Da, even more preferably 20000 to 25000 Da.

Preferred according to the disclosure is linear polyalkyleneimine such as linear polyethyleneimine (PEI).

Cationic polymers (including polycationic polymers) contemplated for use herein include any cationic polymers which are able to electrostatically bind nucleic acid. In one embodiment, cationic polymers contemplated for use herein include any cationic polymers with which nucleic acid can be associated, e.g. by forming complexes with the nucleic acid or forming vesicles in which the nucleic acid is enclosed or encapsulated.

Particles described herein may also comprise polymers other than cationic polymers, i.e., non-cationic polymers and/or anionic polymers. Collectively, anionic and neutral polymers are referred to herein as non-cationic polymers.

Lipid and Lipid-Like Material

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

The term “lipid-like material”, “lipid-like compound” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids. As used herein, the term “lipid” is to be construed to cover both lipids and lipid-like materials unless otherwise indicated herein or clearly contradicted by context.

Specific examples of amphiphilic compounds that may be included in an amphiphilic layer include, but are not limited to, phospholipids, aminolipids and sphingolipids.

In certain embodiments, the amphiphilic compound is a lipid. The term “lipid” refers to a group of organic compounds that are characterized by being insoluble in water, but soluble in many organic solvents. Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as sterol-containing metabolites such as cholesterol.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides. Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

According to the disclosure, lipids and lipid-like materials may be cationic, anionic or neutral. Neutral lipids or lipid-like materials exist in an uncharged or neutral zwitterionic form at a selected pH.

Cationic or Cationically Ionizable Lipids or Lipid-Like Materials

The nucleic acid particles described herein may comprise at least one cationic or cationically ionizable lipid or lipid-like material as particle forming agent. Cationic or cationically ionizable lipids or lipid-like materials contemplated for use herein include any cationic or cationically ionizable lipids or lipid-like materials which are able to electrostatically bind nucleic acid. In one embodiment, cationic or cationically ionizable lipids or lipid-like materials contemplated for use herein can be associated with nucleic acid, e.g. by forming complexes with the nucleic acid or forming vesicles in which the nucleic acid is enclosed or encapsulated.

As used herein, a “cationic lipid” or “cationic lipid-like material” refers to a lipid or lipid-like material having a net positive charge. Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In certain embodiments, a cationic lipid or lipid-like material has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

For purposes of the present disclosure, such “cationically ionizable” lipids or lipid-like materials are comprised by the term “cationic lipid or lipid-like material” unless contradicted by the circumstances.

In one embodiment, the cationic or cationically ionizable lipid or lipid-like material comprises a head group which includes at least one nitrogen atom (N) which is positive charged or capable of being protonated.

Examples of cationic lipids include, but are not limited to 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (βAE-DMRIE), N-(4-carboxybenzyl)-N,N-dimethyl-2,3-bis(oleoyloxy)propan-1-aminium (DOBAQ), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVLS), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-1-yl) 80-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N₁₂-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200).

In some embodiments, the cationic lipid may comprise from about 10 mol % to about 100 mol %, about 20 mol % to about 100 mol %, about 30 mol % to about 100 mol %, about 40 mol % to about 100 mol %, or about 50 mol % to about 100 mol % of the total lipid present in the particle.

Additional Lipids or Lipid-Like Materials

Particles described herein may also comprise lipids or lipid-like materials other than cationic or cationically ionizable lipids or lipid-like materials, i.e., non-cationic lipids or lipid-like materials (including non-cationically ionizable lipids or lipid-like materials). Collectively, anionic and neutral lipids or lipid-like materials are referred to herein as non-cationic lipids or lipid-like materials. Optimizing the formulation of nucleic acid particles by addition of other hydrophobic moieties, such as cholesterol and lipids, in addition to an ionizable/cationic lipid or lipid-like material may enhance particle stability and efficacy of nucleic acid delivery.

An additional lipid or lipid-like material may be incorporated which may or may not affect the overall charge of the nucleic acid particles. In certain embodiments, the additional lipid or lipid-like material is a non-cationic lipid or lipid-like material. The non-cationic lipid may comprise, e.g., one or more anionic lipids and/or neutral lipids. As used herein, an “anionic lipid” refers to any lipid that is negatively charged at a selected pH. As used herein, a “neutral lipid” refers to any of a number of lipid species that exist either in an uncharged or neutral zwitterionic form at a selected pH. In preferred embodiments, the additional lipid comprises one of the following neutral lipid components: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Specific phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), and further phosphatidylethanolamine lipids with different hydrophobic chains.

In certain preferred embodiments, the additional lipid is DSPC or DSPC and cholesterol.

In certain embodiments, the nucleic acid particles include both a cationic lipid and an additional lipid.

In one embodiment, particles described herein include a polymer conjugated lipid such as a pegylated lipid. The term “pegylated lipid” refers to a molecule comprising both a lipid portion and a polyethylene glycol portion. Pegylated lipids are known in the art.

Without wishing to be bound by theory, the amount of the at least one cationic lipid compared to the amount of the at least one additional lipid may affect important nucleic acid particle characteristics, such as charge, particle size, stability, tissue selectivity, and bioactivity of the nucleic acid. Accordingly, in some embodiments, the molar ratio of the at least one cationic lipid to the at least one additional lipid is from about 10:0 to about 1:9, about 4:1 to about 1:2, or about 3:1 to about 1:1.

In some embodiments, the non-cationic lipid, in particular neutral lipid, (e.g., one or more phospholipids and/or cholesterol) may comprise from about 0 mol % to about 90 mol %, from about 0 mol % to about 80 mol %, from about 0 mol % to about 70 mol %, from about 0 mol % to about 60 mol %, or from about 0 mol % to about 50 mol %, of the total lipid present in the particle.

Lipoplex Particles

In certain embodiments of the present disclosure, the RNA described herein may be present in RNA lipoplex particles.

In the context of the present disclosure, the term “RNA lipoplex particle” relates to a particle that contains lipid, in particular cationic lipid, and RNA. Electrostatic interactions between positively charged liposomes and negatively charged RNA results in complexation and spontaneous formation of RNA lipoplex particles. Positively charged liposomes may be generally synthesized using a cationic lipid, such as DOTMA, and additional lipids, such as DOPE. In one embodiment, a RNA lipoplex particle is a nanoparticle.

In certain embodiments, the RNA lipoplex particles include both a cationic lipid and an additional lipid. In an exemplary embodiment, the cationic lipid is DOTMA and the additional lipid is DOPE.

In some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

RNA lipoplex particles described herein have an average diameter that in one embodiment ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In an embodiment, the RNA lipoplex particles have an average diameter that ranges from about 250 nm to about 700 nm. In another embodiment, the RNA lipoplex particles have an average diameter that ranges from about 300 nm to about 500 nm. In an exemplary embodiment, the RNA lipoplex particles have an average diameter of about 400 nm.

The RNA lipoplex particles and compositions comprising RNA lipoplex particles described herein are useful for delivery of RNA to a target tissue after parenteral administration, in particular after intravenous administration. The RNA lipoplex particles may be prepared using liposomes that may be obtained by injecting a solution of the lipids in ethanol into water or a suitable aqueous phase. In one embodiment, the aqueous phase has an acidic pH. In one embodiment, the aqueous phase comprises acetic acid, e.g., in an amount of about 5 mM. Liposomes may be used for preparing RNA lipoplex particles by mixing the liposomes with RNA. In one embodiment, the liposomes and RNA lipoplex particles comprise at least one cationic lipid and at least one additional lipid. In one embodiment, the at least one cationic lipid comprises 1,2-di-O-octadecenyl-3-trimethylammonium propane (DOTMA) and/or 1,2-dioleoyl-3-trimethylammonium-propane (DOTAP). In one embodiment, the at least one additional lipid comprises 1,2-di-(9Z-octadecenoyI)-sn-glycero-3-phosphoethanolamine (DOPE), cholesterol (Chol) and/or 1,2-dioleoyl-sn-glycero-3-phosphocholine (DOPC). In one embodiment, the at least one cationic lipid comprises 1,2-di-O-octadecenyl-3-trimethylammonium propane (DOTMA) and the at least one additional lipid comprises 1,2-di-(9Z-octadecenoyl)-sn-glycero-3-phosphoethanolamine (DOPE). In one embodiment, the liposomes and RNA lipoplex particles comprise 1,2-di-O-octadecenyl-3-trimethylammonium propane (DOTMA) and 1,2-di-(9Z-octadecenoyl)-sn-glycero-3-phosphoethanolamine (DOPE).

Lipid Nanoparticles (LNPs)

In one embodiment, nucleic acid such as RNA described herein is administered in the form of lipid nanoparticles (LNPs). The LNP may comprise any lipid capable of forming a particle to which the one or more nucleic acid molecules are attached, or in which the one or more nucleic acid molecules are encapsulated.

In one embodiment, the LNP comprises one or more cationic lipids, and one or more stabilizing lipids. Stabilizing lipids include neutral lipids and pegylated lipids.

In one embodiment, the LNP comprises a cationic lipid, a neutral lipid, a steroid, a polymer conjugated lipid; and the RNA, encapsulated within or associated with the lipid nanoparticle.

In one embodiment, the LNP comprises from 40 to 55 mol percent, from 40 to 50 mol percent, from 41 to 49 mol percent, from 41 to 48 mol percent, from 42 to 48 mol percent, from 43 to 48 mol percent, from 44 to 48 mol percent, from 45 to 48 mol percent, from 46 to 48 mol percent, from 47 to 48 mol percent, or from 47.2 to 47.8 mol percent of the cationic lipid. In one embodiment, the LNP comprises about 47.0, 47.1, 47.2, 47.3, 47.4, 47.5, 47.6, 47.7, 47.8, 47.9 or 48.0 mol percent of the cationic lipid.

In one embodiment, the neutral lipid is present in a concentration ranging from 5 to 15 mol percent, from 7 to 13 mol percent, or from 9 to 11 mol percent. In one embodiment, the neutral lipid is present in a concentration of about 9.5, 10 or 10.5 mol percent.

In one embodiment, the steroid is present in a concentration ranging from 30 to 50 mol percent, from 35 to 45 mol percent or from 38 to 43 mol percent. In one embodiment, the steroid is present in a concentration of about 40, 41, 42, 43, 44, 45 or 46 mol percent.

In one embodiment, the LNP comprises from 1 to 10 mol percent, from 1 to 5 mol percent, or from 1 to 2.5 mol percent of the polymer conjugated lipid.

In one embodiment, the LNP comprises from 40 to 50 mol percent a cationic lipid; from 5 to 15 mol percent of a neutral lipid; from 35 to 45 mol percent of a steroid; from 1 to 10 mol percent of a polymer conjugated lipid; and the RNA, encapsulated within or associated with the lipid nanoparticle.

In one embodiment, the mol percent is determined based on total mol of lipid present in the lipid nanoparticle.

In one embodiment, the neutral lipid is selected from the group consisting of DSPC, DPPC, DMPC, DOPC, POPC, DOPE, DOPG, DPPG, POPE, DPPE, DMPE, DSPE, and SM. In one embodiment, the neutral lipid is selected from the group consisting of DSPC, DPPC, DMPC, DOPC, POPC, DOPE and SM. In one embodiment, the neutral lipid is DSPC.

In one embodiment, the steroid is cholesterol.

In one embodiment, the polymer conjugated lipid is a pegylated lipid. In one embodiment, the pegylated lipid has the following structure:

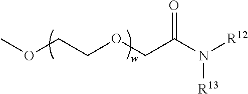

or a pharmaceutically acceptable salt, tautomer or stereoisomer thereof, wherein:
R¹² and R¹³ are each independently a straight or branched, saturated or unsaturated alkyl chain containing from 10 to 30 carbon atoms, wherein the alkyl chain is optionally interrupted by one or more ester bonds; and w has a mean value ranging from 30 to 60. In one embodiment, R¹² and R¹³ are each independently straight, saturated alkyl chains containing from 12 to 16 carbon atoms. In one embodiment, w has a mean value ranging from 40 to 55.

In one embodiment, the average w is about 45. In one embodiment, R¹² and R¹³ are each independently a straight, saturated alkyl chain containing about 14 carbon atoms, and w has a mean value of about 45.

In some embodiments, the cationic lipid component of the LNPs has the structure of Formula (III):

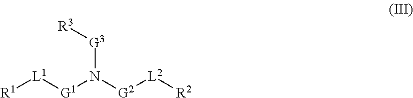

or a pharmaceutically acceptable salt, tautomer, prodrug or stereoisomer thereof, wherein:
one of L¹ or L² is —O(C═O)—, —(C═O)O—, —C(═O)—, —O—, —S(O)ₓ—, —S—S—, C(═O)S—, SC(═O)—, —NR^{a}C(═O)—, —C(═O)NR^{a}—, NR^{a}C(═O)NR^{a}, —OC(═O)NR^{a}— or —NR^{a}C(═O)O—, and the other of L¹ or L² is —O(C═O)—, —(C═O)O—, —C(═O)—, —O—, —S(O)ₓ—, —SS—, —C(═O)S—, SC(═O)—, —NR^{a}C(═O)—, —C(═O)NR^{a}—, NR^{a}C(═O)NR^{a}, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, C(═O)OR⁴, OC(═O)R⁴ or —NR⁵C(═O)R⁴;
R⁴ is C₁-C₁₂ alkyl;
R⁵ is H or C₁-C₆ alkyl; and
x is 0, 1 or 2.

In some of the foregoing embodiments of Formula (III), the lipid has one of the following structures (IIIA) or (IIIB):

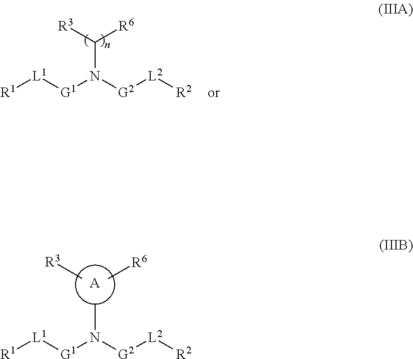

wherein:
A is a 3 to 8-membered cycloalkyl or cycloalkylene ring;
R⁶ is, at each occurrence, independently H, OH or C₁-C₂₄ alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

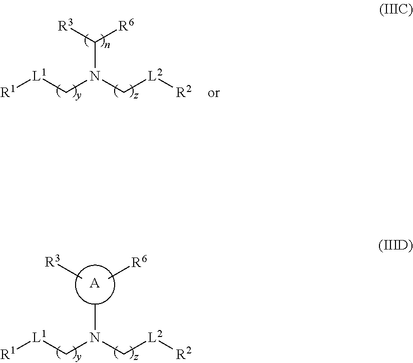

wherein y and z are each independently integers ranging from 1 to 12.

In any of the foregoing embodiments of Formula (III), one of L¹ or L² is O(C═O). For example, in some embodiments each of L¹ and L² are O(C═O). In some different embodiments of any of the foregoing, L¹ and L² are each independently (C═O)O or O(C═O)—. For example, in some embodiments each of L¹ and L² is (C═O)O.

In some different embodiments of Formula (III), the lipid has one of the following structures (IIIE) or (IIIF):

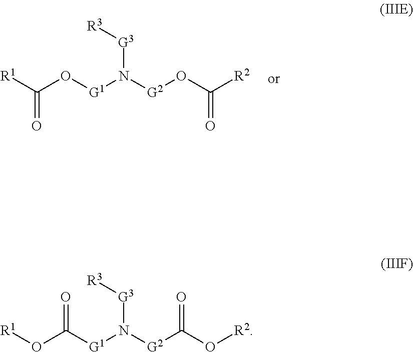

In some of the foregoing embodiments of Formula (III), the lipid has one of the following structures (IIIG), (IIIH), (IIII), or (IIIJ):

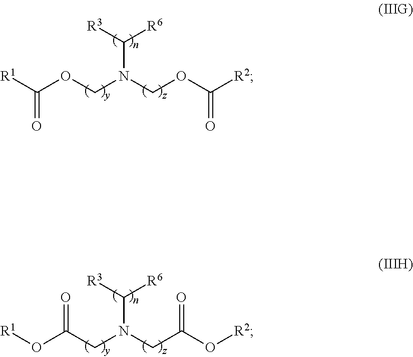

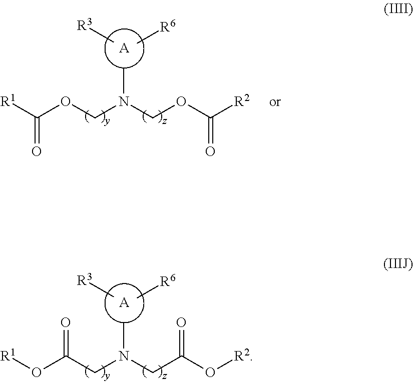

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G³ is unsubstituted. In other embodiments, G3 is substituted. In various different embodiments, G³ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments, R¹ and R² each, independently have the following structure:

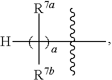

wherein:
R^{7a} and R^{7b} are, at each occurrence, independently H or C₁-C₁₂ alkyl; and
a is an integer from 2 to 12,
wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

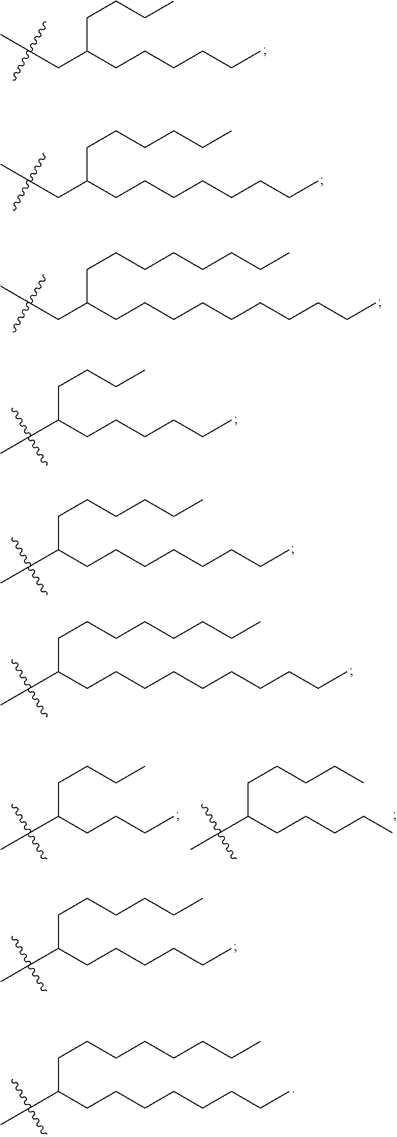

In some of the foregoing embodiments of Formula (III), R³ is OH, CN, C(═O)O R⁴, OC(═O) R⁴ or —NHC(═O) R⁴. In some embodiments, R⁴ is methyl or ethyl.

In various different embodiments, the cationic lipid of Formula (III) has one of the structures set forth in the table below.

Representative Compounds of Formula (III).

| No. | Structure |
|---|---|
| III-1 | 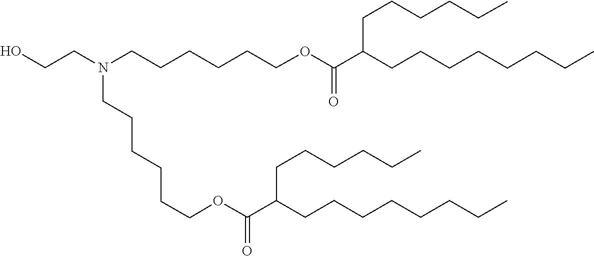 [SMILES: CCCCCCCCC(CCCCCC)C(=O)OCCCCCCN(CCO)CCCCCCOC(=O)C(CCCCCC)CCCCCCCC] |
| III-2 | 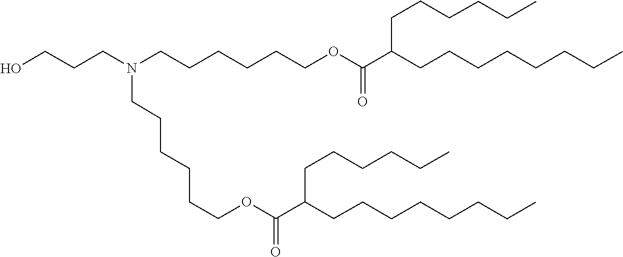 [SMILES: CCCCCCCCC(CCCCCC)C(=O)OCCCCCCN(CCCO)CCCCCCOC(=O)C(CCCCCC)CCCCCCCC] |
| III-3 | 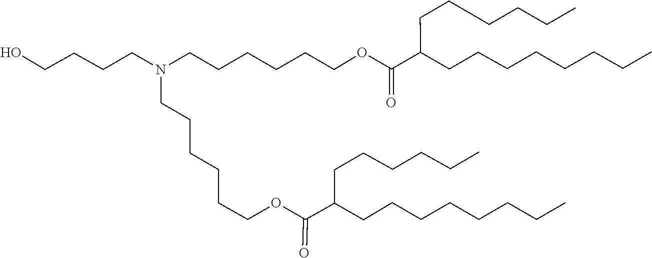 [SMILES: CCCCCCCCC(CCCCCC)C(=O)OCCCCCCN(CCCCO)CCCCCCOC(=O)C(CCCCCCCC)CCCCCCCC] |
| III-4 | 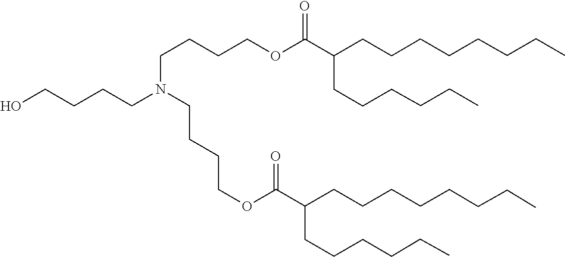 [SMILES: CCCCCCCCC(CCCCCC)C(=O)OCCCCN(CCCCO)CCCCOC(=O)C(CCCCCC)CCCCCCCC] |
| III-5 | 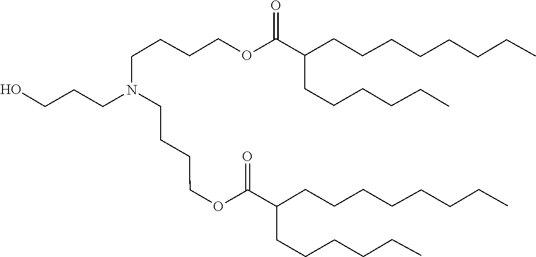 [SMILES: CCCCCCCCC(CCCCCC)C(=O)OCCCCN(CCCO)CCCCOC(=O)C(CCCCCC)CCCCCCCC] |
| III-6 | 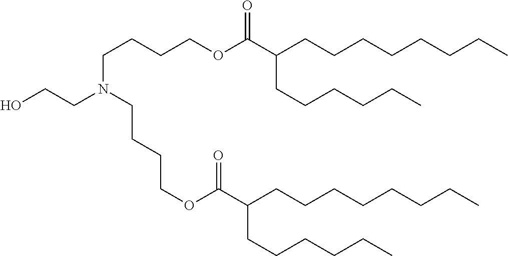 [SMILES: CCCCCCCCC(CCCCCC)C(=O)OCCCCN(CCO)CCCCOC(=O)C(CCCCCC)CCCCCCCC] |
| III-7 | 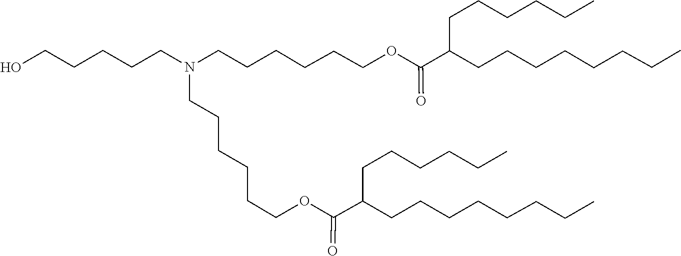 [SMILES: CCCCCCCCC(CCCCCC)C(=O)OCCCCCCN(CCCCCO)CCCCCCOC(=O)C(CCCCCC)CCCCCCCC] |
| III-8 | 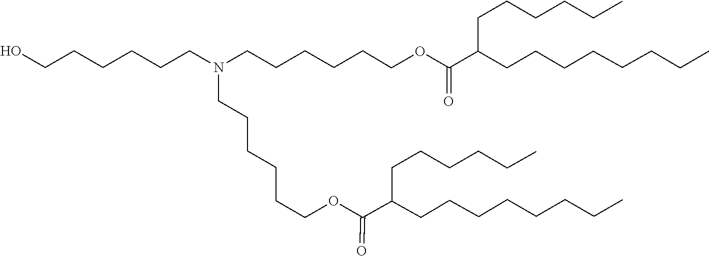 [SMILES: CCCCCCCCC(CCCCCC)C(=O)OCCCCCCN(CCCCCCO)CCCCCCOC(=O)C(CCCCCC)CCCCCCCC] |
| III-9 | 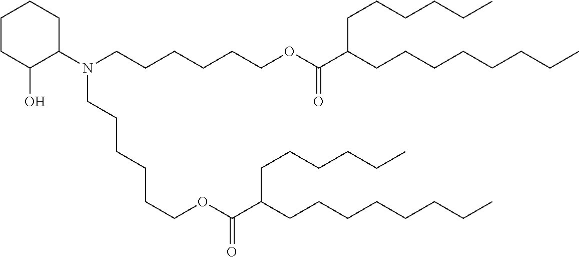 [SMILES: CCCCCCCCC(CCCCCC)C(=O)OCCCCCCN(CCCCCCOC(=O)C(CCCCCC)CCCCCCCC)C1CCCCC1O] |
| III-10 | 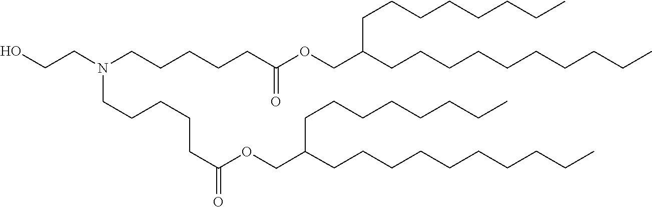 [SMILES: CCCCCCCCCCC(CCCCCCCC)COC(=O)CCCCCN(CCO)CCCCCC(=O)OCC(CCCCCCCC)CCCCCCCCCC] |
| III-11 | 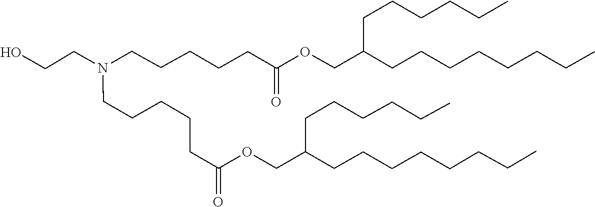 [SMILES: CCCCCCCCC(CCCCCC)COC(=O)CCCCCN(CCO)CCCCCC(=O)OCC(CCCCCC)CCCCCCCC] |
| III-12 | 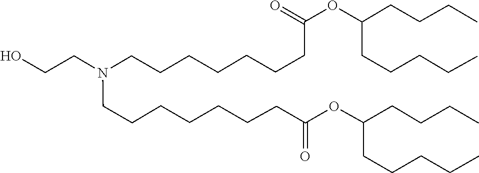 [SMILES: CCCCCC(CCCC)OC(=O)CCCCCCCN(CCO)CCCCCCCC(=O)OC(CCCC)CCCCC] |
| III-13 | 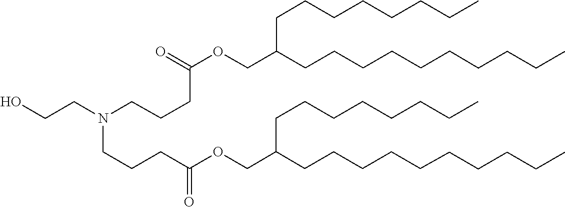 [SMILES: CCCCCCCCCCC(CCCCCCCC)COC(=O)CCCN(CCO)CCCC(=O)OCC(CCCCCCCC)CCCCCCCCCC] |
| III-14 | 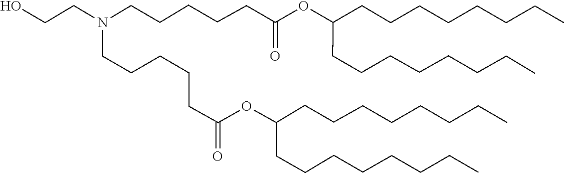 [SMILES: CCCCCCCCC(CCCCCCCC)OC(=O)CCCCCN(CCO)CCCCCC(=O)OC(CCCCCCCC)CCCCCCCC] |
| III-15 | 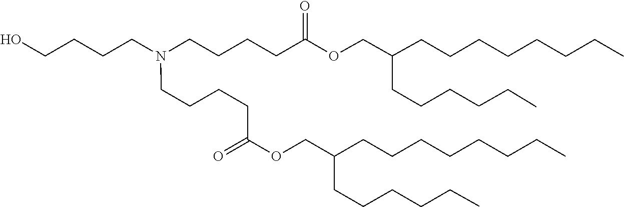 [SMILES: CCCCCCCCC(CCCCCC)COC(=O)CCCCN(CCCCO)CCCCC(=O)OCC(CCCCCC)CCCCCCCC] |
| III-16 | 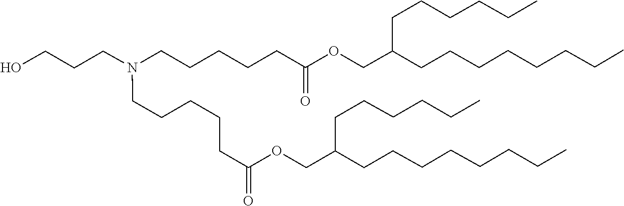 [SMILES: CCCCCCCCC(CCCCCC)COC(=O)CCCCCN(CCCO)CCCCCC(=O)OCC(CCCCCC)CCCCCCCC] |
| III-17 | 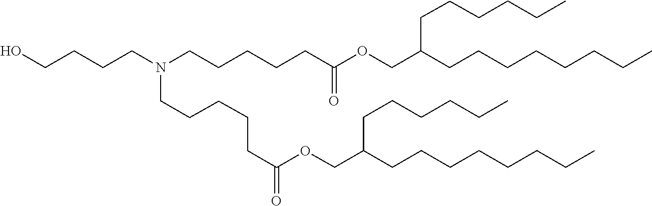 [SMILES: CCCCCCCCC(CCCCCC)COC(=O)CCCCCN(CCCCO)CCCCCC(=O)OCC(CCCCCC)CCCCCCCC] |
| III-18 | 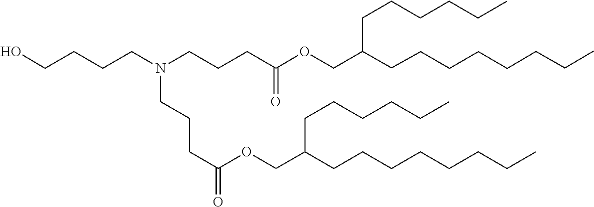 [SMILES: CCCCCCCCC(CCCCCC)COC(=O)CCCN(CCCCO)CCCC(=O)OCC(CCCCCC)CCCCCCCC] |
| III-19 | 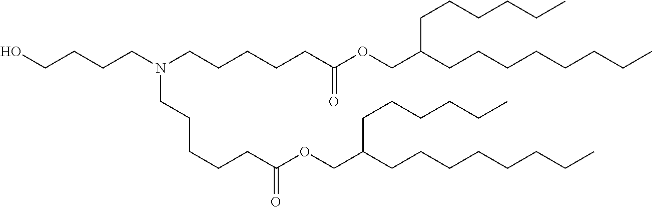 [SMILES: CCCCCCCCC(CCCCCC)COC(=O)CCCCCN(CCCCO)CCCCCC(=O)OCC(CCCCCC)CCCCCCCC] |
| III-20 | 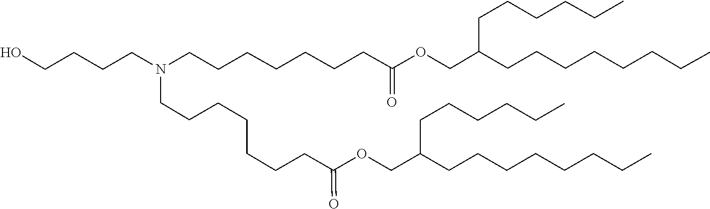 [SMILES: CCCCCCCCC(CCCCCC)COC(=O)CCCCCCCN(CCCCO)CCCCCCCC(=O)OCC(CCCCCC)CCCCCCCC] |
| III-21 | 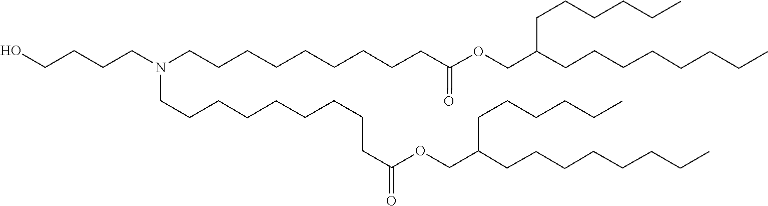 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 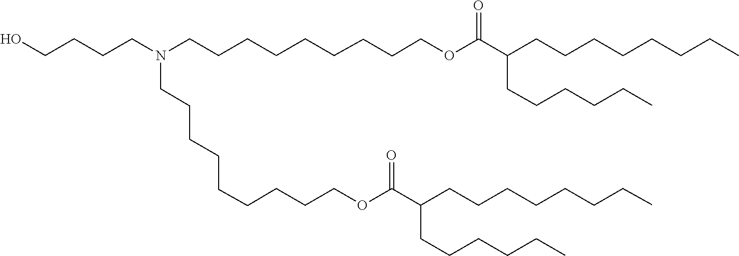 [SMILES: CCCCCCCCC(CCCCCC)C(=O)OCCCCCCCCCN(CCCCO)CCCCCCCCCOC(=O)C(CCCCCC)CCCCCCCC] |
| III-23 | 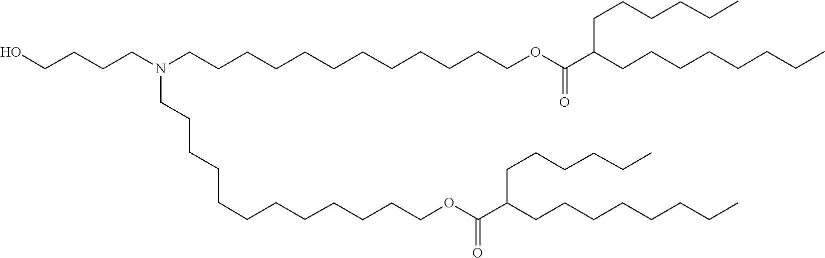 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 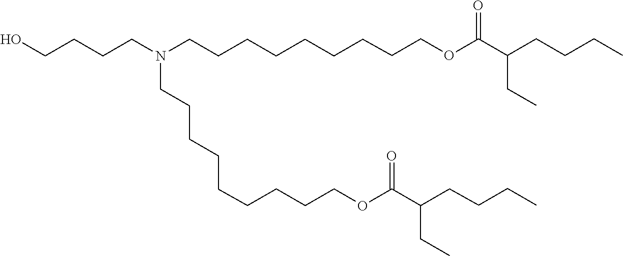 [SMILES: CCCCC(CC)C(=O)OCCCCCCCCCN(CCCCO)CCCCCCCCCOC(=O)C(CC)CCCC] |
| III-25 | 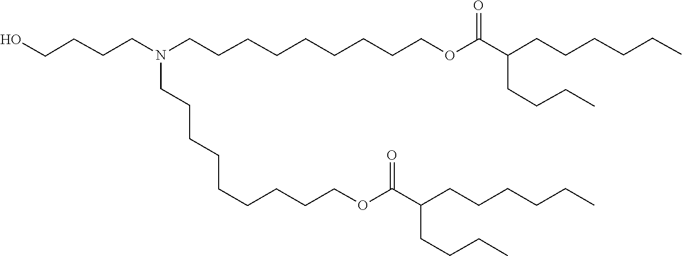 [SMILES: CCCCCCC(CCCC)C(=O)OCCCCCCCCCN(CCCCO)CCCCCCCCCOC(=O)C(CCCC)CCCCCC] |
| III-26 | 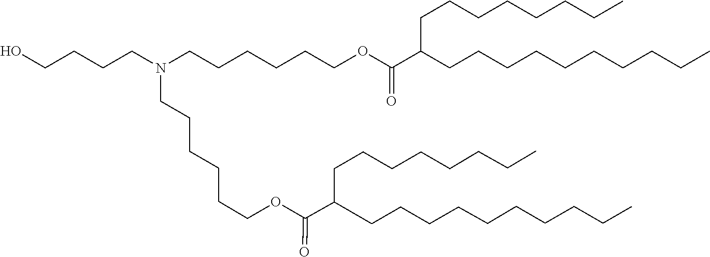 [SMILES: CCCCCCCCCCCC(CCCCCCCC)C(=O)OCCCCCCN(CCCCO)CCCCCCOC(=O)C(CCCCCCCC)CCCCCCCCCC] |
| III-27 | 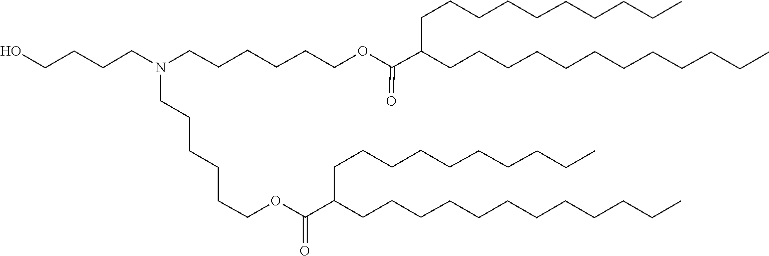 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 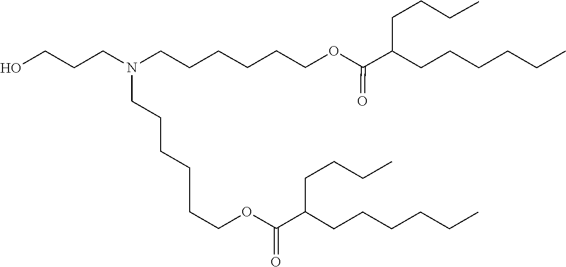 [SMILES: CCCCCCC(CCCC)C(=O)OCCCCCCCN(CCCO)CCCCCCOC(=O)C(CCCC)CCCCCC] |
| III-29 | 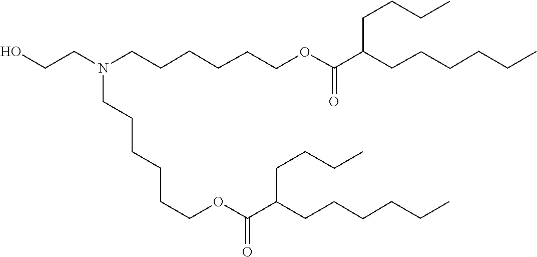 [SMILES: CCCCCCC(CCCC)C(=O)OCCCCCCN(CCO)CCCCCCOC(=O)C(CCCC)CCCCCC] |
| III-30 | 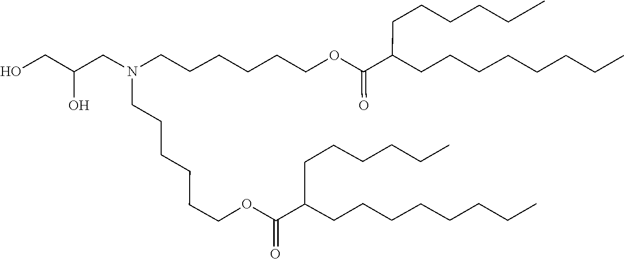 [SMILES: CCCCCCCCC(CCCCCCC)C(=O)OCCCCCCN(CCCCCCOC(=O)C(CCCCCCC)CCCCCCCC)CC(O)CO] |
| III-31 | 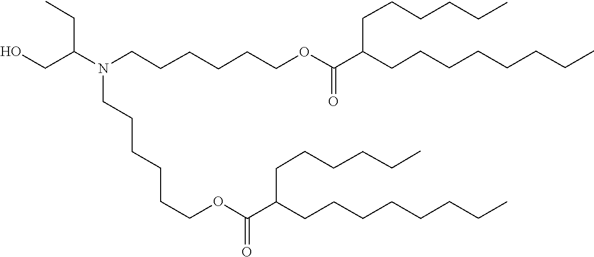 [SMILES: CCCCCCCCC(CCCCCC)C(=O)OCCCCCCN(CCCCCCOC(=O)C(CCCCCC)CCCCCCCC)C(CC)CO] |
| III-32 | 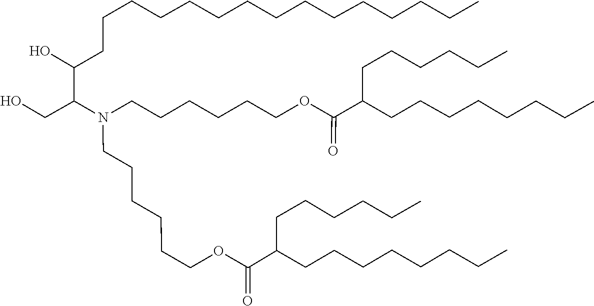 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 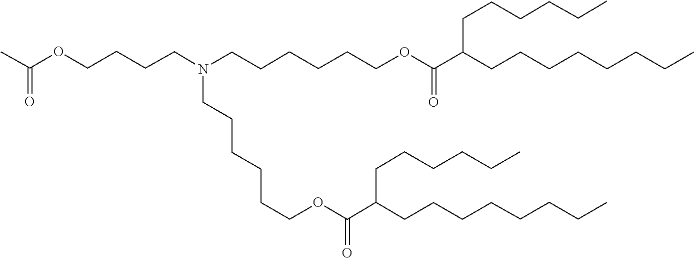 [SMILES: CCCCCCCCC(CCCCCC)C(=O)OCCCCCCN(CCCCCCOC(=O)C(CCCCCC)CCCCCCCC)CCCCOC(C)=O] |
| III-34 | 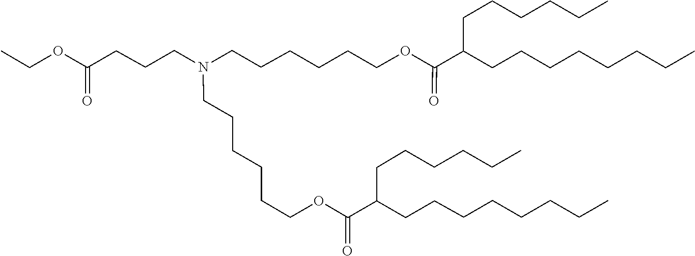 [SMILES: CCCCCCCCC(CCCCCC)C(=O)OCCCCCCN(CCCCCCOC(=O)C(CCCCCC)CCCCCCCC)CCCC(=O)OCC] |
| III-35 | 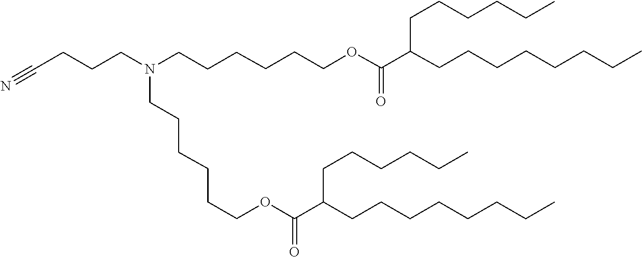 [SMILES: CCCCCCCCC(CCCCCC)C(=O)OCCCCCCN(CCCC#N)CCCCCCOC(=O)C(CCCCCC)CCCCCCCC] |
| III-36 | 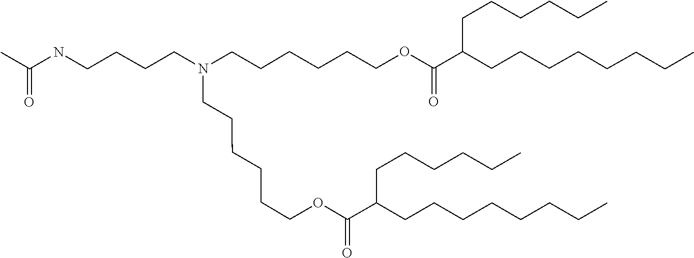 |

In some embodiments, the LNP comprises a lipid of Formula (III), RNA, a neutral lipid, a steroid and a pegylated lipid. In some embodiments, the lipid of Formula (III) is compound III-3. In some embodiments, the neutral lipid is DSPC. In some embodiments, the steroid is cholesterol. In some embodiments, the pegylated lipid is ALC-0159.

ALC-0159:

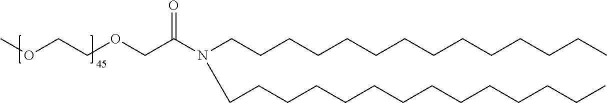

In some embodiments, the cationic lipid is present in the LNP in an amount from about 40 to about 50 mole percent. In one embodiment, the neutral lipid is present in the LNP in an amount from about 5 to about 15 mole percent. In one embodiment, the steroid is present in the LNP in an amount from about 35 to about 45 mole percent. In one embodiment, the pegylated lipid is present in the LNP in an amount from about 1 to about 10 mole percent. In some embodiments, the LNP comprises compound III-3 in an amount from about 40 to about 50 mole percent, DSPC in an amount from about 5 to about 15 mole percent, cholesterol in an amount from about 35 to about 45 mole percent, and ALC-0159 in an amount from about 1 to about 10 mole percent.

In some embodiments, the LNP comprises compound III-3 in an amount of about 47.5 mole percent, DSPC in an amount of about 10 mole percent, cholesterol in an amount of about 40.7 mole percent, and ALC-0159 in an amount of about 1.8 mole percent.

The N/P value is preferably at least about 4. In some embodiments, the N/P value ranges from 4 to 20, 4 to 12, 4 to 10, 4 to 8, or 5 to 7. In one embodiment, the N/P value is about 6.

Pharmaceutical Compositions

The agents described herein may be administered in pharmaceutical compositions or medicaments and may be administered in the form of any suitable pharmaceutical composition.

In one embodiment, the pharmaceutical composition described herein is a composition against coronavirus in a subject.

In one embodiment of all aspects of the invention, the components described herein such as RNA encoding a binding agent may be administered in a pharmaceutical composition which may comprise a pharmaceutically acceptable carrier and may optionally comprise one or more adjuvants, stabilizers etc. In one embodiment, the pharmaceutical composition is for therapeutic or prophylactic treatments, e.g., for use in treating or preventing a coronavirus infection.

The term “pharmaceutical composition” relates to a formulation comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. A pharmaceutical composition is also known in the art as a pharmaceutical formulation.

The pharmaceutical compositions according to the present disclosure are generally applied in a “pharmaceutically effective amount” and in “a pharmaceutically acceptable preparation”. The term “pharmaceutically acceptable” refers to the non-toxicity of a material which does not interact with the action of the active component of the pharmaceutical composition.

The term “pharmaceutically effective amount” or “therapeutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the compositions described herein may depend on various of such parameters. In the case that a reaction in a patient is insufficient with an initial dose, higher doses (or effectively higher doses achieved by a different, more localized route of administration) may be used.

The pharmaceutical compositions of the present disclosure may contain salts, buffers, preservatives, and optionally other therapeutic agents. In one embodiment, the pharmaceutical compositions of the present disclosure comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carriers include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In one embodiment, the pharmaceutical composition of the present disclosure includes isotonic saline.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

In one embodiment, pharmaceutical compositions described herein may be administered intravenously, intraarterially, subcutaneously, intradermally or intramuscularly. In certain embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In a preferred embodiment, the pharmaceutical composition is formulated for intramuscular administration.

In another embodiment, the pharmaceutical composition is formulated for systemic administration, e.g., for intravenous administration.

The term “co-administering” as used herein means a process whereby different compounds or compositions are administered to the same patient. The different compounds or compositions may be administered simultaneously, at essentially the same time, or sequentially.

Treatments

The present invention provides methods and agents for blocking coronavirus S protein binding to ACE2, in particular for neutralizing coronavirus S protein binding to ACE2 in a subject. Methods described herein may comprise administering an effective amount of a composition comprising RNA encoding a binding agent described herein.

In one embodiment, the methods and agents described herein provide a neutralizing effect in a subject to coronavirus, coronavirus infection, or to a disease or disorder associated with coronavirus. The present invention thus provides methods and agents for treating or preventing the infection, disease, or disorder associated with coronavirus.

As used herein, the term “neutralization” refers to an event in which a binding agent binds to a biological activity site of a virus such as a receptor binding protein, thereby inhibiting the viral infection of cells. The term “neutralization” refers, in particular, to a binding agent that can eliminate or significantly reduce virulence (e.g. ability of infecting cells) of viruses of interest.

In one embodiment, the methods and agents described herein are administered to a subject having an infection, disease, or disorder associated with coronavirus. In one embodiment, the methods and agents described herein are administered to a subject at risk for developing the infection, disease, or disorder associated with coronavirus. For example, the methods and agents described herein may be administered to a subject who is at risk for being in contact with coronavirus. In one embodiment, the methods and agents described herein are administered to a subject who lives in, traveled to, or is expected to travel to a geographic region in which coronavirus is prevalent. In one embodiment, the methods and agents described herein are administered to a subject who is in contact with or expected to be in contact with another person who lives in, traveled to, or is expected to travel to a geographic region in which coronavirus is prevalent. In one embodiment, the methods and agents described herein are administered to a subject who has knowingly been exposed to coronavirus through their occupation, or other contact. In one embodiment, a coronavirus is SARS-CoV-1 or SARS-CoV-2. In one embodiment, a coronavirus is SARS-CoV-2.

The therapeutic compounds or compositions of the invention may be administered prophylactically (i.e., to prevent a disease or disorder) or therapeutically (i.e., to treat a disease or disorder) to subjects suffering from, or at risk of (or susceptible to) developing a disease or disorder. Such subjects may be identified using standard clinical methods. In the context of the present invention, prophylactic administration occurs prior to the manifestation of overt clinical symptoms of disease, such that a disease or disorder is prevented or alternatively delayed in its progression. In the context of the field of medicine, the term “prevent” encompasses any activity, which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

In some embodiments, administration of an agent or composition of the present invention may be performed by single administration or boosted by multiple administrations.

The term “disease” refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

In the present context, the term “treatment”, “treating” or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g. mouse, rat, rabbit, dog, cat, cattle, swine, sheep, horse or primate) that can be afflicted with or is susceptible to a disease or disorder but may or may not have the disease or disorder. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

In one embodiment of the disclosure, the aim is to prevent or treat coronavirus infection.

The term “infectious disease” refers to any disease which can be transmitted from individual to individual or from organism to organism, and is caused by a microbial agent (e.g. common cold). Infectious diseases are known in the art and include, for example, a viral disease, a bacterial disease, or a parasitic disease, which diseases are caused by a virus, a bacterium, and a parasite, respectively. In this regard, the infectious disease can be, for example, hepatitis, sexually transmitted diseases (e.g. chlamydia or gonorrhea), tuberculosis, HIV/acquired immune deficiency syndrome (AIDS), diphtheria, hepatitis B, hepatitis C, cholera, severe acute respiratory syndrome (SARS), the bird flu, and influenza.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Example 1: Generation of SARS-CoV-2 and SARS-CoV S1 Protein Targeting Molecules

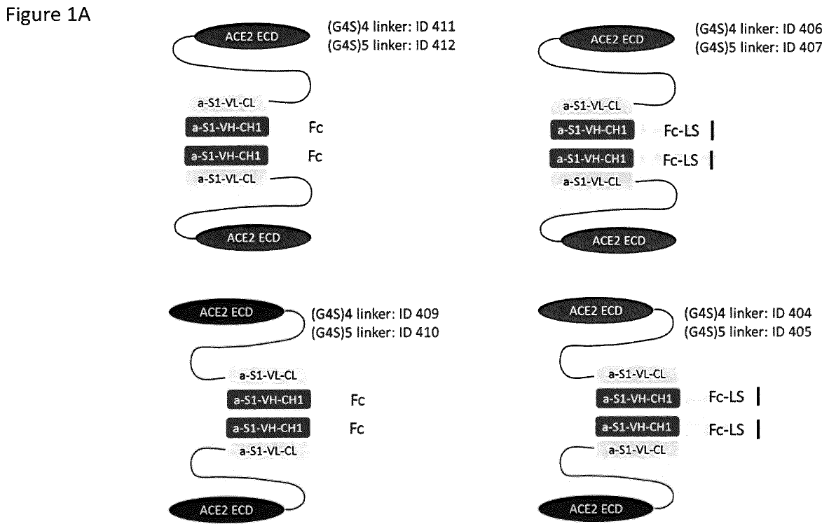

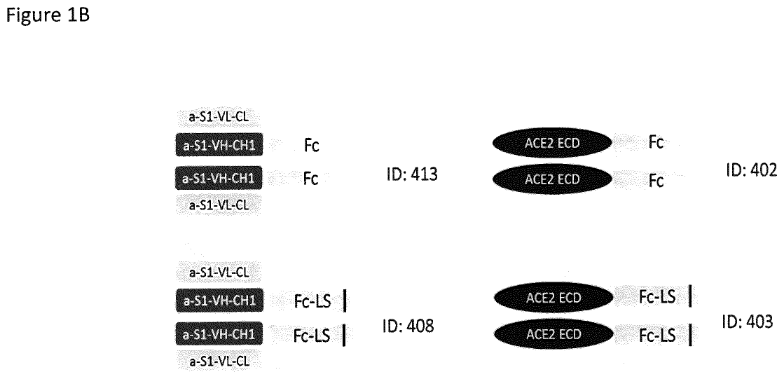

To generate molecules with potent binding and SARS-CoV and SARS-CoV-2 virus neutralizing activities, the light chain of a previously described anti-SARS-CoV S1 protein specific antibody (anti-S1 antibody; ter Meulen et al, 2006) was fused N- or C-terminally to an ACE2 extracellular domain (aa 18-615). Mutations R273Q, H345L, H374N, H378N were introduced into the ACE2 extracellular domain to avoid enzymatic activity and substrate binding (Guy et al, 2005). In the anti-S1-antibody heavy chain of some constructions an LS (M428L/N434S) mutation was introduced which is described to result in a longer half-life in vivo (Zalevsky et al 2009). Plasmid DNAs encoding these protein constructs were transfected into HEK-293 FreeStyle™ cells and proteins purified from the culture supernatants by protein-A affinity and subsequent size exclusion chromatography. FIG. 1 provides an overview on the generated constructs.

### Example 2: Binding of Anti-S1-Antibody-ACE2 Fusion Proteins to Recombinant SARS-CoV2 S1-RBD Protein

The binding potencies of anti-S1-antibody-ACE2 fusion proteins to SARS-CoV2 S1-RBD protein were determined in an ELISA.

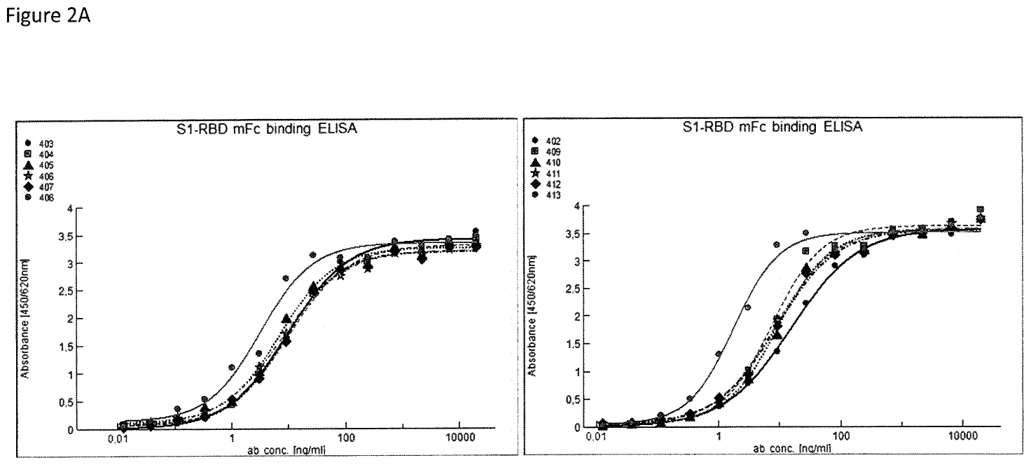

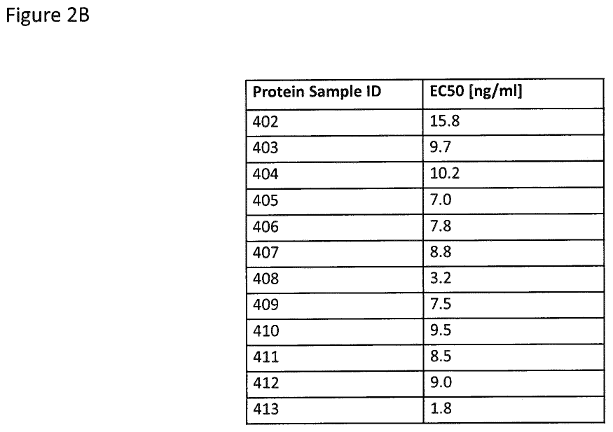

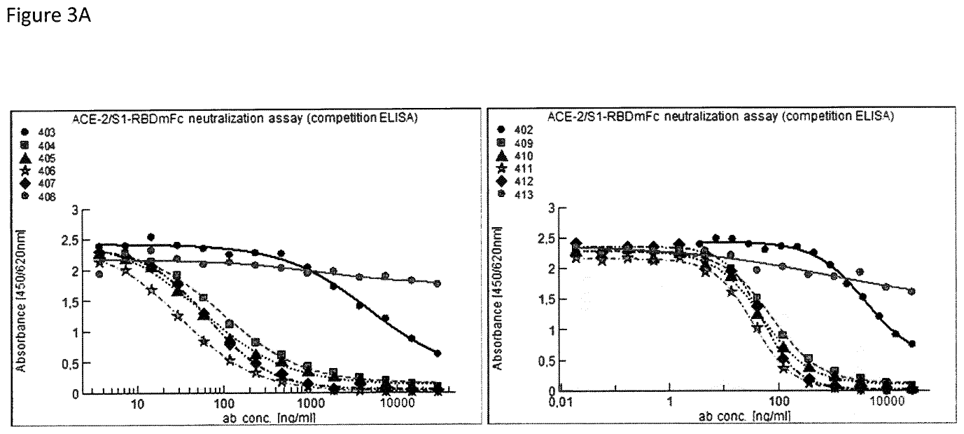

Mouse-Fc-tagged SARS-CoV2 S1-RBD (Sino Biologicals) recombinant protein was coated on 384-well Nunc MaxiSorp™ flat bottom plates at a concentration of 2.5 μg/ml in PBS for 60 minutes at room temperature. After 3 washes with PBS 0.1% Tween (wash buffer), blocking with PBS, 2% BSA, 0.05% Tween for 60 minutes at room temperature and another 3 washes, anti-S1-antibody-ACE2 fusion proteins were added in PBS, 0.5% BSA, 0.05% Tween (ELISA buffer) in concentrations ranging from 20,000 to 0.013 ng/ml and the plate was incubated for 60 minutes at room temperature. As a control, recombinant ACE-2 extracellular domain with human-Fc tag was used. After 3 washes with wash buffer, the horseradish peroxidase coupled detection anti-human IgG, Fcγ fragment specific F(ab′)₂ fragment (Jackson Immuno Research) was added in ELISA buffer at a dilution of 1:2,500. The plate was incubated for 60 minutes at room temperature, washed 6 times with wash buffer before TMB solution (Thermo Fisher Scientific) was added. After 6 minutes HCl was added and the absorbance at wavelengths of 450 and 620 nm recorded using a Tecan Infinite M1000 reader. Fitting curves and EC50 calculation were obtained by using Excel (Microsoft) and XLfit (IDBS). FIG. 2 shows that the the anti-S1-antibody-ACE2 fusion proteins bind to the SARS-CoV2 S1-RBD protein with EC50 values ranging from 7 to 10.2 ng/ml.

### Example 3: Neutralization of SARS-CoV2-S1-RBD Binding to ACE2 by Anti-S1-Antibody-ACE2 Fusion Proteins

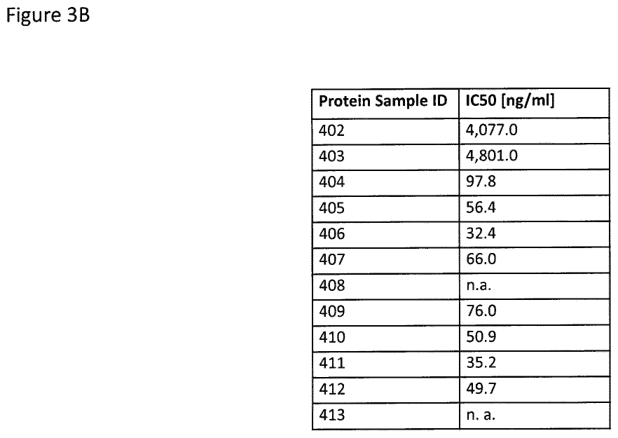

The potency of anti-S1-antibody-ACE2 fusion proteins in neutralizing the SARS-CoV2 S1-RBD binding to the ACE-2 extracellular domain was investigated in a competition ELISA. His-tagged human ACE-2 extracellular domain (Sino Biologicals) recombinant protein was coated on a 384-well Nunc MaxiSorp™ flat bottom plate at a concentration of 2.5 μg/ml in PBS for 60 minutes at room temperature. After 3 washes with PBS 0.1% Tween (wash buffer), the MaxiSorp™ M plate was blocked with PBS, 2% BSA, 0.05% Tween for 60 minutes at room temperature. In a separate polypropylene 384-well plate (Corning) anti-S1-antibody-ACE2 proteins diluted in PBS, 0.5% BSA, 0.05% Tween (ELISA buffer) in concentrations ranging from 30,000 to 0.01 ng/ml were pre-incubated with 30 ng/ml mouse-Fc-tagged SARS-CoV2 S1-RBD (Sino Biologicals) recombinant protein for 60 minutes at room temperature. As a control, recombinant ACE-2 extracellular domain with human-Fc tag was used. After 3 washes with wash buffer, the pre-incubation mix from the Corning plate was transferred onto the MaxiSorp™ plate and incubated for 60 minutes at room temperature. After 3 washes with wash buffer, the horseradish peroxidase coupled detection anti-mouse IgG F(ab′)₂ fragment (Cytiva) was added in ELISA buffer at a dilution of 1:1,000. The plate was incubated for 60 minutes at room temperature, washed 6 times with wash buffer before TMB solution (Thermo Fisher Scientific) was added. After 15 minutes HCl was added and the absorbance at wavelengths of 450 and 620 nm recorded using a Tecan Infinite M1000 reader. Fitting curves and IC50 calculation were obtained by using Excel (Microsoft) and XLfit (IDBS). The data in FIG. 3 demonstrates that the anti-S1-antibody (408, 413) does not block the interaction of the S1-RBD with the ACE2 extracellular domain significantly at the tested concentrations, whereas the hFc-tagged ACE2 extracellular domain (402, 403) blocks the interaction with an IC50 value >4 μg/ml. In contrast, the anti-S1-antibody-ACE2 fusion proteins inhibit this interaction with IC50 values ranging from 32.4 to 97.8 ng/ml and thus with about >40 times increased potency over ACE2-hFc in this assay.

### Example 4: Pseudovirus Neutralization Activity by Anti-S1-Antibody-ACE2 Fusion Proteins

To determine the virus neutralizing activity of anti-S1-antibody-ACE2 proteins a pseudovirus neutralization test (pVNT) was performed. Replication-deficient vesicular stomatitis virus (VSV) that lacks the genetic information for the VSV envelope glycoprotein VSV-G but contains an open-reading frame (ORF) for green fluorescent protein (GFP) was used for SARS-CoV2-S pseudovirus generation. VSV pseudotypes were generated according to a published protocol (PMID: 32142651).

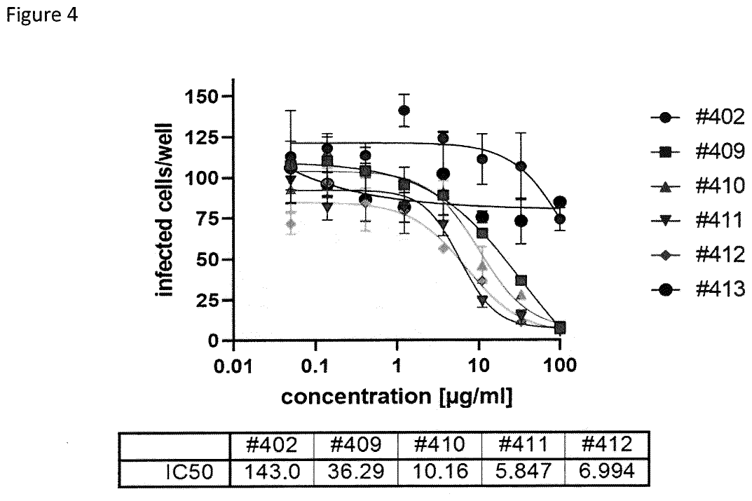

For the pVNT assay, Vero-76 cells were thawed and diluted to 2.67×10⁵ cells/mL in assay medium (DMEM/10% FBS) and seeded in 96-well flat-bottom plates at 4×10⁴ cells per well. Cells were incubated for 4 to 6 hours at 37° C. and 7.5% CO2. VSV/SARS CoV2 pseudovirus was thawed and diluted to obtain 4.8×10³ infectious units [IU]/mL. 30 μL of diluted pseudovirus was added to the wells containing 30 μl anti-S1-antibody-ACE2 fusion proteins, anti-S1-antibody or ACE-2-hFc for final concentrations ranging from 200 to 0.092 μg/ml. Pseudovirus/test protein mix was incubated for 10 min at RT on a microplate shaker at 400 rpm. Pseudovirus/test protein dilution mix was then added to the seeded Vero-76 cells (MOI:0.003), followed by incubation for 16 to 24 hours at 37° C. and 5% CO₂. Each dilution of serum samples was tested in duplicate wells. After the incubation, the cell culture plates were removed from the incubator, placed in an IncuCyte Live Cell Analysis system and incubated for 30 min prior to the analysis. Whole well scanning for brightfield and GFP fluorescence was performed using a 4× objective. Curve fitting and IC50 calculation was done using GraphPad Prism software. FIG. 4 demonstrates that the anti-S1-antibody (413) and the ACE2-hFC (402) do not significantly affect infection of Vero-76 cells by the pseudovirus at the tested concentrations whereas anti-S1-antibody-ACE2 fusion proteins inhibit infection in a dose dependent manner with IC50 values ranging from 5.847 to 36.29 μg/ml.

### Example 5: Binding Affinities of Anti-S1-Antibody-ACE2 Fusion Proteins

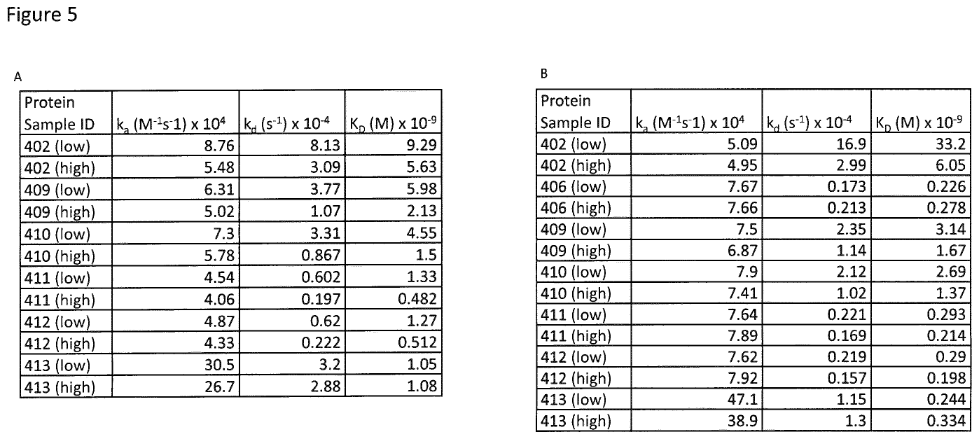

The biochemical affinities of ACE-2-hFc (402), anti-S1-antibody (413) and the anti-S1-mAB-ACE2 fusion proteins (406, 409, 410, 411, 412) to the SARS-CoV-2 S1 Protein were determined by surface plasmon resonance measurements. SARS-CoV-2 S1 protein (HIS tag, active trimer; Acro Biosystems #SPN-C52H8) was immobilized to a CM5 sensor chip surface via an anti-HIS-tag antibody at two different densities (Rmax ^{˜}100 RU and Rmax ^{˜}620 RU). In another experimental series SARS-CoV-2 S1-RBD Protein coupled to a mouse Fc-tag (Sino Biologicals #40592-V05H) was immobilized to a CM5 sensor chip surface via an anti-mouse-Fc antibody at two different densities (Rmax ^{˜}20 RU and Rmax ^{˜}250 RU). The kinetics of the interaction of immobilized SARS-CoV-2 S1 Protein (active trimer) or SARS-CoV-2 S1-RBD Protein with soluble anti-S1-antibody-ACE2 fusion proteins were analysed on a Biacore T200 SPR instrument. Kinetic data were determined using a Langmuir 1:1 binding model. FIGS. 5A and B show that the fusion proteins 406, 411 and 412 have slower on-rates but also slower off-rates in binding to both the SARS-CoV-2 S1 Protein (active trimer) or SARS-CoV-2 S1-RBD Protein compared to the anti-S1 antibody (413). Compared to ACE-2-Fc (402), the off-rates of the fusion proteins are also significantly slower. Therefore, once fusion proteins 406, 411 and 412 have bound to the S1 protein, they remain bound to the S1 protein for a longer time compared to the anti-S1 antibody or the ACE-2-Fc alone and may block the interaction between virus-expressed S1 protein and cell-expressed ACE-2 receptor more persistently than a soluble ACE-2-Fc protein.

### Example 6: Generation of SARS-CoV-2 S1-RBD Binding, Neutralizing Antibodies

To obtain novel, anti-S1 antibodies, New Zealand White rabbits were immunized with either recombinant SARS-CoV-2 S1-RBD-mFc protein or S1-RBD encoding mRNA. Single B-cells were isolated by FACS and cultivated to obtain monoclonal antibodies in the medium supernatant. After 7 days of cultivation, B-cell supernatant were separated from the B-cells to perform binding and functional assays. B-cells were lysed in RNA extraction RLT buffer for RNA extraction, RT-PCR and Sanger sequencing of the antibody heavy and light chain variable regions.

### Example 7: Binding of Antibodies in B-Cell Supernatants to SARS-CoV2-S1 Protein

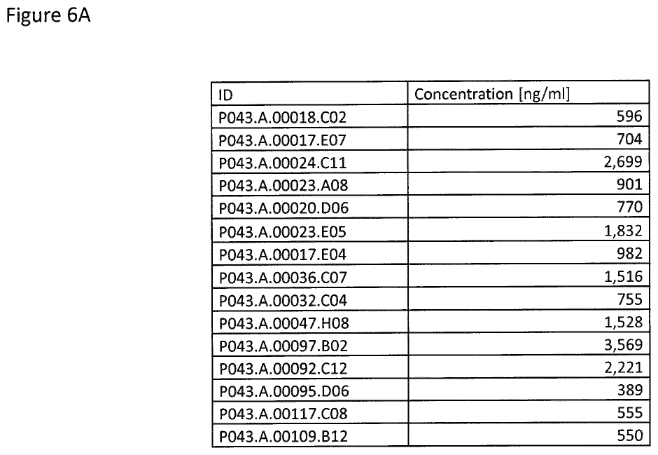

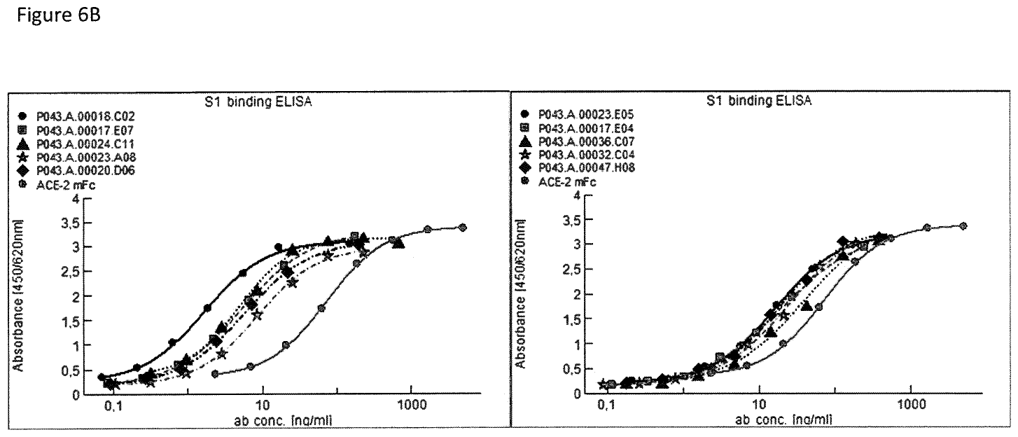

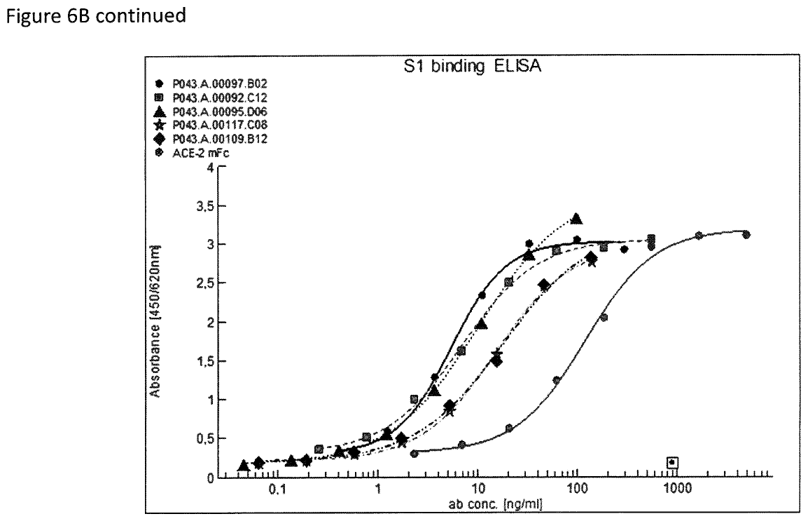

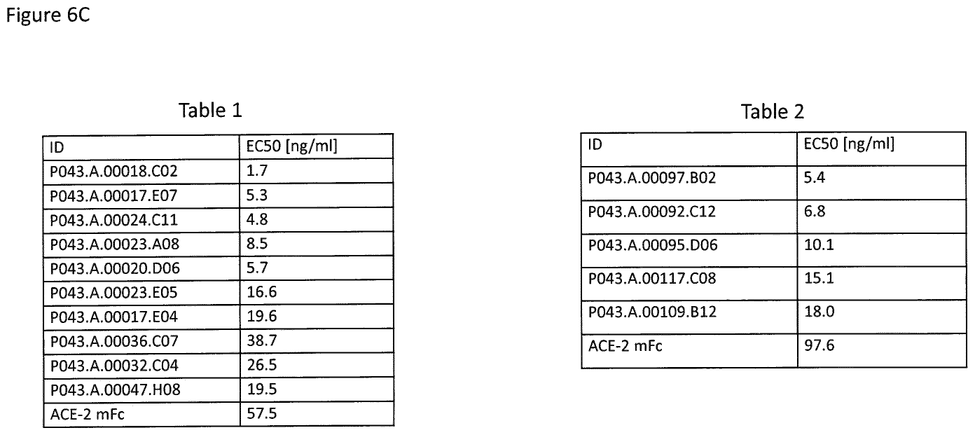

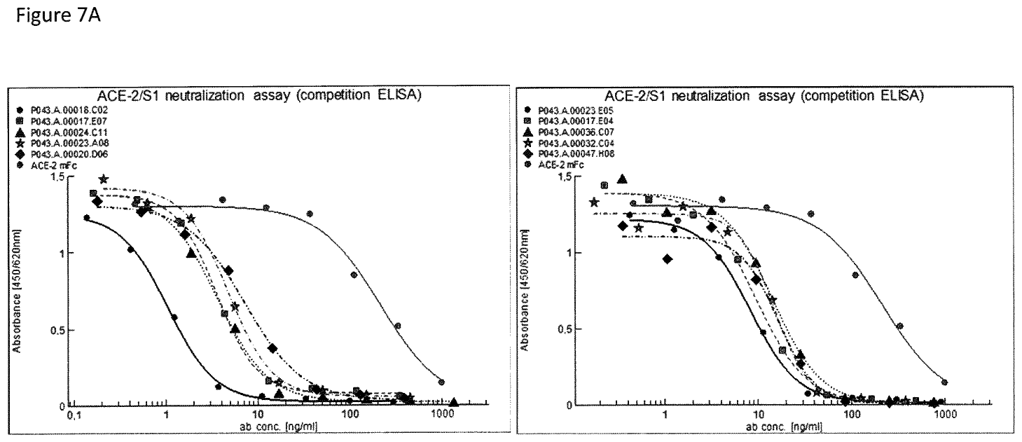

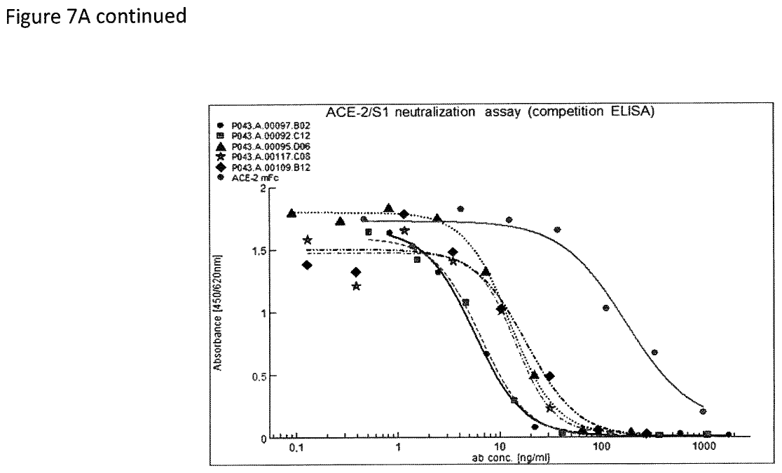

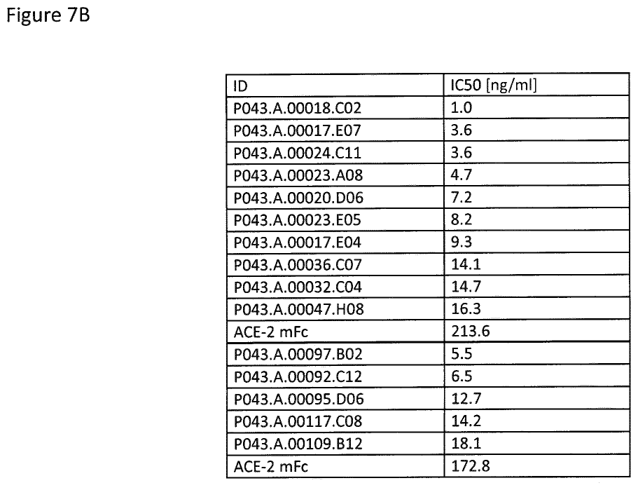

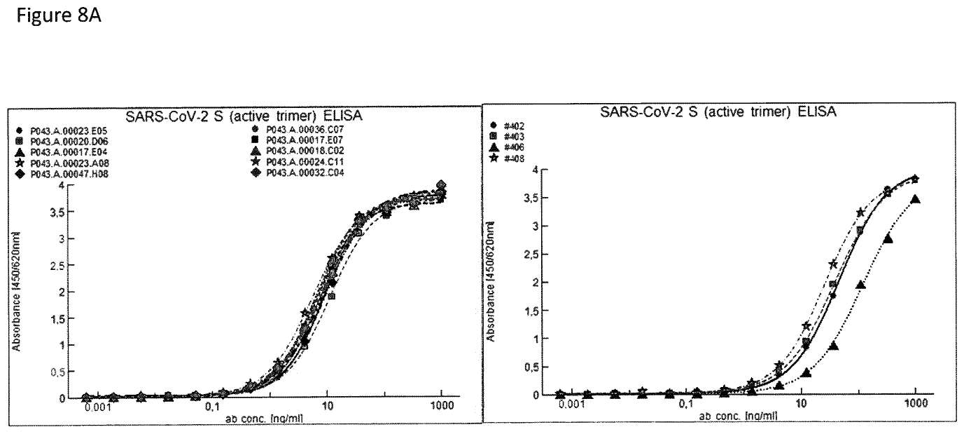

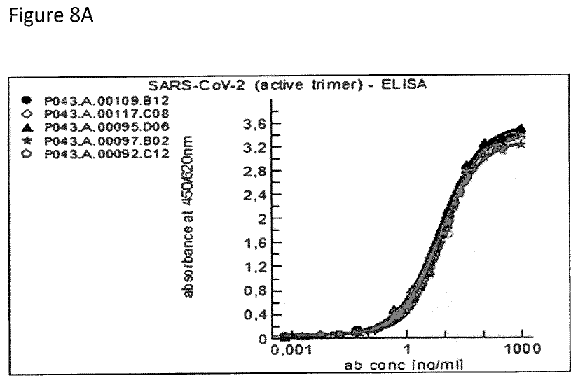

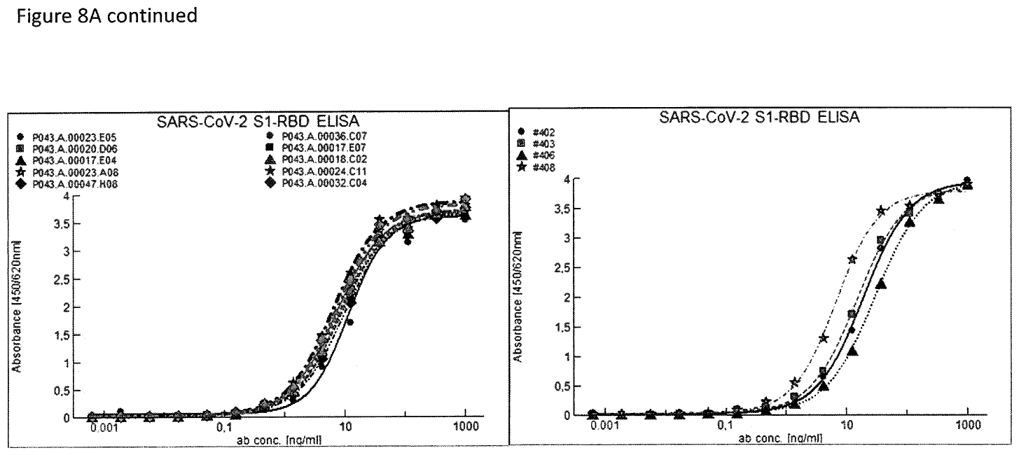

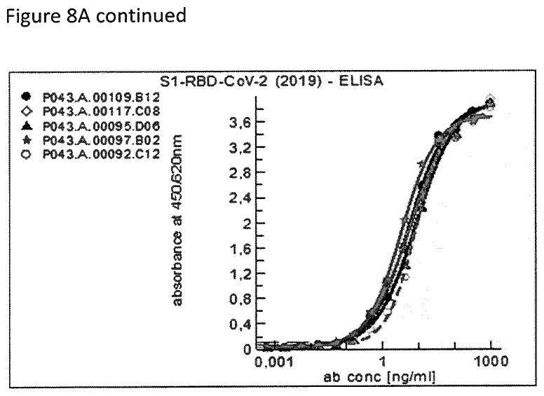

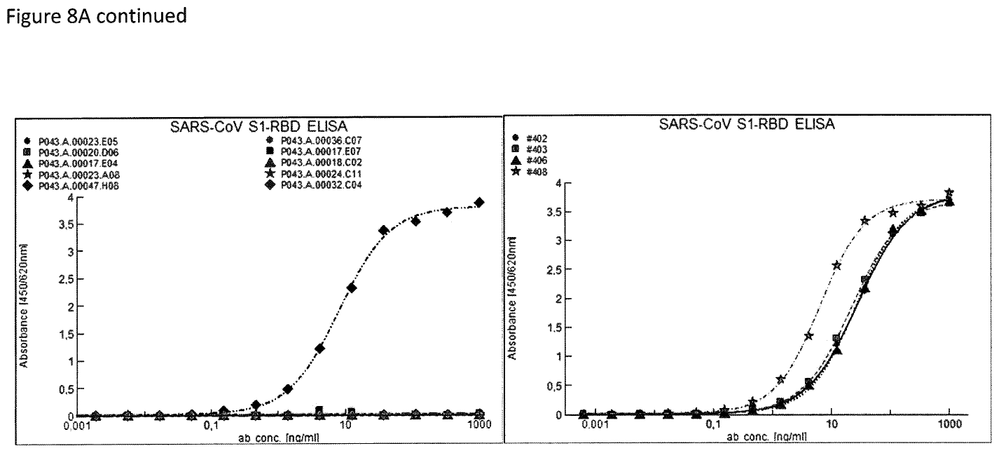

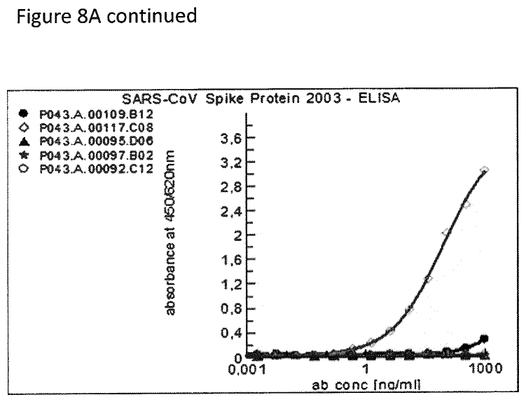

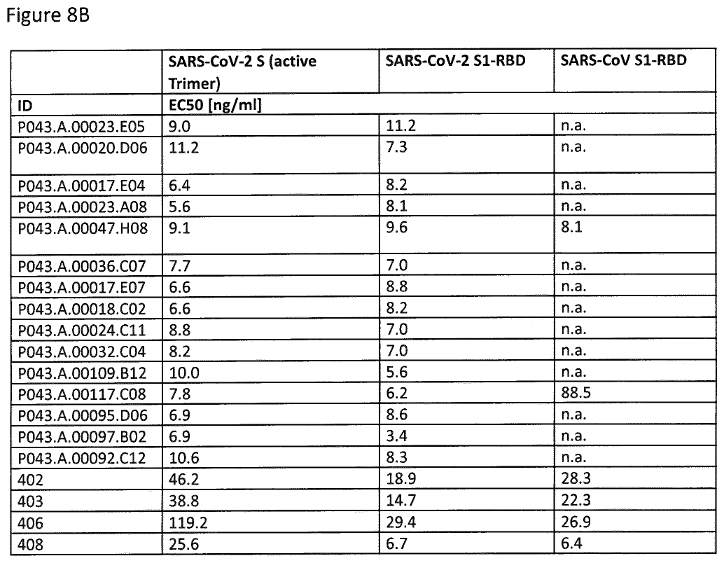

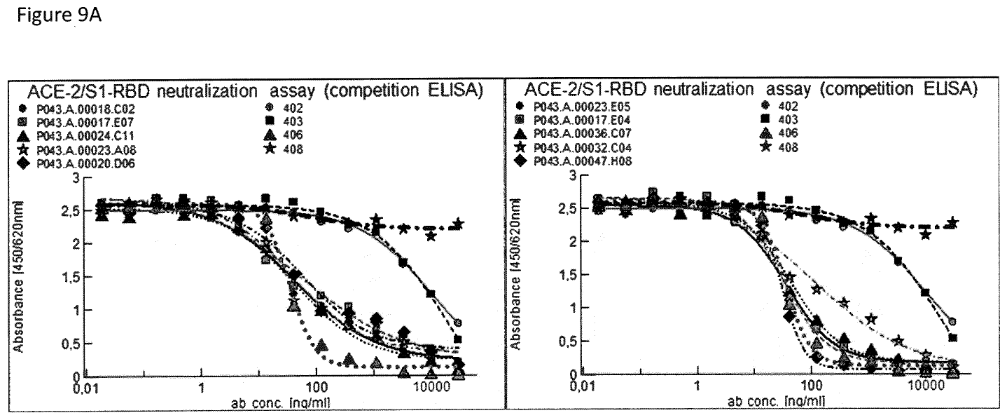

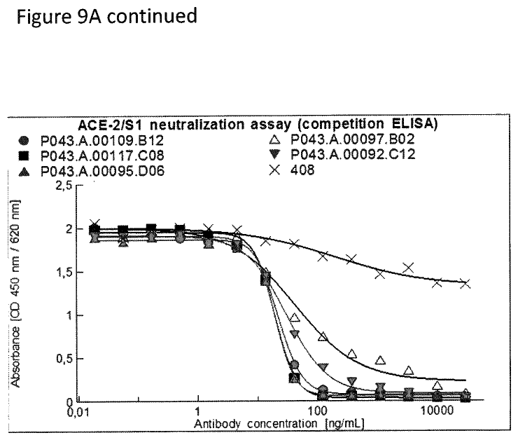

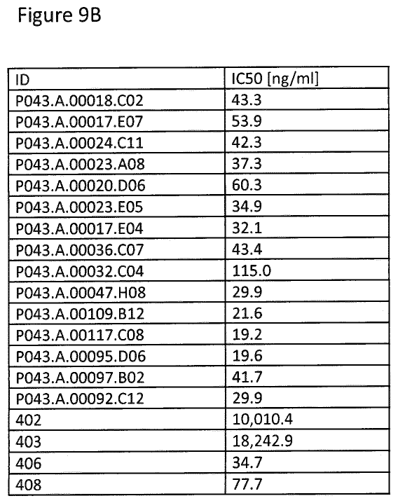

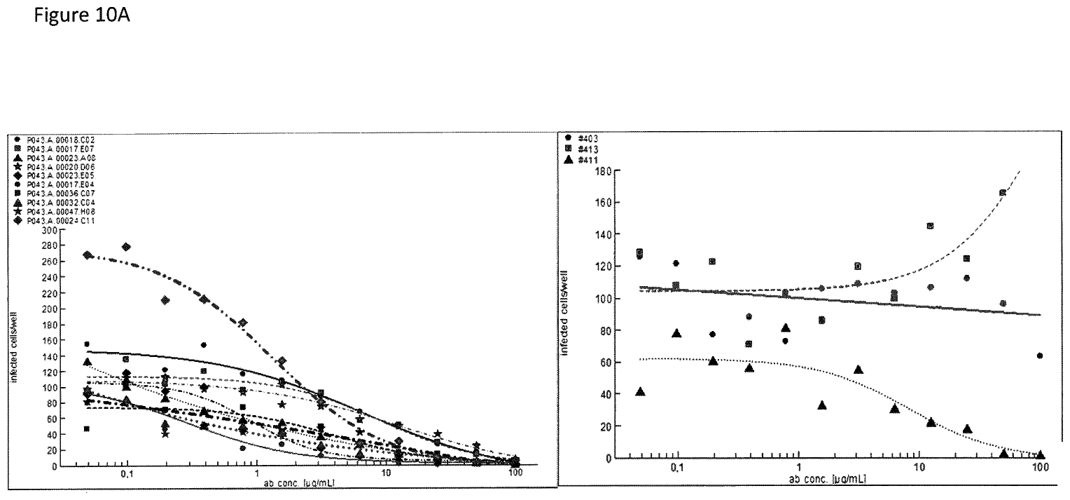

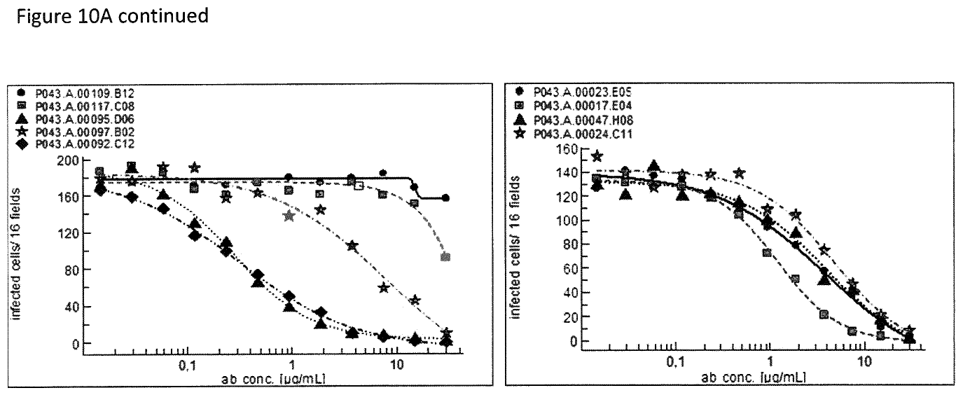

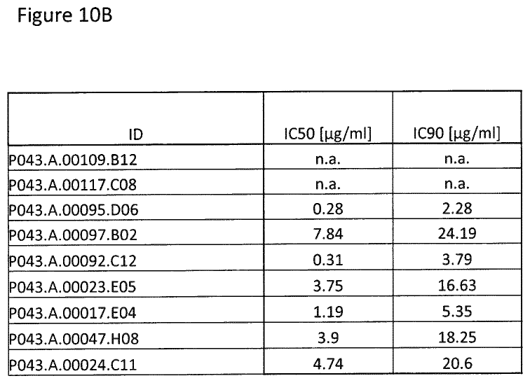

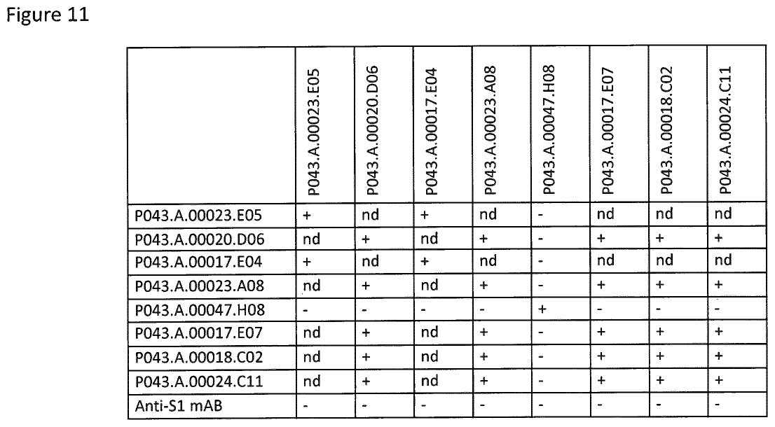

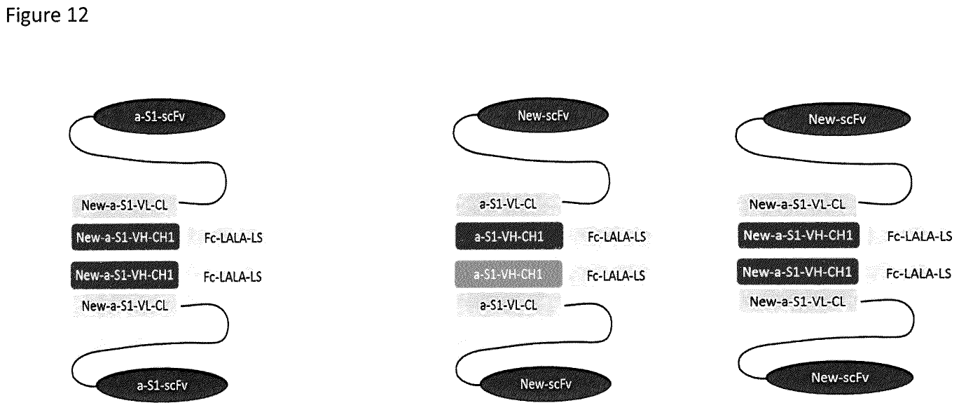

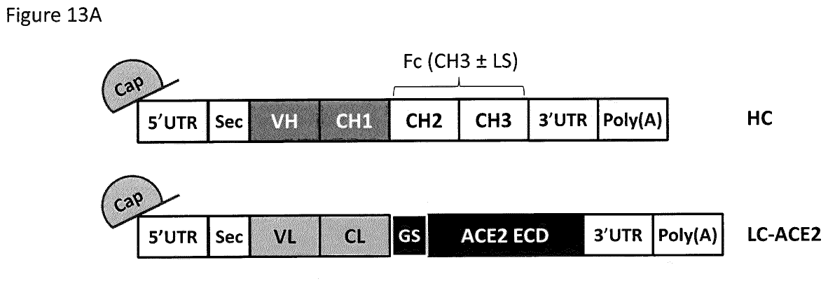

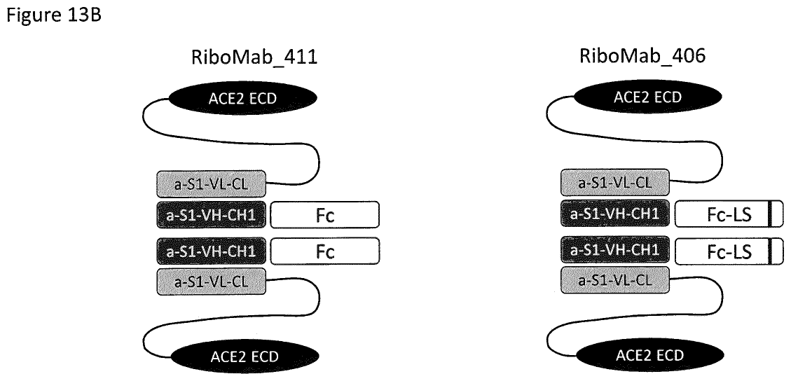

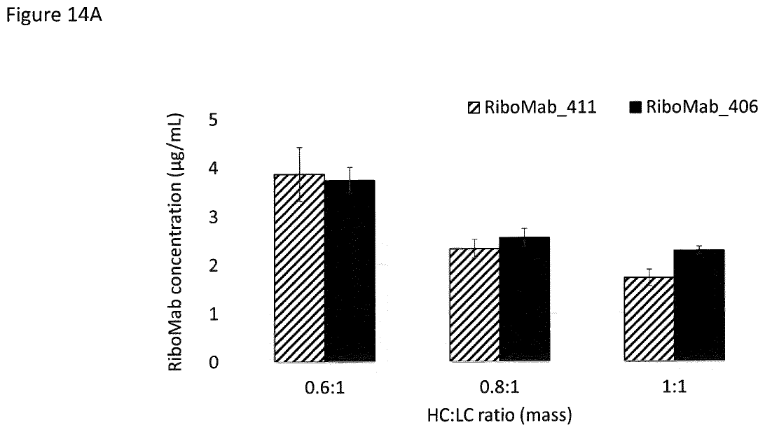

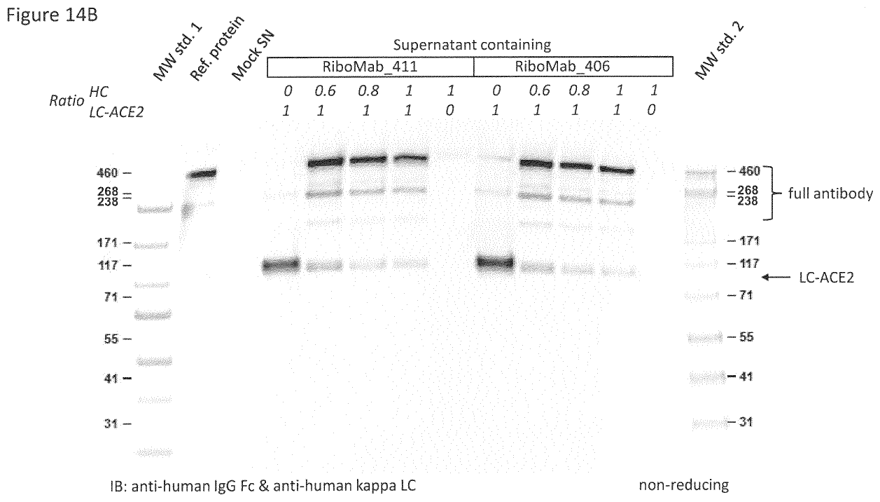

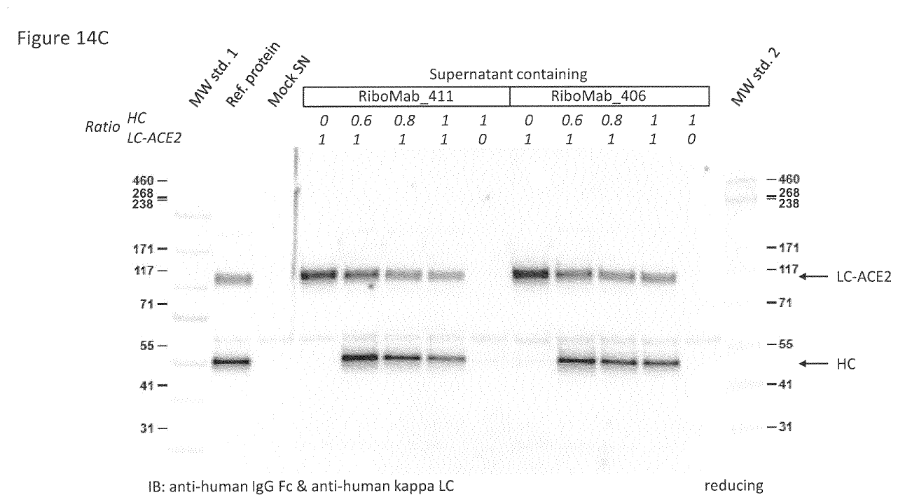

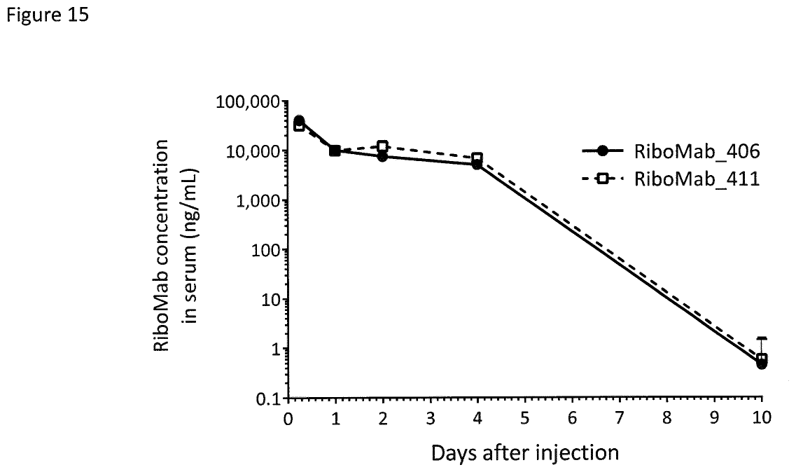

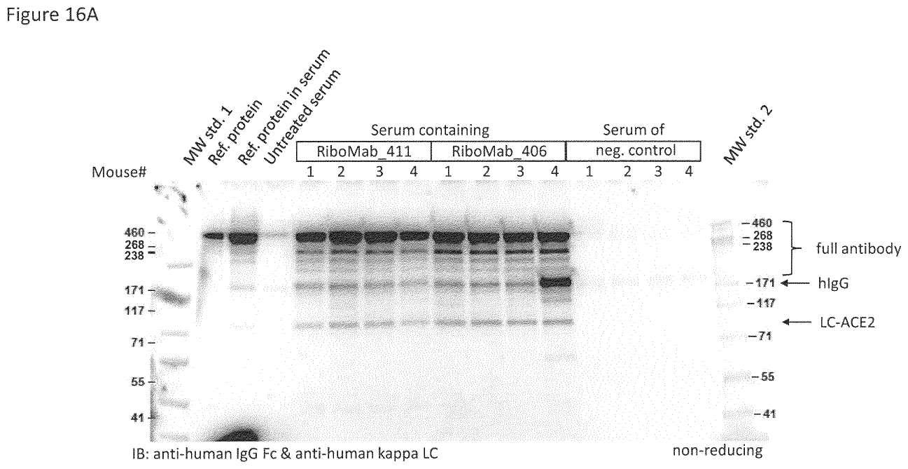

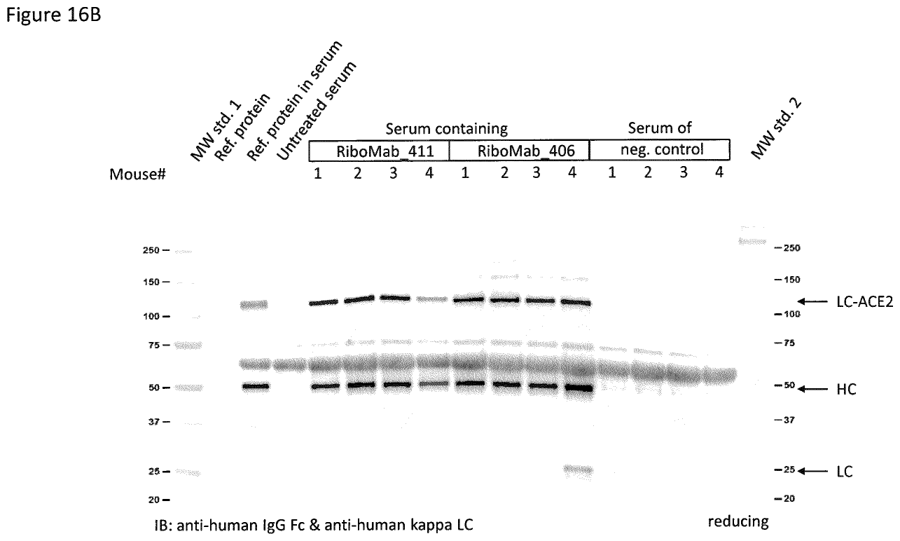

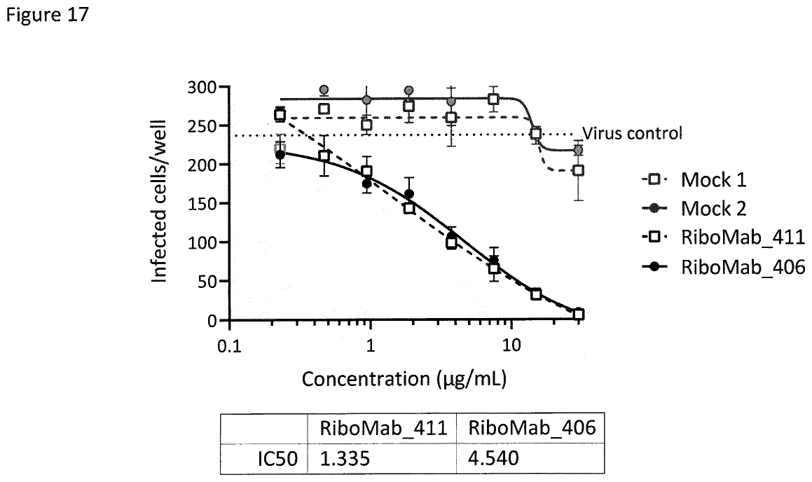

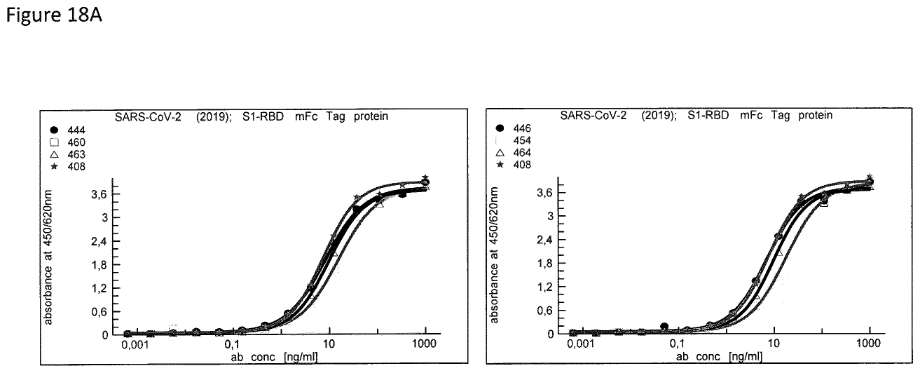

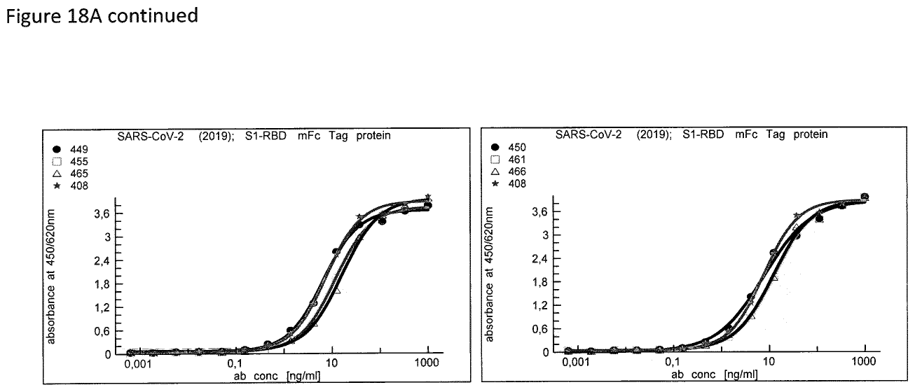

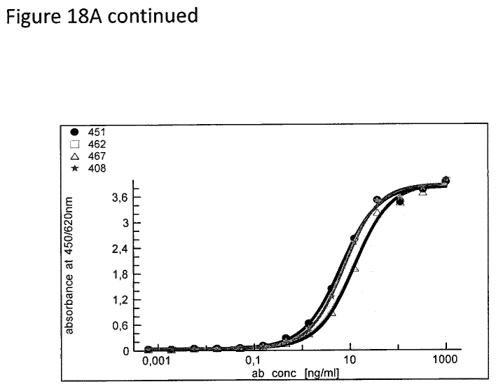

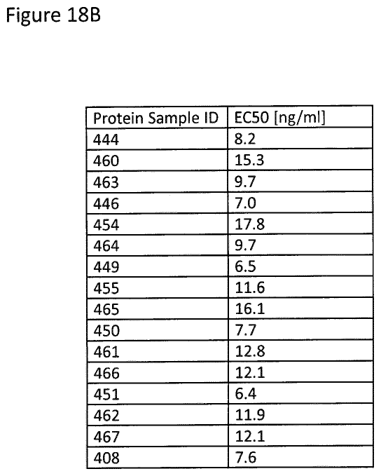

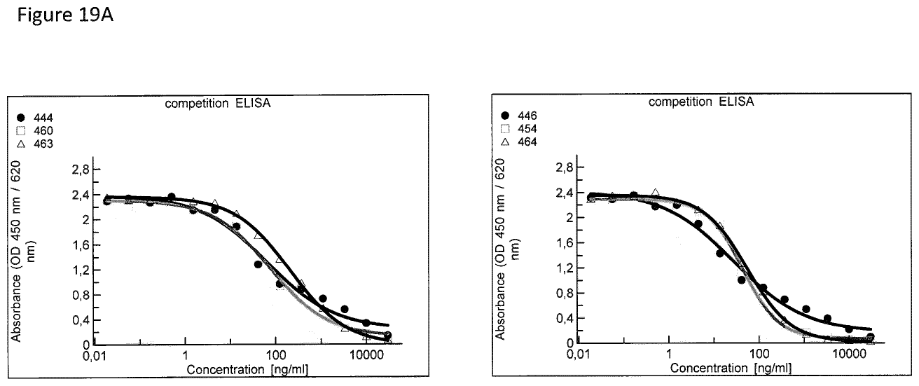

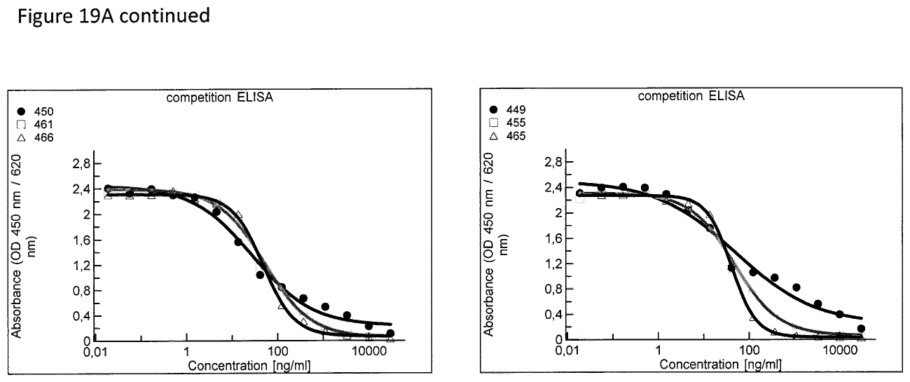

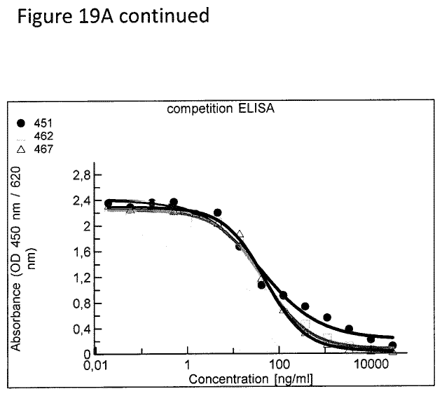

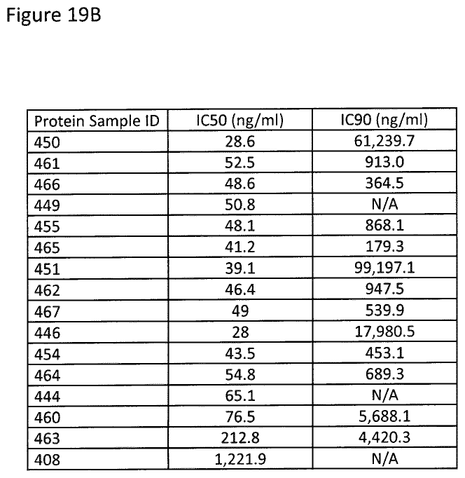

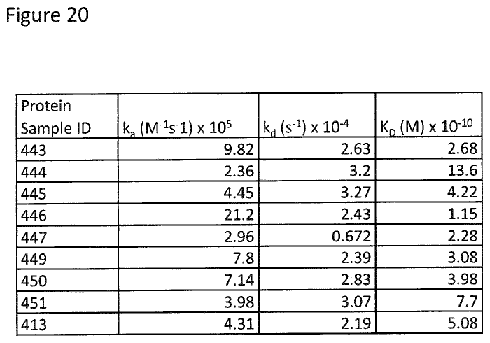

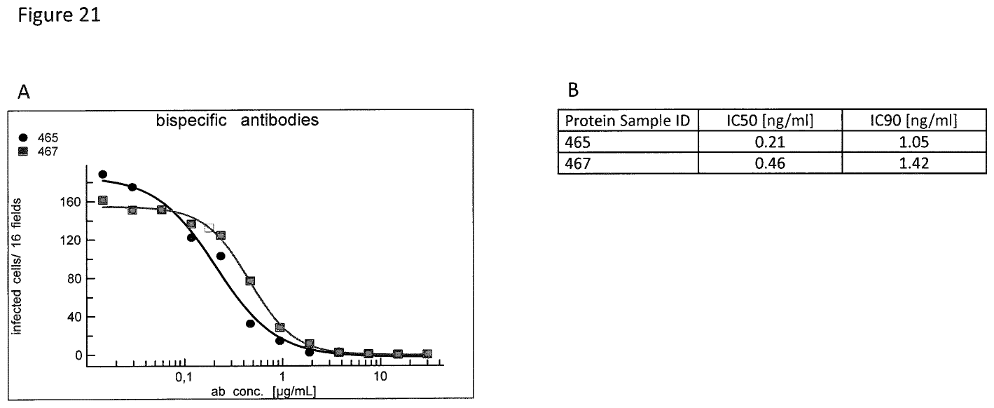

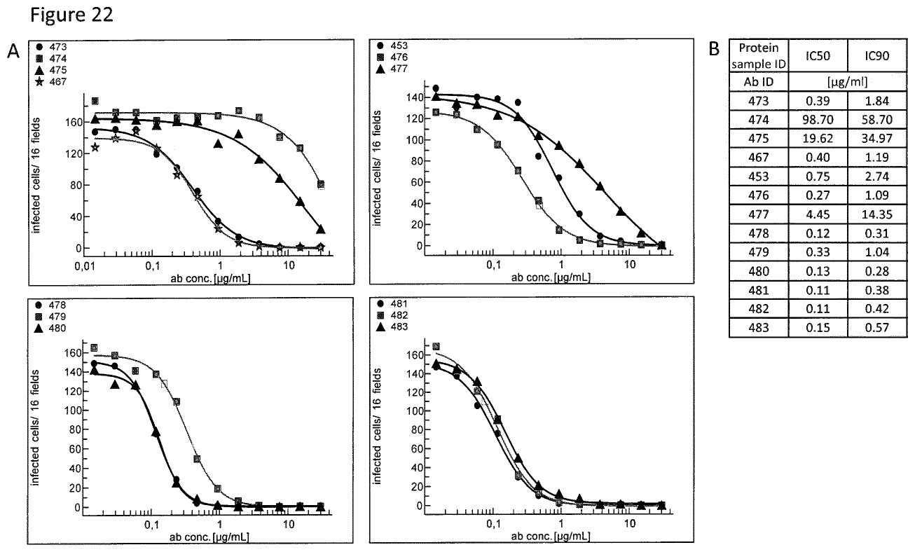

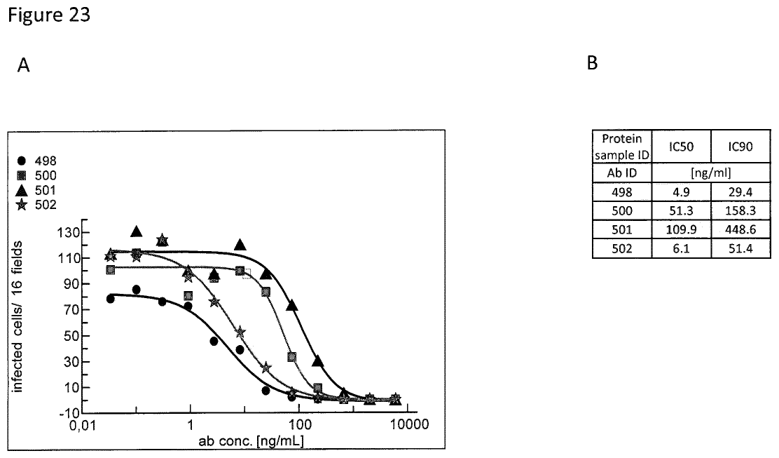

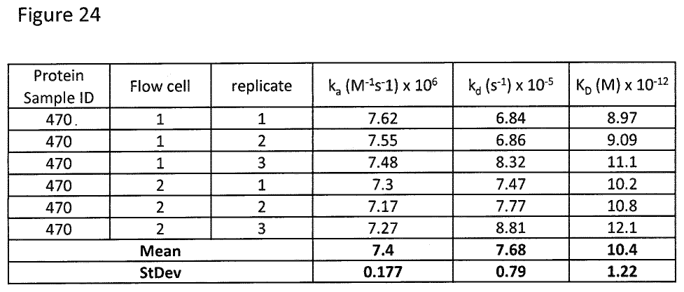

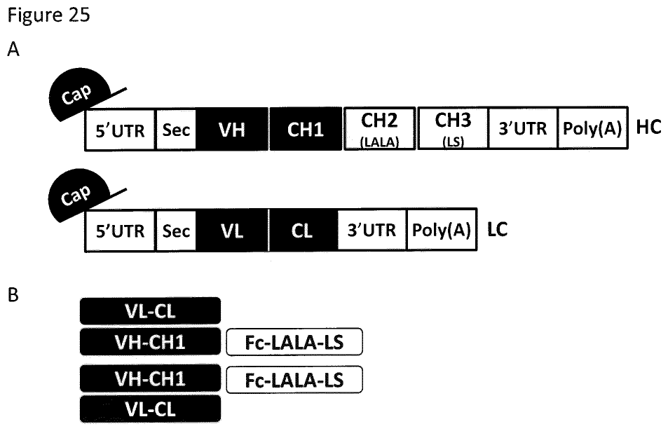

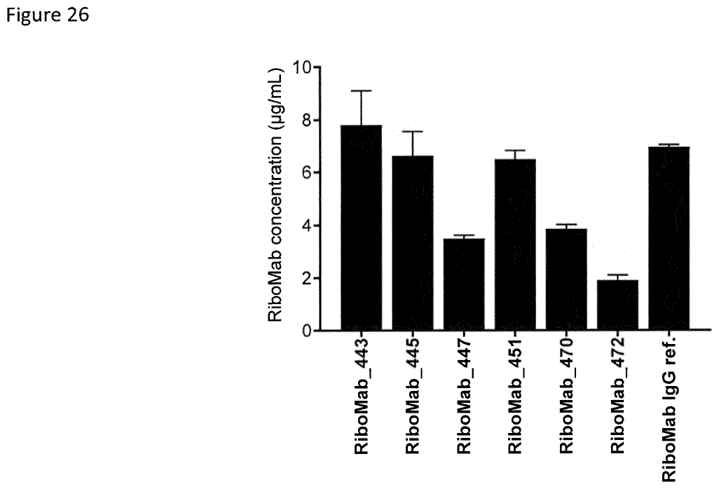

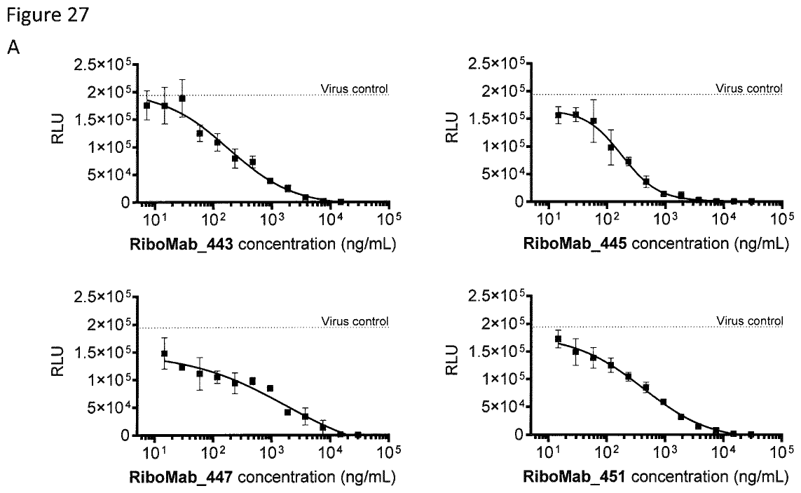

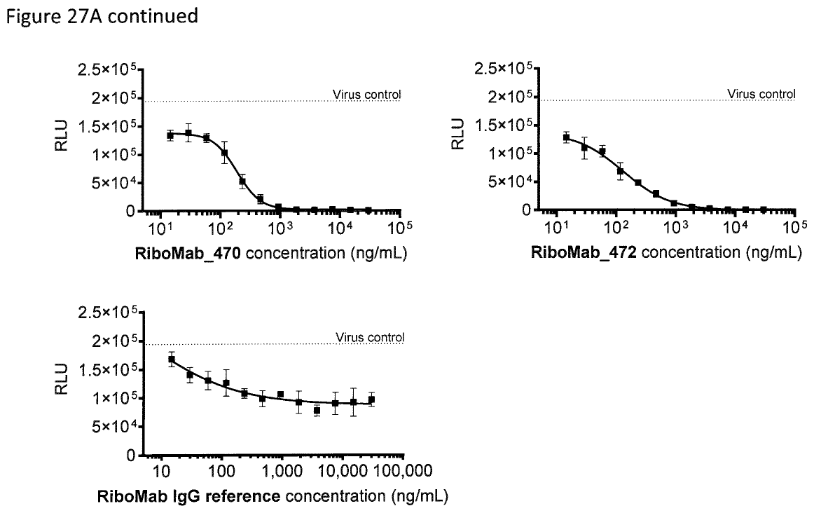

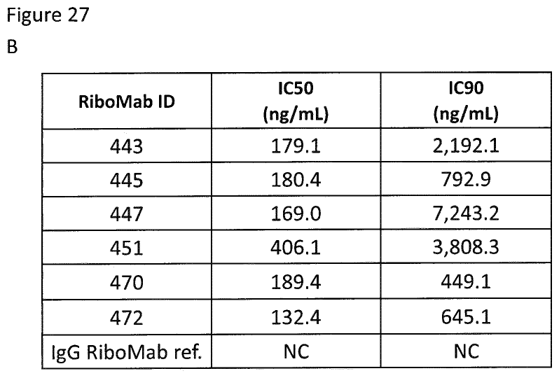

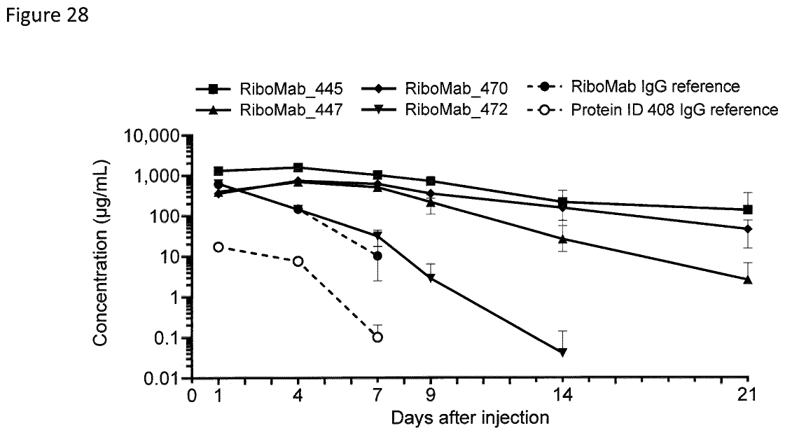

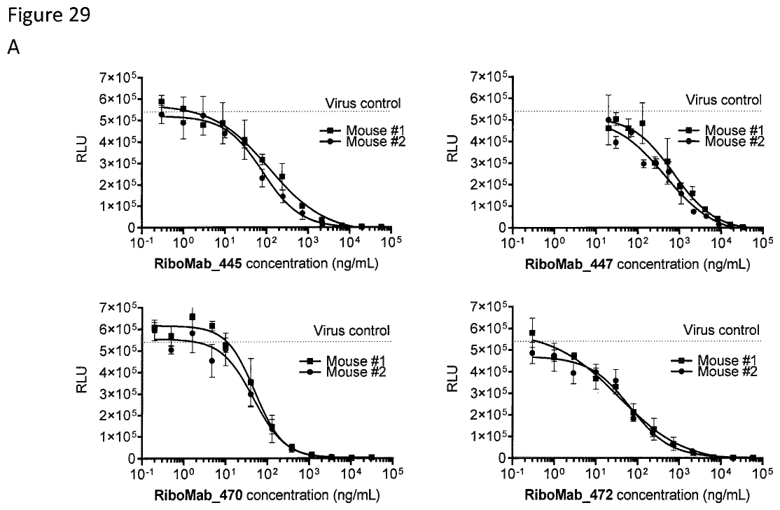

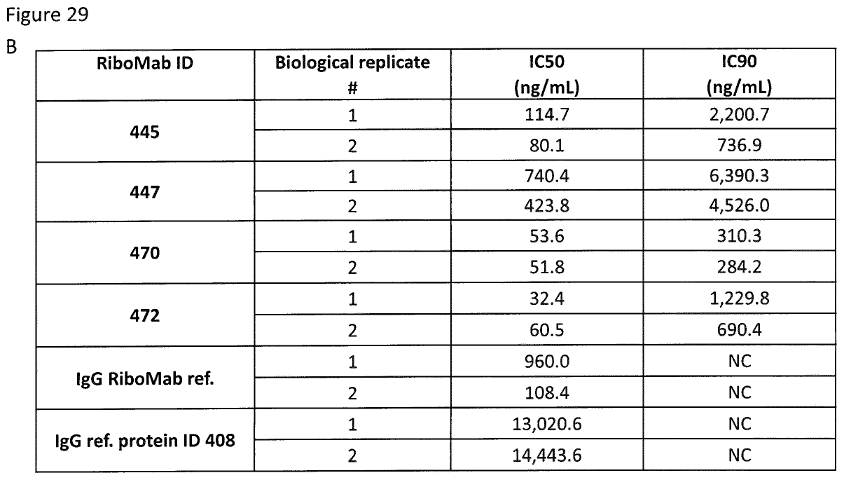

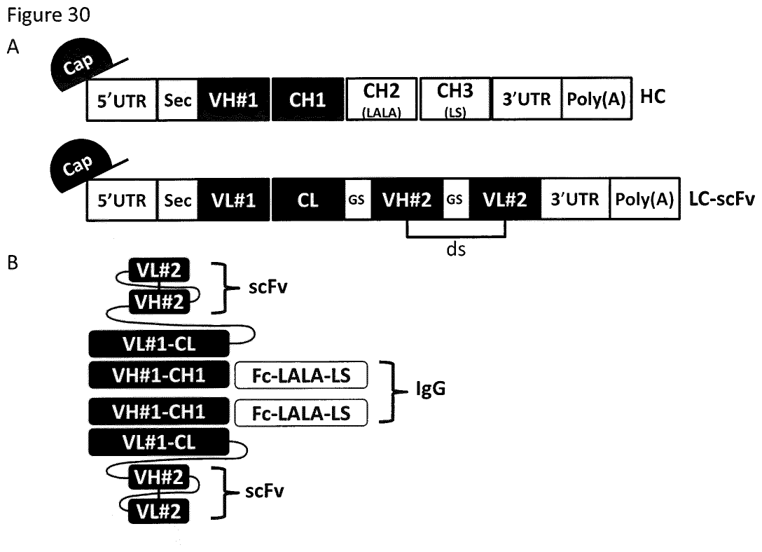

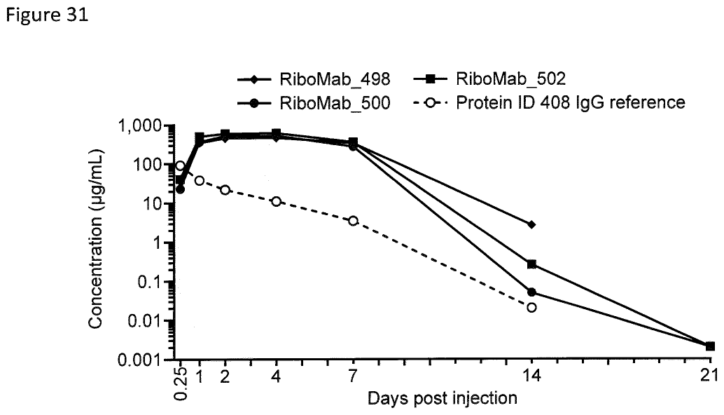

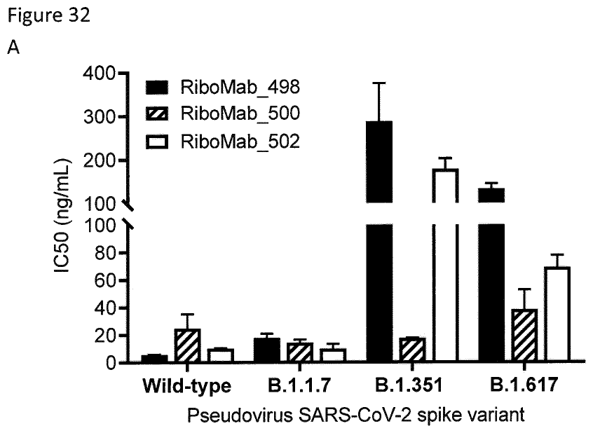

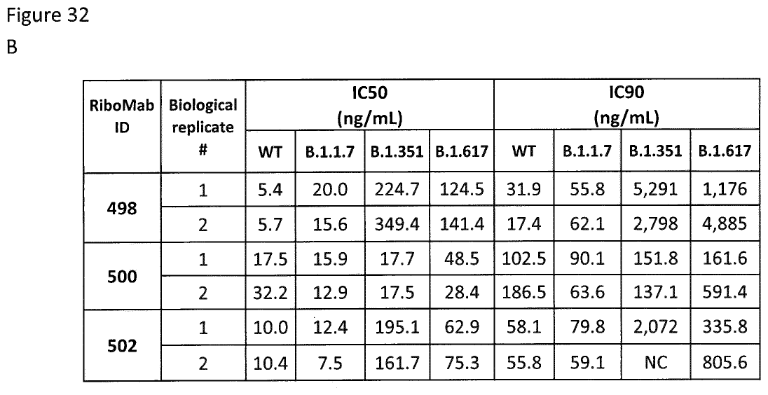

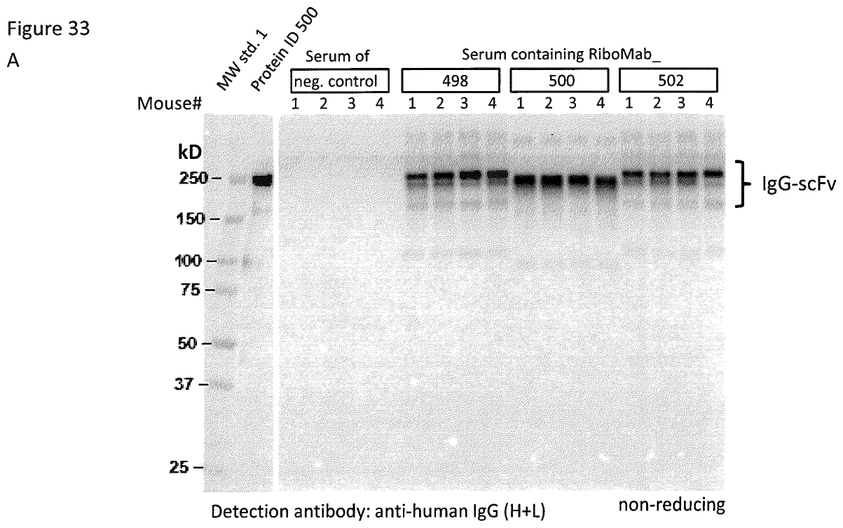

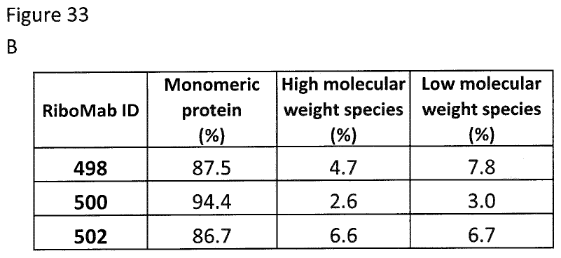

In order to determine the concentration of monoclonal antibodies in B-cell supernatants, a quantitative sandwich ELISA was performed. Briefly, B-cell supernatants were diluted 1:10, 1:30, 1:100, 1:300, 1:100 and 1:3,000 and incubated on plates coated with a goat anti-rabbit-IgG antibody (Sigma-Aldrich). Captured rabbit IgG was detected using a horseradish peroxidase-linked species-specific anti-rabbit-IgG F(ab)₂ Fragment from donkey (GE Healthcare). ODs at 450/620 nm were recorded using a Tecan Infinite M1000 instrument and correlated to standard curves obtained with purified rabbit IgG (Sigma Aldrich). The calculated rIgG concentrations of monoclonal antibodies in B-cell supernatants are summarized in FIG. 6A.

The binding potency of anti-S1-antibodies of the invention in B-cell supernatants to SARS-CoV2 S1 protein has been tested in an ELISA. Human-Fc-tagged SARS-CoV2 S1 recombinant protein (Sino Biologicals) was coated on a 384-well Nunc MaxiSorp™ flat bottom plate at a concentration of 0.875 or 3 μg/ml in PBS for 60 minutes at room temperature. After 3 washes with PBS 0.1% Tween (wash buffer), blocking with PBS, 2% BSA, 0.05% Tween for 60 minutes at room temperature and another 3 washes, anti-S1-antibodies containing B-cell supernatants were added in PBS, 0.5% BSA, 0.05% Tween (ELISA buffer) in concentrations ranging from 1,000 to 0.04 ng/ml and the plate was incubated for 60 minutes at room temperature. As a control, recombinant ACE-2 extracellular domain with mouse-Fc tag (Sino Biologicals) was used in concentration from 5,000 to 2 ng/ml. After 3 washes with wash buffer, the horseradish peroxidase coupled detection anti-rabbit IgG F(ab′)₂ fragment (Cytiva) or for the control the horseradish peroxidase coupled detection anti-mouse IgG F(ab′)₂ fragment (Cytiva) was added in ELISA buffer at a dilution of 1:4,000 or 1:1,000, respectively. The plate was incubated for 60 minutes at room temperature, washed 6 times with wash buffer before TMB solution (Thermo Fisher Scientific) was added. After 6 minutes HCl was added and the absorbance at wavelengths of 450 and 620 nm recorded using a Tecan Infinite M1000 reader. Fitting curves and EC50 calculation were obtained by using Excel (Microsoft) and XLfit (IDBS). The data in FIGS. 6B and C shows that all antibodies secreted in monoclonal B-cell supernatants bind in a dose dependent manner to SARS-CoV2 S1 recombinant protein and with lower EC50 values as compared to mFc-tagged ACE-2 extracellular domain.

### Example 8: Neutralization of the SARS-CoV2-S1—ACE-2 Interaction by Antibodies in B-Cell Supernatants

The potency of anti-S1-antibodies of the invention in B-cell supernatants to neutralize the SARS-CoV2 S1 binding to the ACE-2 extracellular domain was investigated in a competition ELISA. Mouse-Fc-tagged human ACE-2 extracellular domain (Sino Biologicals) recombinant protein was coated on a 384-well Nunc MaxiSorp™ flat bottom plate at a concentration of 2.5 μg/ml in PBS for 60 minutes at room temperature. After 3 washes with PBS 0.1% Tween (wash buffer), the MaxiSorp™ plate was blocked with PBS, 2% BSA, 0.05% Tween for 60 minutes at room temperature. In a separate polypropylene 384-well plate (Corning) anti-S1-antibodies containing B-cell supernatants at a concentration from 2,000 to 0.08 ng/ml were pre-incubated with 60 ng/ml human-Fc-tagged SARS-CoV2 S1 (Sino Biologicals) recombinant protein diluted in PBS, 0.5% BSA, 0.05% Tween (ELISA buffer) for 60 minutes at room temperature. As a control, recombinant ACE-2 extracellular domain with mouse-Fc tag (Sino Biologicals) was used. After 3 washes with wash buffer, the pre-incubation anti-S1 antibody/S1 protein mix from the Corning plate was transferred onto the MaxiSorp™ plate and incubated for 60 minutes at room temperature. After 3 washes with wash buffer, the horseradish peroxidase coupled detection anti-human IgG, Fcγ fragment specific F(ab′)₂ fragment (Jackson Immuno Research) was added in ELISA buffer at a dilution of 1:5,000. The plate was incubated for 60 minutes at room temperature, washed 6 times with wash buffer before TMB solution (Thermo Fisher Scientific) was added. After 15 minutes HCl was added and the absorbance at wavelengths of 450 and 620 nm recorded using a Tecan Infinite M1000 reader. Fitting curves and IC50 calculation were obtained by using Excel (Microsoft) and XLfit (IDBS). The data in FIGS. 7A and B shows that all antibodies secreted in monoclonal B-cell supernatants block the interaction between SARS-CoV2 S1 and ACE-2 recombinant proteins in a dose dependent manner and with significantly lower EC50 values as compared to mFc-tagged ACE-2 extracellular domain.

### Example 9: Binding of Purified hIgG1-LALA-LS Chimeric Antibodies to SARS-CoV2-S/S1-RBD and SARS-CoV-S1-RBD Recombinant Protein

The variable region sequences of the antibodies of the invention were cloned in frame with hIgG1 constant light and heavy chain sequences to obtain chimeric antibody constructs. In the hIgG1 heavy chain sequences mutations L234A and L235A were introduced which are described to reduce Fcγ receptor binding (Hezareh et al. 2001) and the LS (M428L/N434S) mutation. Plasmid DNAs encoding chimeric light and heavy chains were cotransfected into HEK-293 FreeStyle™ cells and antibodies purified from the culture supernatants by protein-A affinity and subsequent size exclusion chromatography.

The potencies of chimeric anti-S1-antibodies of the invention in binding to SARS-CoV S1-RBD, SARS-CoV2 S (active trimer) and SARS-CoV2 S1-RBD protein were determined in an ELISA. His-tagged SARS-CoV S1-RBD (Sino Biologicals), His-tagged SARS-CoV2 S active trimer (AcroBiosystems) and mouse-Fc-tagged SARS-CoV2 S1-RBD (Sino Biologicals) recombinant proteins were coated on 384-well Nunc MaxiSorp™ flat bottom plates at a concentration of 1 μg/ml, 1 μg/ml and 2.5 μg/ml in PBS for 60 minutes at room temperature. After 3 washes with PBS 0.1% Tween (wash buffer), blocking with PBS, 2% BSA, 0.05% Tween for 60 minutes at room temperature and another 3 washes, chimeric anti-S1-antibodies were added in PBS, 0.5% BSA, 0.05% Tween (ELISA buffer) in concentrations ranging from 20,000 to 0.006 ng/ml and the plate was incubated for 60 minutes at room temperature. As a control, recombinant ACE-2 extracellular domain with human-Fc tag (402/403), anti-S1-ACE2 fusion construct 406 and the anti-S1 antibody 408 was used. After 3 washes with wash buffer, the horseradish peroxidase coupled detection anti-human IgG, Fcγ fragment specific F(ab′)₂ fragment (Jackson Immuno Research) was added in ELISA buffer at a dilution of 1:2,500. The plate was incubated for 60 minutes at room temperature, washed 6 times with wash buffer before TMB solution (Thermo Fisher Scientific) was added. After 6 minutes HCl was added and the absorbance at wavelengths of 450 and 620 nm recorded using a Tecan Infinite M1000 reader. Fitting curves and EC50 calculation were obtained by using Excel (Microsoft) and XLfit (IDBS). The data in FIGS. 8A and B demonstrates that the tested chimeric antibodies of the invention all bind to SARS-CoV2 S (active trimer) and the SARS-CoV2 S1-RBD with similar potency characterized by EC50 values between 3.4 and 11.2 ng/ml. These EC50 are lower than those measured for ACE2-hFc and anti-S1-ACE2 fusion construct 406. The chimeric antibodies P043.A.00047.H08 and P043.A.00117.008 are also able to bind to SARS-CoV S1-RBD with a calculated EC50 of 8.1 and 88.5 ng/ml, respectively.

### Example 10: Neutralization of SARS-CoV2-S1-RBD Binding to ACE2 by Chimeric Antibodies of the Invention

The potency of purified, chimeric antibodies of the invention in neutralizing the SARS-CoV2 S1-RBD binding to the ACE-2 extracellular domain was investigated in a competition ELISA as described in example 3. All chimeric antibodies block the interaction of ACE-2 with SARS-CoV2 S1-RBD in a dose-dependent manner. EC50 values range from 19.2 to 115 ng/ml. The inhibition observed by the chimeric of the antibodies of the invention is significantly more potent than the inhibition observed with ACE2-hFc (402/403) (FIGS. 9A and B).

### Example 11: Pseudovirus Neutralization Activity by Purified Antibodies of the Invention

To determine the potencies of chimeric antibodies of the invention to inhibit S1 protein-directed virus infection of cells, a pseudovirus neutralization test (pVNT) was performed as described in example 4 but with the following adaptations.

For the pVNT assay, Vero-76 cells were thawed and diluted to 0.5×10⁶ cells/mL in assay medium (DMEM/10% FBS) and seeded in 384-well flat-bottom tissue culture plates (Corning) at 1×10⁴ cells per well. Cells were incubated for 4 to 6 hours at 37° C. and 5% CO₂. VSV/SARS CoV2 pseudovirus was thawed and diluted to obtain 12×10³ infectious units [IU]/mL. 10 μL of diluted pseudovirus was added to the wells of 384-well V-bottom plates (Corning) containing 10 μl chimeric antibodies of the invention or molecules 403, 413 and 411 for final concentrations ranging from 100 to 0.05 μg/ml. In some experiment, selected antibodies of the invention were tested in concentrations ranging from 30 to 0.01 μg/ml (FIG. 10B). Pseudovirus/test antibody mix was incubated for 10 min at RT on a microplate shaker at 1,200 rpm. Pseudovirus/test protein dilution mix was then added to the seeded Vero-76 cells, followed by incubation for 16 to 24 hours at 37° C. and 5% CO₂. Each dilution of serum samples was tested in triplicate wells. After incubation, the cell culture plates were removed from the incubator, stained with 5 μg/ml Hoechst 33342 (Thermo Fisher Scientific) diluted in assay medium, placed in CellInsight CX5 imaging system and incubated for 10 min prior to the analysis. Whole well scanning for Hoechst and GFP fluorescence was performed using a 4× objective. Curve fitting was done using Excel (Microsoft) and XLfit (IDBS). FIG. 10 demonstrates that the anti-S1-antibody (413) and the ACE2-hFC (403) do not significantly affect infection of Vero-76 cells by the pseudovirus whereas the anti-S1-antibody-ACE2 fusion protein (411) and many chimeric antibodies of the invention inhibit infection in a dose dependent manner. Most of the chimeric antibodies show stronger neutralizing activities than protein 411 by achieving complete neutralization already at lower concentrations. In FIG. 10B the IC50 and IC90 values for selected antibodies of the invention are summarized.

### Example 12: SARS-CoV2-S1-RBD Epitope Competition Among Antibodies of the Invention

The interference of each chimeric antibody with any other chimeric antibody of the invention in binding to the SARS-CoV2 S1-RBD has been tested in a competition ELISA.

One antibody was coated on a 384-well Nunc MaxiSorp™ flat bottom plate at a concentration of 2.5 μg/ml in PBS for 60 minutes at room temperature. After 3 washes with PBS 0.1% Tween (wash buffer), the MaxiSorp™ plate was blocked with PBS, 2% BSA, 0.05% Tween for 60 minutes at room temperature. In a separate polypropylene 384-well plate (Corning) the other antibody at a concentration from 20,000 to 0.01 ng/ml was pre-incubated with 150 ng/ml mouse-Fc-tagged SARS-CoV2 S1-RBD (Sino Biologicals) recombinant protein in PBS, 0.5% BSA, 0.05% Tween (ELISA buffer) for 60 minutes at room temperature. After 3 washes with wash buffer, the pre-incubation mix from the Corning plate was transferred onto the MaxiSorp™ plate and incubated for 60 minutes at room temperature. After 3 washes with wash buffer, the horseradish peroxidase coupled detection anti-mouse IgG F(ab′)₂ fragment (Cytiva) was added in ELISA buffer at a dilution of 1:1,000. The plate was incubated for 60 minutes at room temperature, washed 6 times with wash buffer before TMB solution (Thermo Fisher Scientific) was added. After 6 minutes HCl was added and the absorbance at wavelengths of 450 and 620 nm recorded using a Tecan Infinite M1000 reader. FIG. 11 summarizes the results of the competition ELISA data with (+) indicating competition in S1-binding between two tested antibodies and (−) indicating concomitant binding of two tested antibodies. P043.A.00047.H08 is the only antibody in the set of tested chimeric antibodies of the invention which only competes with itself in the assay but with none of the other antibodies, including the anti-S1-mAB. In contrast, all other chimeric antibodies of the invention do compete amongst each other indicating they have overlapping epitopes on the S1-RBD protein. None of the chimeric antibodies of the invention competes with the anti-S1 mAB.

### Example 13: Generation of IVT-mRNA Based Anti-S1-Antibody-ACE2 Fusion RiboMabs

a. Cloning of Antibody IVT-mRNA Template Vectors and IVT-mRNA Synthesis

For the generation of anti-S1-antibody-ACE2 fusion RiboMabs via in vitro transcribed messenger RNA (IVT-mRNA), we subcloned the DNA sequences of the fully human anti-S1-antibody-ACE2 fusion antibodies ID 406 and ID 411 (described in Example 1) into the IVT-mRNA template vector pST1-hAg-MCS-FI-A30LA70 (BioNTech RNA Pharmaceuticals, Mainz, Germany) using standard techniques. The human alpha globin (hAg) 5′UTR leader sequence has been described elsewhere and the FI sequence is described in patent application “3′UTR Sequences for Stabilization of RNA” (PCT/EP2016/073814). The poly(A) tail-encoding region (A30LA70) consists of 30 adenine codons, a linker (L) and further 70 adenine codons (PCT/EP2015/065357). All antibody domains originate from human IgG1. The following constructs were cloned for the formation of anti-S1-antibody-ACE2 fusion RiboMabs:

RiboMab_406:
pST1-5′hAg-Sec-V_{H}^{anti-S1}-C_{H}1-C_{H}2-C_{H}3^{(Met434Leu, Asn428Ser)}-FI-A30LA70 (HC)
pST1-5′hAg-Sec-V_{L}^{anti-S1}-CL-(G₄S)₄-ACE2-ECD-FI-A30LA70 (LC-ACE2)

RiboMab_411:

pST1-5′hAg-Sec-V_{H}^{anti-S1}-C_{H}1-C_{H}2-C_{H}3-FI-A30LA70 (HC)

pST1-5′hAg-Sec-V_{L}^{anti-S1}-C_{L}-(G₄S)₄-ACE2-ECD-FI-A30LA70 (LC-ACE2)

5′hAg, 5′UTR from human alpha-globin; A, adenine; Asn, asparagine; CL, constant light chain region; CH, constant heavy chain region; ECD, extracellular domain of ACE2; FI, 3′UTR sequence; (G₄S)₄, glycine-serine linker encoding sequence; HC, heavy chain; Leu, leucine; LC, light chain; Met, methionine; pST1, DNA template vector; Sec, secretion signal; Ser, serine; VH, variable heavy chain domain; VL, variable light chain domain.

b. IVT-mRNA Synthesis

To generate templates for in vitro transcription, plasmid DNAs were linearized downstream of the poly(A) tail-encoding region using a class Its restriction endonuclease, thereby generating a template to transcribe RNAs with no additional nucleotides past the poly(A)-tail (Holtkamp, S. et al. (2006) Blood 108 (13), 4009-4017). Linearized template DNAs were purified, spectrophotometrically quantified, and then subjected to in vitro transcription with T7 RNA polymerase essentially as previously described (Grudzien-Nogalska, E. et al. (2013): Synthetic mRNAs with superior translation and stability properties. In: Methods in molecular biology (Clifton, N.J.) 969, 55-72). To minimize immunogenicity, N1-Methylpseudouridine-5′-Triphosphate (TriLink Biotechnologies, San Diego, Calif., USA), short m1ψTP, was incorporated instead of UTP (Kariko, K. et al. (2008) Mol. Ther. 16 (11), 1833-1840) and double-stranded RNA was removed by cellulose purification (Baiersdörfer, M. et al. (2019) Nucleic acids 15, 26-35). RNA was capped with CleanCap413, a Cap1-structure. To this end, in vitro transcription was performed in the presence of 7.5 mM each of ATP, CTP, m1ΨTP, GTP, and 1.5 mM CleanCap413. RNA was purified using magnetic particles (Berensmeier, S. (2006): Magnetic particles for the separation and purification of nucleic acids. In: Applied microbiology and biotechnology 73 (3), 495-504). RNA concentration and quality were assessed by spectrophotometry and analysis on a 2100 Bioanalyzer (Agilent, Santa Clara, Calif., USA). A sketch of the two IVT-mRNAs needed for the formation of a complete antibody molecule is depicted in FIG. 13.

### Example 14: Expression and Protein Integrity of Anti-S1-Antibody-ACE2 Fusion RiboMabs In Vitro

a. Electroporation of Producer Cells

For the production of anti-S1-antibody-ACE2 fusion RiboMabs from IVT-mRNA, 1×10⁷ HEK 2931/17 cells (ATCC CRL®-11268™, LGC Standards GmbH, Wesel, Germany) per mL growing in log-phase were used for electroporation. Cells in X-Vivo 15 medium (LONZA Technologies, Basel, Switzerland) were combined in 4 mm gap cuvettes (VWR, Darmstadt, Germany) with 10 mM Hepes/0.1 mM EDTA buffer (Mock) or with 100 μg/mL of the antibody-encoding IVT-mRNA mixture. The RNA mixture contained a mass ratio of the mRNAs encoding HC and LC-ACE2 of 0:1, 0.6:1, 0.8:1, 1:1, or 1:0, respectively. Cells were immediately electroporated with a BTX ECM830 (BTX Harvard Apparatus, Holliston, Mass., USA) with the following setting: 250 V, 2 pulses, 5 ms. Viable cells were subsequently seeded in Expi293™ medium (Gibco Thermo Fischer Scientific, Darmstadt, Germany) at a density of 2×10⁶/mL in 12-well tissue culture plates (Cellstar®, Greiner Bio-One, Frickenhausen, Germany). After 48 hours of incubation, the supernatant was harvested by centrifugation (10 min, 300×g) and stored at 4° C. until analysis.

b. Quantitation of Anti-S1-Antibody-ACE2 Fusion RiboMabs in Producer Cell Culture Supernatant Via Immunoassay

Anti-S1-antibody-ACE2 fusion RiboMabs in SN from electroporated HEK 293T/17 cells were quantified using a Gyros xPand™ XPA1025 device (Gyros Protein Technologies AB, Uppsala, Sweden). All materials were from Gyros Protein Technologies AB, if not stated otherwise. A sandwich immunoassay was run with Gyrolab® huIgG Kit—Low Titer according to the manufacturer's protocol. The reagent kit components were used with the Gyrolab® Bioaffy 1000 HC CD for protein concentrations in a dynamic range of 20-9,000 ng/mL.

All samples and reagents were centrifuged for 4 min at 12,000×g to sediment any aggregates. SN containing the respective RiboMab molecules were diluted 1:2 in Reagent E buffer. Prepared standards, quality controls, reagents and diluted samples were loaded onto a 96-well plate according to Gyrolab Loading list. Data were generated with the Gyrolab® huIgG Low Titer kit method v2 and the results were evaluated using Gyrolab® Evaluator software.

The HC-to-LC-ACE2 mRNA mass ratio of 0.6:1 yielded the highest anti-S1-antibody-ACE2 fusion RiboMab concentration of app. 5 μg/mL. A ratio of 0.8:1 resulted in approximately 2.5 μg/mL and a ratio of 1:1 in approximately 2 μg/mL. In summary, RiboMab_411 and RiboMab_406 were equally expressed (FIG. 14A).

c. Western Blot Analysis of Anti-S1-Antibody-ACE2 Fusion in Producer Cell Culture Supernatant

SN of HEK 293T/17 cells (Example 13a) were used for the analysis of translation and secretion of anti-S1-antibody-ACE2 fusion RiboMabs. SN and reference protein (spiked in Mock SN) were accomplished with water and 4× Laemmli buffer (Bio-Rad Laboratories, Dreieich, Germany) to a final volume of 21.54 and heated to 95° C. for 5 min without (non-reducing, FIG. 14B) or with (reducing, FIG. 14C) 1M Dithiothreitol (DTT, final concentration 0.1M, Carl Roth GmbH & Co. KG, Karlsruhe, Germany). Prepared SN and the corresponding purified reference protein ID 411 were separated by polyacrylamide gel electrophoresis using 4-15% Criterion™ TGX Stain-Free™ Gel (Bio-Rad). As molecular weight standards, Precision Plus Protein™ All Blue Prestained Protein Standard (Bio-Rad) and Novex™ HiMark™ Pre-Stained Protein Standard (Hi-Mark, Invitrogen/Thermo Fisher Scientific) with a molecular weight range of 10-250 kD and 31-460 kD, respectively, were applied. Western Blot analysis (FIG. 14B, C) was performed according to standard procedures known by the person skilled in the art. Nitrocellulose membranes (Bio-Rad) were blocked with a 5% milk solution (Carl Roth GmbH & Co. KG) for one hour. Proteins were detected with a mixture of the polyclonal antibodies Peroxidase AffiniPure Goat Anti-Human IgG, Fcγ fragment specific (dilution 1:2,000; Jackson ImmunoResearch, Cambridge, UK) and Goat anti-Human Kappa Light Chain Cross-Adsorbed Secondary Antibody, HRP (dilution 1:200; Invitrogen/Thermo Fisher Scientific, Darmstadt, Germany) diluted in 3% BSA Fraction V solution (Eurobio Scientific, Les Ulis, France). Membranes were subsequently incubated with a 1:1 mixture of Clarity Western Peroxide Reagent and Clarity Western Luminol/Enhancer Reagent (Bio-Rad) and were recorded with a VILBER Fusion X imaging device (Vilber Lourmat, Eberhardzell, Germany). Data was analyzed with Image Lab Software (Bio-Rad). Signals of RiboMab_411 and RiboMab_406 were detected at approximately 200-460 kD (full antibody) and 100 kD (LC-ACE2) under non-reducing and 100 kD (LC-ACE2) and 50 kD (HC) under reducing conditions, respectively, as compared to the internal molecular weight standards.

In summary, both anti-S1-antibody-ACE2 fusion RiboMabs were efficiently translated from IVT-mRNA and secreted into the SN.

### Example 15: Estimation of Pharmacokinetic Behavior of Anti-S1-Antibody-ACE2 Fusion RiboMabs in Mice

To determine the approximate half-life and clearance of anti-S1-antibody-ACE2 fusion RiboMabs in mice, we used female Balb/cJRj (Janvier Labs, Le Genest-Saint-Isle, France) mice at 11 weeks of age. For injection, an RNA mixture ratio of HC-to-LC-ACE2 of 0.925:1 had been encapsulated in liver-targeting cationic lipid nanoparticles (LNP) (Jayaraman, M. et al. (2012), Angewandte Chemie (International ed. in English) 51 (34), 8529-8533). 30 μg RNA-LNP encoding RiboMab_406 or RiboMab_411 was intravenously injected per mouse. Group sizes comprised 12 mice per RNA-LNP with four mice corresponding to one time point of blood retrieval. Serum of mice bled 14 days before injection served as baseline. Further time points for blood retrieval were set at 6, 24, 48, 96, 240 hours. Blood was directly collected in Microvette 500Z Gel tubes (Sarstedt, Nürmbrecht, Germany) and serum was separated via centrifugation as known by the person skilled in the art. Serum was harvested, immediately flash-frozen in liquid nitrogen and stored at −65 to −85° C. until use.

Anti-S1-antibody-ACE2 fusion RiboMab concentrations in mouse sera were quantified using Gyros xPand™ XPA1025 immunoassay device (Gyros Protein Technologies AB). All materials were from Gyros Protein Technologies AB, if not stated otherwise. A sandwich immunoassay was run using Gyrolab® Generic PK Kit or Gyrolab® Generic TK Kit according to the manufacturer's protocol. The capture antibody (Reagent A, included in the Gyrolab® Generic TK Kit) and the detection antibody anti-Kappa light chain Alexa Fluor® 647 (Abcam, Cambridge, UK) were used with the Gyrolab® Bioaffy 1000 HC CD for protein concentrations in a dynamic range of 0.5-1,000 ng/mL or Gyrolab® Bioaffy 20 HC CD in a dynamic range of 40-80.000 ng/mL, respectively.

Samples were diluted in Reagent F (included in the Gyrolab Generic TK Kit) at least 1:10 by volume. Data was generated with the Gyrolab® Generic PK kit method v1 or Gyrolab® Generic TK kit method v1.

As demonstrated in FIG. 15, maximal anti-S1-antibody-ACE2 fusion RiboMab serum concentrations of 30-40 μg/ml (RiboMab_406 and RiboMab_411, respectively) were reached within six hours post intravenous injection. 5-7 μg/mL (RiboMab_411 and RiboMab_406, respectively) were detected at 96 hours post injection. RiboMab concentrations measured thereafter were below 1 ng/mL.

In summary, both RiboMabs are similarly expressed in vivo and show a similar pharmacokinetic behavior. Higher RiboMab concentrations are expected to be reached with the optimal HC-to-LC-ACE2 ratio of 0.6:1 as described in Example 14b. Of note, a half-time extension by the LS-mutation in the Fc part of RiboMab_406 cannot be analyzed in Balb/cJRj mice. To investigate LS mutation-driven half-time extension, a human neonatal Fc receptor (FcRn)-transgenic mouse strain has to be used.

### Example 16: Expression and Protein Integrity of Anti-S1-Antibody-ACE2 Fusion RiboMabs In Vivo

The integrity of the secreted anti-S1-antibody-ACE2 fusion RiboMab_406 and _411 in Balb/cJRj mouse sera (Example 15) was investigated via Western Blot analysis in principle as described in Example 14c with the difference that the sera were purified via Melon Gel™ (Thermo Fisher Scientific) prior to sample preparation. 10 ng of purified reference protein ID 411 was loaded with and without serum of untreated mice. As negative control, serum from Balb/cJRj mice injected with luciferase-encoding RNA-LNP was used.

Signals of RiboMabs were detected at approximately 200-460 kD (full antibody) and 100 kD (LC-ACE2) under non-reducing (FIG. 16A) conditions and at approximately 100 kD (LC-ACE2) and 50 kD (HC) under reducing conditions (FIG. 16B), respectively, as compared to the internal molecular weight standards. In one mouse (RiboMab_406, mouse #4) we detected bands of the same intensity as the full anti-S1-antibody-ACE2 fusion RiboMab at presumably the weight of the human anti-S1-antibody without ACE2 (^{˜}170 kD) under non-reducing conditions and an additional free LC (25 kD) under reducing conditions, supporting the hypothesis of an IgG-only molecule.

In conclusion, both RiboMabs are comparably expressed in vitro and in vivo, exhibit stable production and are cleared from the murine systemic circulation in a similar fashion.

### Example 17: Pseudovirus Neutralization Activity by RiboMab_411 and 406

To determine the virus neutralizing activity of RiboMab_406 and 411 a pVNT was performed. Supernatant of HEK 293 T/17 cells electroporated with RiboMab_406 or 411 encoding RNAs served as test items and of HEK 293 T/17 cells electroporated with the respective HC only served as Mock control (electroporation described in Example 14a). All SN were 60-fold concentrated with Amicon Ultra-0.5 30 KDa (Merck Millipore, Darmstadt, Germany).

The assay was performed as described in Example 4. 30 IA of diluted pseudovirus was added to the wells containing 304 anti-S1-antibody-ACE2 fusion RiboMab_411 or 406 in SN in a two-fold, eight-point serial dilution ranging from 60 to 0.46 μg/mL (final concentration=30 to 0.23 μg/mL) and Mock SN. Pseudovirus/test dilution mix was added to the seeded Vero-76 cells (MOI: 0.003), followed by incubation for 24 hours at 37° C. and 5% CO₂.

FIG. 17 demonstrates that Mock SN does not significantly affect infection of Vero-76 cells by the pseudovirus whereas anti-S1-antibody-ACE2 fusion RiboMab_411 and 406 inhibit infection in a dose dependent manner with IC50 values ranging from 1.34 to 4.54 μg/mL.

### Example 18: Binding of Bispecific Anti-S1-Antibody-scFv Fusion Proteins to Recombinant SARS-CoV2 S1-RBD Protein

To test the binding of bispecific anti-S1-antibody-scFv fusion proteins to the SARS-CoV2 S1-RBD protein an ELISA was done as described in example 2. As controls the anti-S1 antibody and the antibodies of the invention were used. FIGS. 18A and B shows that the anti-S1-antibody-scFv fusion proteins bind in a dose-dependent manner to the SARS-CoV2 S1-RBD protein and with largely similar EC50 values when compared to the anti-S1 antibody (408) and the antibodies of the invention (444, 446, 449, 450 and 451).

### Example 19: Neutralization of SARS-CoV2-S1-RBD Binding to ACE2 by Anti-S1-Antibody-scFv Fusion Proteins

The potency of anti-S1-antibody-scFv fusion proteins in neutralizing the SARS-CoV2 S1-RBD binding to the ACE-2 extracellular domain was investigated in a competition ELISA as described in example 3. As controls the anti-S1 antibody and the antibodies of the invention were used. FIGS. 19A and B demonstrate that increasing concentrations of the anti-S1-antibody-scFv fusion proteins inhibit the interaction of ACE-2 ECD with the SARS-CoV2 S1-RBD protein. In contrast there is only a slight inhibition observed when using the anti-S1 antibody control. In comparison to the antibodies of the invention (444, 446, 449, 450 and 451), the anti-S1-antibody-scFv fusion proteins show significantly lower IC90 values and therefore a more potent blockage of the ACE-2 S1-RBD interaction.

### Example 20: Binding Affinities of S1 Targeting Antibodies of the Invention

The biochemical affinities of S1 targeting antibodies of the invention were determined by surface plasmon resonance measurements. SARS-CoV-2 S1-RBD Protein coupled to a mouse Fc-tag (Sino Biologicals #40592-V05H) was immobilized to a CM5 sensor chip surface via an anti-mouse-Fc antibody at a density of Rmax ^{˜}10 RU. The kinetics of the interaction of SARS-CoV-2 S1-RBD Protein with S1 targeting antibodies of the invention were analysed on a Biacore T200 SPR instrument. Kinetic data were determined using a Langmuir 1:1 binding model. FIG. 20 shows that the K_{D} values for the antibodies of the invention range from 115 to 1360 pM. The antibody with the Protein sample ID 447 shows a particularly low dissociation rate of 0.672×10⁻⁴ s⁻¹ when compared to the other tested antibodies.

### Example 21: Pseudovirus Neutralization Activity by Anti-S1-Antibody-scFv Fusion Proteins

To determine the potencies of bispecific anti-S1-antibody-scFv fusion antibodies to inhibit S1 protein-directed virus infection of cells, a pseudovirus neutralization test (pVNT) was performed as described in example 11. Bispecific antibodies were tested at concentrations ranging from 30 to 0.01 μg/ml. FIG. 21 shows that the bispecific antibodies 465 and 467 efficiently block S1-mediated pseudovirus infection. IC50 and IC90 values are summarized in FIG. 21B. FIG. 22 shows that potent neutralization is achieved with constructs 478, 480, 481, 482 and 483 with IC90 values ranging from 0.57 to 0.28 μg/ml.

### Example 22: Pseudovirus Neutralization Activity by Anti-S1-Antibody-scFv Fusion Proteins 498, 500, 501 and 502

To determine the potencies of bispecific anti-S1-antibody-scFv fusion antibodies 498, 500, 501 and 502 to inhibit S1 protein-directed virus infection of cells, a pseudovirus neutralization test (pVNT) was performed as described in example 11. Bispecific antibodies were tested at concentrations ranging from 6000 to 0.03 ng/ml. FIG. 23 shows that the bispecific antibodies 498 and 502 provide the most potent neutralization with IC90 values of 29.4 and 51.4 ng/ml, respectively.

### Example 23: Binding Affinities of S1 Targeting Antibody 470 of the Invention

The biochemical affinity of S1 targeting antibody 470 of the invention was determined by surface plasmon resonance measurements. SARS-CoV-2 S1-RBD Protein coupled to a mouse Fc-tag (Sino Biologicals #40592-V05H) was immobilized to a CM5 sensor chip surface via an anti-mouse-Fc antibody at a density of Rmax ^{˜}10 RU. The qualitative and quantitative affinity of the interaction of SARS-CoV-2 S1-RBD Protein with the S1 targeting antibody 470 of the invention was analysed on a Biacore T200 SPR instrument using multicycle kinetics measuring three replicates on two different flow cells. Kinetic data were determined using a Langmuir 1:1 binding model. FIG. 24 shows that the calculated mean K_{D} value from multicycle kinetics for the antibody 470 of the invention is 10.4 pM.

### Example 24: Generation of DNA Templates and IVT-mRNA Encoding Anti-SARS-Cov-2 IgG RiboMabs

a. Cloning of Antibody IVT-mRNA Template Vectors

For the generation of chimeric anti-SARS-Cov-2 IgG RiboMabs via in vitro transcribed messenger RNA (IVT-mRNA), the VH and VL DNA sequences of the rabbit anti-SARS-Cov-2 antibodies ID 443, 445, 447, 451, 470 and 472 (listed in Table 1) were subcloned into the IVT-mRNA template vector pST1-hAg-MCS-FI-A30LA70 (BioNTech SE, Mainz, Germany) using standard techniques. Further details are described in Example 13a. Apart from the rabbit VH and VL domains, all other antibody domains originated from human IgG1. The following constructs were cloned for the formation of anti-SARS-Cov-2 IgG RiboMabs:

RiboMab_443/445/447/451/470/472 of the invention and anti-SARS-Cov-2 IgG reference:
pST1-5′hAg-Sec-VH-CH1-CH2^{(Leu234Ala, Leu235Ala)}-CH3^{(Met434Leu, Asn428Ser)}-FI-A30LA70 (HC)
pST1-5′hAg-Sec-VL-CL-FI-A30LA70 (LC)

5′hAg, 5′UTR from human alpha-globin; A, adenine; Ala, alanine; Asn, asparagine; CL, constant light chain region; CH, constant heavy chain region; FI, 3′UTR sequence; HC, heavy chain; L, linker; Leu, leucine; LC, light chain; Met, methionine; pST1, DNA template vector; Sec, secretion signal; Ser, serine; VH, variable heavy chain domain; VL, variable light chain domain.

b. IVT-mRNA Synthesis

The generation of IVT-mRNA is described under Example 13b. FIG. 25 shows sketches of the two IVT-mRNAs needed (FIG. 25A) for the formation of a complete antibody molecule and the IgG RiboMab itself (FIG. 25B).

### Example 25: Expression and Protein Integrity of Anti-SARS-Cov-2 IgG RiboMabs In Vitro

a. Electroporation of Producer Cells

The production of anti-SARS-CoV-2 IgG RiboMabs from IVT-mRNA was performed as described in Example 14a. The RNA mixtures contained mRNAs encoding HC and LC at mass ratios of 1.5:1, respectively.

b. Immunoassay-Based Quantitation of Anti-SARS-CoV-2 IgG RiboMabs in Producer Cell Culture Supernatant

Anti-SARS-CoV-2 IgG RiboMabs in SN from electroporated HEK 293T/17 cells were in principle quantified as described in Example 14b. Determination of protein concentrations in a dynamic range of 4-3,000 ng/mL was performed with Gyrolab® Bioaffy 1000 HC CD and the Gyrolab huIgG Kit—Low Titer components Reagent A (capture reagent) and Reagent B (detection reagent). Data were generated with the Gyrolab® huIgG Low Titer Kit method v1.

The anti-SARS-CoV-2 IgG RiboMab concentrations ranged from approximately 2 to 8 μg/mL, with RiboMab_443 showing the highest and RiboMab_472 showing the lowest expression yield (FIG. 26).

### Example 26: Pseudovirus Neutralization Activity by In Vitro Expressed Anti-SARS-CoV-2 IgG RiboMabs

To determine the virus neutralizing activity of anti-SARS-CoV-2 IgG RiboMab_443/445/447/451/470 and 472 expressed in vitro by HEK 293/T17 (#CL-11268™, American Type culture collection [ATCC]) via electroporation (Example 25), a pVNT assay was performed. Replication-deficient VSV that lacks the genetic information for the VSV envelope glycoprotein VSV-G but contains an ORF for luciferase protein was used for SARS-CoV-2-S(S=spike protein) pseudovirus generation. Here, only pseudovirus carrying the wild-type spike protein of SARS-CoV-2 (first variant as identified in Wuhan, China) was used. VSV pseudotypes were generated according to a published protocol (PMID: 32142651).

All SN samples were concentrated with Amicon Ultra-0.5 centrifugal filter units with a cut-off of 30 KDa (Merck Millipore, Darmstadt, Germany) to yield approximately similar RiboMab concentrations of 200 to 300 μg/ml in HEK 293/T17 cell culture SN samples.

For the pVNT assay, VERO 76 cells (#CL-1587™, American Type culture collection) were thawed and diluted to 5×10⁵ cells/mL in assay medium (DMEM [Gibco/ThermoFisher Scientific, Darmstadt, Germany]/10% FBS [Merck/Sigma-Aldrich, Darmstadt, Germany]) and seeded in white 384-well flat-bottom plates (Greiner Bio-One GmbH, Frickenhausen, Germany) at a density of 1×10⁴ cells per well in 20 μL assay medium. Cells were incubated for 4 hours at 37° C. and 7.5% CO₂. VSV/SARS-CoV-2 pseudovirus was thawed and diluted to obtain approximately 50 IU per well. Anti-SARS-CoV-2 IgG RiboMab-containing SN was diluted in assay medium in 96-well V-bottom plates (Greiner Bio-One GmbH, Frickenhausen, Germany) in 12-step, 2-fold serial dilutions with a total of 40 μL per dilution. 10 μL of diluted pseudovirus and 10 μL per RiboMab dilution were combined per well of a 384-deep well bottom plate (Greiner Bio-One GmbH, Frickenhausen, Germany) for final IgG RiboMab concentrations ranging from 15 to 7×10³ μg/ml (RiboMab_443) or 30 to 1×10⁻² μg/mL (all other RiboMabs). Pseudovirus/RiboMab mixtures were incubated in triplicates on a microplate shaker at 1,200 rpm for 10 min and without shaking for additional 5 min. Of these pseudovirus/RiboMab dilution mixtures, 10 μL were added to the seeded VERO 76 cells, followed by incubation for 18 hours at 37° C. and 5% CO₂. After the incubation, the cell culture plates were removed from the incubator and equilibrated to room temperature. 30 μL of BioGlo™ luciferine solution (Promega, Germany) was added per well and incubated at room temperature and protected from light for 5 min. Relative luminescence light units (RLU) were measured in a Tecan Infinite M200 Pro microplate reader (Tecan, Männedorf, Switzerland). Luminescence is here inversely indicative for the inhibition of viral infection. Curve fitting was done using Graph Pad Prism software. IC50 and IC90 calculation was done using XLfit add-in (IDBS) for Excel (Microsoft).

FIG. 27A demonstrates that the in vitro expressed anti-SARS-Cov-2 IgG RiboMabs of the invention inhibit infection in a dose dependent manner with IC50 values ranging from 132.4 to 406.1 ng/ml (FIG. 27B). The IgG RiboMab reference (corresponding to ID 408) did not significantly affect infection of VERO 76 cells by the pseudovirus at the tested concentrations. IC90 values ranged from 449.1 to 7,243.2 ng/mL, with RiboMab_470 showing the lowest IC90.

### Example 27: Estimation of Pharmacokinetic Behavior of Anti-SARS-CoV-2 IgG RiboMabs in Mice

a. RNA-LNP Administration into Mice and Serum Preparation

To investigate the pharmacokinetic behavior of IgG RiboMabs in mice, female Balb/cJRj (Janvier Labs, Le Genest-Saint-Isle, France) mice at nine weeks of age were used. For injection, an RNA mixture ratio of HC-to-LC of 1.5:1 had been encapsulated in liver-targeting cationic lipid nanoparticles (LNP) (Jayaraman, M. et al. (2012), Angewandte Chemie (International ed. in English) 51 (34), 8529-8533). Stock solutions of RNA-LNPs (1 mg/mL) were thawed at room temperature and diluted with 1×DPBS (Gibco/ThermoFisher Scientific, Germany) for injections. 30 μg RNA-LNP encoding RiboMab_445, RiboMab_447, RiboMab_470 or RiboMab_472 of the invention was intravenously injected per mouse in a volume of 150 μL. 30 μg RNA-LNP encoding for luciferase served as negative control and 100 μg of the protein ID 408 as IgG protein reference. Group sizes comprised four mice per RNA-LNP. Blood retrieval time points were set at 24, 96, 168, 216, 336 and 504 hours (1, 4, 7, 9, 14 and 21 days). Blood was directly collected in Microvette 500Z Gel tubes (Sarstedt, Nürmbrecht, Germany) and serum was separated via centrifugation as known by trained lab personnel. Serum was harvested, immediately flash-frozen in liquid nitrogen and stored at −65 to −85° C. until use.

b. Immunoassay-Based Quantitation of Anti-SARS-CoV-2 IgG RiboMabs in Mouse Serum

Anti-SARS-CoV-2 IgG RiboMab concentrations in mouse sera were quantified using a Gyros xPand™ XPA1025 immunoassay device (Gyros Protein Technologies AB, Uppsala, Sweden). All materials were from Gyros Protein Technologies AB, if not stated otherwise. A sandwich immunoassay was run using Gyrolab® Generic PK or TK Kit according to the manufacturer's protocol. The capture reagent (Reagent A, included in the Gyrolab® PK or TK Kit) and the detection antibody (Reagent B, included in the Gyrolab® PK or TK Kit) were used with the Gyrolab® Bioaffy 1000 HC CD for protein concentrations in a dynamic range of 1.4-333 ng/mL or Gyrolab® Bioaffy 20 HC CD in a dynamic range of 111-243,000 ng/mL, respectively. Samples were diluted in Reagent F (included in the Gyrolab® PK Kit) at least 1:10 by volume. Data was generated with the Gyrolab® Generic PK or TK Kit method v1.

As demonstrated in FIG. 28, maximal anti-SARS-CoV-2 IgG RiboMab serum concentrations of 1.6 mg/mL (RiboMab_445), 686 μg/mL (RiboMab_447), 741 μg/mL (RiboMab_470) and 627 μg/mL (RiboMab_472) were reached within 24 hours (RiboMab_472) or 96 hours (all other RiboMabs of the invention) post intravenous injection. RiboMabs_445, 447 and 470 could be detected for up to 504 hours, RiboMab_472 for up to 336 hours. The IgG RiboMab reference (based on protein ID 408 including the LALA mutation in CH2) showed a Cmax of 610 μg/mL (24 hours post injection) with a detectability of up to 168 hours. The IgG protein reference (protein ID 408) showed a fast clearance with a detectability for 168 hours. No protein was detected in the luciferase encoding RNA-LNP control group.

In summary, all four tested anti-SARS-CoV-2 IgG RiboMabs of the invention encoded by RNA generated with new antibody sequences (Example 6) are expressed at high titers in vivo. Apart from RiboMab_447, all IgG RiboMabs of the invention showed high titers for at least 21 days.

### Example 28: Pseudovirus Neutralization Activity by In Vivo Expressed Anti-SARS-CoV-2 IgG RiboMabs

To determine the virus neutralizing activity of anti-SARS-CoV-2 IgG RiboMab_445/447/470 and 472 expressed in vivo (Example 27a), a pVNT assay was performed as described in Example 26 with the following deviations: (i) RiboMab-containing mouse serum (without a centrifugal concentration step) from two mice per group harvested 24 hours post RNA-LNP injection was used as test items. 12-step, 3-fold (RiboMab_445/470/472) or 2-fold (RiboMab_447) serial dilutions were applied. Starting concentrations differed for each mouse with a minimum of 30.6 μg/mL (RiboMab_470) to a maximum of 59.8 μg/mL (RiboMab_445 and 472). Accordingly, serial dilutions ranged from approximately 31 to 2×10⁻⁴ μg/ml (RiboMab_470), 60 to 3×10⁻⁴ μg/mL (RiboMab_445 and 472) and 35 to 2×10⁻² μg/mL (RiboMab_447).

FIG. 29A demonstrates that the in vivo expressed anti-SARS-Cov-2 IgG RiboMabs of the invention inhibit infection in a dose dependent manner with IC50 values ranging from approximately 47 to 582 ng/ml (means of two biological replicates, FIG. 29B). The IgG RiboMab reference (sequence corresponding to ID 408) elicits a significantly higher IC50 and no calculable IC90. The IgG reference protein ID 408 did not significantly affect infection of VERO 76 cells by the SARS-CoV-2 wild-type pseudovirus at the tested concentrations. IC90 values ranged from 297 to 5,458 ng/mL (means of two biological replicates), with RiboMab_470 showing the lowest IC90.

### Example 29: Generation of DNA Template Vectors and IVT-mRNA Encoding Anti-SARS-CoV-2 IgG-scFv Bispecific RiboMabs

a. Cloning of Antibody IVT-mRNA Template Vectors

For the generation of chimeric anti-SARS-CoV-2-antibody IgG-scFv bispecific RiboMabs, the VH and VL sequences of RiboMab_443/445 and 470 (Examples 25-28) were selected and combined (shown below and in FIG. 30 as VH^{#1}, VL^{#1} and VH^{#2}, VL^{#2}). The idea of the IgG-scFv molecules is an accelerated neutralization by the simultaneous binding to two epitopes of the SARS-CoV-2 spike protein. To generate the IVT-mRNA encoding the HC and the light chain with a linked single chain variable fragment (IgG-scFv), the VH and VL DNA sequences of the rabbit anti-SARS-CoV-2 antibodies ID 443, 445 and 470 (listed in table 2) were subcloned into the IVT-mRNA template vector pST1-hAg-MCS-FI-A30LA70 (BioNTech RNA Pharmaceuticals, Mainz, Germany) using standard techniques. Further details regarding cloning are described in Example 13a. To link the scFv domains to the LC and to link the VH and VL in the scFv, glycine-serine linkers were used. Apart from the rabbit VH and VL domains, all other antibody domains originated from human IgG1. The following constructs were cloned for the formation of anti-SARS-CoV-2 IgG-scFv bispecific RiboMabs of the invention:

RiboMab_498, 500 and 502:
pST1-5′hAg-Sec-VH^{#1}-CH1-CH2^{(Leu234Ala, Leu235Ala)}-CH3^{(Met434Leu, Asn428Ser)}-FI-A30LA70 (HC)
pST1-5′hAg-Sec-VL^{#1}-CL-GS-VH^{#2}-GS-VL^{#2}-FI-A30LA70 (LC-scFv)

#1, antigen binding sequence of first anti-SARS-CoV-2 antibody; #2, antigen binding sequence of second anti-SARS-CoV-2 antibody; 5′hAg, 5′UTR from human alpha-globin; A, adenine; Ala, alanine; Asn, asparagine; CL, constant light chain region; CH, constant heavy chain region; FI, 3′UTR sequence; GS, glycine-serine linker encoding sequence; HC, heavy chain; L, linker; Leu, leucine; LC, light chain; Met, methionine; pST1, DNA template vector; scFv, single chain variable fragment; Sec, secretion signal; Ser, serine; VH, variable heavy chain domain; VL, variable light chain domain.

The resulting RiboMabs are composed of the VH, VL domains from ID 443 (IgG) and ID 470 (scFv), named RiboMab_498, ID 470 (IgG) and ID 443 (scFv), named RiboMab_500 and ID 445 (IgG) and ID 470 (scFv), named RiboMab_502.

b. IVT-mRNA Synthesis

The generation of IVT-mRNA is described under Example 13b. A sketch of the two IVT-mRNAs needed for the formation of a complete antibody molecule (FIG. 30B) is depicted in FIG. 30A.

### Example 30: Estimation of Pharmacokinetic Behavior of Anti-SARS-CoV-2 IgG-scFv Bispecific RiboMabs in Mice

a. RNA-LNP Administration into Mice and Serum Preparation

To investigate the pharmacokinetic behavior of IgG-scFv bispecific RiboMabs in mice, female Balb/cJRj (Janvier Labs, Le Genest-Saint-Isle, France) mice at 7 weeks of age were used. For injection, an RNA mixture ratio of HC-to-LC-scFv of 0.8:1 had been encapsulated in liver-targeting cationic lipid nanoparticles (LNP) (Jayaraman, M. et al. (2012), Angewandte Chemie (International ed. in English) 51 (34), 8529-8533). Stock solutions of RNA-LNPs (1 mg/mL) were thawed at room temperature and diluted with 1×DPBS (Gibco/ThermoFisher Scientific, Germany) for injections. 30 μg RNA-LNP encoding RiboMab_498, RiboMab_500 and RiboMab_502 of the invention was intravenously injected per mouse in a volume of 100 μL. 30 μg RNA-LNP encoding for luciferase served as negative control and 250 μg of the protein ID 408 as IgG protein reference. Group sizes comprised four mice per RNA-LNP. Blood retrieval time points were set at 6, 24, 48, 96, 168, 336 and 504 hours (0.25, 1, 2, 4, 7, 14 and 21 days). Blood was directly collected in Microvette 500Z Gel tubes (Sarstedt, Nürmbrecht, Germany) and serum was separated via centrifugation as known by the trained lab personnel. Serum was harvested, immediately flash-frozen in liquid nitrogen and stored at −65 to −85° C. until use.

b. Immunoassay-Based Quantitation of Anti-SARS-CoV-2 IgG-scFv Bispecific RiboMabs in Mouse Serum

Anti-SARS-CoV-2 IgG-scFv bispecific RiboMab concentrations in mouse sera were quantified using a Gyros xPand™ XPA1025 immunoassay device (Gyros Protein Technologies AB, Uppsala, Sweden) as described in Example 27b.

As demonstrated in FIG. 31, maximal anti-SARS-CoV-2 IgG-scFv RiboMab serum concentrations of 460 μg/mL (RiboMab_498), 521 μg/mL (RiboMab_500), and 608 μg/mL (RiboMab_502) were reached within 96 hours post intravenous injection. RiboMabs_500 and 502 could be detected for up to 504 hours (21 days), RiboMab_498 for up to 336 hours (14 days). The IgG protein reference (protein ID 408) showed a detectability for 336 hours. No protein was detected in the luciferase encoding RNA-LNP control group.

In summary, all three tested anti-SARS-CoV-2 IgG-scFv bispecific RiboMabs of the invention encoded by RNA generated with new anti-SARS-CoV-2 spike protein antibody sequences (Example 6) are expressed at high titers in vivo in mouse with approximate half-lives of 10 to 11 days.

### Example 31: Pseudovirus Neutralization Activity by In Vivo Expressed Anti-SARS-CoV-2 IgG-scFv Bispecific RiboMabs

To determine the virus neutralizing activity of anti-SARS-CoV-2 IgG-scFv bispecific RiboMab_498/500 and 502 of the invention expressed in vivo, RiboMabs were generated in Balb/cJRj mice under the same conditions as described in Example 30a but with only two mice per test item. Mice were sacrificed 24 hours post intravenous injection to generate maximum serum volumes for a pVNT assay that was performed as described in Example 26 with the following deviations: (i) RiboMab-containing mouse serum (without a concentration step) was used as test items in a 12-step, 4-fold serial dilution with a concentration range of 5 to 1×10⁻⁶ μg/mL. (ii) Besides pseudovirus carrying the wild-type spike protein of SARS-CoV-2 also pseudovirus with the B.1.1.7 (Alpha variant), B.1.315 (Beta variant) and B.1.617 spike proteins were tested. (iii) A BMG microplate reader (BMG LABTECH GmbH, Ortenberg, Germany) was used for the RLU read-out.

FIG. 32A compares the IC50 values of the bispecific IgG-scFv RiboMabs of the invention against the different SARS-CoV-2 variants. RiboMab_500 shows in general the highest neutralizing efficacy against all tested virus spike protein variants with IC50 values between 12.9 to 66.4 ng/mL (FIG. 32B). RiboMabs_498 and 502 show high neutralizing efficacy with low IC50 values towards the wild-type and 8.1.1.7 variants (5.4 to 10.4 ng/mL) but significantly higher IC50 values towards the 8.1.351 (161.7 to 349.4 ng/mL) and B.1.617 (62.9 to 141.4 ng/mL). For all SARS-CoV-2 spike protein variants, IC90 values ranged from 17.4 to 4,885 ng/mL for RiboMab_498, from 63.6 to 591.4 ng/mL for RiboMab_500 and from 55.8 to 2,072 ng/mL for RiboMab_502.

In summary, RiboMab_500 of the invention demonstrates the overall highest virus neutralizing efficacy.

### Example 32: Integrity of In Vivo Expressed Anti-SARS-CoV-2 IgG-scFv Bispecific RiboMabs

Western Blot Analysis of Anti-SARS-Cov-2 IgG-scFv Bispecific RiboMabs in Mouse Serum

Mouse serum samples generated 24 hours post RNA-LNP injection (Example 30) were used for the analysis of anti-SARS-CoV-2 IgG-scFv RiboMabs of the invention. Serum samples were diluted 1:100 in Melon Gel Purification Buffer (Thermo Fisher Scientific, Darmstadt, Germany). Purified ID 500 protein in DPBS (Thermo Fisher Scientific) was used as reference protein. All samples were topped up with DPBS and 4× Laemmli buffer (Bio-Rad Laboratories, Dreieich, Germany) to a final volume of 15 μL and heated to 95° C. for 5 min under non-reducing conditions. The heat-treated samples and the corresponding purified reference protein ID 500 were separated by polyacrylamide gel electrophoresis using 4-15% Criterion™ TGX Stain-Free™ Gel (Bio-Rad). As molecular weight standards, Precision Plus Protein™ Dual Xtra Prestained Protein Standards (Bio-Rad) with a molecular weight range of 10-250 kD was used. Western Blot analysis (FIG. 33A) was performed according to standard procedures by trained lab personnel. Nitrocellulose membranes (Bio-Rad) were blocked with a 5% milk solution (Carl Roth GmbH & Co. KG) for one hour. Protein bands on the blotted membrane were detected using a mixture of Peroxidase-conjugated Goat Anti-Human IgG (H+L) (dilution 1:500; Jackson ImmunoResearch, Cambridge, UK) diluted in 3% BSA Fraction V solution (Eurobio Scientific, Les Ulis, France). Membranes were subsequently incubated with a 1:1 mixture of Clarity Western Peroxide Reagent and Clarity Western Luminol/Enhancer Reagent (Bio-Rad), and imaged on a VILBER Fusion X imaging device (Vilber Lourmat, Eberhardzell, Germany). Data was analyzed with Image Lab Software (Bio-Rad). RiboMab signals were detected at approximately 250 kD (full antibody), as compared to the internal molecular weight standard.

Fractions of monomeric protein, high and low molecular weight (HMW, LMW) species were quantified using ImageLab Software (Bio-Rad, Dreieich, Germany). For a relative protein quantification, areas of single protein bands were defined and containing pixel signals were integrated. As summarized in FIG. 33B, monomeric species were detected with 87.5%, 94.4% and 86.7%, HMW species with 4.7%, 2.6% and 6.6% and LMW with 7.8, 3.0 and 6.7% for RiboMab_498, 500 and 502 respectively. Notably, LMW species bands correspond to the pattern of purified recombinant IgG-scFv protein ID 500 and belong therefor to the normal antibody pattern.

In summary, IgG-scFv bispecific RiboMabs of the invention were correctly assembled and folded to primarily monomeric proteins with minimal formation of HMW species.

1. A binding agent comprising at least a first binding domain binding to a coronavirus spike protein (S protein) and a second binding domain binding to the coronavirus S protein, wherein the first and second binding domains bind to different epitopes of the coronavirus S protein.

2. The binding agent of claim 1, wherein the binding agent is a multispecific such as a bispecific binding agent.

3. The binding agent of claim 1 or 2, wherein the first binding domain comprises a heavy chain variable region (VH).

4. The binding agent of claim 3, wherein the VH comprises a HCDR3 comprising a sequence selected from the group consisting of SEQ ID NO: 4, 12, 20, 28, 36, 44, 52, 60, 68, 76, 84, 92, 100, 108, 116, and 124.

5. The binding agent of claim 3 or 4, wherein the VH comprises a HCDR2 comprising a sequence selected from the group consisting of SEQ ID NO: 3, 11, 19, 27, 35, 43, 51, 59, 67, 75, 83, 91, 99, 107, 115, and 123.

6. The binding agent of any one of claims 3 to 5, wherein the VH comprises a HCDR1 comprising a sequence selected from the group consisting of SEQ ID NO: 2, 10, 18, 26, 34, 42, 50, 58, 66, 74, 82, 90, 98, 106, 114, and 122.

7. The binding agent of any one of claims 3 to 6, wherein the VH is selected from the group consisting of:
(i) a VH comprising a HCDR1 comprising the sequence of SEQ ID NO: 2, a HCDR2 comprising the sequence of SEQ ID NO: 3, and a HCDR3 comprising the sequence of SEQ ID NO: 4;
(ii) a VH comprising a HCDR1 comprising the sequence of SEQ ID NO: 10, a HCDR2 comprising the sequence of SEQ ID NO: 11, and a HCDR3 comprising the sequence of SEQ ID NO: 12;
(iii) a VH comprising a HCDR1 comprising the sequence of SEQ ID NO: 18, a HCDR2 comprising the sequence of SEQ ID NO: 19, and a HCDR3 comprising the sequence of SEQ ID NO: 20;
(iv) a VH comprising a HCDR1 comprising the sequence of SEQ ID NO: 26, a HCDR2 comprising the sequence of SEQ ID NO: 27, and a HCDR3 comprising the sequence of SEQ ID NO: 28;
(v) a VH comprising a HCDR1 comprising the sequence of SEQ ID NO: 34, a HCDR2 comprising the sequence of SEQ ID NO: 35, and a HCDR3 comprising the sequence of SEQ ID NO: 36;
(vi) a VH comprising a HCDR1 comprising the sequence of SEQ ID NO: 42, a HCDR2 comprising the sequence of SEQ ID NO: 43, and a HCDR3 comprising the sequence of SEQ ID NO: 44;
(vii) a VH comprising a HCDR1 comprising the sequence of SEQ ID NO: 50, a HCDR2 comprising the sequence of SEQ ID NO: 51, and a HCDR3 comprising the sequence of SEQ ID NO: 52;
(viii) a VH comprising a HCDR1 comprising the sequence of SEQ ID NO: 58, a HCDR2 comprising the sequence of SEQ ID NO: 59, and a HCDR3 comprising the sequence of SEQ ID NO: 60;
(ix) a VH comprising a HCDR1 comprising the sequence of SEQ ID NO: 66, a HCDR2 comprising the sequence of SEQ ID NO: 67, and a HCDR3 comprising the sequence of SEQ ID NO: 68;
(x) a VH comprising a HCDR1 comprising the sequence of SEQ ID NO: 74, a HCDR2 comprising the sequence of SEQ ID NO: 75, and a HCDR3 comprising the sequence of SEQ ID NO: 76;
(xi) a VH comprising a HCDR1 comprising the sequence of SEQ ID NO: 82, a HCDR2 comprising the sequence of SEQ ID NO: 83, and a HCDR3 comprising the sequence of SEQ ID NO: 84;
(xii) a VH comprising a HCDR1 comprising the sequence of SEQ ID NO: 90, a HCDR2 comprising the sequence of SEQ ID NO: 91, and a HCDR3 comprising the sequence of SEQ ID NO: 92;
(xiii) a VH comprising a HCDR1 comprising the sequence of SEQ ID NO: 98, a HCDR2 comprising the sequence of SEQ ID NO: 99, and a HCDR3 comprising the sequence of SEQ ID NO: 100;
(xiv) a VH comprising a HCDR1 comprising the sequence of SEQ ID NO: 106, a HCDR2 comprising the sequence of SEQ ID NO: 107, and a HCDR3 comprising the sequence of SEQ ID NO: 108;
(xv) a VH comprising a HCDR1 comprising the sequence of SEQ ID NO: 114, a HCDR2 comprising the sequence of SEQ ID NO: 115, and a HCDR3 comprising the sequence of SEQ ID NO: 116; and
(xvi) a VH comprising a HCDR1 comprising the sequence of SEQ ID NO: 122, a HCDR2 comprising the sequence of SEQ ID NO: 123, and a HCDR3 comprising the sequence of SEQ ID NO: 124.

8. The binding agent of any one of claims 1 to 7, wherein the first binding domain comprises a light chain variable region (VL).

9. The binding agent of claim 8, wherein the VL comprises a LCDR3 comprising a sequence selected from the group consisting of SEQ ID NO: 8, 16, 24, 32, 40, 48, 56, 64, 72, 80, 88, 96, 104, 112, 120, and 128.

10. The binding agent of claim 8 or 9, wherein the VL comprises a LCDR2 comprising a sequence selected from the group consisting of SEQ ID NO: 7, 15, 23, 31, 39, 47, 55, 63, 71, 79, 87, 95, 103, 111, 119, and 127.

11. The binding agent of any one of claims 8 to 10, wherein the VL comprises a LCDR1 comprising a sequence selected from the group consisting of SEQ ID NO: 6, 14, 22, 30, 38, 46, 54, 62, 70, 78, 86, 94, 102, 110, 118, and 126.

12. The binding agent of any one of claims 8 to 11, wherein the VL is selected from the group consisting of:
(i) a VL comprising a LCDR1 comprising the sequence of SEQ ID NO: 6, a LCDR2 comprising the sequence of SEQ ID NO: 7, and a LCDR3 comprising the sequence of SEQ ID NO: 8;
(ii) a VL comprising a LCDR1 comprising the sequence of SEQ ID NO: 14, a LCDR2 comprising the sequence of SEQ ID NO: 15, and a LCDR3 comprising the sequence of SEQ ID NO: 16;
(iii) a VL comprising a LCDR1 comprising the sequence of SEQ ID NO: 22, a LCDR2 comprising the sequence of SEQ ID NO: 23, and a LCDR3 comprising the sequence of SEQ ID NO: 24;
(iv) a VL comprising a LCDR1 comprising the sequence of SEQ ID NO: 30, a LCDR2 comprising the sequence of SEQ ID NO: 31, and a LCDR3 comprising the sequence of SEQ ID NO: 32;
(v) a VL comprising a LCDR1 comprising the sequence of SEQ ID NO: 38, a LCDR2 comprising the sequence of SEQ ID NO: 39, and a LCDR3 comprising the sequence of SEQ ID NO: 40;
(vi) a VL comprising a LCDR1 comprising the sequence of SEQ ID NO: 46, a LCDR2 comprising the sequence of SEQ ID NO: 47, and a LCDR3 comprising the sequence of SEQ ID NO: 48;
(vii) a VL comprising a LCDR1 comprising the sequence of SEQ ID NO: 54, a LCDR2 comprising the sequence of SEQ ID NO: 55, and a LCDR3 comprising the sequence of SEQ ID NO: 56;
(viii) a VL comprising a LCDR1 comprising the sequence of SEQ ID NO: 62, a LCDR2 comprising the sequence of SEQ ID NO: 63, and a LCDR3 comprising the sequence of SEQ ID NO: 64;
(ix) a VL comprising a LCDR1 comprising the sequence of SEQ ID NO: 70, a LCDR2 comprising the sequence of SEQ ID NO: 71, and a LCDR3 comprising the sequence of SEQ ID NO: 72;
(x) a VL comprising a LCDR1 comprising the sequence of SEQ ID NO: 78, a LCDR2 comprising the sequence of SEQ ID NO: 79, and a LCDR3 comprising the sequence of SEQ ID NO: 80;
(xi) a VL comprising a LCDR1 comprising the sequence of SEQ ID NO: 86, a LCDR2 comprising the sequence of SEQ ID NO: 87, and a LCDR3 comprising the sequence of SEQ ID NO: 88;
(xii) a VL comprising a LCDR1 comprising the sequence of SEQ ID NO: 94, a LCDR2 comprising the sequence of SEQ ID NO: 95, and a LCDR3 comprising the sequence of SEQ ID NO: 96;
(xiii) a VL comprising a LCDR1 comprising the sequence of SEQ ID NO: 102, a LCDR2 comprising the sequence of SEQ ID NO: 103, and a LCDR3 comprising the sequence of SEQ ID NO: 104;
(xiv) a VL comprising a LCDR1 comprising the sequence of SEQ ID NO: 110, a LCDR2 comprising the sequence of SEQ ID NO: 111, and a LCDR3 comprising the sequence of SEQ ID NO: 112;
(xv) a VL comprising a LCDR1 comprising the sequence of SEQ ID NO: 118, a LCDR2 comprising the sequence of SEQ ID NO: 119, and a LCDR3 comprising the sequence of SEQ ID NO: 120; and
(xvi) a VL comprising a LCDR1 comprising the sequence of SEQ ID NO: 126, a LCDR2 comprising the sequence of SEQ ID NO: 127, and a LCDR3 comprising the sequence of SEQ ID NO: 128.

13. The binding agent of any one of claims 1 to 12, wherein the first binding domain comprises a VH and a VL selected from the group consisting of:
(i) a VH comprising a HCDR1 comprising the sequence of SEQ ID NO: 2, a HCDR2 comprising the sequence of SEQ ID NO: 3, and a HCDR3 comprising the sequence of SEQ ID NO: 4 and a VL comprising a LCDR1 comprising the sequence of SEQ ID NO: 6, a LCDR2 comprising the sequence of SEQ ID NO: 7, and a LCDR3 comprising the sequence of SEQ ID NO: 8;
(ii) a VH comprising a HCDR1 comprising the sequence of SEQ ID NO: 10, a HCDR2 comprising the sequence of SEQ ID NO: 11, and a HCDR3 comprising the sequence of SEQ ID NO: 12 and a VL comprising a LCDR1 comprising the sequence of SEQ ID NO: 14, a LCDR2 comprising the sequence of SEQ ID NO: 15, and a LCDR3 comprising the sequence of SEQ ID NO: 16;
(iii) a VH comprising a HCDR1 comprising the sequence of SEQ ID NO: 18, a HCDR2 comprising the sequence of SEQ ID NO: 19, and a HCDR3 comprising the sequence of SEQ ID NO: 20 and a VL comprising a LCDR1 comprising the sequence of SEQ ID NO: 22, a LCDR2 comprising the sequence of SEQ ID NO: 23, and a LCDR3 comprising the sequence of SEQ ID NO: 24;
(iv) a VH comprising a HCDR1 comprising the sequence of SEQ ID NO: 26, a HCDR2 comprising the sequence of SEQ ID NO: 27, and a HCDR3 comprising the sequence of SEQ ID NO: 28 and a VL comprising a LCDR1 comprising the sequence of SEQ ID NO: 30, a LCDR2 comprising the sequence of SEQ ID NO: 31, and a LCDR3 comprising the sequence of SEQ ID NO: 32;
(v) a VH comprising a HCDR1 comprising the sequence of SEQ ID NO: 34, a HCDR2 comprising the sequence of SEQ ID NO: 35, and a HCDR3 comprising the sequence of SEQ ID NO: 36 and a VL comprising a LCDR1 comprising the sequence of SEQ ID NO: 38, a LCDR2 comprising the sequence of SEQ ID NO: 39, and a LCDR3 comprising the sequence of SEQ ID NO: 40;
(vi) a VH comprising a HCDR1 comprising the sequence of SEQ ID NO: 42, a HCDR2 comprising the sequence of SEQ ID NO: 43, and a HCDR3 comprising the sequence of SEQ ID NO: 44 and a VL comprising a LCDR1 comprising the sequence of SEQ ID NO: 46, a LCDR2 comprising the sequence of SEQ ID NO: 47, and a LCDR3 comprising the sequence of SEQ ID NO: 48;
(vii) a VH comprising a HCDR1 comprising the sequence of SEQ ID NO: 50, a HCDR2 comprising the sequence of SEQ ID NO: 51, and a HCDR3 comprising the sequence of SEQ ID NO: 52 and a VL comprising a LCDR1 comprising the sequence of SEQ ID NO: 54, a LCDR2 comprising the sequence of SEQ ID NO: 55, and a LCDR3 comprising the sequence of SEQ ID NO: 56;
(viii) a VH comprising a HCDR1 comprising the sequence of SEQ ID NO: 58, a HCDR2 comprising the sequence of SEQ ID NO: 59, and a HCDR3 comprising the sequence of SEQ ID NO: 60 and a VL comprising a LCDR1 comprising the sequence of SEQ ID NO: 62, a LCDR2 comprising the sequence of SEQ ID NO: 63, and a LCDR3 comprising the sequence of SEQ ID NO: 64;
(ix) a VH comprising a HCDR1 comprising the sequence of SEQ ID NO: 66, a HCDR2 comprising the sequence of SEQ ID NO: 67, and a HCDR3 comprising the sequence of SEQ ID NO: 68 and a VL comprising a LCDR1 comprising the sequence of SEQ ID NO: 70, a LCDR2 comprising the sequence of SEQ ID NO: 71, and a LCDR3 comprising the sequence of SEQ ID NO: 72;
(x) a VH comprising a HCDR1 comprising the sequence of SEQ ID NO: 74, a HCDR2 comprising the sequence of SEQ ID NO: 75, and a HCDR3 comprising the sequence of SEQ ID NO: 76 and a VL comprising a LCDR1 comprising the sequence of SEQ ID NO: 78, a LCDR2 comprising the sequence of SEQ ID NO: 79, and a LCDR3 comprising the sequence of SEQ ID NO: 80;
(xi) a VH comprising a HCDR1 comprising the sequence of SEQ ID NO: 82, a HCDR2 comprising the sequence of SEQ ID NO: 83, and a HCDR3 comprising the sequence of SEQ ID NO: 84 and a VL comprising a LCDR1 comprising the sequence of SEQ ID NO: 86, a LCDR2 comprising the sequence of SEQ ID NO: 87, and a LCDR3 comprising the sequence of SEQ ID NO: 88;
(xii) a VH comprising a HCDR1 comprising the sequence of SEQ ID NO: 90, a HCDR2 comprising the sequence of SEQ ID NO: 91, and a HCDR3 comprising the sequence of SEQ ID NO: 92 and a VL comprising a LCDR1 comprising the sequence of SEQ ID NO: 94, a LCDR2 comprising the sequence of SEQ ID NO: 95, and a LCDR3 comprising the sequence of SEQ ID NO: 96;
(xiii) a VH comprising a HCDR1 comprising the sequence of SEQ ID NO: 98, a HCDR2 comprising the sequence of SEQ ID NO: 99, and a HCDR3 comprising the sequence of SEQ ID NO: 100 and a VL comprising a LCDR1 comprising the sequence of SEQ ID NO: 102, a LCDR2 comprising the sequence of SEQ ID NO: 103, and a LCDR3 comprising the sequence of SEQ ID NO: 104;
(xiv) a VH comprising a HCDR1 comprising the sequence of SEQ ID NO: 106, a HCDR2 comprising the sequence of SEQ ID NO: 107, and a HCDR3 comprising the sequence of SEQ ID NO: 108 and a VL comprising a LCDR1 comprising the sequence of SEQ ID NO: 110, a LCDR2 comprising the sequence of SEQ ID NO: 111, and a LCDR3 comprising the sequence of SEQ ID NO: 112;
(xv) a VH comprising a HCDR1 comprising the sequence of SEQ ID NO: 114, a HCDR2 comprising the sequence of SEQ ID NO: 115, and a HCDR3 comprising the sequence of SEQ ID NO: 116 and a VL comprising a LCDR1 comprising the sequence of SEQ ID NO: 118, a LCDR2 comprising the sequence of SEQ ID NO: 119, and a LCDR3 comprising the sequence of SEQ ID NO: 120; and
(xvi) a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128.

14. The binding agent of any one of claims 1 to 13, wherein the first binding domain comprises a VH comprising a sequence having at least 70%, at least 75%, at least 80%, at least 85%, at least 90%, at least 95%, at least 97%, at least 99%, or 100% identity to a sequence selected from the group consisting of SEQ ID NO: 1, 9, 17, 25, 33, 41, 49, 57, 65, 73, 81, 89, 97, 105, 113, and 121.

15. The binding agent of any one of claims 1 to 14, wherein the first binding domain comprises a VL comprising a sequence having at least 70%, at least 75%, at least 80%, at least 85%, at least 90%, at least 95%, at least 97%, at least 99%, or 100% identity to a sequence selected from the group consisting of SEQ ID NO: 5, 13, 21, 29, 37, 45, 53, 61, 69, 77, 85, 93, 101, 109, 117, and 125.

16. The binding agent of any one of claims 1 to 15, wherein the first binding domain comprises a VH and a VL selected from the group consisting of:
(i) a VH comprising a sequence having at least 70%, at least 75%, at least 80%, at least 85%, at least 90%, at least 95%, at least 97%, at least 99%, or 100% identity to the sequence of SEQ ID NO: 1 and a VL comprising a sequence having at least 70%, at least 75%, at least 80%, at least 85%, at least 90%, at least 95%, at least 97%, at least 99%, or 100% identity to the sequence of SEQ ID NO: 5;
(ii) a VH comprising a sequence having at least 70%, at least 75%, at least 80%, at least 85%, at least 90%, at least 95%, at least 97%, at least 99%, or 100% identity to the sequence of SEQ ID NO: 9 and a VL comprising a sequence having at least 70%, at least 75%, at least 80%, at least 85%, at least 90%, at least 95%, at least 97%, at least 99%, or 100% identity to the sequence of SEQ ID NO: 13;
(iii) a VH comprising a sequence having at least 70%, at least 75%, at least 80%, at least 85%, at least 90%, at least 95%, at least 97%, at least 99%, or 100% identity to the sequence of SEQ ID NO: 17 and a VL comprising a sequence having at least 70%, at least 75%, at least 80%, at least 85%, at least 90%, at least 95%, at least 97%, at least 99%, or 100% identity to the sequence of SEQ ID NO: 21;
(iv) a VH comprising a sequence having at least 70%, at least 75%, at least 80%, at least 85%, at least 90%, at least 95%, at least 97%, at least 99%, or 100% identity to the sequence of SEQ ID NO: 25 and a VL comprising a sequence having at least 70%, at least 75%, at least 80%, at least 85%, at least 90%, at least 95%, at least 97%, at least 99%, or 100% identity to the sequence of SEQ ID NO: 29;
(v) a VI-1 comprising a sequence having at least 70%, at least 75%, at least 80%, at least 85%, at least 90%, at least 95%, at least 97%, at least 99%, or 100% identity to the sequence of SEQ ID NO: 33 and a VL comprising a sequence having at least 70%, at least 75%, at least 80%, at least 85%, at least 90%, at least 95%, at least 97%, at least 99%, or 100% identity to the sequence of SEQ ID NO: 37;
(vi) a VH comprising a sequence having at least 70%, at least 75%, at least 80%, at least 85%, at least 90%, at least 95%, at least 97%, at least 99%, or 100% identity to the sequence of SEQ ID NO: 41 and a VL comprising a sequence having at least 70%, at least 75%, at least 80%, at least 85%, at least 90%, at least 95%, at least 97%, at least 99%, or 100% identity to the sequence of SEQ ID NO: 45;
(vii) a VH comprising a sequence having at least 70%, at least 75%, at least 80%, at least 85%, at least 90%, at least 95%, at least 97%, at least 99%, or 100% identity to the sequence of SEQ ID NO: 49 and a VL comprising a sequence having at least 70%, at least 75%, at least 80%, at least 85%, at least 90%, at least 95%, at least 97%, at least 99%, or 100% identity to the sequence of SEQ ID NO: 53;
(viii) a VII comprising a sequence having at least 70%, at least 75%, at least 80%, at least 85%, at least 90%, at least 95%, at least 97%, at least 99%, or 100% identity to the sequence of SEQ ID NO: 57 and a VL comprising a sequence having at least 70%, at least 75%, at least 80%, at least 85%, at least 90%, at least 95%, at least 97%, at least 99%, or 100% identity to the sequence of SEQ ID NO: 61;
(ix) a VH comprising a sequence having at least 70%, at least 75%, at least 80%, at least 85%, at least 90%, at least 95%, at least 97%, at least 99%, or 100% identity to the sequence of SEQ ID NO: 65 and a VL comprising a sequence having at least 70%, at least 75%, at least 80%, at least 85%, at least 90%, at least 95%, at least 97%, at least 99%, or 100% identity to the sequence of SEQ ID NO: 69;
(x) a VH comprising a sequence having at least 70%, at least 75%, at least 80%, at least 85%, at least 90%, at least 95%, at least 97%, at least 99%, or 100% identity to the sequence of SEQ ID NO: 73 and a VL comprising a sequence having at least 70%, at least 75%, at least 80%, at least 85%, at least 90%, at least 95%, at least 97%, at least 99%, or 100% identity to the sequence of SEQ ID NO: 77;
(xi) a VH comprising a sequence having at least 70%, at least 75%, at least 80%, at least 85%, at least 90%, at least 95%, at least 97%, at least 99%, or 100% identity to the sequence of SEQ ID NO: 81 and a VL comprising a sequence having at least 70%, at least 75%, at least 80%, at least 85%, at least 90%, at least 95%, at least 97%, at least 99%, or 100% identity to the sequence of SEQ ID NO: 85;
(xii) a VH comprising a sequence having at least 70%, at least 75%, at least 80%, at least 85%, at least 90%, at least 95%, at least 97%, at least 99%, or 100% identity to the sequence of SEQ ID NO: 89 and a VL comprising a sequence having at least 70%, at least 75%, at least 80%, at least 85%, at least 90%, at least 95%, at least 97%, at least 99%, or 100% identity to the sequence of SEQ ID NO: 93;
(xiii) a VH comprising a sequence having at least 70%, at least 75%, at least 80%, at least 85%, at least 90%, at least 95%, at least 97%, at least 99%, or 100% identity to the sequence of SEQ ID NO: 97 and a VL comprising a sequence having at least 70%, at least 75%, at least 80%, at least 85%, at least 90%, at least 95%, at least 97%, at least 99%, or 100% identity to the sequence of SEQ ID NO: 101;
(xiv) a VH comprising a sequence having at least 70%, at least 75%, at least 80%, at least 85%, at least 90%, at least 95%, at least 97%, at least 99%, or 100% identity to the sequence of SEQ ID NO: 105 and a VL comprising a sequence having at least 70%, at least 75%, at least 80%, at least 85%, at least 90%, at least 95%, at least 97%, at least 99%, or 100% identity to the sequence of SEQ ID NO: 109;
(xv) a VH comprising a sequence having at least 70%, at least 75%, at least 80%, at least 85%, at least 90%, at least 95%, at least 97%, at least 99%, or 100% identity to the sequence of SEQ ID NO: 113 and a VL comprising a sequence having at least 70%, at least 75%, at least 80%, at least 85%, at least 90%, at least 95%, at least 97%, at least 99%, or 100% identity to the sequence of SEQ ID NO: 117; and
(xvi) a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125.

17. The binding agent of any one of claims 1 to 16, wherein the second binding domain comprises an extracellular domain (ECD) of ACE2 protein or a variant thereof, or a fragment of the ECD of ACE2 protein or the variant thereof.

18. The binding agent of any one of claims 1 to 17, wherein the variant of the ECD of ACE2 protein or the fragment of the ECD of ACE2 protein or the variant thereof binds to the coronavirus S protein.

19. The binding agent of claim 17 or 18, wherein the second binding domain comprises a sequence having at least 70%, at least 75%, at least 80%, at least 85%, at least 90%, at least 95%, at least 97%, at least 99%, or 100% identity to the sequence of SEQ ID NO: 129.

20. The binding agent of any one of claims 1 to 16, wherein the second binding domain comprises a heavy chain variable region (VH).

21. The binding agent of claim 20, wherein the VH of the second binding domain comprises a HCDR3 comprising a sequence selected from the group consisting of SEQ ID NO: 4, 12, 20, 28, 36, 44, 52, 60, 68, 76, 84, 92, 100, 108, 116, and 124.

22. The binding agent of claim 20 or 21, wherein the VII of the second binding domain comprises a HCDR2 comprising a sequence selected from the group consisting of SEQ ID NO: 3, 11, 19, 27, 35, 43, 51, 59, 67, 75, 83, 91, 99, 107, 115, and 123.

23. The binding agent of any one of claims 20 to 22, wherein the VH of the second binding domain comprises a HCDR1 comprising a sequence selected from the group consisting of SEQ ID NO: 2, 10, 18, 26, 34, 42, 50, 58, 66, 74, 82, 90, 98, 106, 114, and 122.

24. The binding agent of any one of claims 20 to 23, wherein the VH of the second binding domain is selected from the group consisting of:
(i) a VH comprising a HCDR1 comprising the sequence of SEQ ID NO: 2, a HCDR2 comprising the sequence of SEQ ID NO: 3, and a HCDR3 comprising the sequence of SEQ ID NO: 4;
(ii) a VH comprising a HCDR1 comprising the sequence of SEQ ID NO: 10, a HCDR2 comprising the sequence of SEQ ID NO: 11, and a HCDR3 comprising the sequence of SEQ ID NO: 12;
(iii) a VII comprising a HCDR1 comprising the sequence of SEQ ID NO: 18, a HCDR2 comprising the sequence of SEQ ID NO: 19, and a HCDR3 comprising the sequence of SEQ ID NO: 20;
(iv) a VH comprising a HCDR1 comprising the sequence of SEQ ID NO: 26, a HCDR2 comprising the sequence of SEQ ID NO: 27, and a HCDR3 comprising the sequence of SEQ ID NO: 28;
(v) a VII comprising a HCDR1 comprising the sequence of SEQ ID NO: 34, a HCDR2 comprising the sequence of SEQ ID NO: 35, and a HCDR3 comprising the sequence of SEQ ID NO: 36;
(vi) a VH comprising a HCDR1 comprising the sequence of SEQ ID NO: 42, a HCDR2 comprising the sequence of SEQ ID NO: 43, and a HCDR3 comprising the sequence of SEQ ID NO: 44;
(vii) a VH comprising a HCDR1 comprising the sequence of SEQ ID NO: 50, a HCDR2 comprising the sequence of SEQ ID NO: 51, and a HCDR3 comprising the sequence of SEQ ID NO: 52;
(viii) a VH comprising a HCDR1 comprising the sequence of SEQ ID NO: 58, a HCDR2 comprising the sequence of SEQ ID NO: 59, and a HCDR3 comprising the sequence of SEQ ID NO: 60;
(ix) a VH comprising a HCDR1 comprising the sequence of SEQ ID NO: 66, a HCDR2 comprising the sequence of SEQ ID NO: 67, and a HCDR3 comprising the sequence of SEQ ID NO: 68;
(x) a VH comprising a HCDR1 comprising the sequence of SEQ ID NO: 74, a HCDR2 comprising the sequence of SEQ ID NO: 75, and a HCDR3 comprising the sequence of SEQ ID NO: 76;
(xi) a VH comprising a HCDR1 comprising the sequence of SEQ ID NO: 82, a HCDR2 comprising the sequence of SEQ ID NO: 83, and a HCDR3 comprising the sequence of SEQ ID NO: 84;
(xii) a VH comprising a HCDR1 comprising the sequence of SEQ ID NO: 90, a HCDR2 comprising the sequence of SEQ ID NO: 91, and a HCDR3 comprising the sequence of SEQ ID NO: 92;
(xiii) a VH comprising a HCDR1 comprising the sequence of SEQ ID NO: 98, a HCDR2 comprising the sequence of SEQ ID NO: 99, and a HCDR3 comprising the sequence of SEQ ID NO: 100;
(xiv) a VH comprising a HCDR1 comprising the sequence of SEQ ID NO: 106, a HCDR2 comprising the sequence of SEQ ID NO: 107, and a HCDR3 comprising the sequence of SEQ ID NO: 108;
(xv) a VH comprising a HCDR1 comprising the sequence of SEQ ID NO: 114, a HCDR2 comprising the sequence of SEQ ID NO: 115, and a HCDR3 comprising the sequence of SEQ ID NO: 116; and
(xvi) a VH comprising a HCDR1 comprising the sequence of SEQ ID NO: 122, a HCDR2 comprising the sequence of SEQ ID NO: 123, and a HCDR3 comprising the sequence of SEQ ID NO: 124.

25. The binding agent of any one of claims 1 to 16 or 20 to 24, wherein the second binding domain comprises a light chain variable region (VL).

26. The binding agent of claim 25, wherein the VL of the second binding domain comprises a LCDR3 comprising a sequence selected from the group consisting of SEQ ID NO: 8, 16, 24, 32, 40, 48, 56, 64, 72, 80, 88, 96, 104, 112, 120, and 128.

27. The binding agent of claim 25 or 26, wherein the VL of the second binding domain comprises a LCDR2 comprising a sequence selected from the group consisting of SEQ ID NO: 7, 15, 23, 31, 39, 47, 55, 63, 71, 79, 87, 95, 103, 111, 119, and 127.

28. The binding agent of any one of claims 25 to 27, wherein the VL of the second binding domain comprises a LCDR1 comprising a sequence selected from the group consisting of SEQ ID NO: 6, 14, 22, 30, 38, 46, 54, 62, 70, 78, 86, 94, 102, 110, 118, and 126.

29. The binding agent of any one of claims 25 to 28, wherein the VL of the second binding domain is selected from the group consisting of:
(i) a VL comprising a LCDR1 comprising the sequence of SEQ ID NO: 6, a LCDR2 comprising the sequence of SEQ ID NO: 7, and a LCDR3 comprising the sequence of SEQ ID NO: 8;
(ii) a VL comprising a LCDR1 comprising the sequence of SEQ ID NO: 14, a LCDR2 comprising the sequence of SEQ ID NO: 15, and a LCDR3 comprising the sequence of SEQ ID NO: 16;
(iii) a VL comprising a LCDR1 comprising the sequence of SEQ ID NO: 22, a LCDR2 comprising the sequence of SEQ ID NO: 23, and a LCDR3 comprising the sequence of SEQ ID NO: 24;
(iv) a VL comprising a LCDR1 comprising the sequence of SEQ ID NO: 30, a LCDR2 comprising the sequence of SEQ ID NO: 31, and a LCDR3 comprising the sequence of SEQ ID NO: 32;
(v) a VL comprising a LCDR1 comprising the sequence of SEQ ID NO: 38, a LCDR2 comprising the sequence of SEQ ID NO: 39, and a LCDR3 comprising the sequence of SEQ ID NO: 40;
(vi) a VL comprising a LCDR1 comprising the sequence of SEQ ID NO: 46, a LCDR2 comprising the sequence of SEQ ID NO: 47, and a LCDR3 comprising the sequence of SEQ ID NO: 48;
(vii) a VL comprising a LCDR1 comprising the sequence of SEQ ID NO: 54, a LCDR2 comprising the sequence of SEQ ID NO: 55, and a LCDR3 comprising the sequence of SEQ ID NO: 56;
(viii) a VL comprising a LCDR1 comprising the sequence of SEQ ID NO: 62, a LCDR2 comprising the sequence of SEQ ID NO: 63, and a LCDR3 comprising the sequence of SEQ ID NO: 64;
(ix) a VL comprising a LCDR1 comprising the sequence of SEQ ID NO: 70, a LCDR2 comprising the sequence of SEQ ID NO: 71, and a LCDR3 comprising the sequence of SEQ ID NO: 72;
(x) a VL comprising a LCDR1 comprising the sequence of SEQ ID NO: 78, a LCDR2 comprising the sequence of SEQ ID NO: 79, and a LCDR3 comprising the sequence of SEQ ID NO: 80;
(xi) a VL comprising a LCDR1 comprising the sequence of SEQ ID NO: 86, a LCDR2 comprising the sequence of SEQ ID NO: 87, and a LCDR3 comprising the sequence of SEQ ID NO: 88;
(xii) a VL comprising a LCDR1 comprising the sequence of SEQ ID NO: 94, a LCDR2 comprising the sequence of SEQ ID NO: 95, and a LCDR3 comprising the sequence of SEQ ID NO: 96;
(xiii) a VL comprising a LCDR1 comprising the sequence of SEQ ID NO: 102, a LCDR2 comprising the sequence of SEQ ID NO: 103, and a LCDR3 comprising the sequence of SEQ ID NO: 104;
(xiv) a VL comprising a LCDR1 comprising the sequence of SEQ ID NO: 110, a LCDR2 comprising the sequence of SEQ ID NO: 111, and a LCDR3 comprising the sequence of SEQ ID NO: 112;
(xv) a VL comprising a LCDR1 comprising the sequence of SEQ ID NO: 118, a LCDR2 comprising the sequence of SEQ ID NO: 119, and a LCDR3 comprising the sequence of SEQ ID NO: 120; and
(xvi) a VL comprising a LCDR1 comprising the sequence of SEQ ID NO: 126, a LCDR2 comprising the sequence of SEQ ID NO: 127, and a LCDR3 comprising the sequence of SEQ ID NO: 128.

30. The binding agent of any one of claims 1 to 16 or 20 to 29, wherein the second binding domain comprises a VH and a VL selected from the group consisting of:
(i) a VH comprising a HCDR1 comprising the sequence of SEQ ID NO: 2, a HCDR2 comprising the sequence of SEQ ID NO: 3, and a HCDR3 comprising the sequence of SEQ ID NO: 4 and a VL comprising a LCDR1 comprising the sequence of SEQ ID NO: 6, a LCDR2 comprising the sequence of SEQ ID NO: 7, and a LCDR3 comprising the sequence of SEQ ID NO: 8;
(ii) a VH comprising a HCDR1 comprising the sequence of SEQ ID NO: 10, a HCDR2 comprising the sequence of SEQ ID NO: 11, and a HCDR3 comprising the sequence of SEQ ID NO: 12 and a VL comprising a LCDR1 comprising the sequence of SEQ ID NO: 14, a LCDR2 comprising the sequence of SEQ ID NO: 15, and a LCDR3 comprising the sequence of SEQ ID NO: 16;
(iii) a VH comprising a HCDR1 comprising the sequence of SEQ ID NO: 18, a HCDR2 comprising the sequence of SEQ ID NO: 19, and a HCDR3 comprising the sequence of SEQ ID NO: 20 and a VL comprising a LCDR1 comprising the sequence of SEQ ID NO: 22, a LCDR2 comprising the sequence of SEQ ID NO: 23, and a LCDR3 comprising the sequence of SEQ ID NO: 24;
(iv) a VH comprising a HCDR1 comprising the sequence of SEQ ID NO: 26, a HCDR2 comprising the sequence of SEQ ID NO: 27, and a HCDR3 comprising the sequence of SEQ ID NO: 28 and a VL comprising a LCDR1 comprising the sequence of SEQ ID NO: 30, a LCDR2 comprising the sequence of SEQ ID NO: 31, and a LCDR3 comprising the sequence of SEQ ID NO: 32;
(v) a VH comprising a HCDR1 comprising the sequence of SEQ ID NO: 34, a HCDR2 comprising the sequence of SEQ ID NO: 35, and a HCDR3 comprising the sequence of SEQ ID NO: 36 and a VL comprising a LCDR1 comprising the sequence of SEQ ID NO: 38, a LCDR2 comprising the sequence of SEQ ID NO: 39, and a LCDR3 comprising the sequence of SEQ ID NO: 40;
(vi) a VH comprising a HCDR1 comprising the sequence of SEQ ID NO: 42, a HCDR2 comprising the sequence of SEQ ID NO: 43, and a HCDR3 comprising the sequence of SEQ ID NO: 44 and a VL comprising a LCDR1 comprising the sequence of SEQ ID NO: 46, a LCDR2 comprising the sequence of SEQ ID NO: 47, and a LCDR3 comprising the sequence of SEQ ID NO: 48;
(vii) a VH comprising a HCDR1 comprising the sequence of SEQ ID NO: 50, a HCDR2 comprising the sequence of SEQ ID NO: 51, and a HCDR3 comprising the sequence of SEQ ID NO: 52 and a VL comprising a LCDR1 comprising the sequence of SEQ ID NO: 54, a LCDR2 comprising the sequence of SEQ ID NO: 55, and a LCDR3 comprising the sequence of SEQ ID NO: 56;
(viii) a VH comprising a HCDR1 comprising the sequence of SEQ ID NO: 58, a HCDR2 comprising the sequence of SEQ ID NO: 59, and a HCDR3 comprising the sequence of SEQ ID NO: 60 and a VL comprising a LCDR1 comprising the sequence of SEQ ID NO: 62, a LCDR2 comprising the sequence of SEQ ID NO: 63, and a LCDR3 comprising the sequence of SEQ ID NO: 64;
(ix) a VH comprising a HCDR1 comprising the sequence of SEQ ID NO: 66, a HCDR2 comprising the sequence of SEQ ID NO: 67, and a HCDR3 comprising the sequence of SEQ ID NO: 68 and a VL comprising a LCDR1 comprising the sequence of SEQ ID NO: 70, a LCDR2 comprising the sequence of SEQ ID NO: 71, and a LCDR3 comprising the sequence of SEQ ID NO: 72;
(x) a VH comprising a HCDR1 comprising the sequence of SEQ ID NO: 74, a HCDR2 comprising the sequence of SEQ ID NO: 75, and a HCDR3 comprising the sequence of SEQ ID NO: 76 and a VL comprising a LCDR1 comprising the sequence of SEQ ID NO: 78, a LCDR2 comprising the sequence of SEQ ID NO: 79, and a LCDR3 comprising the sequence of SEQ ID NO: 80;
(xi) a VH comprising a HCDR1 comprising the sequence of SEQ ID NO: 82, a HCDR2 comprising the sequence of SEQ ID NO: 83, and a HCDR3 comprising the sequence of SEQ ID NO: 84 and a VL comprising a LCDR1 comprising the sequence of SEQ ID NO: 86, a LCDR2 comprising the sequence of SEQ ID NO: 87, and a LCDR3 comprising the sequence of SEQ ID NO: 88;
(xii) a VH comprising a HCDR1 comprising the sequence of SEQ ID NO: 90, a HCDR2 comprising the sequence of SEQ ID NO: 91, and a HCDR3 comprising the sequence of SEQ ID NO: 92 and a VL comprising a LCDR1 comprising the sequence of SEQ ID NO: 94, a LCDR2 comprising the sequence of SEQ ID NO: 95, and a LCDR3 comprising the sequence of SEQ ID NO: 96;
(xiii) a VH comprising a HCDR1 comprising the sequence of SEQ ID NO: 98, a HCDR2 comprising the sequence of SEQ ID NO: 99, and a HCDR3 comprising the sequence of SEQ ID NO: 100 and a VL comprising a LCDR1 comprising the sequence of SEQ ID NO: 102, a LCDR2 comprising the sequence of SEQ ID NO: 103, and a LCDR3 comprising the sequence of SEQ ID NO: 104;
(xiv) a VH comprising a HCDR1 comprising the sequence of SEQ ID NO: 106, a HCDR2 comprising the sequence of SEQ ID NO: 107, and a HCDR3 comprising the sequence of SEQ ID NO: 108 and a VL comprising a LCDR1 comprising the sequence of SEQ ID NO: 110, a LCDR2 comprising the sequence of SEQ ID NO: 111, and a LCDR3 comprising the sequence of SEQ ID NO: 112;
(xv) a VH comprising a HCDR1 comprising the sequence of SEQ ID NO: 114, a HCDR2 comprising the sequence of SEQ ID NO: 115, and a HCDR3 comprising the sequence of SEQ ID NO: 116 and a VL comprising a LCDR1 comprising the sequence of SEQ ID NO: 118, a LCDR2 comprising the sequence of SEQ ID NO: 119, and a LCDR3 comprising the sequence of SEQ ID NO: 120; and
(xvi) a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128.

31. The binding agent of any one of claims 1 to 16 or 20 to 30, wherein the second binding domain comprises a VH comprising a sequence having at least 70%, at least 75%, at least 80%, at least 85%, at least 90%, at least 95%, at least 97%, at least 99%, or 100% identity to a sequence selected from the group consisting of SEQ ID NO: 1, 9, 17, 25, 33, 41, 49, 57, 65, 73, 81, 89, 97, 105, 113, and 121.

32. The binding agent of any one of claims 1 to 16 or 20 to 31, wherein the second binding domain comprises a VL comprising a sequence having at least 70%, at least 75%, at least 80%, at least 85%, at least 90%, at least 95%, at least 97%, at least 99%, or 100% identity to a sequence selected from the group consisting of SEQ ID NO: 5, 13, 21, 29, 37, 45, 53, 61, 69, 77, 85, 93, 101, 109, 117, and 125.

33. The binding agent of any one of claims 1 to 16 or 20 to 32, wherein the second binding domain comprises a VH and a VL selected from the group consisting of:
(i) a VH comprising a sequence having at least 70%, at least 75%, at least 80%, at least 85%, at least 90%, at least 95%, at least 97%, at least 99%, or 100% identity to the sequence of SEQ ID NO: 1 and a VL comprising a sequence having at least 70%, at least 75%, at least 80%, at least 85%, at least 90%, at least 95%, at least 97%, at least 99%, or 100% identity to the sequence of SEQ ID NO: 5;
(ii) a VH comprising a sequence having at least 70%, at least 75%, at least 80%, at least 85%, at least 90%, at least 95%, at least 97%, at least 99%, or 100% identity to the sequence of SEQ ID NO: 9 and a VL comprising a sequence having at least 70%, at least 75%, at least 80%, at least 85%, at least 90%, at least 95%, at least 97%, at least 99%, or 100% identity to the sequence of SEQ ID NO: 13;
(iii) a VH comprising a sequence having at least 70%, at least 75%, at least 80%, at least 85%, at least 90%, at least 95%, at least 97%, at least 99%, or 100% identity to the sequence of SEQ ID NO: 17 and a VL comprising a sequence having at least 70%, at least 75%, at least 80%, at least 85%, at least 90%, at least 95%, at least 97%, at least 99%, or 100% identity to the sequence of SEQ ID NO: 21;
(iv) a VH comprising a sequence having at least 70%, at least 75%, at least 80%, at least 85%, at least 90%, at least 95%, at least 97%, at least 99%, or 100% identity to the sequence of SEQ ID NO: 25 and a VL comprising a sequence having at least 70%, at least 75%, at least 80%, at least 85%, at least 90%, at least 95%, at least 97%, at least 99%, or 100% identity to the sequence of SEQ ID NO: 29;
(v) a VH comprising a sequence having at least 70%, at least 75%, at least 80%, at least 85%, at least 90%, at least 95%, at least 97%, at least 99%, or 100% identity to the sequence of SEQ ID NO: 33 and a VL comprising a sequence having at least 70%, at least 75%, at least 80%, at least 85%, at least 90%, at least 95%, at least 97%, at least 99%, or 100% identity to the sequence of SEQ ID NO: 37;
(vi) a VH comprising a sequence having at least 70%, at least 75%, at least 80%, at least 85%, at least 90%, at least 95%, at least 97%, at least 99%, or 100% identity to the sequence of SEQ ID NO: 41 and a VL comprising a sequence having at least 70%, at least 75%, at least 80%, at least 85%, at least 90%, at least 95%, at least 97%, at least 99%, or 100% identity to the sequence of SEQ ID NO: 45;
(vii) a VH comprising a sequence having at least 70%, at least 75%, at least 80%, at least 85%, at least 90%, at least 95%, at least 97%, at least 99%, or 100% identity to the sequence of SEQ ID NO: 49 and a VL comprising a sequence having at least 70%, at least 75%, at least 80%, at least 85%, at least 90%, at least 95%, at least 97%, at least 99%, or 100% identity to the sequence of SEQ ID NO: 53;
(viii) a VH comprising a sequence having at least 70%, at least 75%, at least 80%, at least 85%, at least 90%, at least 95%, at least 97%, at least 99%, or 100% identity to the sequence of SEQ ID NO: 57 and a VL comprising a sequence having at least 70%, at least 75%, at least 80%, at least 85%, at least 90%, at least 95%, at least 97%, at least 99%, or 100% identity to the sequence of SEQ ID NO: 61;
(ix) a VH comprising a sequence having at least 70%, at least 75%, at least 80%, at least 85%, at least 90%, at least 95%, at least 97%, at least 99%, or 100% identity to the sequence of SEQ ID NO: 65 and a VL comprising a sequence having at least 70%, at least 75%, at least 80%, at least 85%, at least 90%, at least 95%, at least 97%, at least 99%, or 100% identity to the sequence of SEQ ID NO: 69;
(x) a VH comprising a sequence having at least 70%, at least 75%, at least 80%, at least 85%, at least 90%, at least 95%, at least 97%, at least 99%, or 100% identity to the sequence of SEQ ID NO: 73 and a VL comprising a sequence having at least 70%, at least 75%, at least 80%, at least 85%, at least 90%, at least 95%, at least 97%, at least 99%, or 100% identity to the sequence of SEQ ID NO: 77;
(xi) a VH comprising a sequence having at least 70%, at least 75%, at least 80%, at least 85%, at least 90%, at least 95%, at least 97%, at least 99%, or 100% identity to the sequence of SEQ ID NO: 81 and a VL comprising a sequence having at least 70%, at least 75%, at least 80%, at least 85%, at least 90%, at least 95%, at least 97%, at least 99%, or 100% identity to the sequence of SEQ ID NO: 85;
(xii) a VH comprising a sequence having at least 70%, at least 75%, at least 80%, at least 85%, at least 90%, at least 95%, at least 97%, at least 99%, or 100% identity to the sequence of SEQ ID NO: 89 and a VL comprising a sequence having at least 70%, at least 75%, at least 80%, at least 85%, at least 90%, at least 95%, at least 97%, at least 99%, or 100% identity to the sequence of SEQ ID NO: 93;
(xiii) a VH comprising a sequence having at least 70%, at least 75%, at least 80%, at least 85%, at least 90%, at least 95%, at least 97%, at least 99%, or 100% identity to the sequence of SEQ ID NO: 97 and a VL comprising a sequence having at least 70%, at least 75%, at least 80%, at least 85%, at least 90%, at least 95%, at least 97%, at least 99%, or 100% identity to the sequence of SEQ ID NO: 101;
(xiv) a VH comprising a sequence having at least 70%, at least 75%, at least 80%, at least 85%, at least 90%, at least 95%, at least 97%, at least 99%, or 100% identity to the sequence of SEQ ID NO: 105 and a VI comprising a sequence having at least 70%, at least 75%, at least 80%, at least 85%, at least 90%, at least 95%, at least 97%, at least 99%, or 100% identity to the sequence of SEQ ID NO: 109;
(xv) a VH comprising a sequence having at least 70%, at least 75%, at least 80%, at least 85%, at least 90%, at least 95%, at least 97%, at least 99%, or 100% identity to the sequence of SEQ ID NO: 113 and a VL comprising a sequence having at least 70%, at least 75%, at least 80%, at least 85%, at least 90%, at least 95%, at least 97%, at least 99%, or 100% identity to the sequence of SEQ ID NO: 117; and
(xvi) a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125.

34. The binding agent of any one of claims 1 to 33, wherein:
(i) the first binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128,
and the second binding domain comprises a VH comprising a HCDR1 comprising the sequence of SEQ ID NO: 34, a HCDR2 comprising the sequence of SEQ ID NO: 35, and a HCDR3 comprising the sequence of SEQ ID NO: 36 and a VL comprising a LCDR1 comprising the sequence of SEQ ID NO: 38, a LCDR2 comprising the sequence of SEQ ID NO: 39, and a LCDR3 comprising the sequence of SEQ ID NO: 40;
(ii) the first binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128,
and the second binding domain comprises a VH comprising a HCDR1 comprising the sequence of SEQ ID NO: 2, a HCDR2 comprising the sequence of SEQ ID NO: 3, and a HCDR3 comprising the sequence of SEQ ID NO: 4 and a VL comprising a LCDR1 comprising the sequence of SEQ ID NO: 6, a LCDR2 comprising the sequence of SEQ ID NO: 7, and a LCDR3 comprising the sequence of SEQ ID NO: 8;
(iii) the first binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128,
and the second binding domain comprises a VH comprising a HCDR1 comprising the sequence of SEQ ID NO: 10, a HCDR2 comprising the sequence of SEQ ID NO: 11, and a HCDR3 comprising the sequence of SEQ ID NO: 12 and a VL comprising a LCDR1 comprising the sequence of SEQ ID NO: 14, a LCDR2 comprising the sequence of SEQ ID NO: 15, and a LCDR3 comprising the sequence of SEQ ID NO: 16;
(iv) the first binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128,
and the second binding domain comprises a VH comprising a HCDR1 comprising the sequence of SEQ ID NO: 26, a HCDR2 comprising the sequence of SEQ ID NO: 27, and a HCDR3 comprising the sequence of SEQ ID NO: 28 and a VL comprising a LCDR1 comprising the sequence of SEQ ID NO: 30, a LCDR2 comprising the sequence of SEQ ID NO: 31, and a LCDR3 comprising the sequence of SEQ ID NO: 32;
(v) the first binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128,
and the second binding domain comprises a VH comprising a HCDR1 comprising the sequence of SEQ ID NO: 42, a HCDR2 comprising the sequence of SEQ ID NO: 43, and a HCDR3 comprising the sequence of SEQ ID NO: 44 and a VL comprising a LCDR1 comprising the sequence of SEQ ID NO: 46, a LCDR2 comprising the sequence of SEQ ID NO: 47, and a LCDR3 comprising the sequence of SEQ ID NO: 48;
(vi) the first binding domain comprises a VH comprising a HCDR1 comprising the sequence of SEQ ID NO: 34, a HCDR2 comprising the sequence of SEQ ID NO: 35, and a HCDR3 comprising the sequence of SEQ ID NO: 36 and a VL comprising a LCDR1 comprising the sequence of SEQ ID NO: 38, a LCDR2 comprising the sequence of SEQ ID NO: 39, and a LCDR3 comprising the sequence of SEQ ID NO: 40,
and the second binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128;
(vii) the first binding domain comprises a VH comprising a HCDR1 comprising the sequence of SEQ ID NO: 26, a HCDR2 comprising the sequence of SEQ ID NO: 27, and a HCDR3 comprising the sequence of SEQ ID NO: 28 and a VL comprising a LCDR1 comprising the sequence of SEQ ID NO: 30, a LCDR2 comprising the sequence of SEQ ID NO: 31, and a LCDR3 comprising the sequence of SEQ ID NO: 32,
and the second binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128;
(viii) the first binding domain comprises a VH comprising a HCDR1 comprising the sequence of SEQ ID NO: 42, a HCDR2 comprising the sequence of SEQ ID NO: 43, and a HCDR3 comprising the sequence of SEQ ID NO: 44 and a VI comprising a LCDR1 comprising the sequence of SEQ ID NO: 46, a LCDR2 comprising the sequence of SEQ ID NO: 47, and a LCDR3 comprising the sequence of SEQ ID NO: 48,
and the second binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128;
(ix) the first binding domain comprises a VH comprising a HCDR1 comprising the sequence of SEQ ID NO: 2, a HCDR2 comprising the sequence of SEQ ID NO: 3, and a HCDR3 comprising the sequence of SEQ ID NO: 4 and a VL comprising a LCDR1 comprising the sequence of SEQ ID NO: 6, a LCDR2 comprising the sequence of SEQ ID NO: 7, and a LCDR3 comprising the sequence of SEQ ID NO: 8,
and the second binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128;
(x) the first binding domain comprises a VH comprising a HCDR1 comprising the sequence of SEQ ID NO: 10, a HCDR2 comprising the sequence of SEQ ID NO: 11, and a HCDR3 comprising the sequence of SEQ ID NO: 12 and a VL comprising a LCDR1 comprising the sequence of SEQ ID NO: 14, a LCDR2 comprising the sequence of SEQ ID NO: 15, and a LCDR3 comprising the sequence of SEQ ID NO: 16,
and the second binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128;
(xi) the first binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128,
and the second binding domain comprises a VH comprising a HCDR1 comprising the sequence of SEQ ID NO: 18, a HCDR2 comprising the sequence of SEQ ID NO: 19, and a HCDR3 comprising the sequence of SEQ ID NO: 20 and a VL comprising a LCDR1 comprising the sequence of SEQ ID NO: 22, a LCDR2 comprising the sequence of SEQ ID NO: 23, and a LCDR3 comprising the sequence of SEQ ID NO: 24;
(xii) the first binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128,
and the second binding domain comprises a VH comprising a HCDR1 comprising the sequence of SEQ ID NO: 50, a HCDR2 comprising the sequence of SEQ ID NO: 51, and a HCDR3 comprising the sequence of SEQ ID NO: 52 and a VL comprising a LCDR1 comprising the sequence of SEQ ID NO: 54, a LCDR2 comprising the sequence of SEQ ID NO: 55, and a LCDR3 comprising the sequence of SEQ ID NO: 56;
(xiii) the first binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128,
and the second binding domain comprises a VH comprising a HCDR1 comprising the sequence of SEQ ID NO: 106, a HCDR2 comprising the sequence of SEQ ID NO: 107, and a HCDR3 comprising the sequence of SEQ ID NO: 108 and a VL comprising a LCDR1 comprising the sequence of SEQ ID NO: 110, a LCDR2 comprising the sequence of SEQ ID NO: 111, and a LCDR3 comprising the sequence of SEQ ID NO: 112;
(xiv) the first binding domain comprises a VH comprising a HCDR1 comprising the sequence of SEQ ID NO: 18, a HCDR2 comprising the sequence of SEQ ID NO: 19, and a HCDR3 comprising the sequence of SEQ ID NO: 20 and a VL comprising a LCDR1 comprising the sequence of SEQ ID NO: 22, a LCDR2 comprising the sequence of SEQ ID NO: 23, and a LCDR3 comprising the sequence of SEQ ID NO: 24,
and the second binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128;
(xv) the first binding domain comprises a VH comprising a HCDR1 comprising the sequence of SEQ ID NO: 50, a HCDR2 comprising the sequence of SEQ ID NO: 51, and a HCDR3 comprising the sequence of SEQ ID NO: 52 and a VL comprising a LCDR1 comprising the sequence of SEQ ID NO: 54, a LCDR2 comprising the sequence of SEQ ID NO: 55, and a LCDR3 comprising the sequence of SEQ ID NO: 56,
and the second binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128;
(xvi) the first binding domain comprises a VH comprising a HCDR1 comprising the sequence of SEQ ID NO: 106, a HCDR2 comprising the sequence of SEQ ID NO: 107, and a HCDR3 comprising the sequence of SEQ ID NO: 108 and a VL comprising a LCDR1 comprising the sequence of SEQ ID NO: 110, a LCDR2 comprising the sequence of SEQ ID NO: 111, and a LCDR3 comprising the sequence of SEQ ID NO: 112,
and the second binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128;
(xvii) the first binding domain comprises a VH comprising a HCDR1 comprising the sequence of SEQ ID NO: 106, a HCDR2 comprising the sequence of SEQ ID NO: 107, and a HCDR3 comprising the sequence of SEQ ID NO: 108 and a VL comprising a LCDR1 comprising the sequence of SEQ ID NO: 110, a LCDR2 comprising the sequence of SEQ ID NO: 111, and a LCDR3 comprising the sequence of SEQ ID NO: 112,
and the second binding domain comprises a VH comprising a HCDR1 comprising the sequence of SEQ ID NO: 18, a HCDR2 comprising the sequence of SEQ ID NO: 19, and a HCDR3 comprising the sequence of SEQ ID NO: 20 and a VL comprising a LCDR1 comprising the sequence of SEQ ID NO: 22, a LCDR2 comprising the sequence of SEQ ID NO: 23, and a LCDR3 comprising the sequence of SEQ ID NO: 24;
(xviii) the first binding domain comprises a VH comprising a HCDR1 comprising the sequence of SEQ ID NO: 106, a HCDR2 comprising the sequence of SEQ ID NO: 107, and a HCDR3 comprising the sequence of SEQ ID NO: 108 and a VL comprising a LCDR1 comprising the sequence of SEQ ID NO: 110, a LCDR2 comprising the sequence of SEQ ID NO: 111, and a LCDR3 comprising the sequence of SEQ ID NO: 112,
and the second binding domain comprises a VH comprising a HCDR1 comprising the sequence of SEQ ID NO: 50, a HCDR2 comprising the sequence of SEQ ID NO: 51, and a HCDR3 comprising the sequence of SEQ ID NO: 52 and a VL comprising a LCDR1 comprising the sequence of SEQ ID NO: 54, a LCDR2 comprising the sequence of SEQ ID NO: 55, and a LCDR3 comprising the sequence of SEQ ID NO: 56;
(xix) the first binding domain comprises a VH comprising a HCDR1 comprising the sequence of SEQ ID NO: 106, a HCDR2 comprising the sequence of SEQ ID NO: 107, and a HCDR3 comprising the sequence of SEQ ID NO: 108 and a VL comprising a LCDR1 comprising the sequence of SEQ ID NO: 110, a LCDR2 comprising the sequence of SEQ ID NO: 111, and a LCDR3 comprising the sequence of SEQ ID NO: 112,
and the second binding domain comprises a VH comprising a HCDR1 comprising the sequence of SEQ ID NO: 42, a HCDR2 comprising the sequence of SEQ ID NO: 43, and a HCDR3 comprising the sequence of SEQ ID NO: 44 and a VL comprising a LCDR1 comprising the sequence of SEQ ID NO: 46, a LCDR2 comprising the sequence of SEQ ID NO: 47, and a LCDR3 comprising the sequence of SEQ ID NO: 48;
(xx) the first binding domain comprises a VH comprising a HCDR1 comprising the sequence of SEQ ID NO: 18, a HCDR2 comprising the sequence of SEQ ID NO: 19, and a HCDR3 comprising the sequence of SEQ ID NO: 20 and a VL comprising a LCDR1 comprising the sequence of SEQ ID NO: 22, a LCDR2 comprising the sequence of SEQ ID NO: 23, and a LCDR3 comprising the sequence of SEQ ID NO: 24,
and the second binding domain comprises a VH comprising a HCDR1 comprising the sequence of SEQ ID NO: 106, a HCDR2 comprising the sequence of SEQ ID NO: 107, and a HCDR3 comprising the sequence of SEQ ID NO: 108 and a VL comprising a LCDR1 comprising the sequence of SEQ ID NO: 110, a LCDR2 comprising the sequence of SEQ ID NO: 111, and a LCDR3 comprising the sequence of SEQ ID NO: 112;
(xxi) the first binding domain comprises a VH comprising a HCDR1 comprising the sequence of SEQ ID NO: 50, a HCDR2 comprising the sequence of SEQ ID NO: 51, and a HCDR3 comprising the sequence of SEQ ID NO: 52 and a VL comprising a LCDR1 comprising the sequence of SEQ ID NO: 54, a LCDR2 comprising the sequence of SEQ ID NO: 55, and a LCDR3 comprising the sequence of SEQ ID NO: 56,
and the second binding domain comprises a VH comprising a HCDR1 comprising the sequence of SEQ ID NO: 106, a HCDR2 comprising the sequence of SEQ ID NO: 107, and a HCDR3 comprising the sequence of SEQ ID NO: 108 and a VL comprising a LCDR1 comprising the sequence of SEQ ID NO: 110, a LCDR2 comprising the sequence of SEQ ID NO: 111, and a LCDR3 comprising the sequence of SEQ ID NO: 112;
(xxii) the first binding domain comprises a VH comprising a HCDR1 comprising the sequence of SEQ ID NO: 42, a HCDR2 comprising the sequence of SEQ ID NO: 43, and a HCDR3 comprising the sequence of SEQ ID NO: 44 and a VL comprising a LCDR1 comprising the sequence of SEQ ID NO: 46, a LCDR2 comprising the sequence of SEQ ID NO: 47, and a LCDR3 comprising the sequence of SEQ ID NO: 48,
and the second binding domain comprises a VH comprising a HCDR1 comprising the sequence of SEQ ID NO: 106, a HCDR2 comprising the sequence of SEQ ID NO: 107, and a HCDR3 comprising the sequence of SEQ ID NO: 108 and a VI comprising a LCDR1 comprising the sequence of SEQ ID NO: 110, a LCDR2 comprising the sequence of SEQ ID NO: 111, and a LCDR3 comprising the sequence of SEQ ID NO: 112;
(xxiii) the first binding domain comprises a VH comprising a HCDR1 comprising the sequence of SEQ ID NO: 122, a HCDR2 comprising the sequence of SEQ ID NO: 123, and a HCDR3 comprising the sequence of SEQ ID NO: 124 and a VL comprising a LCDR1 comprising the sequence of SEQ ID NO: 126, a LCDR2 comprising the sequence of SEQ ID NO: 127, and a LCDR3 comprising the sequence of SEQ ID NO: 128,
and the second binding domain comprises a sequence having at least 70%, at least 75%, at least 80%, at least 85%, at least 90%, at least 95%, at least 97%, at least 99%, or 100% identity to the sequence of SEQ ID NO: 129;
(xxiv) the first binding domain comprises a VH comprising a HCDR1 comprising the sequence of SEQ ID NO: 18, a HCDR2 comprising the sequence of SEQ ID NO: 19, and a HCDR3 comprising the sequence of SEQ ID NO: 20 and a VL comprising a LCDR1 comprising the sequence of SEQ ID NO: 22, a LCDR2 comprising the sequence of SEQ ID NO: 23, and a LCDR3 comprising the sequence of SEQ ID NO: 24,
and the second binding domain comprises a VH comprising a HCDR1 comprising the sequence of SEQ ID NO: 82, a HCDR2 comprising the sequence of SEQ ID NO: 83, and a HCDR3 comprising the sequence of SEQ ID NO: 84 and a VL comprising a LCDR1 comprising the sequence of SEQ ID NO: 86, a LCDR2 comprising the sequence of SEQ ID NO: 87, and a LCDR3 comprising the sequence of SEQ ID NO: 88;
(xxv) the first binding domain comprises a VH comprising a HCDR1 comprising the sequence of SEQ ID NO: 82, a HCDR2 comprising the sequence of SEQ ID NO: 83, and a HCDR3 comprising the sequence of SEQ ID NO: 84 and a VL comprising a LCDR1 comprising the sequence of SEQ ID NO: 86, a LCDR2 comprising the sequence of SEQ ID NO: 87, and a LCDR3 comprising the sequence of SEQ ID NO: 88,
and the second binding domain comprises a VH comprising a HCDR1 comprising the sequence of SEQ ID NO: 18, a HCDR2 comprising the sequence of SEQ ID NO: 19, and a HCDR3 comprising the sequence of SEQ ID NO: 20 and a VL comprising a LCDR1 comprising the sequence of SEQ ID NO: 22, a LCDR2 comprising the sequence of SEQ ID NO: 23, and a LCDR3 comprising the sequence of SEQ ID NO: 24;
(xxvi) the first binding domain comprises a VH comprising a HCDR1 comprising the sequence of SEQ ID NO: 82, a HCDR2 comprising the sequence of SEQ ID NO: 83, and a HCDR3 comprising the sequence of SEQ ID NO: 84 and a VL comprising a LCDR1 comprising the sequence of SEQ ID NO: 86, a LCDR2 comprising the sequence of SEQ ID NO: 87, and a LCDR3 comprising the sequence of SEQ ID NO: 88,
and the second binding domain comprises a VH comprising a HCDR1 comprising the sequence of SEQ ID NO: 50, a HCDR2 comprising the sequence of SEQ ID NO: 51, and a HCDR3 comprising the sequence of SEQ ID NO: 52 and a VL comprising a LCDR1 comprising the sequence of SEQ ID NO: 54, a LCDR2 comprising the sequence of SEQ ID NO: 55, and a LCDR3 comprising the sequence of SEQ ID NO: 56; or
(xxvii) the first binding domain comprises a VH comprising a HCDR1 comprising the sequence of SEQ ID NO: 50, a HCDR2 comprising the sequence of SEQ ID NO: 51, and a HCDR3 comprising the sequence of SEQ ID NO: 52 and a VL comprising a LCDR1 comprising the sequence of SEQ ID NO: 54, a LCDR2 comprising the sequence of SEQ ID NO: 55, and a LCDR3 comprising the sequence of SEQ ID NO: 56,
and the second binding domain comprises a VH comprising a HCDR1 comprising the sequence of SEQ ID NO: 82, a HCDR2 comprising the sequence of SEQ ID NO: 83, and a HCDR3 comprising the sequence of SEQ ID NO: 84 and a VL comprising a LCDR1 comprising the sequence of SEQ ID NO: 86, a LCDR2 comprising the sequence of SEQ ID NO: 87, and a LCDR3 comprising the sequence of SEQ ID NO: 88.

35. The binding agent of any one of claims 1 to 34, wherein:
(i) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 33 and a VL comprising a sequence having at least 70%, at least 75%, at least 80%, at least 85%, at least 90%, at least 95%, at least 97%, at least 99%, or 100% identity to the sequence of SEQ ID NO: 37;
(ii) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 1 and a VL comprising a sequence having at least 70%, at least 75%, at least 80%, at least 85%, at least 90%, at least 95%, at least 97%, at least 99%, or 100% identity to the sequence of SEQ ID NO: 5;
(iii) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 9 and a VL comprising a sequence having at least 70%, at least 75%, at least 80%, at least 85%, at least 90%, at least 95%, at least 97%, at least 99%, or 100% identity to the sequence of SEQ ID NO: 13;
(iv) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 25 and a VL comprising a sequence having at least 70%, at least 75%, at least 80%, at least 85%, at least 90%, at least 95%, at least 97%, at least 99%, or 100% identity to the sequence of SEQ ID NO: 29;
(v) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 41 and a VL comprising a sequence having at least 70%, at least 75%, at least 80%, at least 85%, at least 90%, at least 95%, at least 97%, at least 99%, or 100% identity to the sequence of SEQ ID NO: 45;
(vi) the first binding domain comprises, a VH comprising a sequence having at least 70%, at least 75%, at least 80%, at least 85%, at least 90%, at least 95%, at least 97%, at least 99%, or 100% identity to the sequence of SEQ ID NO: 33 and a VL comprising a sequence having at least 70%, at least 75%, at least 80%, at least 85%, at least 90%, at least 95%, at least 97%, at least 99%, or 100% identity to the sequence of SEQ ID NO: 37,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125;
(vii) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 25 and a VL comprising a sequence having at least 70%, at least 75%, at least 80%, at least 85%, at least 90%, at least 95%, at least 97%, at least 99%, or 100% identity to the sequence of SEQ ID NO: 29,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125;
(viii) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 41 and a VL comprising a sequence having at least 70%, at least 75%, at least 80%, at least 85%, at least 90%, at least 95%, at least 97%, at least 99%, or 100% identity to the sequence of SEQ ID NO: 45,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125;
(ix) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 1 and a VL comprising a sequence having at least 70%, at least 75%, at least 80%, at least 85%, at least 90%, at least 95%, at least 97%, at least 99%, or 100% identity to the sequence of SEQ ID NO: 5,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125;
(x) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 9 and a VL comprising a sequence having at least 70%, at least 75%, at least 80%, at least 85%, at least 90%, at least 95%, at least 97%, at least 99%, or 100% identity to the sequence of SEQ ID NO: 13,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125;
(xi) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 17 and a VL comprising a sequence having at least 70%, at least 75%, at least 80%, at least 85%, at least 90%, at least 95%, at least 97%, at least 99%, or 100% identity to the sequence of SEQ ID NO: 21;
(xii) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 49 and a VL comprising a sequence having at least 70%, at least 75%, at least 80%, at least 85%, at least 90%, at least 95%, at least 97%, at least 99%, or 100% identity to the sequence of SEQ ID NO: 53;
(xiii) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 105 and a VL comprising a sequence having at least 70%, at least 75%, at least 80%, at least 85%, at least 90%, at least 95%, at least 97%, at least 99%, or 100% identity to the sequence of SEQ ID NO: 109;
(xiv) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 17 and a VL comprising a sequence having at least 70%, at least 75%, at least 80%, at least 85%, at least 90%, at least 95%, at least 97%, at least 99%, or 100% identity to the sequence of SEQ ID NO: 21,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125;
(xv) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 49 and a VL comprising a sequence having at least 70%, at least 75%, at least 80%, at least 85%, at least 90%, at least 95%, at least 97%, at least 99%, or 100% identity to the sequence of SEQ ID NO: 53,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125;
(xvi) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 105 and a VL comprising a sequence having at least 70%, at least 75%, at least 80%, at least 85%, at least 90%, at least 95%, at least 97%, at least 99%, or 100% identity to the sequence of SEQ ID NO: 109,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125;
(xvii) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 105 and a VL comprising a sequence having at least 70%, at least 75%, at least 80%, at least 85%, at least 90%, at least 95%, at least 97%, at least 99%, or 100% identity to the sequence of SEQ ID NO: 109,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 17 and a VL comprising a sequence having at least 70%, at least 75%, at least 80%, at least 85%, at least 90%, at least 95%, at least 97%, at least 99%, or 100% identity to the sequence of SEQ ID NO: 21;
(xviii) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 105 and a VL comprising a sequence having at least 70%, at least 75%, at least 80%, at least 85%, at least 90%, at least 95%, at least 97%, at least 99%, or 100% identity to the sequence of SEQ ID NO: 109,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 49 and a VL comprising a sequence having at least 70%, at least 75%, at least 80%, at least 85%, at least 90%, at least 95%, at least 97%, at least 99%, or 100% identity to the sequence of SEQ ID NO: 53;
(xix) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 105 and a VL comprising a sequence having at least 70%, at least 75%, at least 80%, at least 85%, at least 90%, at least 95%, at least 97%, at least 99%, or 100% identity to the sequence of SEQ ID NO: 109,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 41 and a VL comprising a sequence having at least 70%, at least 75%, at least 80%, at least 85%, at least 90%, at least 95%, at least 97%, at least 99%, or 100% identity to the sequence of SEQ ID NO: 45;
(xx) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 17 and a VL comprising a sequence having at least 70%, at least 75%, at least 80%, at least 85%, at least 90%, at least 95%, at least 97%, at least 99%, or 100% identity to the sequence of SEQ ID NO: 21,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 105 and a VL comprising a sequence having at least 70%, at least 75%, at least 80%, at least 85%, at least 90%, at least 95%, at least 97%, at least 99%, or 100% identity to the sequence of SEQ ID NO: 109;
(xxi) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 49 and a VL comprising a sequence having at least 70%, at least 75%, at least 80%, at least 85%, at least 90%, at least 95%, at least 97%, at least 99%, or 100% identity to the sequence of SEQ ID NO: 53,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 105 and a VL comprising a sequence having at least 70%, at least 75%, at least 80%, at least 85%, at least 90%, at least 95%, at least 97%, at least 99%, or 100% identity to the sequence of SEQ ID NO: 109;
(xxii) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 41 and a VL comprising a sequence having at least 70%, at least 75%, at least 80%, at least 85%, at least 90%, at least 95%, at least 97%, at least 99%, or 100% identity to the sequence of SEQ ID NO: 45,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 105 and a VL comprising a sequence having at least 70%, at least 75%, at least 80%, at least 85%, at least 90%, at least 95%, at least 97%, at least 99%, or 100% identity to the sequence of SEQ ID NO: 109;
(xxiii) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 121 and a VL comprising a sequence having at least 70%, at least 75%, at least 80%, at least 85%, at least 90%, at least 95%, at least 97%, at least 99%, or 100% identity to the sequence of SEQ ID NO: 125,
and the second binding domain comprises a sequence having at least 70%, at least 75%, at least 80%, at least 85%, at least 90%, at least 95%, at least 97%, at least 99%, or 100% identity to the sequence of SEQ ID NO: 129;
(xxiv) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 17 and a VL comprising a sequence having at least 70%, at least 75%, at least 80%, at least 85%, at least 90%, at least 95%, at least 97%, at least 99%, or 100% identity to the sequence of SEQ ID NO: 21,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 81 and a VL comprising a sequence having at least 70%, at least 75%, at least 80%, at least 85%, at least 90%, at least 95%, at least 97%, at least 99%, or 100% identity to the sequence of SEQ ID NO: 85;
(xxv) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 81 and a VL comprising a sequence having at least 70%, at least 75%, at least 80%, at least 85%, at least 90%, at least 95%, at least 97%, at least 99%, or 100% identity to the sequence of SEQ ID NO: 85,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 17 and a VL comprising a sequence having at least 70%, at least 75%, at least 80%, at least 85%, at least 90%, at least 95%, at least 97%, at least 99%, or 100% identity to the sequence of SEQ ID NO: 21;
(xxvi) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 81 and a VL comprising a sequence having at least 70%, at least 75%, at least 80%, at least 85%, at least 90%, at least 95%, at least 97%, at least 99%, or 100% identity to the sequence of SEQ ID NO: 85,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 49 and a VL comprising a sequence having at least 70%, at least 75%, at least 80%, at least 85%, at least 90%, at least 95%, at least 97%, at least 99%, or 100% identity to the sequence of SEQ ID NO: 53; or
(xxvii) the first binding domain comprises a VH comprising a sequence having at least 70%, at least 75%, at least 80%, at least 85%, at least 90%, at least 95%, at least 97%, at least 99%, or 100% identity to the sequence of SEQ ID NO: 49 and a VL comprising a sequence having at least 70%, at least 75%, at least 80%, at least 85%, at least 90%, at least 95%, at least 97%, at least 99%, or 100% identity to the sequence of SEQ ID NO: 53,
and the second binding domain comprises a VH comprising a sequence having at least 70%, at least 75%, at least 80%, at least 85%, at least 90%, at least 95%, at least 97%, at least 99%, or 100% identity to the sequence of SEQ ID NO: 81 and a VL comprising a sequence having at least 70%, at least 75%, at least 80%, at least 85%, at least 90%, at least 95%, at least 97%, at least 99%, or 100% identity to the sequence of SEQ ID NO: 85.

36. The binding agent of any one of claims 1 to 35, which comprises a heavy chain and a light chain forming the first binding domain.

37. The binding agent of any one of claims 1 to 36, which comprises two heavy chains and two light chains, wherein each of the heavy chains together with one of the light chains forms a first binding domain.

38. The binding agent of claim 36 or 37, wherein the heavy chain comprises a VH.

39. The binding agent of any one of claims 36 to 38, wherein the light chain comprises a VL.

40. The binding agent of any one of claims 36 to 39, wherein the heavy chain comprises a fragment crystallizable (Fc) region.

41. The binding agent of any one of claims 36 to 40, wherein a heavy chain is associated with a light chain.

42. The binding agent of any one of claims 37 to 41, wherein the heavy chains are covalently and/or non-covalently associated.

43. The binding agent of any one of claims 37 to 42, wherein the two heavy chains are identical and the two light chains are identical.

44. The binding agent of any one of claims 36 to 43, which comprises a full-length antibody or a full-length antibody-like molecule comprising first binding domains.

45. The binding agent of any one of claims 1 to 44, which comprises two first binding domains.

46. The binding agent of claim 45, wherein the two first binding domains bind to the same epitope.

47. The binding agent of any one of claims 1 to 16 or 20 to 46, wherein the second binding domain comprises a single-chain variable fragment (scFv).

48. The binding agent of any one of claims 1 to 47, wherein the first and second binding domains are covalently linked, either directly or through a linker.

49. The binding agent of claim 48, wherein the linker is a glycine-serine (GS) linker.

50. The binding agent of any one of claims 1 to 49, which comprises two heavy chains and two light chains forming a full-length antibody or a full-length antibody-like molecule comprising two first binding domains, wherein each of the light chains is linked to a second binding domain.

51. The binding agent of claim 50, wherein the C-terminus of each of the light chains is linked to the N-terminus of a second binding domain.

52. The binding agent of claim 50, wherein the N-terminus of each of the light chains is linked to the C-terminus of a second binding domain.

53. An antibody, comprising a heavy chain variable region (VH), wherein the VH comprises one or more selected from the group consisting of:
(i) a HCDR3 comprising a sequence selected from the group consisting of SEQ ID NO: 4, 12, 20, 28, 36, 44, 52, 60, 68, 76, 84, 92, 100, 108, and 116;
(ii) a HCDR2 comprising a sequence selected from the group consisting of SEQ ID NO: 3, 11, 19, 27, 35, 43, 51, 59, 67, 75, 83, 91, 99, 107, and 115; and
(iii) a HCDR1 comprising a sequence selected from the group consisting of SEQ ID NO: 2, 10, 18, 26, 34, 42, 50, 58, 66, 74, 82, 90, 98, 106, and 114.

54. The antibody of claim 53, wherein the VH is selected from the group consisting of:
(i) a VH comprising a HCDR1 comprising the sequence of SEQ ID NO: 2, a HCDR2 comprising the sequence of SEQ ID NO: 3, and a HCDR3 comprising the sequence of SEQ ID NO: 4;
(ii) a VH comprising a HCDR1 comprising the sequence of SEQ ID NO: 10, a HCDR2 comprising the sequence of SEQ ID NO: 11, and a HCDR3 comprising the sequence of SEQ ID NO: 12;
(iii) a VH comprising a HCDR1 comprising the sequence of SEQ ID NO: 18, a HCDR2 comprising the sequence of SEQ ID NO: 19, and a HCDR3 comprising the sequence of SEQ ID NO: 20;
(iv) a VH comprising a HCDR1 comprising the sequence of SEQ ID NO: 26, a HCDR2 comprising the sequence of SEQ ID NO: 27, and a HCDR3 comprising the sequence of SEQ ID NO: 28;
(v) a VH comprising a HCDR1 comprising the sequence of SEQ ID NO: 34, a HCDR2 comprising the sequence of SEQ ID NO: 35, and a HCDR3 comprising the sequence of SEQ ID NO: 36;
(vi) a VH comprising a HCDR1 comprising the sequence of SEQ ID NO: 42, a HCDR2 comprising the sequence of SEQ ID NO: 43, and a HCDR3 comprising the sequence of SEQ ID NO: 44;
(vii) a VH comprising a HCDR1 comprising the sequence of SEQ ID NO: 50, a HCDR2 comprising the sequence of SEQ ID NO: 51, and a HCDR3 comprising the sequence of SEQ ID NO: 52;
(viii) a VH comprising a HCDR1 comprising the sequence of SEQ ID NO: 58, a HCDR2 comprising the sequence of SEQ ID NO: 59, and a HCDR3 comprising the sequence of SEQ ID NO: 60;
(ix) a VH comprising a HCDR1 comprising the sequence of SEQ ID NO: 66, a HCDR2 comprising the sequence of SEQ ID NO: 67, and a HCDR3 comprising the sequence of SEQ ID NO: 68;
(x) a VH comprising a HCDR1 comprising the sequence of SEQ ID NO: 74, a HCDR2 comprising the sequence of SEQ ID NO: 75, and a HCDR3 comprising the sequence of SEQ ID NO: 76;
(xi) a VH comprising a HCDR1 comprising the sequence of SEQ ID NO: 82, a HCDR2 comprising the sequence of SEQ ID NO: 83, and a HCDR3 comprising the sequence of SEQ ID NO: 84;
(xii) a VH comprising a HCDR1 comprising the sequence of SEQ ID NO: 90, a HCDR2 comprising the sequence of SEQ ID NO: 91, and a HCDR3 comprising the sequence of SEQ ID NO: 92;
(xiii) a VH comprising a HCDR1 comprising the sequence of SEQ ID NO: 98, a HCDR2 comprising the sequence of SEQ ID NO: 99, and a HCDR3 comprising the sequence of SEQ ID NO: 100;
(xiv) a VH comprising a HCDR1 comprising the sequence of SEQ ID NO: 106, a HCDR2 comprising the sequence of SEQ ID NO: 107, and a HCDR3 comprising the sequence of SEQ ID NO: 108; and
(xv) a VH comprising a HCDR1 comprising the sequence of SEQ ID NO: 114, a HCDR2 comprising the sequence of SEQ ID NO: 115, and a HCDR3 comprising the sequence of SEQ ID NO: 116.

55. An antibody, comprising a light chain variable region (VL), wherein the VL comprises one or more selected from the group consisting of:
(i) a LCDR3 comprising a sequence selected from the group consisting of SEQ ID NO: 8, 16, 24, 32, 40, 48, 56, 64, 72, 80, 88, 96, 104, 112, and 120;
(ii) a LCDR2 comprising a sequence selected from the group consisting of SEQ ID NO: 7, 15, 23, 31, 39, 47, 55, 63, 71, 79, 87, 95, 103, 111, and 119; and
(iii) a LCDR1 comprising a sequence selected from the group consisting of SEQ ID NO: 6, 14, 22, 30, 38, 46, 54, 62, 70, 78, 86, 94, 102, 110, and 118.

56. The antibody of claim 55, wherein the VL is selected from the group consisting of:
(i) a VL comprising a LCDR1 comprising the sequence of SEQ ID NO: 6, a LCDR2 comprising the sequence of SEQ ID NO: 7, and a LCDR3 comprising the sequence of SEQ ID NO: 8;
(ii) a VL comprising a LCDR1 comprising the sequence of SEQ ID NO: 14, a LCDR2 comprising the sequence of SEQ ID NO: 15, and a LCDR3 comprising the sequence of SEQ ID NO: 16;
(iii) a VL comprising a LCDR1 comprising the sequence of SEQ ID NO: 22, a LCDR2 comprising the sequence of SEQ ID NO: 23, and a LCDR3 comprising the sequence of SEQ ID NO: 24;
(iv) a VL comprising a LCDR1 comprising the sequence of SEQ ID NO: 30, a LCDR2 comprising the sequence of SEQ ID NO: 31, and a LCDR3 comprising the sequence of SEQ ID NO: 32;
(v) a VL comprising a LCDR1 comprising the sequence of SEQ ID NO: 38, a LCDR2 comprising the sequence of SEQ ID NO: 39, and a LCDR3 comprising the sequence of SEQ ID NO: 40;
(vi) a VL comprising a LCDR1 comprising the sequence of SEQ ID NO: 46, a LCDR2 comprising the sequence of SEQ ID NO: 47, and a LCDR3 comprising the sequence of SEQ ID NO: 48;
(vii) a VL comprising a LCDR1 comprising the sequence of SEQ ID NO: 54, a LCDR2 comprising the sequence of SEQ ID NO: 55, and a LCDR3 comprising the sequence of SEQ ID NO: 56;
(viii) a VL comprising a LCDR1 comprising the sequence of SEQ ID NO: 62, a LCDR2 comprising the sequence of SEQ ID NO: 63, and a LCDR3 comprising the sequence of SEQ ID NO: 64;
(ix) a VL comprising a LCDR1 comprising the sequence of SEQ ID NO: 70, a LCDR2 comprising the sequence of SEQ ID NO: 71, and a LCDR3 comprising the sequence of SEQ ID NO: 72;
(x) a VL comprising a LCDR1 comprising the sequence of SEQ ID NO: 78, a LCDR2 comprising the sequence of SEQ ID NO: 79, and a LCDR3 comprising the sequence of SEQ ID NO: 80;
(xi) a VL comprising a LCDR1 comprising the sequence of SEQ ID NO: 86, a LCDR2 comprising the sequence of SEQ ID NO: 87, and a LCDR3 comprising the sequence of SEQ ID NO: 88;
(xii) a VL comprising a LCDR1 comprising the sequence of SEQ ID NO: 94, a LCDR2 comprising the sequence of SEQ ID NO: 95, and a LCDR3 comprising the sequence of SEQ ID NO: 96;
(xiii) a VL comprising a LCDR1 comprising the sequence of SEQ ID NO: 102, a LCDR2 comprising the sequence of SEQ ID NO: 103, and a LCDR3 comprising the sequence of SEQ ID NO: 104;
(xiv) a VL comprising a LCDR1 comprising the sequence of SEQ ID NO: 110, a LCDR2 comprising the sequence of SEQ ID NO: 111, and a LCDR3 comprising the sequence of SEQ ID NO: 112; and
(xv) a VL comprising a LCDR1 comprising the sequence of SEQ ID NO: 118, a LCDR2 comprising the sequence of SEQ ID NO: 119, and a LCDR3 comprising the sequence of SEQ ID NO: 120.

57. The antibody of any one of claims 53 to 56, wherein the antibody comprises a VH and a VL selected from the group consisting of:
(i) a VH comprising a HCDR1 comprising the sequence of SEQ ID NO: 2, a HCDR2 comprising the sequence of SEQ ID NO: 3, and a HCDR3 comprising the sequence of SEQ ID NO: 4 and a VL comprising a LCDR1 comprising the sequence of SEQ ID NO: 6, a LCDR2 comprising the sequence of SEQ ID NO: 7, and a LCDR3 comprising the sequence of SEQ ID NO: 8;
(ii) a VII comprising a HCDR1 comprising the sequence of SEQ ID NO: 10, a HCDR2 comprising the sequence of SEQ ID NO: 11, and a HCDR3 comprising the sequence of SEQ ID NO: 12 and a VL comprising a LCDR1 comprising the sequence of SEQ ID NO: 14, a LCDR2 comprising the sequence of SEQ ID NO: 15, and a LCDR3 comprising the sequence of SEQ ID NO: 16;
(iii) a VH comprising a HCDR1 comprising the sequence of SEQ ID NO: 18, a HCDR2 comprising the sequence of SEQ ID NO: 19, and a HCDR3 comprising the sequence of SEQ ID NO: 20 and a VL comprising a LCDR1 comprising the sequence of SEQ ID NO: 22, a LCDR2 comprising the sequence of SEQ ID NO: 23, and a LCDR3 comprising the sequence of SEQ ID NO: 24;
(iv) a VH comprising a HCDR1 comprising the sequence of SEQ ID NO: 26, a HCDR2 comprising the sequence of SEQ ID NO: 27, and a HCDR3 comprising the sequence of SEQ ID NO: 28 and a VL comprising a LCDR1 comprising the sequence of SEQ ID NO: 30, a LCDR2 comprising the sequence of SEQ ID NO: 31, and a LCDR3 comprising the sequence of SEQ ID NO: 32;
(v) a VH comprising a HCDR1 comprising the sequence of SEQ ID NO: 34, a HCDR2 comprising the sequence of SEQ ID NO: 35, and a HCDR3 comprising the sequence of SEQ ID NO: 36 and a VL comprising a LCDR1 comprising the sequence of SEQ ID NO: 38, a LCDR2 comprising the sequence of SEQ ID NO: 39, and a LCDR3 comprising the sequence of SEQ ID NO: 40;
(vi) a VH comprising a HCDR1 comprising the sequence of SEQ ID NO: 42, a HCDR2 comprising the sequence of SEQ ID NO: 43, and a HCDR3 comprising the sequence of SEQ ID NO: 44 and a VL comprising a LCDR1 comprising the sequence of SEQ ID NO: 46, a LCDR2 comprising the sequence of SEQ ID NO: 47, and a LCDR3 comprising the sequence of SEQ ID NO: 48;
(vii) a VH comprising a HCDR1 comprising the sequence of SEQ ID NO: 50, a HCDR2 comprising the sequence of SEQ ID NO: 51, and a HCDR3 comprising the sequence of SEQ ID NO: 52 and a VL comprising a LCDR1 comprising the sequence of SEQ ID NO: 54, a LCDR2 comprising the sequence of SEQ ID NO: 55, and a LCDR3 comprising the sequence of SEQ ID NO: 56;
(viii) a VH comprising a HCDR1 comprising the sequence of SEQ ID NO: 58, a HCDR2 comprising the sequence of SEQ ID NO: 59, and a HCDR3 comprising the sequence of SEQ ID NO: 60 and a VL comprising a LCDR1 comprising the sequence of SEQ ID NO: 62, a LCDR2 comprising the sequence of SEQ ID NO: 63, and a LCDR3 comprising the sequence of SEQ ID NO: 64;
(ix) a VH comprising a HCDR1 comprising the sequence of SEQ ID NO: 66, a HCDR2 comprising the sequence of SEQ ID NO: 67, and a HCDR3 comprising the sequence of SEQ ID NO: 68 and a VL comprising a LCDR1 comprising the sequence of SEQ ID NO: 70, a LCDR2 comprising the sequence of SEQ ID NO: 71, and a LCDR3 comprising the sequence of SEQ ID NO: 72;
(x) a VH comprising a HCDR1 comprising the sequence of SEQ ID NO: 74, a HCDR2 comprising the sequence of SEQ ID NO: 75, and a HCDR3 comprising the sequence of SEQ ID NO: 76 and a VL comprising a LCDR1 comprising the sequence of SEQ ID NO: 78, a LCDR2 comprising the sequence of SEQ ID NO: 79, and a LCDR3 comprising the sequence of SEQ ID NO: 80;
(xi) a VH comprising a HCDR1 comprising the sequence of SEQ ID NO: 82, a HCDR2 comprising the sequence of SEQ ID NO: 83, and a HCDR3 comprising the sequence of SEQ ID NO: 84 and a VL comprising a LCDR1 comprising the sequence of SEQ ID NO: 86, a LCDR2 comprising the sequence of SEQ ID NO: 87, and a LCDR3 comprising the sequence of SEQ ID NO: 88;
(xii) a VH comprising a HCDR1 comprising the sequence of SEQ ID NO: 90, a HCDR2 comprising the sequence of SEQ ID NO: 91, and a HCDR3 comprising the sequence of SEQ ID NO: 92 and a VL comprising a LCDR1 comprising the sequence of SEQ ID NO: 94, a LCDR2 comprising the sequence of SEQ ID NO: 95, and a LCDR3 comprising the sequence of SEQ ID NO: 96;
(xiii) a VH comprising a HCDR1 comprising the sequence of SEQ ID NO: 98, a HCDR2 comprising the sequence of SEQ ID NO: 99, and a HCDR3 comprising the sequence of SEQ ID NO: 100 and a VI comprising a LCDR1 comprising the sequence of SEQ ID NO: 102, a LCDR2 comprising the sequence of SEQ ID NO: 103, and a LCDR3 comprising the sequence of SEQ ID NO: 104;
(xiv) a VH comprising a HCDR1 comprising the sequence of SEQ ID NO: 106, a HCDR2 comprising the sequence of SEQ ID NO: 107, and a HCDR3 comprising the sequence of SEQ ID NO: 108 and a VL comprising a LCDR1 comprising the sequence of SEQ ID NO: 110, a LCDR2 comprising the sequence of SEQ ID NO: 111, and a LCDR3 comprising the sequence of SEQ ID NO: 112; and
(xv) a VH comprising a HCDR1 comprising the sequence of SEQ ID NO: 114, a HCDR2 comprising the sequence of SEQ ID NO: 115, and a HCDR3 comprising the sequence of SEQ ID NO: 116 and a VL comprising a LCDR1 comprising the sequence of SEQ ID NO: 118, a LCDR2 comprising the sequence of SEQ ID NO: 119, and a LCDR3 comprising the sequence of SEQ ID NO: 120.

58. The antibody of any one of claims 53 to 57, wherein the antibody comprises a VH comprising a sequence having at least 70%, at least 75%, at least 80%, at least 85%, at least 90%, at least 95%, at least 97%, at least 99%, or 100% identity to a sequence selected from the group consisting of SEQ ID NO: 1, 9, 17, 25, 33, 41, 49, 57, 65, 73, 81, 89, 97, 105, and 113.

59. The antibody of any one of claims 53 to 58, wherein the antibody comprises a VL comprising a sequence having at least 70%, at least 75%, at least 80%, at least 85%, at least 90%, at least 95%, at least 97%, at least 99%, or 100% identity to a sequence selected from the group consisting of SEQ ID NO: 5, 13, 21, 29, 37, 45, 53, 61, 69, 77, 85, 93, 101, 109, and 117.

60. The antibody of any one of claims 53 to 59, wherein the antibody comprises a VH and a VL selected from the group consisting of:
(i) a VH comprising a sequence having at least 70%, at least 75%, at least 80%, at least 85%, at least 90%, at least 95%, at least 97%, at least 99%, or 100% identity to the sequence of SEQ ID NO: 1 and a VL comprising a sequence having at least 70%, at least 75%, at least 80%, at least 85%, at least 90%, at least 95%, at least 97%, at least 99%, or 100% identity to the sequence of SEQ ID NO: 5;
(ii) a VH comprising a sequence having at least 70%, at least 75%, at least 80%, at least 85%, at least 90%, at least 95%, at least 97%, at least 99%, or 100% identity to the sequence of SEQ ID NO: 9 and a VL comprising a sequence having at least 70%, at least 75%, at least 80%, at least 85%, at least 90%, at least 95%, at least 97%, at least 99%, or 100% identity to the sequence of SEQ ID NO: 13;
(iii) a VH comprising a sequence having at least 70%, at least 75%, at least 80%, at least 85%, at least 90%, at least 95%, at least 97%, at least 99%, or 100% identity to the sequence of SEQ ID NO: 17 and a VL comprising a sequence having at least 70%, at least 75%, at least 80%, at least 85%, at least 90%, at least 95%, at least 97%, at least 99%, or 100% identity to the sequence of SEQ ID NO: 21;
(iv) a VH comprising a sequence having at least 70%, at least 75%, at least 80%, at least 85%, at least 90%, at least 95%, at least 97%, at least 99%, or 100% identity to the sequence of SEQ ID NO: 25 and a VL comprising a sequence having at least 70%, at least 75%, at least 80%, at least 85%, at least 90%, at least 95%, at least 97%, at least 99%, or 100% identity to the sequence of SEQ ID NO: 29;
(v) a VH comprising a sequence having at least 70%, at least 75%, at least 80%, at least 85%, at least 90%, at least 95%, at least 97%, at least 99%, or 100% identity to the sequence of SEQ ID NO: 33 and a VL comprising a sequence having at least 70%, at least 75%, at least 80%, at least 85%, at least 90%, at least 95%, at least 97%, at least 99%, or 100% identity to the sequence of SEQ ID NO: 37;
(vi) a VH comprising a sequence having at least 70%, at least 75%, at least 80%, at least 85%, at least 90%, at least 95%, at least 97%, at least 99%, or 100% identity to the sequence of SEQ ID NO: 41 and a VL comprising a sequence having at least 70%, at least 75%, at least 80%, at least 85%, at least 90%, at least 95%, at least 97%, at least 99%, or 100% identity to the sequence of SEQ ID NO: 45;
(vii) a VH comprising a sequence having at least 70%, at least 75%, at least 80%, at least 85%, at least 90%, at least 95%, at least 97%, at least 99%, or 100% identity to the sequence of SEQ ID NO: 49 and a VL comprising a sequence having at least 70%, at least 75%, at least 80%, at least 85%, at least 90%, at least 95%, at least 97%, at least 99%, or 100% identity to the sequence of SEQ ID NO: 53;
(viii) a VH comprising a sequence having at least 70%, at least 75%, at least 80%, at least 85%, at least 90%, at least 95%, at least 97%, at least 99%, or 100% identity to the sequence of SEQ ID NO: 57 and a VL comprising a sequence having at least 70%, at least 75%, at least 80%, at least 85%, at least 90%, at least 95%, at least 97%, at least 99%, or 100% identity to the sequence of SEQ ID NO: 61;
(ix) a VH comprising a sequence having at least 70%, at least 75%, at least 80%, at least 85%, at least 90%, at least 95%, at least 97%, at least 99%, or 100% identity to the sequence of SEQ ID NO: 65 and a VL comprising a sequence having at least 70%, at least 75%, at least 80%, at least 85%, at least 90%, at least 95%, at least 97%, at least 99%, or 100% identity to the sequence of SEQ ID NO: 69;
(x) a VH comprising a sequence having at least 70%, at least 75%, at least 80%, at least 85%, at least 90%, at least 95%, at least 97%, at least 99%, or 100% identity to the sequence of SEQ ID NO: 73 and a VL comprising a sequence having at least 70%, at least 75%, at least 80%, at least 85%, at least 90%, at least 95%, at least 97%, at least 99%, or 100% identity to the sequence of SEQ ID NO: 77;
(xi) a VH comprising a sequence having at least 70%, at least 75%, at least 80%, at least 85%, at least 90%, at least 95%, at least 97%, at least 99%, or 100% identity to the sequence of SEQ ID NO: 81 and a VL comprising a sequence having at least 70%, at least 75%, at least 80%, at least 85%, at least 90%, at least 95%, at least 97%, at least 99%, or 100% identity to the sequence of SEQ ID NO: 85;
(xii) a VH comprising a sequence having at least 70%, at least 75%, at least 80%, at least 85%, at least 90%, at least 95%, at least 97%, at least 99%, or 100% identity to the sequence of SEQ ID NO: 89 and a VL comprising a sequence having at least 70%, at least 75%, at least 80%, at least 85%, at least 90%, at least 95%, at least 97%, at least 99%, or 100% identity to the sequence of SEQ ID NO: 93;
(xiii) a VH comprising a sequence having at least 70%, at least 75%, at least 80%, at least 85%, at least 90%, at least 95%, at least 97%, at least 99%, or 100% identity to the sequence of SEQ ID NO: 97 and a VL comprising a sequence having at least 70%, at least 75%, at least 80%, at least 85%, at least 90%, at least 95%, at least 97%, at least 99%, or 100% identity to the sequence of SEQ ID NO: 101;
(xiv) a VH comprising a sequence having at least 70%, at least 75%, at least 80%, at least 85%, at least 90%, at least 95%, at least 97%, at least 99%, or 100% identity to the sequence of SEQ ID NO: 105 and a VL comprising a sequence having at least 70%, at least 75%, at least 80%, at least 85%, at least 90%, at least 95%, at least 97%, at least 99%, or 100% identity to the sequence of SEQ ID NO: 109; and
(xv) a VH comprising a sequence having at least 70%, at least 75%, at least 80%, at least 85%, at least 90%, at least 95%, at least 97%, at least 99%, or 100% identity to the sequence of SEQ ID NO: 113 and a VL comprising a sequence having at least 70%, at least 75%, at least 80%, at least 85%, at least 90%, at least 95%, at least 97%, at least 99%, or 100% identity to the sequence of SEQ ID NO: 117.

61. A recombinant nucleic acid which encodes a binding agent of any one of claims 1 to 52 or an antibody of any one of claims 53 to 60.

62. The recombinant nucleic acid of claim 61 which is RNA.

63. A cell transfected with a recombinant nucleic acid of claim 61 or 62.

64. The cell of claim 63, wherein the cell expresses the binding agent or the antibody.

65. A pharmaceutical composition comprising a binding agent of any one of claims 1 to 52, an antibody of any one of claims 53 to 60, or a recombinant nucleic acid of claim 61 or 62.

66. The binding agent of any one of claims 1 to 52, the antibody of any one of claims 53 to 60, or the recombinant nucleic acid of claim 61 or 62 for therapeutic use.

67. The binding agent, the antibody, or the recombinant nucleic acid of claim 66, wherein the therapeutic use comprises a therapeutic or prophylactic treatment of a coronavirus infection in a subject.

68. The binding agent, the antibody, or the recombinant nucleic acid of claim 66 or 67, wherein the therapeutic use comprises neutralizing coronavirus in a subject.

69. The binding agent, the antibody, or the recombinant nucleic acid of claim 67 or 68, wherein the subject is human.

70. The binding agent of any one of claims 1 to 52, and 66 to 69, the antibody of any one of claims 53 to 60, and 66 to 69, the recombinant nucleic acid of any one of claims 61, 62, and 66 to 69, the cell of claim 63 or 64, or the pharmaceutical composition of claim 65, wherein the coronavirus is a betacoronavirus.

71. The binding agent of any one of claims 1 to 52, and 66 to 70, the antibody of any one of claims 53 to 60, and 66 to 70, the recombinant nucleic acid of any one of claims 61, 62, and 66 to 70, the cell of any one of claims 63, 64, and 70, or the pharmaceutical composition of claim 65 or 70, wherein the coronavirus is a sarbecovirus.

72. The binding agent of any one of claims 1 to 52, and 66 to 71, the antibody of any one of claims 53 to 60, and 66 to 71, the recombinant nucleic acid of any one of claims 61, 62, and 66 to 71, the cell of any one of claims 63, 64, 70, and 71, or the pharmaceutical composition of any one of claims 65, 70, and 71, wherein the coronavirus is SARS-CoV-1 and/or SARS-CoV-2.

73. A method of treating or preventing a coronavirus infection comprising administering to a subject the binding agent of any one of claims 1 to 52, the antibody of any one of claims 53 to 60, the recombinant nucleic acid of claim 61 or 62, or the pharmaceutical composition of claim 65.
